SEQUENCE LISTING (7 sequences)

SEQ ID NO: 1 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 2 - protein, 153 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFI

SEQ ID NO: 3 - DNA, 22 bp, synthetic construct
  source 1..22: mol_type = other DNA
accttgcttaatgggtgtagat

SEQ ID NO: 4 - DNA, 20 bp, synthetic construct
  source 1..20: mol_type = other DNA
ctgcaaaaatatcccccggc

SEQ ID NO: 5 - DNA, 22 bp, synthetic construct
  source 1..22: mol_type = other DNA
cagagacaagaggatggctagg

SEQ ID NO: 6 - DNA, 20 bp, synthetic construct
  source 1..20: mol_type = other DNA
ttccgggagcccatcatctg

SEQ ID NO: 7 - DNA, 160324 bp, synthetic construct
  source 1..160324: mol_type = other DNA
  misc_difference 712: note = a, c, t, g, unknown or other
  misc_difference 741: note = a, c, t, g, unknown or other
  misc_difference 807: note = a, c, t, g, unknown or other
  misc_difference 847: note = a, c, t, g, unknown or other
  misc_difference 883: note = a, c, t, g, unknown or other
  misc_difference 133488: note = a, c, t, g, unknown or other
  misc_difference 133524: note = a, c, t, g, unknown or other
  misc_difference 133564: note = a, c, t, g, unknown or other
  misc_difference 133630: note = a, c, t, g, unknown or other
  misc_difference 133659: note = a, c, t, g, unknown or other
agcccgggccccccgcgggcggggcggcgcgcaaaaaaggcgggcggcggtccgggcggcgtgcgcgcgcgcggcgggcgttggggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggaaaacgggccccccccgaaacacaccccccgggggtcgcgcgcggccctttaaagcgcggcggcgcagcccgggccccccgcggccgagacgagcgagttagacaggcaagcactactcgcctctgcacgcacatgcttgcctgtcaaactctaccaccccggcacgctctctgtctccatggcccgccgccgccgccatcgcggcccccgccgcccccggccgcccgggcccacgggcgccgtcccaaccgcacagtcccaggtaaccctcgaccctagatgcgccgacccggaaacgccatataaggagcaggaaggatcccccgccggaacagaccttatttgggcagcgccttatatggagtggcccaatatggccctgccgcttccggctctgggaggangggcgagcgggggttggggcgggggcaanctgggaactccaggcgcctggcccgggaggccactgctgctgttccaatactaggctttccaggancctgagcgctcgcgatgccggagcgggtcgcagggtggangtgcccaccactcttggatgggagggcttcacgtcnctccgggtcctcccggccggtccttccatattagggcttcctgcttcccatatatggccatgtacgtcacgcggaagcgggcccgtgctgttccagacccttgaaatagaggccgattcggggagtcgcgagagatcccagcgcgcagaacttggggagccgccgccgcgattcgccgccgccgccagcttccggtcgagtcgaggtcgaggtcgacggtatcgataagcttgggctgcaggtcgatcgactctaggtttaaacagatctggatctcctaggaagctggccgcacaaagtggtaccgtaccggatcggcgcggatccgacatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctcgcgagctaagagggagggcagagggagcctgctcacctgcggagacgtggaagagaatcccgggcctatgtcgacgtcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtcgacgtatacttaagagctccgctcgagcggggtaccagatctgtcgacgatccggggatcctgcgcggggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatgggagaccaccgtgaaacgcccaccaaatattgcccaaggtcttacataagaaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaacttttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcctctagagtcgacctgcaggtttaaacgcggccgccagcgcggcggggcccggccaaccagcgtccgccgagtcttcggggcccggcccattgggcgggagttaccgcccaatgggccgggccgcccacttcccggtatggtaattaaaaacttgcaagaggccttgttccgcttcccggtatggtaattagaaactcattaatgggcggccccggccgcccttcccgcttccggcaattcccgcggcccttaatgggcaaccccggtattccccgcctcccgcgccgcgcgtaaccactcccctggggttccgggttatgctaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggtgggaggggcatgctaatggggttctttgggggacaccgggttggtcccccaaatcgggggccgggccgtgcatgctaatgatattctttgggggcgccgggttggtccccggggacggggccgccccgcggtgggcctgcctcccctgggacgcgcggccattgggggaatcgtcactgccgcccctttggggaggggaaaggcgtggggtataagttagccctggcccgacagtctggtcgcatttgcacctcggcactcggagcgagacgcagcagccaggcagactcgggccgccccctctccgcatcaccacagaagccccgcctacgttgcgacccccagggaccctccgtccgcgaccctccaaccgcatacgacccccatggagccccgccccggagcgagtacccgccggcctgagggccgcccccagcgcgaggtgaggggccgggcgccatgtctggggcgccatattggggggcgccatattggggggcgccatgttgggggacccccgacccttacactggaaccggccgccatgttgggggacccccactcatacacgggagccgggcgccatgttagggggcgtggaaccccgtgacactatatatacagggaccgggggcgccatgttagggggcgcggaaccccctgaccctatatatacagggaccggggtcgccctgttgggggtcgccatgtgaccccctgactttatatatacagaccccccaacacatacacatggcccctttgactcagacgcagggcccggggtcgccgtgggaccccctgactcatacacagagacacgcccccacaacaaacacacagggaccggggtcgccgtgttagggggcgtggtccccactgactcatacgcagggcccccttactcacacgcatctaggggggtggggaggagccgcccgccatatttgggggacgccgtgggacccccgactccggtgcgtctggagggcgggagaagagggaagaagaggggtcgggatccaaaggacggacccagaccacctttggttgcagacccctttctcccccctcttccgaggccagcaggggggcaggactttgtgaggcggggggggggagagggggaactcgtgggcgctgattgacgcgggaaatccccccattcttacccgcccccctttttttccccttagcccgccccggatgtctgggtgtttccctgcgaccgagacctgccggacagcagcgactctgaggcggagaccgaagtgggggggcggggggacgccgaccaccatgacgacgactccgcctccgaggcggacagcacggacacggaactgttcgagacggggctgctggggccgcagggcgtggatgggggggcggtctcgggggggagccccccccgcgaggaagaccccggcagttgcgggggcgccccccctcgagaggacggggggagcgacgagggcgacgtgtgcgccgtgtgcacggatgagatcgcgccccacctgcgctgcgacaccttcccgtgcatgcaccgcttctgcatcccgtgcatgaaaacctggatgcaattgcgcaacacctgcccgctgtgcaacgccaagctggtgtacctgatagtgggcgtgacgcccagcgggtcgttcagcaccatcccgatcgtgaacgacccccagacccgcatggaggccgaggaggccgtcagggcgggcacggccgtggactttatctggacgggcaatcagcggttcgccccgcggtacctgaccctgggggggcacacggtgagggccctgtcgcccacccaccctgagcccaccacggacgaggatgacgacgacctggacgacggtgaggcgggggggcggcgaggaccctgggggaggaggaggaggaggggggagggaggaataggcgggcgggggggcgaggaaagggcgggcgcggaaagggagggcctgggagggggcgtaacctgatcgcgccccccgttgtctcttgcagcagactacgtaccgcccgccccccgccggacgccccgcgcccccccacgcagaggcgccgccgcgccccccgtgacgggcggggcgtctcacgcagccccccagccggccgcggctcggacagcgcccccctcggcgcccatcgggccacacggcagcagtaacaccaacaccaccaccaacagcagcggcggcggcggcggctcccgccagtcgcgagccgcggcgccgcggggggcgtctggcccctccgggggggttggggttggggttggggttgttgaagcggaggcggggcggccgaggggccggacgggcccccttgtcaacagacccgccccccttgcaaacaacagagaccccatagtgatcagcgactcccccccggcctctccccacaggccccccgcggcgcccatgccaggctccgccccccgccccgggcccaccgcgtcctcggccgcgtcgggacccgcgcgcccccgcgcggccgtggccccgtgcgtgcgagcgccgcctccggggcccggcccccgcgccccggcccccgcggacgcgcgccgtgtgccccagtcgcactcgtccctggctcaggccgcgaaccaagaacagagtctgtgccgggcgcgtgcgacggtggcgcgcggctcgggggggccgggcgtggagggtggacacgggccctcccgcggcgccgccccctccggcgcccccccgctcccctccgccgcctctgtcgagcaggaggcggcggtgcgtccgaggaagaggcgcgggtcgggccaggaaaacccctccccccagtccacgcgtccccccctcgcgccggcaggggccaagagggcggcgacgcaccccccctccgactcagggccgggggggcgcggccagggtgggcccgggacccccctgacgtcctcggcggcctccgcctcttcctcctctgcctcttcctcctcggccccgactcccgcgggggccgcctcttccgccgccggggccgcgtcctcctccgcttccgcctcctcgggcggggccgtcggtgccctgggagggagacaagaggaaacctccctcggcccccgcgctgcttctgggccgcgggggccgaggaagtgtgcccggaagacgcgccacgcggagacttccggggccgcccccgcgggcggcctcacgcgctacctgcccatctcgggggtctctagcgtggtcgccctgtcgccttacgtgaacaagacgatcacgggggactgcctgcccatcctggacatggagacggggaacatcggggcgtacgtggtcctggtggaccagacgggaaacatggcgacccggctgcgggccgcggtccccggctggagccgccgcaccctgctccccgagaccgcgggtaaccacgtgatgccccccgagtacccgacggcccccgcgtcggagtggaacagcctctggatgacccccgtggggaacatgctgttcgaccagggcaccctagtgggcgccctggacttccgcagcctgcggtctcggcacccgtggtccggggagcagggggcgtcgacccgggacgagggaaaacaataagggacgccccccgtgtttgtggggaggggggggtcgggcgctgggtggtctctggccgcgcccactacaccagccaatccgtgtcggggaggggaaaagtgaaagacacgggcaccacacaccagcgggtctttagtgttggccctaataaaaaactcaggggatttttgctgtctattgggaaataaaggtttacttttgtatcttttccctgtctgtgttggatggatctcgggggtgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgccatgttgggcaggctctggtgttaaccacagagccgcggcccgggctgcctgaccaccgatccccgaaagcatcctgccactggcatggagccagaaccacagtgggttgggtgtgggtgttaagtttccgcgagcgcctgcccgcccggactgacctggcctctggccgccacaaagggcgggggggggttaactacactatagggcaacaaaggacgggaggggtggcggggcgggacggggcgcccaaaagggggtcggccacaccacagacgtgggtgttggggggtggggcggaggggtggggggggagacagaaacaggaacatagttagaaaacaagaatgcggtgcagccagagaatcacaggagacgaggggatgggcgtgttggttaccaacccacacccaggcatgctcggtggtatgaaggagggggggcggtgcttcttagagaccgccgggggacgtggggttggtgtgcagaggcacgcgcacccgcgtcggccaggtgggccggtaccccatcccccctcccccgacccttcccacccccgcgtgccagagatcaccccggtcccccggcacccgccactcctccatatcctcgctttaggaacaactttagggggggtacacacgcgccgtgcatttccttccacaccccccctcccccgcactcccccccccccggcagtaagacccaagcatagagagccaggcacaaaaacacaggcggggtgggacacatgccttcttggagtacgtgggtcattggcgtggggggttacagcgacaccggccgaccccctggcggtcttccagccggcccttagataagggggcagttggtggtcggacgggtaagtaacagagtctgactaagggtgggagggggggaaaagaacgggctggtgtgctgtaacacgagcccacccgcgagtggcgtggccgaccttagcctctggggcgccccctgtcgtttgggtcccccccctctattggggagaagcaggtgtctaacctacctggaaacgcggcgtctttgttgaaccacaccggggcgcccttgacgagtgggataacgggggaggaagggagggaggagggtactgggggtgaagaaggggggggggggagaagcgagaacaggaaaggcgacggagcccgacaaaacaccgagaaaaaaaaaccacagcgcatgcgccgggccgttgtggggccccgggccggggccccttgggtccgccggggccccgggccgggccgccacgggggccggccgttggcggtaaccccgattgtttatctcaggccccgggccgggaacccggaaaagcctccggggggcctttttcgcgtcgcgtgccggcgagcgggcccggacggggcccggaccgccgcggtcgggggccccctcgtcccgggccgtacgcggccttcgccccgtgaggggacagacgaacgaaacattccggcgacggaacgaaaaacaccccagacgggttaaagaaacagaaaccgcaaccccccccacccccgaaacggggaaaacaaaaaacagaccagcggccggccggcgcttagggggaggatgtcgccgacgccccttggccgccccggctgcaggggggcccggagagccgcggcacccggacgcgcccggaaagtctttcgcaccacccgcgatcggcacggccgcgcccccgcttttataaaggctcagatgacgcagcaaaaacaggccacagcaccacgtgggtaggtgatgtaattttattttcctcgtctgcggcctaatggatttccgggcgcggtgcccctgtctgcagagcacttaacggattgatatctcgcgggcacgcgcgcccttaatggaccggcgcggggcggggggccggatacccacacgggcgggggggggggtgtcgcgggccgtctgctggcccgcggccacataaacaatgactctgggcctttctgcctctgccgcttgtgtgtgcgcgcgccggctctgcggtgtcggcggcggctgcggcggctgcggcggccgccgtgttcggtctcggtagccggccggcgggtggactcgcggggggccggagggtggaaggcaggggggtgtaggatgggtatcaggacttccacttcccgtccttccatcccccgttcccctcggttgttcctcgcccccccccacaccccgccgctttccgttggggttgttattgttgtcgggatcgtgcgggccgggggtcgccggggcaggggcgggggcgggggtgctcgtcgatcgaccgggctcagtgggggcgtggggtgggggggaaaaggcgaagagactgggggtgggggggggtgtcgggggtggctgtttttttttgtgggtgttttttgtggctgttcccgtcccccgtcacccccctccctccgtccccccgtcgcgggtgtttgtgtttgtttattccgacatcggtttatttaaataaacacagccgttctgcgtgtctgttcttgcgtgtggctgggggcttatatgtggggtcccgggggcgggatggggtttagcggcggggggcggcgcgccggacggggcgctggagataacggcccccggggaacgggggaccggggctgggtctcccgaggtgggtgggtgggcggcggtggccgggccgggccgggccgggtgggcggggtttggaaaaacgaggaggaggaggaggagaaggagggggggggagacggggggaaagcaaggacacggcccggggggtgggagcgcgggccgggccgctcgtaagagccgcgacccggccgccggggagcgttgtcgccgtcggtctgccggcccccgtccctcccttttttgaccaaccagcgcccccccccctcaccaccattcctaccaccaccaccaccaccgacacctcccgcacacccccgcccacactccccccccccacccaacccgcaccacgagcacgggttgggggtagcaggggatcaaaggggggcaaggccggcggggcggttcgggggcgggggcgggagaccgagtaggccccgcccatccgcggcccctcccggcagccacgccccccagcgtcgggtgtcacggggaaagagcagggggagaggggagagggggggagaggggagagggggggagaggggagagggggggagaggggagagggggggagaggggagagggggggagaggggagagggggggagaggggagagggggggagaggggagagggggggagaggggagagggggggagaggggagagggggggagagggggtatataaaccaacgaaaagcgcgggaacggggatacggggcttgtgtggcacgacgtcgtggttgtgttactgggcaaacacttggggactgtaggtttctgtgggtgccgaccctaggcgctatggggattttgggttgggtcgggcttattgccgttggggttttgtgtgtgcgggggggcttgtcttcaaccgaatatgttattcggagtcgggtggctcgagaggtgggggatatattaaaggtgccttgtgtgccgctcccgtctgacgatcttgattggcgttacgagaccccctcggctataaactatgctttgatagacggtatatttttgcgttatcactgtcccggattggacacggtcttgtgggataggcatgcccagaaggcatattgggttaacccctttttatttgtggcgggttttttggaggacttgagtcaccccgcatttcctgccaacacccaggaaacagaaacgcgcttggccctttataaagagatacgccaggcgctggacagtcgcaagcaggccgccagccacacacctgtgaaggctgggtgtgtgaactttgactattcgcgcacccgccgctgtgtagggcgacaggatttgggacctaccaacggaacgtctggacggaccccggttctgccgccggacgatgaagcgggcctgcagccgaagcccctcaccacgccgccgcccatcatcgccacgttggaccccaccccgcgacgggacgccgccgcaaaaagcagacgccgacgaccccactcccggcgcatctaatgatgccgcgacggaaacccgtccgggttcggggggcgaaccggccgcctgtcgctcgtcagggccggcgggcgctcctcgccgccctagaggctgtcccgctggtgtgacgttttcctcgtccgcgccccccgaccctcccatggatttaacaaacgggggggtgtcgcctgtggcgacctcggcgcctctggactggaccacgtttcggcgtgtgtttctgatcgacgacgcgtggcggcccctgttggagcctgagctggcgaaccccttaaccgcccacctcctgaccgaatataatcgtcggtgccagaccgaagaggtgctgccgccgcgggaggatgtgttttcgtggactcgttattgcacccccgacgaggtgcgcgtggttatcatcggccaggacccatatcaccaccccggccaggcgcacggacttgcgtttagcgtgcgcgcgaacgtgccgcctcccccgagtcttcggaatgtcttggcggccgtcaagaactgttatcccgaggcacggatgagcggccacggttgcctggaaaagtgggcgcgggacggcgtcctgttactaaacacgaccctgaccgtcaagcgcggggcggcggcgtcccactctagaatcggttgggaccgcttcgtgggcggagttatccgccggttggccgcgcgccgccccggcctggtgtttatgctctggggcgcacatgcccagaatgccatcaggccggaccctcgggtccattgcgtcctcaagttttcgcacccgtcgcccctctccaaggttccgttcggaacatgccagcatttcctcgtggcgaatcgatatctcgagacccggtcgatttcacccatcgactggtcggtttgaaaggcatcgacgtccggggttttcgtctgtgggggcttttgggtatttccgatgaataaagacggttaatggttaaacctctggtctcatacgggtcggtgatgtcgggcgtcgggggagagggagttccctctgcgcttgcgattctagcctcgtggggctggacgttcgacacgccaaaccacgagtcagggatatcgccagatacgactcccgcagattccattcggggggccgctgtggcctcacctgaccaacctttacacgggggcccggaacgggaggccacagcgccgtctttctccccaacgcgcgcggatgacggcccgccctgtaccgacgggccctacgtgacgtttgataccctgtttatggtgtcgtcgatcgacgaattagggcgtcgccagctcacggacaccatccgcaaggacctgcggttgtcgctggccaagtttagcattgcgtgcaccaagacctcctcgttttcgggaaacgccccgcgccaccacagacgcggggcgttccagcgcggcacgcgggcgccgcgcagcaacaaaagccttcagatgtttgtgttgtgcaaacgcacccacgccgctcgagtgcgagagcagcttcgggtcgttattcagtcccgcaagccgcgcaagtattacacgcgatcttcggacgggcggctctgccccgccgtccccgtgttcgtccacgagttcgtctcgtccgagccaatgcgcctccaccgagataacgtcatgctggcctcgggggccgagtaaccgcccccccgcgccaccctcactgcccgtcgcgcgtgtttgatgttaataaataacgcataaattatcttaatagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccactagtccagtgtggtggaattgcccttggatcctcaatggcccagcgcaacggcatgtcgccgcgccccccgcccctcggtcgcggccgcggggccggagggccttcgggggttggttcctctcctccttcttcttgtgtgccgatgggagcgccgtccacagcgggcactggtgcgagtgctgcggctacgacgacgccgggccacggcgtccaccgggtagaaccccgcgggccgccgggcgcccctccgagtagcgacaacaatggcaacttttggcacggcccggagcgcctgttgctgtctcagattccggtggagcgccaggcgctgacggagctggaataccaggccatgggcgccgtgtggcgcgcggcgtttttggccaacagcacgggccgcgccatgcgcaagtggtcgcagcgcgacgcgggcacgctgctgccgctcggacggccgtacggattctacgcgcgggtgacgccgcgcagccagatgaacggcgtgggcgcgacggacctgcgtcaactgtcgccgcgggacgcgtggatcgtactggtggctaccgtggtgcacgaggtggaccccgcagccgacccgacgttgggcgacaaggccggccatcccgagggtctgtgcgcgcaggacggactgtacctggcgctgggcgccgggttccgcgtgttcgtgtacgacctggcaaacaacacgctgatcctagcggcgcgcgacgcggacgagtggtttcggcacggcgcgggcgaggtggtgcggctgtaccgctgcaaccggctgggcgtgggcaccccgcgcgcgacgctgctgcctcagccggcgctccgacagacgttgctgcgcgccgaggaggcgacggcgctcggacgggagctgcgccggcggtgggccggcacgacggtggcgctgcagacgccgggcaggcgactgcagccgatggtactgctgggcgcgtggcaggagctggcgcagtacgagccgttcgcgtcggcgccgcaccccgcgtcgctgctgacggccgtgcgtcggcacctgaaccagcgtctgtgctgcggctggctggcgctgggcgcggtgctgcccgcgcggtggctgggctgcgcggcggggccggcgacggggacggcggcggggacgacgtcgccgccagcggcgagcggcacggagacggaggccgccggcggggacgcgccgtgcgcgatagcgggagccgtggggtccgctgtacctgtgcctccgcagccgtacggcgccgccggcgggggcgcgatttgcgtgcctaacgcggacgcgcacgcggtggtcggggcggacgcggcagcagcagcggcgccgacggtgatggtgggttcgacagcgatggcgggtccggcggcgtcggggaccgtgccgcgcgccatgctggtggtgctgctggacgagctgggcgccgtgttcgggtactgcccgctggacgggcacgtgtacccgctggcggcggagctgtcgcactttctgcgcgcgggcgtgctgggcgcgctggcgctgggacgcgagtcggcgcccgccgccgaggccgcgcggcggctgctgcccgagctggaccgcgagcagtgggagcggccgcgctgggacgcgctgcacctgcacccgcgcgccgcgctgtgggcgcgcgagccgcacgggcagtgggagttcatgtttcgcgaacaacgcggtgaccccataaatgatcccctcgcatttcgtctttcggacgctcgaactctcggtctcgacctcaccaccgtcatgacagagcgtcaaagtcaattgcccgaaaagtatatcggtttctatcagattaggaaacctccttggctcatggaacaacctccacccccatctcgccaaaccaaaccggacgctgcaacgatgcccccaccgctcagtgctcaggcaagcgtcagctacgcgctccgatacgatgacgagtcctggcgcccgctcagcacagttgacgaccacaaagcctggttggatctcgacgaatcacattgggtcctcggggacagccgacccgacgatataaaacaacgcagactgctgaaggccactcaacgacgaggcgccgaaatcgacagacccatgcctgtcgtgcctgaagaatgttacgaccaacgcttcactaccgaaggccaccaggtcatcccgttgtgcgcgtccgaacccgaggatgacgacgaagatcctacctacgacgaattgccgtcgcgcccaccccagaaacataagccgccagacaaacctccgcgcttatgcaaaacgggccccggcccacctccgctgccgccaaagcaacggcacggttccaccgacggaaaagtttctgcgccccgacagtcggagcatcataaaagacagacccgaccgccaaggccgccaccgcccaaattcggggatagaaccgcggcccatctctcgcaaaatatgcgggacatgtacctcgatatgtgtacatcttcgggccacaggccacggccgccagcacctccgcggccgaaaaaatgtcaaacacacgcccctcaccacgttcatcataagcttaagggcaattctgcagatatccagcacagtggcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctagtattaaggaatttggctggttgtttgttgtctttaatggaccgcccgcagggggggtggcatttcagtgtcgggtgacgagcgcgatccggccgggatcctaggaccccaaaagtttgtctgcgtattccagggcggggctcagttgaatctcccgcagcacctctaccagcaggtccgcggtgggctggagaaactcggccgtcccggggcaggcggtcgtcgggggtggaggcgcggcgcccaccccgtgtgccgcgcctggcgtctcctctgggggcgacccgtaaatggttgcagtgatgtaaatggtgtccgcggtccagaccacggtcaaaatgccggccgtggcgctccgggcgctttcgccgcgcgaggagctgacccaggagtcgaacggatacgcgtacatatgggcgtcccacccgcgttcgagcttctggttgctgtcccggcctataaagcggtaggcacaaaattcggcgcgacagtcgataatcaccaacagcccaatgggggtgtgttggataacaacgcctccgcgcggcaggcggtcctggcgctcccggccccgtaccatgatcgcgcgggtgccgtactcaaaaacatgcaccacctgcgcggcgtcgggcagtgcgctggtcagcgaggccctggcgtggcataggctatacgcgatggtcgtctgtggattggacatctcgcggtgggtagtgagtcccccgggccgggttcggtggaactgtaaggggacggcgggttaatatacaatgaccacgttcggatcgcgcagagccgatagtatgtgcttactaatgacgtcatcgcgctcgtggcgctcccggagcggatttaagttcatgcgaaggaattcggaggaggtggtgcgggacatggccacgtacgcgctgttgaggcgcaggttgccgggcgtaaagcagatggcgaccttgtccaggctaaggccctgggagcgcgtgatggtcatggcaagcttggagctgatgccgtagtcggcgtttatggccatggccagctccgtagagtcaatggactcgacaaactcgctgatgttggtgttgacgacggacatgaagccgtgttggtcccgcaagaccacgtaaggcaggggggcctcttccagtaactcggccacgttggccgtcgcgtgccgcctccgcagctcgtccgcaaaggcaaacacccgtgcgtacgtgtatcccatgagcgtataattgtccgtctgcagggcgacggacatcagccccccgcgcggcgagccggtcagcatctcgcagccccggaagataacgttgtccacgtacgtgctaaagggggcgccttcaaatgcctccccaaagagctcttggaggattcggaatctcccgaggaaggcccgcttcagcagcgcaaactgggtgtgaacggcggcggtggtctccggttccccgggggtgtagtggcagtaaaacacgtcgagctgttgttcgtccagccccgcgaaaataacgtcgaggtcgtcgtcgggaaaatcgtccgggcccccgtcccgcggccccagttgcttaaaatcaaacgcacgctcgccgggggcgcctgcgtcggccattaccgacgcctgcgtcggcacccccgaagatttggggcgcagagacagaatctccgccgttagttctcccatgcgggcgtaggcgagggtcctctgggtcgcatccaggcccgggcgctgcagaaagttgtaaaaggagataagcccgctaaatatgagccgcgacaggaacctgtaggcaaactccaccgaagtctccccctgagtctttacaaagctgtcgtcacgcaacactgcctcgaaggcccggaacgtcccactaaacccaaaaaccagttttcgcaggcgcgcggttaccgcgatctggctgttgaggacgtaagtgacgtcgttgcgggccacgaccagctgctgtttgctgtgcacctcgcagcgcatgtgccccgcgtcctggtcctggctctgcgagtagttggtgatgcggctggcgttggccgtgagccacttttcaatagtcaggccgggctggtgtgtcagccgtcggtattcgtcaaactccttgaccgacacgaacgtaagcacggggagggtgaacacgacaaactccccctcacgggtcaccttcaggtaggcgtggagcttggccatgtacgcgctcacctctttgtgggaggagaacaaccgcgtccagccggggaggttggcggggttggtgatgtagttttccgggacgacgaagcgatccacgaactgcatgtgctcctcggtgatgggtaggccgtactccagcaccttcatgaggttaccgaactcgtgctcgatgcaccgtttgttgttaataaaaatggcccagctatacgagaggcgggcgtactcccgcagcgtgcggttgcagatgaggtacgtgagcacgttctcgctctggcggacggaacaccgcagtttctggtgctcgaaggtcgactccagggacgccgtctgtgtcggcgagcccacacacaccaacacgggccgcaggcgggccgcgtactggggggtgtggtacagggcgttaatcatccaccagcaatacaccacggccgtgaggaggtgacgcccaaggagcccggcctcgtcgatgacgatcacgttgctgcgggtaaaggccggcagcgccccgtgggtggccggggccaaccgcgtcagggcgccctcggccaaccccagggtccgttccagggcggccagggcgcgaaactcgttccgcgactcctcgcccccggaggcggccagggtgcgcttcgtgaggtccaaaatcacctcccagtagtacgtcagatctcgtcgctgcaggtcctccagcgaggcggggttgctggtcagggtgtacgggtactgccccagttgggcctggacgtgattcccgcgaaacccaaattcatgaaagatggtgttgatgggtcggctgagaaaggcgcccgagagtttggcgtacatgttttgggccgcaatgcgcgtggcgcccgtcaccacacagtccaagacctcgttgattgtctgcacgcacgtgctctttccggagccagcgttgccggtgataagatacaccgcgaacggaaactccctgaggggcaggcctgcgggggactctaaggccgccacgtcccggaaccactgcagacggggcacttgcgctccgtcgagctgttgttgcgagagctctcggatgcgcttaaggattggctgcaccccgtgcatagacgtaaaatttaaaaaggcctcggccctccctggaacggctggtcggtccccgggttgctgaaggtgcggcgggccgggtctctgtccgtctagctggcgctccccgccggccgccgccatgaccgcaccacgctcgcgggcccccactacgcgtgcgcggggggacacggaagcgctgtgctcccccgaggacggctgggtaaaggttcaccccacccccggtacgatgctgttccgcgagattctccacgggcagctggggtataccgagggccagggggtgtacaacgtcgtccggtccagcgaggcgaccacccggcagctgcaggcggcgatctttcacgcgctcctcaacgccaccacttaccgggacctcgaggcggactggctcggccacgtggcggcccgcggtctgcagccccaacggctggttcgccggtacaggaacgcccgggaggcggatatcgccggggtggccgagcgggtgttcgacacgtggcggaacacgcttaggacgacgctgctggactttgcccacgggttggtcgcctgctttgcgccgggcggcccgagcggcccgtcaagcttccccaaatatatcgactggctgacgtgcctggggttggtccccatattacgcaagcgacaagaagggggtgtgacgcagggtctgagggcgtttctcaagcagcacccgctgacccgccagctggccacggtcgcggaggccgcggagcgcgccggccccgggttttttgagctggcgctggccttcgactccacgcgcgtggcggactacgaccgcgtgtatatttactacaaccaccgccggggcgactggctcgtgcgagaccccatcagcgggcagcgcggagaatgtctggtgctgtggcctcccttgtggaccggggaccgtctggtcttcgattcgcccgtacagcggctgtttcccgagatcgtcgcgtgtcactccctccgggaacacgcgcacgtctgccggctgcgcaataccgcgtccgtcaaggtgctgctggggcgcaagagcgacagcgagcgcggggtggccggcgccgcgcgggtcgttaacaaggtgttgggggaggacgacgagaccaaggccgggtcggccgcctcgcgcctcgtgcggcttatcatcaacatgaagggcatgcgccacgtaggcgacattaacgacactgtgcgtgcctacctcgacgaggccggggggcacctgatagacgccccggccgtcgacggtaccctcccgggattcggcaagggcggaaacagccgcgggtctgcgggccaggaccagggggggcgggcgccgcagcttcgccaggccttccgcacggccgtggttaacaacatcaacggcgtgttggagggctatataaataacctgtttggaaccatcgagcgcctgcgcgagaccaacgcgggcctggcgacccagttgcaggagcgcgaccgcgagctccggcgcgcaacatcgggggccctggagcgccagcagcgcgcggccgacctggcggccgagtccgtgaccgggggatgcggcagccgccctgcgggggcggacctgctccgggccgactatgacattatcgacgtcagcaagtccatggacgacgacacgtacgtcgccaacagttttcagcacccgtacatcccttcgtacgcccaggacctggagcgcctgtcgcgcctctgggagcacgagctggtgcgctgtttcaaaattctgtgtcaccgcaacaaccagggccaagagacgtcgatctcgtactccagcggggcgatcgccgcattcgtcgccccctactttgagtcagtgcttcgggccccccgggtaggcgcgcccatcacgggctccgatgtcatcctgggggaggaggagttatgggatgcggtgtttaagaaaacccgcctgcaaacgtacctgacagacatcgcggccctgttcgtcgcggacgtccagcacgcagcgctgcccccgcccccctccccggtcggcgccgatttccggcccggcgcgtccccgcggggccggtccagatcgcggtcgcccggaagaactgcgcgaggcgcgccggaccagggcgggggcatcgggcaccgggatggccgccgcgacggccgacgatgaggggtcggccgccaccatcctcaagcaggccatcgccggggaccgcagcctggtcgaggcggccgaggcgattagccagcagacgctgctccgcctggcctgcgaggtgcgccaggtcggcgaccgccagccgcggtttaccgccaccagcatcgcgcgcgtcgacgtcgcgcctgggtgccggttgcggttcgttctggacgggagtcccgaggacgcctatgtgacgtcggaggattactttaagcgctgctgcggccagtccagttatcgcggcttcgcggtggcggtcctgacggccaacgaggaccacgtgcacagcctggccgtgccccccctcgttctgctgcaccggttctccctgttcaaccccagggacctcctggactttgagcttgcctgtctgctgatgtacctggagaactgcccccgaagccacgccaccccgtcgacctttgccaaggttctggcgtggctcggggtcgcgggtcgccgcacgtccccattcgaacgcgttcgctgccttttcctccgcagttgccactgggtcctaaacacactcatgttcatggtgcacgtaaaaccgttcgacgacgagttcgtcctgccccactggtacatggcccggtacctgctggccaacaacccgccccccgttctctcggccctgttctgtgccaccccgacaagctcctcattccggctgccggggccgcccccccgctccgactgcgtggcctataaccccgccgggatcatggggagctgctgggcgtcggaggaggtgcgcgcgcctctggtctattggtggctttcggagaccccaaaacgacagacgtcgtcgctgttttatcagttttgttgaattttaggaaataaacccggttttgtttctgtggcctcccgacggatgcgcgtgtccttactccgtcttggtgggtgggtggctgtgtatggcgtcccatctgtgcggggaggggggcaagtcggcacgtattcggacagactcaagcacacacgggggagcgctcttgtctcagggcaatgtttttattggtcaaactcaggcaaacagaaacgacatcttgtcgtcaaagggatacacaaacttccccccctcgccccatactcccgccagcaccccggtaaacaccaactcaatctcgcgcaggatttcgcgcaggtgatgagcgcagtccacgggggggagcacaaggggccgcgggtatagatcgacggggacgccgaccgactccccgcctccgggacagacacgcacgacgcgccgccagtagtgctctgcgtccagcaaggcgccgccgcggaaggcagtggggggcaaggggtcgctggcctcaaagggggacacccgaacgctccagtactccgcgtccaaccgtttattaaacgcgtccaagataaggcggtcgcaggcgtcctccataaggccccgggccgtgagtgcgtcctcctccggcacgcctgccgttgtcaggcccaggacccgtcgcagcgtgtcgcgtacgaccccggccgccgtggtgtacgcgggcccgcggagaggaaatcccccaagatggtcagtgttgtcgcgggagttccagaaccacactcccgcctggctccaggcgactgcgtgggtgtagacgccctcgagggccaggcacagtgggtgccgcagccggaggccgttggccctaagcacggctcccacggccgtctcgatggcccgccgggcgtcctcgatcaccccggaagccgcatccgcgtcttgggggtccacgttaaagacaccccagaacgcacccccatcgcccccgcagaccgcgaacttcaccgagctggccgtctcctcgatctgcaggcagacggcggccattaccccacccaggagctgccgcagcgcagggcaggcgttgcacgtgtccgggaccaggcgctccaagacggccccggcccagggctctgagggagcggccaccaccagcgcgtccagtcttgctaggcccgtccggccgtgggggtccgccagcccgctccccccgaggtcggccagggccgccaggagctgggcgcgaagtccggggaagcaaaaccgcgccgtccagacgggcccgacggccgcgggcgggtctaacagttggatgattttagtggcgggatgccaccgcgccaccgcctcccgcactgcgggcaggaggcatccggctgccgccgaggccacgccgggccaggctcgcggggggaggacgaccctgacccccaccgcgggccaggcccccaggagcgcggcgtaagcggccgcggccccgcgcaccaggtcccgtgccgactcggccgtggccggcacggtgaacgtgggccaacccggaaaccccaggacggcaaagtacgggacgggtcccccccggacctcaaactcgggccccagaaaggcaaagacgggggccagggccccgggggcggcgtggaccgtggtatgccactgccggaaaagggcgacgagcgccggcgcggagaacttctcgccggcgcttacaaagtagtcgtaatcgcggggcagcagcacccgtgccgtgactcgttgcgggtgcccgcgtggccgcaggcccacctcgcacacctcgaccaggtccccgaacgcgccctccttcttgatcggcggaaacgcaagagtctggtattcgcgcgcaaatagcgcggttccggtggtgatgttaacggtcagcgaagcggtggacgcgcactggggggtgtcgcgaatggccgccaggcgcgcccacgccagccgcgcgtcgggatgctcggcaacgcgcgccgccagggccatagggtcgatgtcaatgttggcctccgcgaccaggagagcggcgcgaggggcggcgggcgggccccacgacgctctctcaactttcaccaccagtcccgtgcgtgggtccgagccgatacgcagcggggcgaacagggccaccggcccggtctggcgctccagggccgccaggacgcacgcgtacagcgcccgccacagagtcgggttctccaggggctccagcggggaggcggccggcgtcgtcgcggcgcgggcggccgccacgacggcctggacggagacgtccgcggagccgtagaaatcccgcagctccgtcgcggtgacggagacctccgcaaagcgcgcgcgaccctcccctgcggcgttgcgacatacaaaatacaccagggcgtggaagtactcgcgagcgcgggggggcagccataccgcgtaaagggtaatggcgctgacgctctcctccacccacacgatatctgcggtgtccatcgcacggcccctaaggatcacgggcggtctgtgggtcccatgctgccgtgcctggccgggcccggtgggttgcggaaaccggtgacgggggggggggcggtttttggggttggggtgggaaacggcccgggtccgggggccaacttggcccctcggtgcgttccggcaacagcgccgccggtccgcggacgaccacgtaccgaacgagtgcggtcccgagacttatagggtgctaaagttcaccgccccctgcatcatgggccaggcctcggtggggagctccgacagcgccgcctccaggatgatgtcagcgttggggttggcgctggatgagtgcgtgcgcaaacagcgcccccacgcgggcacgcgtagcttgaagcgcgcgcccgcaaactcccgcttgtgggccataagcagggcgtacagctgcctgtgggtccggcaggcgctgtggtcgatgtggtgggcgtccaacaaccccacgattgtctgtttggtgaggtttttaacgcgccccgccccgggaaacgtctgcgtgcttttggccatctgcacgccaaacagttcgccccagattatcttgaacagcgccaccgcgtggtccgtctcactaacggacccgcgcgggggacagccgcttagggcgtcggcgacgcgcttgacggcttcctccgagagcagaagtccgtcggttacgttacagtggcccagttcgaacaccagctgcatgtagcggtcgtagtggggggtcagcaggtccagcacgtcatcggggccgaaggtcctcccagatcccccggccgccgagtcccaatgcaggcgcgcggccatggtgctgcacaggcacaacagctcccagacaggggttacgttcagggtggggggcagggccacgagctccagctctccggtgacgttgatcgtggggatgacgcccgtggcgtagtggtcatagatccgccgaaatatggcgctgctgcgggtggccatgggaacgcggagacaggcctccagcaacgccaggtaaataaaccgcgtgcgtcccatcaggctgttgaggttgcgcatgagcgcgacaatttccgccggcgcgacatcggaccggaggtatttttcgacgaaaagacccacctcctccgtctcggcggcctgggccggcagcgacgcctcgggatcccggcaccgcagctcccgtagatcgcgctgggccctgagggcgtcgaaatgtacgccccgcaaaaacagacagaagtcctttggggtcagggtatcgtcgtgtccccagaagcgcacgcgtatgcagtttagggtcagcagcatgtgaaggatgttaaggctgtccgagagacacgccagcgtgcatctctcaaagtagtgtttgtaacggaatttgttgtagatgcgcgacccccgccccagcgacgtgtcgcatgccgacgcgtcacagcgccccttgaaccggcgacacagcaggtttgtgacctgggagaactgcgcgggccactggccgcaggaactgaccacgtggttcaggagcatgggcgtaaagacgggctccgagcgcgccccggagccgtccatgtaaatcagtagctcccccttgcggagggtgcgcacccgtcccagggactggtacacggacaccatgtccggtccgtagttcatgggtttcacgtaggcgaacatgccatcaaagtgcaggggatcgaagctgaggcccacggttacgaccgtcgtgtatataaccacgcggtattggccccacgtggtcacgtccccgaggggggtgagcgagtgaagcaacagcacgcggtccgtaaactgacggcagaaccgggccacgatctccgcgaaggagaccgtcgacgaaaaaatgcagatgttatcgcccccgccaaggcgcgcttccagctccccaaagaacgtggccccccgggcgtccggagaggcgtccggagacgggccgctcggcggcccgggcgggcgcagggcagcctgcaggagctcggtccccagacgcgggagaaacaggcaccggcgcgccgaaaacccgggcatggcgtactcgccgaccaccacatgcacgtttttttcgccccggagaccgcacaggaagtccaccaactgcgcgttggcggttgcgtccatggcgatgatccgaggacatgtgcgcagcaggcgtagcattaacgcatccacgcggcccagttgctgcatcgttggcgaatagagctggcccagcgtcgacataacctcgtccagaacgaggacgtcgtagttgttcagaaggttggggcccacgcgatgaaggctttccacctggacgataagtcggtggaaggggcggtcgttcataatgtaattggtggatgagaagtaggtgacaaagtcgaccaggcctgactcagcgaaccgcgtcgccagggtctgggtaaaactccgacgacaggagacgacgagcacactcgtgtccggagagtggatcgcttcccgcagccagcggatcagcgcggtagtttttcccgaccccattggcgcgcggaccacagtcacgcacctggccgtcggggcgctcgcgttggggaaggtgacgggtccgtgctgctgccgctcgatcgttgttttcgggtgaacccggggcacccattcggccaaatcccccccgtataacatccgcgctagcgatacgctcgacgtgtactgttcgcactcgtcgtccccaatgggacgcccggcccccagaggatcccccgactccgcgccccccacgaaaggcatgaccggggcgcggacggcgtggtgggtctggtgtgtgcaggtggcgacgtttgtggtctctgcggtctgcgtcacggggctcctcgtcctggcctctgtgttccgggcacggtttccctgcttttacgccacggcgagctcttatgccggggttaactccacggccgaggtgcgcgggggtgtagccgtgcccctcaggttggacacgcagagccttgtgggcacttatgtaatcacggccgtattgttgttggccgcggccgtgtatgccgtggtcggcgccgtgacctcccgctacgaccgcgccctggacgcgggccgccgtctggctgcggcccgcatggccatgccgcacgccacgctgatcgccggaaacgtctgctcttggttgctgcagatcaccgtcctgctgctggcccatcgcatcagccagctggcccacctggtttacgtcctgcactttgcgtgtctggtgtattttgcggcccatttttgcaccaggggggtcctgagcgggacgtatctgcgtcaggtgcacggcctgatggagctggccccgacccatcatcgcgtcgtcggcccggctcgcgccgtgctgacaaacgccttgctgttgggcgtcttcctgtgcacggccgacgccgcggtatccctgaataccatcgccgcgttcaactttaatttttcggccccgggcatgctcatctgcctgaccgtgctgttcgccattctcgtcgtatcgctgttgttggtggtcgagggggtgttgtgtcactacgtgcgcgtgttggtgggcccccacctgggggccgttgccgccacgggcatcgtcggcctggcctgcgagcactattacaccaacggctactacgtggtggagacgcagtggccgggggcacagacgggagtgcgcgtcgccctcgccctggtcgccgcctttgccctcggcatggccgtgctccgctgcacccgcgcctatctgtatcacaggcggcaccacaccaaattttttatgcgcatgcgcgacacgcgacaccgcgcacattccgccctcaagcgcgtacgcagttccatgcgcggatcgcgagacggccgccacaggcccgcgcccggcagcccgcccgggattcccgaatatgcggaagacccctacgcgatctcatacggcggccagctcgaccggtacggagattccgacggggagccgatttacgacgaggtggcggacgaccaaaccgacgtattgtacgccaagatacaacacccgcggcacctgcccgacgacgagcccatctatgacaccgttggggggtacgaccccgagcccgccgaggaccccgtgtacagcaccgtccgccgttggtagctgtttggttccgttttaataaaccgtttgtgtttaacccgaccgtggtgtatgtctggtgtgtggcgtccgatcccgttactatcaccgtccccccccctcaaccccggcgattgtgggttttttaaaaacgacacgcgtgcgaccgtatacagaacattattttggtttttattcgctatcggacatggggggtggaaactgggtggcggggcaggcgcctccgggggtccgccggtgagtgtggcgcgagggggggtccgacgaacgcaggcgcggtctccccggggcccgcgtaaccacgcgcatatccgggggcacgtagaaattaccttcctcttcggactcgatatccacgacgtcaaagtcgtgggcggtcagcgagacgacctccccgtcgtcggtgatgaggacgttgtttcggcagcagcagggccgggccccggagaacgagaggcccatagctcggcgagcgtgtcgtcgaacgccaggcggctgcttcgctggatggccttatagatctccggatcgatgcggacgggggtaatgatcagggcgatcggaacggcctggttcgggagaatggacgccttgctgggtcctgcggccccgagagccccggcgccgtcctccaggcggaacgttacgccctcctccgcgctggtgcggtgcctgccgataaacgtcaccagatgcgggtggggggggcagtcggggaagtggctgtcgagcacgtagccctgcaccaagatctgcttaaagttcgggtggcgggggttcgcgaagacgggctcgcggcggaccagatccccggagctccaggacacgggggagatggtgtggcgtccgaggtcgggggcgccaaacagaagcacctccgagacaacgccgctatttaactccaccaaggcccgatccgcggcggagcaccgccttttttcgcccgaggcgtgggcctctgaccaggcctggtcttgcgtgacgagagcctcctccgggccggggacgcgcccgggcgcgaagtatcgcacgctgggcttcgggatcgaccggataaatgcccggaacgcctccggggaccggtgtgccatcaagtcctcgtacgcggaggccgtggggtcgctggggtccatggggtcgaaagcgtacttggcccggcatttgacctcgtaaaaggccaggggggtcttggggactggggccaggtagccgtgaatgtcccgaggacagacgagaatatccagggacgccccgaccatccccgtgtgaccgtccatgaggaccccacacgtatgcacgttctcttcggcgaggtcgctgggttcgtggaagataaagcgccgcgtgtcggcgccggcctcgccgccgtcgtccgcgcggcccacgcagtagcgaaacagcaggcttcgggccgtcggctcgttcacccgcccgaacatcaccgccgaagactgtacatccggtcgcaggctggcgttgtgcttcagccactggggcgagaaacacggaccctgggggccccagcggagggtggatgcggtcgtgaggccccgccggagcagggcccatagctggcagtcggcctggttttgcgtggccgcctcgtaaaaccccatgaggggccggggcgccacggcgtccgcggcggccgggggggcgcggcgcgtcaggcgccataggtgccggccgagtccgcggtccaccatacccgcctcctcgaggaccacggccagggaacacagataatccaggcgggcccagaggggaccgatggccagaggggcgcggacgccgcgcagcaacccgcgcaggtggcgctcgaacgtctcggctagtatatgggagggcagcgcgttggggatcaccgacgccgaccacatagagtcaaggtccggggagtcgggatcggcgtccgggtcgcgggcgtgggtgcccccaggagatagcggaatgtccggggtcggaggcccggaggcgtcagaaagtgccggcgacgcggcccggggcttttcgtctgcggtgtcggtggcgtgctgatcacgtggggggttatcgggcgaatgggagctcgggtccacagctgacgtcgtctggggtggggggggcaggggacggaaggtggttgtcagcggaagactgttagggcgggggcgcttgggggggctgtcggggccacgaggggtgtcctcggccagggcccagggacgcttagtcacggtgcgtcccggcggacatgctgggcctaccgtggactccatttccgagacgacgtggggggagcggtggttgagcgcgccgccgggtgaacgctgattctcacgacagcgcgtgccgcgcgcacgggttggtgtgacacaggcgggacaccagcaccaggagaggcttaagctcgggaggcagcgccaccgacgacagtatcgccttgtgtgtgtgctggtaatttatacaccgatccgtaaacgcgcgccgaatcttgggattgcggaggtggcgccggatgccctctgggacgtcatacgccaggccgtgggtgttggtctcggccgagttgacaaacagggctgggtgcagcacgcagcgataggcgagcagggccagggcgaagtccggcgacagctggttgttgaaatactggtaaccgggaaaccgggtcacgggtacgcccaggctcggggcgacgtacacgctaaccaccaactccagcagcgtctggcccagggcgtacaggtcaaccgctagcccgacgtcgtgcttcaggcggtggttggtaaattcggcccgttcgttgttaaggtatttcaccaacagctccgggggctggttatacccgtgacccaccagggtgtgaaagttggctgtggttagggcggtgggcatgccaaacatccggggggacttgaggtccggctcctggaggcaaaactgcccccgggcgatcgtggagttggagttgagggtgacgaggctaaagtcggcgaggacggcccgccggagcgagacggcgtccgaccgcagcatgacgaggatgttggcgcacttgatatccaggtggctgatcccgcaggtggtgtttaaaaacacaacggcacgggccagctccgtgaagcactggtggagggccgtcgagaccgaggggtttgttgtgcgcagggacgccagttggccgatatacttaccgaggtccatgtcgtacgcggggaacactatctgtcgttgttgcagcgagaacccgaggggcgcgatgaagccgcggatgttgtgggtgcggccggcgcgtagagcgcactccccgaccaacagggtcgcgatgagctcaacggcaaaccactccttttcctttatggtcttaacggcaagcttatgttcgcgaatcagttggacttcgccgtatcccccagaccccccgaagcttcgggccccggggatctcgagggtcgtgtagtgtagggcggggttgatggcgaacacggggctgcatagcttgcggatgcgcgtgagggtgaggatgtgcgagggggacgaggggggtgcggttaacgccgcctgggatctgcgcaggggcgggcggttcagtttggccgccgtaccgggcgcctcgggggacgcgcggcgatgagacgagcggctcattcgccatcgggatagtcccgcgcgaagccgctcgcggaggccggatcggtggcggcacccgtgggaggagcgggagacggcggcgttctggagagaggggccgctggggcgcccggaggccccatgggggttggagtgtatgtaggatgcgagccaatccttgaaggaccgttggcgtgcaccttgggggctgaggttagctgccacatgaccagcaggtcgctgtctgcgggactcatccatccttcggccaggtcgccgtctccccacagagaagcgttggtcgctgcctcctcgagttgctcctcctggtccgcaagacgatcgtccacggcgtccaggcgctcaccaagcgccggatcgaggtaccgtcggtgtgcggttagaaagtcacgacgcgccgcttgctcctccacgcgaattttaacacaggtcgcgcgctgtcgcatcatctctaagcgcgcgcgggactttagccgcgcctccaattccaagtgggccgcctttgcagccataaaggcgccaacaaaccgaggatcttgggtgctgacgccctcccggtgcagctgcagggtctggtccttgtaaatctcggctcggaggtgcgtctcggccaggcgtcggcgcagggccgcgtgggcggcatctcggtccattccgccaccctgcgggcgacccgggggtgctctgatagtctcgcgtgcccaaggcccgtgatcggggtacttcgccgccgcgacccgccacccggtgtgcgcgatgtttggtcagcagctggcgtccgacgtccagcagtacctggagcgcctcgagaaacagaggcaacttaaggtgggcgcggacgaggcgtcggcgggcctcacaatgggcggcgatgccctacgagtgccctttttagatttcgcgaccgcgacccccaagcgccaccagaccgtggtcccgggcgtcgggacgctccacgactgctgcgagcactcgccgctcttctcggccgtggcgcggcggctgctgtttaatagcctggtgccggcgcaactaaaggggcgtgatttcgggggcgaccacacggccaagctggaattcctggcccccgagttggtacgggcggtggcgcgactgcggtttaaggagtgcgcgccggcggacgtggtgcctcagcgtaacgcctactatagcgttctgaacacgtttcaggccctccaccgctccgaagcctttcgccagctggtgcactttgtgcgggactttgcccagctgcttaaaacctccttccgggcctccagcctcacggagaccacgggcccccccaaaaaacgggccaaggtggacgtggccacccacggccggacgtacggcacgctggagctgttccaaaaaatgatccttatgcacgccacctactttctggccgccgtgctcctcggggaccacgcggagcaggtcaacacgttcctgcgtctcgtgtttgagatccccctgtttagcgacgcggccgtgcgccacttccgccagcgcgccaccgtgtttctcgtcccccggcgccacggcaagacctggtttctggtgcccctcatcgcgctgtcgctggcctcctttcgggggatcaagatcggctacacggcgcacatccgcaaggcgaccgagccggtgtttgaggagatcgacgcctgcctgcggggctggttcggttcggcccgagtggaccacgttaaaggggaaaccatctccttctcgtttccggacgggtcgcgcagtaccatcgtgtttgcctccagccacaacacaaacgtaagtcctcttttctttcgcatggctctcccaaggggccccgggtcgacccgacccacacccacccacccacatacacacacaaccagacgcgggaggaaagtctgccccgtgggcactgatttttattcgggatcgcttgaggaggcccgggcaacggcccgggcaacggtggggcaactcgtagcaaataggcgactgatgtacgaagagaagacacacaggcgccacccggcgctggtcggggggatgttgtccgcgccgcaccgtcccccgacgacctcttgcagacggtccgtgatgcaaggacggcggggggcctgcagcagggtgaccgtatccacgggatggccaaagagaagcggacacaggctagcatccccctggaccgccagggtacactgggccatcttggcccacagacacggggcgacgcagggacaggactccgttacgacggaggagagccacagtgcgttggcggaatcgatgtggggcggcggggcgcaggactcgcagccccccgggtggttagtgatcctggccaggagccatcccagatggcgggccctgcttcccggtggacagagcgaccccaggtcgctgtccatggcccagcagtagatctggccgctggggaggtgccaccaggcccccgggcccaaggcgcagcacgcgcccggctccgggggggtcttcgcggggaccagatacgcgccatccagctcgccgaccactggctcctccgcgagctgttcggtggttgggtcgggggtttcctccgggggggtggccgcccgtatgcgggcgaacgtgagggtgcacaggagcggggtcagggggtgcgtcacgctccggaggtggacgatcgcgcagtagcggcgctcgcggttaaagaaaaagagggcaaagaaggtgttcgggggcaaccgcagcgccttggggcgcgtcagatacagaaaaatctcgcagaagagggcgcgcccggggtctgggttaggaagggccacctgacacagaggctcggtgaggaccgttagacaccgaaagatcttgagccgctcgtccgcccgaacgacgcgccacacaaagacggagttgacaatgcgcgcgatagagtcgacgtccgtccccaggtcgtcgactctgtcgcgcgtgccgcgagctccggcccgggaatccggccggggcaaggtccccgggggaccaggcggcgccaggggccgccggggtcccagctgcgccatgccgggggcggggggagggcaaaccccagaggcgggggccaacggcgcggggaggagtggatgggcgaggtggccgggggaaggcgcccgctagcgagaacggccgttcccggacgacaccttgcgacaaaacctaaggacagcggcccgcgcgacggggtccgagaggctaaggtaggccgcgatgttaatggtgaacgcaaagccgccgggaaagacaactatgccacagaggcggcgattaaaccccaggcagaggtaggcgtagctttccccgggcaggtattgctcgcagaccctgcgtggggctgtggaggggacggcctccatgaagcgacatttactctgctcgcgtttactgacgtcaccatccatcgccacggcgattggacgattgttaagccgcagcgtgtctccgcttgtgctgtagtagtcaaaaacgtaatggccgtcggagtcggcaaagcgggccgggaggtcgtcgccgagcgggacgacccgccgcccccgaccgccccgtccccccaggtgtgccaggacggccagggcatacgcggtgtgaaaaaaggcgtcgggggcggtcccctcgacggcgcgcatcaggttctcgaggagaatggggaagcgcctggtcacctcccccagccacgcgcgttggtcggggccaaagtcatagcgcaggcgctgtgagattcgcgggccgccctgaagcgcggcccggatggcctggcccagggcccggaggcacgccagatgtatgcgcgcggtaaaggcgacctcggcggcgatgtcaaagggcggcaggacggggcgcgggtggcgcaggggcacctcgagcgcgggaaagcggagcagcagctccgcctgcccagcgggagacagctggtgggggcgcacgacgcgttctgcggcgcaggcctcggtcagggccgtggccagcgccgaggacagcagcggagggcgggcgcgtcgcccgccccacgccactgagttctcgtaggagacgacgacgaagcgctgcttggttccgtagtggtggcgcaggaccacggagatagaacgacggctccacagccagtccggccggtcgccgccggccagggcttcccatccgcgatccaaccactcgaccagcgaccgcggctttgtggtaccaggggtaagggttagaacgtcgttcaggatgtcctcgcccccgggcccgtggggcgctggggccacaaagcggcccccgccggggggctccagacccgccagcaccgcatctgcgtcagccgcccccatggcgcccccgctgacggcctggtgaaccagggcgccctggcggagccccgatgcaacgccacaggccgcacgcccggtccgagcgcggaccgggtggcggcgggtgacgtcctgcactgcccgctgaaccaacgcgaggatctcctcgttctcctgtgcgatggacacgtcctgggccgcggtcgtgtcgccgccgggggccgtcagctgctcctccggggagatgggggggtcggacgccccgacgatgggcgggtctgcgggcgcccccgcgtggggccgggccaagggctgcggacgcggggacgcgctttcccccagacccatggacaggtgggccgcggcctccttcgcggccggcggggcggcggcgccaagcagagcgacgtagcggcacaaatgccgacagacgcgcatgatgcgcgtgctgtcggccgcgtagcgcgtgttgggggggacgagctcgtcgtaactaaacagaatcacgcgggcacagctcgcccccgagccccacgcgaggcgcagcgccgccacggcgtacgggtcatagacgccctgcgcgtcacacaccacgggcagggagacgaacaaccccccggcgctggacgcacgcggaaggaggccagggtgtgccggcacgacgggggccagaagctcccccaccgcatccgcgggcacgtaggcggcaaacgccgtgcaccacggggtacagtcgccggtggcatgagcccgagtctggatttcgacctggaagtttgcggccgtcccgagtccggggcggccgcgcatcagggcggccagagggattcccgcggccgccaggcactcgctggatatgatgacgtgaaccaaagacgagggccgacccgggccgtggccgagatcgtactggacctcgttggccaagtgcgcgttcatggttcggggtgggtgtgggtgtgtaggcgatgcgggtcccccgagtccgcgggaagggcgtgggtttggcgcgcgtatgcgtattcgccaacggaggcgtgcgtgcttatgcgcggcgcgtttcttctgtctccagggaatccgaggccaggactttaacctgctctttgtcgacgaggccaactttattcgcccggatgcggtccagacgattatgggctttctcaaccaggccaactgcaagattatcttcgtgtcgtccaccaacaccgggaaggccagtacgagctttttgtacaacctccgcggggccgccgacgagcttctcaacgtggtgacctatatatgcgatgatcacatgccgagggtggtgacgcacacaaacgccacggcctgttcttgttatatcctcaacaagcccgttttcatcacgatggacggggcggttcgccggaccgccgatttgtttctggccgattccttcatgcaggagatcatcgggggccaggccagggagaccggcgacgaccggcccgttctgaccaagtctgcgggggagcggtttctgttgtaccgcccctcgaccaccaccaacagcggcctcatggcccccgatttgtacgtgtacgtggatcccgcgttcacggccaacacccgagcctccgggaccggcgtcgctgtcgtcgggcggtaccgcgacgattatatcatctttgccctggagcacttttttctccgcgcgctcacgggctcggcccccgccgacatcgcccgctgcgtcgtccacagtctgacgcaggtcctggccttgcatcccggggcgtttcgcggcgtccgggtggcggtcgagggaaatagcagccaggactcggccgtcgccatcgccacgcacgtgcacacagagatgcaccgcctactggcctcggagggggccgacgcgggctcgggccccgagcttctcttctaccactgcgagcctcccgggagcgcggtgctgtacccctttttcctgctcaacaaacagaagacgcccgcctttgaacactttattaaaaagtttaactccgggggcgtcatggcctcccaggagatcgtttccgcgacggtgcgcctgcagaccgacccggtcgagtatctgctcgagcagctgaataacctcaccgaaaccgtctcccccaacactgacgtccgtacgtattccggaaaacggaacggcgcctcggatgaccttatggtcgccgtcattatggccatctaccttgcggcccaggccggacctccgcacacattcgctcccatcacacgcgtttcgtgagcgcccaataaacacacccaggtatgctacgcacgaccacggtgtcgcctgttaaggggggggaagggggtgttggcgggaagcgtgggaacacgggggattctctcacgaccggcaccagtaccacccccctgtgaacacagaaacccaacccaaatcccataaacatacgacacacaggcatattttggaatttcttgggtttttatttatttaggtatgctggggtttctccctggatgcccaccccccacccccccccgtgggtctagccgggccttagggatagcgtataacgggggccatgtctccggaccgcacaacggccgcgccgtcaaaggtgcacacccgaaccacgggagccagggccaaggtgtctcctagttggcccgcgtgggtcagccaggcgacgagcgcctcgtaaagcggcagccttcgctctccatcctgcaccagggccggggcttcggggtgaatgagctgggcggcctcccgcgtgacactctgcatctgcaggagagcgttcacgtacccgtcctgggcacttagcgcaaagagccgggggattagcgtaaggatgatggtggttccctccgtgatcgagtaaaccatgttaaggaccagcgatcgcagctcggcgtttacgggaccgagttgttggacgtccgccagcagcgagaggcgactcccgttgtagtacagcacgttgaggtctggcagccctccggggtttctggggctggggttcaggtcccggatgcccctggccacgagccgcgccacgatttcgcgcgccaggggcgatggaagcggaacgggaaaccgcaacgtgaggtccagcgaatccaggcgcacgtccgtcgcttggccctcgaacacgggcgggacgaggctgatggggtccccgttacagagatctacgggggaggtgttgcgaaggttaacggtgccggcgtgggtgaggcccacgtccaggggacaggcgacgattcgcgtgggaagcacccgggtgatgaccgcggggaagcgccttcggtacgccagcaacaaccccaacgtgtcgggactgacgcctccggagacgaaggattcgtgcgccacgtcggccagcgtcagttgccggcggatggtcggcaggaataccacccgcccttcgcagcgctgcagcgccgccgcatcggggcgcgagatgcccgagggtatcgcgatgtcagtttcaaagccgtccgccagcatggcgccgatccacgcggcagggagtgcagtggtggttcgggtggcgggaggagcgcggtgggggtcagcggcgtagcagagacgggcgaccaacctcgcataggacggggggtgggtcttagggggttgggaggcgacagggaccccagagcatgcgcggggaggtctgtcgggcccagacgcaccgagagcgaatccgtccacggagtcccggtctgggttttatggggcccggccctcggaatcgcggcttgtcggcggggacaaagggggcggggctagggggcttgcggaaacagaagacgcgtgggataaaagaatcgcactaccccaaggaagggcggggcggtttattacagagccagtcccttgagcggggatgcgtcatagacgagatactgcgcgaagtgggtctcccgcgcgtgggcttccccgttgcgggcgctgcggaggagggcggggtcgctggcgcaggtgagcgggtaggcctcctgaaacaggccacacgggtcctccacgagttcgcggcaccccggggggcgcttaaactgtacgtcgctggcggcggtggccgtggacaccgccgaacccgtctccacgatcaggcgctccaggcagcgatgtttggcggcgatgtcggccgacgtaaagaacttaaagcaggggctgagcaccggcgaggccccgttgaggtggtaggccccgttatagagcaggtccccgtacgaaaatcgctgcgacgcccacgggttggccgtggccgcaaaggcccgggacgggtcgctctggccgtggtcgtacatgagggcggtgacatccccctccttgtcccccgcgtaaacgcccccggcggcgcgtccccgggggttgcagggccggcggaagtagttgacgtcggtcgacacgggggtggcgataaactcacacacggcgtcctggccgtggtccatccctgcgcgccgcggcacctgggcgcacccgaacacggggacgggctgggccggccccaggcggtttcccgccacgaccgcgttccgcaggtacacggctgccgcgttgtccaggagagggggagccccgcggcccaggtaaaagttttggggaaggttgcccatgtcggtgacggggttgcggacggttgccgtggccacgacggcggtgtagcccacgcccaggtccacgttcccgcgcggctgggtgagcgtgaagtttacccccccgccagtttcgtgccgggccacctggagctggcccaggaagtacgcctccgacgcgcgctccgagaacagcatgttctcagtcacaaagcggtcctgtcggacgacggtgaacccaaacccgggatggaggcccgtcttgagctgatgatgcaaggccacgggactgatcttgaagtaccccgccatgagcgcgtaggtcagcgcgttctccccggccgcgctctcgcggacgtgctgcacgacgggctgtcggatcgacgaaaagtagttggcccccagagccggggggaccagggggacctgccgcgacaggtcgcgcagggccggggggaaattgggcgcgttcgccacgtggtcggccccggcgaacagcgcgttgacgggaagggggtaaaaatagtcgccattttggatggtatggtccagatgctggggggccatcagcaggattccggcgtgcaacgccccgtcgaatatgcgcatgttggtggtggacgcggtgttggcgcccgcgtcgggcgccgccgagcagagcagcgccgttgtgcgttcggccatgttgtgggccagcacctgcagcgtgagcatggcgggcccgtccactaccacgcgcccgttgtgaaacatggcgttgaccgtgttggccaccagattggccgggtgcagggggtgcgcggggtccgtcacggggtcgctggggcactcctcgccgggggcgatctccgggaccaccatgttctgcagggtggcgtatacgcggtcgaagcgaacccccgcggtgcagcagcggccccgcgagaaggcgggcaccatcacgtagtagtaaatcttgtggtgcacggtccagtccgccccccggtgcggccggtcatccgcggcgtccgcggctcgggcctgggtgttgtgcagcagctggccgtcgttgcggttgaagtccgcggtcgccacgttacatgccgccgcgtacacggggtcgtggccccccgcgctaacccggcagtcgcgatggcggtccagggccgcgcgccgcatcagggcgtcacagtcccacacgaggggtggcagcagcgccgggtctcgcattaggtgattcagctcggcttgcgcctgcccgcccagctccgggccggtcagggtaaagtcatcaaccagctgggccagggcctcgacgtgcgccaccaggtcccggtacacggccatgcactcctcgggaaggtctcccccgaggtaggtcacgacgtacgagaccagcgagtagtcgttcacgaacgccgcgcaccgcgtgttgttccagtagctggtgatgcactggacaacgagccgggccagggcgcagaagacgtgctcgctgccgtgtatggcggcctgcagcaggtaaaacaccgccgggtagttgcggtcgtcgaacgccccgcgaacggcggcgatggtggcgggggccatggcgtggcgtcccacccccagctccaggccccgggcgtcccggaacgccgccggacatagcgccaggggcaagttgccgttcaccacgcgccaggtggcctggatctcccccgggccggccgggggaacgtccccccccggcagctccacgtcggccacccccacgaagaagtcgaacgcggggtgcagctcaagagccaggttggcgttgtcgggctgcataaactgctccggggtcatctggccttccgcgacccatcggacccgcccgtgggccaggcgctgcccccaggcgttcaaaaacagctgctgcatgtctgcggcggggccggccggggccgccacgtacgccccgtacggattggcggcttcgacggggtcgcggttaaggcccccgaccgccgcgtcaacgttcatcagcgaagggtggcacacggtcccgatcgcgtgttccagagacaggcgcagcacctggcggtccttcccccaaaaaaacagctggcggggcgggaaggcgcggggatccgggtggccgggggcggggactaggtccccggcgtgcgcggcaaaccgttccatgaccggattgaacaggcccaggggcaggacgaacgtcaggtccatggcgcccaccagggggtagggaacgttggtggcggcgtagatgcgcttctccagggcctccagaaagaccagcttctcgccgatggacaccagatccgcgcgcacgcgcgtcgtctggggggcgctctcgagctcgtccagcgtctgccggttcaggtcgagctgctcctcctgcatctccagcaggtggcggcccacgtcgtccagacttcgcacggccttgcccatcacgagcgccgtgaccaggttggccccgttcaggaccatctcgccgtacgtcaccggcacgtcggcttcggtgtcctccactttcaggaaggactgcaggaggcgctgtttgatcggggctgtggtgactagcaccccgtcgaccggccgcccgcgcgtgtcggcatgcgtcagacggggcacggccacggagggctgcgtggccgtggtgaggtccacgagccaggcctcgacggcctcccggcggtggcccgccttgcccaggaaaaagctcgtctcgcagaagcttcgctttagctcggcgaccagggtcgcccgggccaccctggtggccaggcggccgttgtccaggtatcgttgcatcggcaacaacaaagccaggggcggcgccttttccagcagcacgtgcagcatctggtcggccgtgccgcgctcaaacgccccgaggacggcctggacgttgcgagcgagctgttggatggcgcgcaactggcgatgcgcgctgatacccgtcccgtccagggcctcccccgtgagcagggcgatggcctcggtggccaggctgaaggcggcgttcagggcccggcggtcgataatcttggtcatgtaattgtgtgtgggttgctcgatggggtgcgggccgtcgcgggcaatcagcggctggtggacctcgaactgtacgcgcccctcgttcatgtaggccagctccggaaacttggtacacacgcacgccaccgacaacccgagctccagaaagcgcacgagcgacagggtgttgcaatacgaccccagcagggcgtcgaactcgacgtcgtacaggctgtttgcatcggagcgcacgcgggaaaaaaaatcgaacaggcgtcgatgcgacgccacctcgatcgtgctaaggagggacccggtcggcaccatggccgcggcataccggtatcccggagggtcgcggttgggagcggccatggggtcgcgtggagatcggctgtctctagcgatattggcccggggaggctaagatccaccccaacgcccggccacccgtgtacgtgcccgacggcccaaggtccaccgaaagacacgacggacccggacccaaagaggcgggggatgctgtgtgagaggccgggtgtcggtcgggggggaaaggcaccgggagaaggctgcggcctcgttccaggagaacccagtgtccccaacagacccggggacgtgggatccccggccttatatacccccccccgccccacccccgttagaacgcgacgggtgcattcaagatggccctggtccaaaagcgtgccaggaagaaattggcagaggcggcaaagctgtccgccgccgccacccacatcgaggccccggccgcacaggctatccccagggcccgtgtgcgcaggggatcggtgggtggcagcatttggttggtggcgataaagtggaaaagcccgtccggactgaaggtctcgtgggcggcggcgaacaaggcacacagggccgtgcctcccaaaaacacggacatcccccaaaacacgggcgccgacaacggcagacgatccctcttgatgttaacgtacaggaggagcgcccgcaccgcccacgtaacgtagtagccgacgatggcggccaggatacaggccggcgccaccacccttccggtcagcccgtaatacatgcccgctgccaccatctccaacggcttcaggaccaaaaacgaccaaaggaacagaatcacgcgctttgaaaagaccggctgggtatggggcggaagacgcgagtatgccgaactgacaaaaaaatcagaggtgccgtacgaggacaatgaaaactgttcctccagcggcagttctccctcctcccccccgaaggcggcctcgtcgaccagatctcgatccaccagaggaaggtcatcccgcatggtcatggggtgtgcggtggaggtggggagaccgaaaccgcaaagggtcgcttacgtcagcaggatcccgagatcaaagacacccgggttcttgcacaaacaccacccgggttgcatccgcggaggcgagtgttttgataaggccgttccgcgccttgatataacctttgatgttgaccacaaaacccggaatttacgcctacgccccaatgcccacgcaagatgaggtaggtaacccccccgtgggtgtgacgttgcgtttagttcattggaggccaaggggaaaaatggggtggggaggaaacggaaaacccagtaggccgtgtcgggaacacgcccggggttgtcctcaaaaggcagggtccatactacggaagccgtcgttgtattcgagacctgcctgtgcgacgcacgtcggggttgcctgtgtccggttcggcccccaccgcgtgcggcacgcacgaggacgagtccgcgtgctttattggcgttccaagcgttgccctccagtttctgttgtcggtgttcccccatacccacgcccacatccaccgtagggggcctctgggccgtgttacgtcgccgcccgcgatggagcttagctacgccaccaccatgcactaccgggacgttgtgttttacgtcacaacggaccgaaaccgggcctactttgtgtgcggggggtgtgtttattccgtggggcggccgtgtgcctcgcagcccggggagattgccaagtttggtctggtcgttcgagggacaggcccagacgaccgcgtggtcgccaactatgtacgaagcgaactccgacaacgcggcctgcaggacgtgcgtcccattggggaggacgaggtgtttctggacagcgtgtgtcttctaaacccgaacgtgagctccgagctggatgtgattaacacgaacgacgtggaagtgctggacgaatgtctggccgagtactgcacctcgctgcgaaccagcccgggtgtgctaatatccgggctgcgcgtgcgggcgcaagacagaatcatcgagttgtttgaacacccaacgatagtcaacgtttcctcgcactttgtgtataccccgtccccatacgtgttcgccctggcccaggcgcacctcccccggctcccgagctcgctggaggccctggtgagcggcctgtttgacggcatccccgccccacgccagccacttgacgcccacaacccgcgcacggatgtggttatcacgggccgccgcgccccacgacccatcgccgggtcgggggcggggtcggggggcgcgggcgccaagcgggccaccgtcagcgagttcgtgcaagtcaaacacattgaccgcgtgggccccgctggcgtttcgccggcgcctccgccaaacaacaccgactcgagttccctggtgcccggggcccaggattccgccccgcccggccccacgctaagggagctgtggtgggtgttttatgccgcagaccgggcgctggaggagccccgcgccgactctggcctcacccgcgaggaggtacgtgccgtacgtgggttccgggagcaggcgtggaaactgtttggctccgcgggggccccgcgggcgtttatcggggccgcgttgggcctgagccccctccaaaagctggccgtttactactatatcatccaccgagagaggcgcctgtcccccttccccgcgctagtccggctcgtaggccggtacacacagcgccacggcctgtacgtccctcggcccgacgacccagtcttggccgatgccatcaacgggctggttcgcgacgcgctggcggccggaaccacagccgagcagctcctcatgttcgaccttctccccccaaaggacgtgccggtgggaagcgacgtgcaggccgacagcaccgctctgctgcgctttatagaatcgcaacgtctcgccgtccccgggggggtgatctcccccgagcacgtcgcgtaccttggtgcgttcctgagcgtgctgtacgctggccgcgggcgcatgtccgcagcaacgcacaccgcgcggctgacaggggtgacctccctggtgctagcggtgggtgacgtggaccgtctttccgcgtttgaccgcggagcggcgggcgcggccagccgcacgcgggccgccgggtacctggatgtgcttcttaccgttcgtctcgctcgctccaaacacggacagtctgtgtaacagaccccaataaacgtatgtcgctaccacacccttgtgtgtcaatggacgcctctccgggggggaagggaaaacaaagaggggctgggggagcggcaccactggggcctgaacaaacaaacaaaccacagacacggttacagtttattcggtcgggcggataaacggccgaagccacgccccctttattcgcgtctccaaaaaaacgggacacttgtccggagaacctttaggatgccagccagggcggcggtaatcataaccacgcccagcgcagaggcggccagaaacccgggcgcaattgcggccacgggctgcgtgtcaaaggctagcaaatgaatgacggttccgtttggaaatagcaacaaggccgtggacggcacgtcgctcgaaaacacgctcggggcgccctccgtcggcccggcggcgatttgctgctgtgtgttgtccgtatccaccagcaacacagacatgacctccccggctggggtgtagcgcataaacacggcccccacgagccccaggtcgcgctggttttgggtgcgcaccagccgcttggactcgatatcccgggtggagccttcgcatgtcgcggtgaggtaggttaggaacagtgggcgtcggacgtcgacgccggtgagcttgtagccgatcccccggggcagaggggagtgggtgacgacgtagctggcgttgtgggtgatgggtaccaggatccgtggctcgacgttggcagactgccccccgcaccgatgtgaggcctcagggacgaaggcgcggatcagggcgttgtagtgtgcccagcgcgtcagggtcgaggcgaggccgtgggtctgctgggccaggacttcgaccggggtctcggatcgggtggcttgagccagcgcgtccaggataaacacgctctcgtctagatcaaagcgcagggaggccgcgcatggcgaaaagtggtccggaagccaaaagagggttttctggtggtcggcccgggccagcgcggtccggaggtcggcgttggtcgctgcggcgacgtcggacgtacacagggccgatgctatcagaaggctccggcgggcgcgttcccgctgcaccgccgaggggacgcccgccaagaacggctgccggaggacagccgaggcgtaaaatagcgcccggtggacgaccggggtggtcagcacgcggccccctagaaactcggcatacagggcgtcgatgagatgggctgcgctgggcgccactgcgtcgtacgccgaggggctatccagcacgaaggccagctgatagcccagcgcgtgtaatgccaagctctgttcgcgctccagaatctcggccaccaggtgctggagccgagcctctagctgcaggcgggccgtgggatccaagactgacacattaaaaaacacagaatccgcggcacagcccgcggccccgcgggcggccaacccggcaagcgcgcgcgagtgggccaaaaagcctagcaggtcggagaggcagaccgcgccgtttgcgtgggcggcgttcacgaaagcaaaacccgacgtcgcgagcagccccgttaggcgccagaagagaggggggcgcgggccctgctcggcgcccgcgtcccccgagaaaaactccgcgtatgcccgcgacaggaactgggcgtagttcgtgccctcctccgggtagccgcccacgcggcggagggcgtccagcgcggagccgttgtcggcccgcgtcagggaccctaggacaaagacccgataccgggggccgcccgggggcccgggaagagcccccggggggttttcgtccgcggggtccccgacccgatctagcgtctggcccgcggggaccaccatcacttccaccggagggctgtcgtgcatggatatcacgagccccatgaattcccgcccgtagcgcgcgcgcaccagcgcggcatcgcacccgagcaccagctcccccgtcgtccagatgcccacgggccacgtcgaggccgacggggagaaatacacgtacctacctggggatctcaacaggccccgggtggccaaccaggtcgtggacgcgttgtgcaggtgcgtgatgtccagctccgtcgtcgggtgccgccgggccccaaccggcggtcgggggggcggtgtatcacgcggcccgcttgggtggctcgccgtcgccacgttgtctccccgcgggaacgtcagggcctcggggtcagggacggccgaaaacgttacccaggcccgggaacgcagcaacacggaggcgactggattgtacaagagacccttaaggggggcgaccgaggggggaggctgggcggtcggctcgaccgtggtgggggcgggcaggctcgcgttcgggggccggccgagcaggtaggtcttcgggatgtaaagcagctggccggggtcccgcggaaactcggccgtggtgaccaatacaaaacaaaagcgctcctcgtaccagcgaagaaggggcagagatgccgtagtcaggtttagttcgtccggcggcgccagaaatccgcgcggtggtttttgggggtcgggggtgtttggcagccacagacgcccggtgttcgtgtcgcgccagtacatgcggtccatgcccaggccatccaaaaaccatgggtctgtctgctcagtccagtcgtggacctgaccccacgcaacgcccaaaataataacccccacgaaccataaaccattccccatgggggaccccgtccctaacccacggggcccgtggctatggcagggcttgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgggcaaacgtgcgcgccaggtcgcagatcgtcggtatggagccgggggtggtgacgtgggtctggaccatcccggaggtaagttgcagcagggcgtcccggcagccggcgggcgattggtcgtaatccaggataaagacgtgcatgggacggaggcgtttggccaagacgtccaaggcccaggcaaacacgttgtacaggtcgccgttgggggccagcaactcgggggcccgaaacagggtaaataacgtgtccccgatatggggtcgtgggcccgcgttgctctggggctcggcaccctggggcggcacggccgtccccgaaagctgtccccaatcctcccgccacgacccgccgccctgcagataccgcaccgtattggcaagcagcccgtaaacgcggcgaatcgcggccagcatagccaggtcaagccgctcgccggggcgctggcgtttggccaggcggtcgatgtgtctgtcctccggaagggcccccaacacgatgtttgtgccgggcaaggtcggcgggatgagggccacgaacgccagcacggcctggggggtcatgctgcccataaggtatcgcgcggccgggtagcacaggagggcggcgatgggatggcggtcgaagatgagggtgagggccgggggcggggcatgtgagctcccagcctcccccccgatatgaggagccagaacggcgtcggtcacggcataaggcatgcccattgttatctgggcgcttgtcattaccaccgccgcgtccccggccgatatctcaccctggtcgaggcggtgttgtgtggtgtagatgttcgcgattgtctcggaagcccccagcacctgccagtaagtcatcggctcgggtacgtagacgatatcgtcgcgcgaacccagggccaccagcagttgcgtggtggtggttttccccatcccgtgaggaccgtctatataaacccgcagtagcgtgggcattttctgctccaggcggacttccgtggcttcttgctgccggcgagggcgcaacgccgtacgtcggttgctatggccgcgagaacgcgcagcctggtcgaacgcagacgcgtgttgatggcaggggtacgaagccatacgcgcttctacaaggcgcttgccgaagaggtgcgggagtttcacgccaccaagatctgcggcacgctgttgacgctgttaagcgggtcgctgcagggtcgctcggtgttcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagctgcgccactccctgaagctcctgcagtccctcgcgcctccgggtgacaagatagtgtacctgtgccccgtcctggtgtttgtcgcccaacggacgctccgcgtcagccgcgtgacccggctcgtcccgcagaaggtctccggtaatatcaccgcagtcgtgcggatgctccagagcctgtccacgtatacggtccccatggagcctaggacccagcgagcccgtcgccgccgcggcggcgccgcccgggggtctgcgagcagaccgaaaaggtcacactctggggcgcgcgacccgcccgagtcagcggcccgccagttaccacccgccgaccaaacccccgcctccacggagggcgggggggtgcttaagaggatcgcggcgctcttctgcgtgcccgtggccaccaagaccaaaccccgagccgcctccgaatgagagtgtttcgttccttccccctccccccgcgtcagacaaaccctaaccaccgcttaagcggcccccgcgaggtccgaagactcatttggatccggcgggagccacccgacaacagcccccgggttttcccacgccagacgccggtccgctgtgccatcgcgccccctcatcccaccccccatcttgtccccaaataaaacaaggtctggtagttaggacaacgaccgcagttctcgtgtgttattttcgctctccgcctctcgcagatggacccgtactgcccatttgacgctctggacgtctgggaacacaggcgcttcatagtcgccgattcccgaaacttcatcacccccgagttcccccgggacttttggatgtcgcccgtctttaacctcccccgggagacggcggcggagcaggtggtcgtcctacaggcccagcgcacagcggctgccgctgccctggagaacgccgccatgcaggcggccgagctccccgtcgatatcgagcgccggttacgcccgatcgaacggaacgtgcacaagatcgcaggcgccctggaggcgctggagacggcggcggccgccgccgaagaggcggatgccgcgcgcggggatgagccggcgggtgggggcgacgggggggcgcccccgagtctggccgtcgcggagatggaggtccagatcgtgcgcaacgacccgccgctacgatacgacaccaacctccccgtggatctgctacacatggtgtacgcgggccgcggggcgaccggatcgtcgggggtggtgttcgggacctggtaccgcactatccaggaccgcaccatcacggactttcccctgaccacccgcagtgccgactttcgggacggccgtatgtccaagaccttcatgacggcgctggtactgtccctgcagtcgtgcggccggctgtatgtgggccagcgccactattccgccttcgagtgcgccgtgttgtgtctctacctgctgtaccgaaacacgcacggggccgccgacgatagcgaccgcgctccggtcacgttcggggatctgctgggccggctgccccgctacctggcgtgcctggccgcggtgatcgggaccgagggcggccggccacagtaccgctaccgcgacgacaagctccccaagacgcagttcgcggccggcgggggccgctacgaacacggagcgctggcgtcgcacatcgtgatcgccacgctgatgcaccacggggtgctcccggcggccccgggggacgtcccccgggacgcgagcacccacgttaaccccgacggcgtggcgcaccacgacgacataaaccgcgccgccgccgcgttcctcagccggggccacaacctattcctgtgggaggaccagactctgctgcgggcaaccgcgaacaccataacggccctgggcgttatccagcggctcctcgcgaacggcaacgtgtacgcggaccgcctcaacaaccgcctgcagctgggcatgctgatccccggagccgtcccttcggaggccatcgcccgtggggcctccgggtccgactcgggggccatcaagagcggagacaacaatctggaggcgctatgtgccaattacgtgcttccgctgtaccgggccgacccggcggtcgagctgacccagctgtttcccggcctggccgccctgtgtcttgacgcccaggcggggcggccggtcgggtcgacgcggcgggtggtggatatgtcatcgggggcccgccaggcggcgctggtgcgcctcaccgccctggaactcatcaaccgcacccgcacaaaccccacccccgtgggggaggttatccacgcccacgacgccctggcgatccaatacgaacaggggcttggcctgctggcgcagcaggcacgcattggcttgggctccaacaccaagcgtttctccgcgttcaacgttagcagcgactacgacatgttgtactttttatgtctggggttcattccacagtacctgtcggcggtttagtgggtggtgggcgaggggggagggggcattagggagaaagaacaagagcctccgttgggttttctttgtgcctgtactcaaaaggtcataccccgtaaacggcgggctccagtcccggcccggcggttggcgtgaacgcaacggcgggagctgggttagcgtttagtttagcattcgctctcgcctttccgcccgccccccgaccgttgcgccttttttttttttcgtccaccaaagtctctgtgggtgcgcgcatggcagccgatgccccgggagaccggatggaggagcccctgccagacagggccgtgcccatttacgtggctgggtttttggccctgtatgacagcggggactcgggcgagttggcattggatccggatacggtgcgtgcggccctgcctccggataacccactcccgattaacgtggaccaccgcgctggctgcgaggtggggcgggtgctggccgtggtcgacgacccccgcgggccgttttttgtgggactgatcgcctgcgtgcaactggagcgcgtcctcgagacggccgccagcgctgcgattttcgagcgccgcgggccgccgctctcccgggaggagcgcctgttgtacctgatcaccaactacctgccctcggtctccctggccacaaaacgcctggggggcgaggcgcaccccgatcgcacgctgttcgcgcacgtcgcgctgtgcgcgatcgggcggcgcctcggcactatcgtcacctacgacaccggtctcgacgccgccatcgcgccctttcgccacctgtcgccggcgtctcgcgagggggcgcggcgactggccgccgaggccgagctcgcgctgtccggacgcacctgggcgcccggcgtggaggcgctgacccacacgctgctttccaccgccgttaacaacatgatgctgcgggaccgctggagcctggtggccgagcggcggcggcaggccgggatcgccggacacacctacctccaggcgagcgaaaaattcaaaatgtggggggcggagcctgtttccgcgccggcgcgcgggtataagaacggggccccggagtccacggacataccgcccggctcgatcgctgccgcgccgcagggtgaccggtgcccaatcgtccgtcagcgcggggtcgcctcgcccccggtactgccccccatgaaccccgttccggcatcgggcaccccggcccccgcgccgcccggcgacgggagctacctgtggatcccggcctcccattacaaccagctcgtcgccggccacgccgcgccccaaccccagccgcattccgcgtttggtttcccggctgcggcgggggccgtggcctatgggcctcacggcgcgggtctttcccagcattaccctccccacgtcgcccatcagtatcccggggtgctgttctcgggacccagcccactcgaggcgcagatagccgcgttggtgggggccatagccgcggaccgccaggcgggcggtcagacggccgcgggagaccctggggtccgggggtcgggaaagcgtcgccggtacgaggcggggccgtcggagtcctactgcgaccaggacgaaccggacgcggactacccgtactaccccggggaggctcgaggcgggccgcgcggggtcgactctcggcgcgcggcccgccagtctcccgggaccaacgagaccatcacggcgctgatgggggcggtgacgtctctgcagcaggaactggcgcacatgcgggctcggaccagcgccccctatggaatgtacacgccggtggcgcactatcgccctcaggtgggggagccggaaccaacaacgacccacccggccctttgtcccccggaggccgtgtatcgccccccaccacacagcgccccctacggtcctccccagggtccggcgtcccatgcccccactcccccgtatgccccagctgcctgcccgccaggcccgccaccgcccccatgtccttccacccagacgcgcgcccctctaccgacggagcccgcgttcccccccgccgccaccggatcccaaccggaggcatccaacgcggaggccggggcccttgtcaacgccagcagcgcagcacacgtggacgttgacacggcccgcgccgccgatttgttcgtctctcagatgatgggggcccgctgattcgccccggtctttggtaccatgggatgtcttactgtatatctttttaaataaaccaggtaataccaaataagacccattggtgtatgttctttttttattgggaggcgcgggtaggcgggtagctttacaatgcaaaagccttcgacgtggaggaaggcgtggggggggaatcggcactgaccaagggggtccgttttgtcacgggaaaggaaagaggaaacaggccgcggacacccgggggagtttatgtgttcccttttctttcttcccacacacacaaaaggcgtaccaaacaaacaaaccaaaagatgcacatgcggtttaacacccgtggtttttatttacaacaaaccccccgtcacaggtcgtcctcgtcggcgtcaccgtctttgttgggaacttgggtgtagttggtgttgcggcgcttgcgcatgaccatgtcggtgaccttggcgctgagcagcgcgctcgtgcccttcttcttggccttgtgttccgtgcgctccatggcagacaccagggccatgtaccgtatcatctcccgggcctcggctagcttggcctcgtcaaagtcgccgccctcctcgccctccccggacgcgtccgggttggtggggttcttgagctccttggtggttagcgggtacagggccttcatggggttgctctgcagccgcatgacgtagcgaaaggcgaagaaagccgccgccaggccggccaggaccaacagacccacggccagcgccccaaaggggttggacatgaaggaggacacgcccgacacggccgataccacgccgcccacgatgcccatcaccaccttgccgaccgcgcgccccaggtcgcccatcccctcgaagaacgcgcccaggcccgcgaacatggcggcgttggcgtcggcgtggatgaccgtgtcgatgtcggcgaagcgcaggtcgtgcagctggttgcggcgctggacctccgtgtagtccagcaggccgctgtccttgatctcgtggcgggtgtacacctccagggggacaaactcgtgatcctccagcatggtgatgttgaggtcgatgaaggtgctgacggtggtgatgtcggcgcggctcagctggtgggagtacgcgtactcctcgaagtacacgtagcccccgccgaaggtgaagtagcgccggtgtcccacggtgcacggctcgatcgcatcgcgcgtcagccgcagctcgttgttctcccccagctgcccctcgaccaacgggccctggtcttcgtaccgaaagctgaccagggggcggctgtagcaggccccgggccgcgagctgatgcgcatcgagttttggacgatcacgttgtccgcggcgaccggcacgcacgtggagacggccatcacgtcgccgagcatccgcgcgctcacccgccggcccacggtggccgaggcgatggcgttggggttcagcttgcgggcctcgttccacagggtcagctcgtgattctgcagctcgcaccacgcgatggcaacgcggcccaacatatcgttgacatggcgctgtatgtggttgtacgtaaactgcagccgggcgaactcgatggaggaggtggtcttgatgcgctccacggacgcgttggcgctggccccgggcggcgggggcgtggggtttgggggcttgcggctctgctctcggaggtgttcccgcacgtacagctccgcgagcgtgttgctgagaaggggctggtacgcgatcagaaagcccccattggccaggtagtactgcggctggcccaccttgatgtgcgtcgcgttgtacctgcgggcgaagatgcggtccatggcgtcgcgggcgtccttgccgatgcagtcccccaggtccacgcgcgagagcgggtactcggtcaggttggtggtgaaggtggtggatatggcgtcggaggagaatcggaaggagccgccgtactcggagcgcagcatctcgtccacctcctgccacttggtcatggtgcagaccgacgggcgctttggcacccagtcccaggccacggtgaacttgggggtcgtgagcaggttccgggtggtcggcgccgtggcccgggccttggtggtgaggtcgcgcgcgtagaagccgtcaacctgcttgaagcggtcggcggcgtagctggtgtgttcggtgtgcgacccctcccggtagccgtaaaacggggacatgtacacaaagtcgccagtcgccagcacaaactcgtcgtacgggtacaccgagcgcgcgtccacctcctcgacgatgcagtttaccgtcgtcccgtaccggtggaacgcctccacccgcgaggggttgtacttgaggtcggtggtgtgccagccccggctcgtgcgggtcgcggcgttggccggtttcagctccatgtcggtctcgtggtcgtcccggtgaaacgcggtggtctccaggttgttgcgcacgtacttggccgtggaccgacagacccccttggcgttgatcttgtcgatcacctcctcgaaggggacgggggcgcggtcctcaaagatccccataaactgggagtagcggtggccgaaccacacctgcgaaacggtgacgtctttgtagtacatggtggccttgaacttgtacggggcgatgttctccttgaagaccaccgcgatgccctccgtgtagttctgaccctcgggccgggtcgggcagcggcgcggctgctcgaactgcaccaccgtggcgcccgtggggggtgggcacacgtaaaagtttgcatcggtgttctccgccttgatgtcccgcaggtgctcgcgcagggtggcgtggcccgcggcgacggtcgcgttgtcgccggcggggcgcggcggcggtgggtttttcggttttttgttcttcttcggtttcgtgtcccccgttggggcggggccaggggcgggcggcgccggagtggcaggtcccccgttcgccgcctgggtcgcggccgcgaccccaggcgtgccgggggaactcggagccgccgacgccaccaggacccccagcgtcaaccccaagagcgcccatacgacgaaccaccggcacccccgcgcgggggcgccctggcgcatggcgggactacgggggcccgtcgtgccccccgtcaggtagcctgggggcgaggtgctggaggaccgagtagaggatcgagaaaacgtctcggtcgtagaccacgaccgaccgggggccgatacagccgtcgggggcgctctcgacgatggccaccagcggacagtcggagtcgtacgtgagatatacgccgggcgggtaacggtaacgaccttcggaggtcgggcggctgcagtccgggcggcgcaactcgagctccccgcaccggtagaccgaggcaaagagtgtggtggcgataatcagctcgcgaatatatcgccaggcggcgcgctgagtgggcgttattccggaaatgccgtcaaaacagtaaaacctctgaaattcgctgacggcccaatcagcacccgagccccccgcccccatgatgaaccgggcgagctcctccttcaggtgcggcaggagccccacgttctcgacgctgtaatacagcgcggtgttggggggctgggcgaagctgtgggtggagtgatcaaagaggggcccgttgacgagctcgaagaagcgatgggtgatgctggggagcagggccgggtccacctggtgtcgcaggagagacgctcgcatgaaccggtgcgcgtcgaacacgcccggcgccgagcggttgtcgatgaccgtgcccgcgcccgccgtcagggcgcagaagcgcgcgcgcgccgcaaagccgttggcgaccgcggcgaacgtcgcgggcagcacctcgccgtggacgctgacccgcagcatcttctcgagctccccgcgctgctcgcggacgcagcgccccaggctggccaacgaccgcttcgtcaggcggtccgcgtacagccgccgtcgctcccgcacgtccgcggccgcttgcgtggcgatgtccccccacgtctcgggcccctgccccccgggcccgcggcgacggtcttcgtcctcgcccccgcccccgggagctcccaacccccgtgccccttcctctacggcgacacggtccccgtcgtcgtcggggcccgcgccgcccttgggcgcgtccgccgcgccccccgcccccatgcgcgccagcacgcgacgcagcgcctcctcgtcgcactgttcggggctgacgaggcgccgcaagagcggcgtcgtcaggtggtggtcgtagcacgcgcggatgagcgcctcgatctgatcgtcgggtgacgtggcctgaccgccgattattagggcgtccaccatatccagcgccgccaggtggctcccgaacgcgcgatcgaaatgctccgcccgccgcccgaacagcgccagttccacggccaccgcggcggtctcctgctgcaactcgcgccgcgccagcgcggtcaggttgctggcaaacgcgtccatggtggtctggccggcgcggtcgccggacgcgagccagaatcgcaattcgctgatggcgtacaggccgggcgtggtggcctgaaacacgtcgtgcgcctccagcagggcgtcggcctccttgcggaccgagtcgttctcgggcgacgggtggggctgcccgtcgccccccgcggtccgggccagcgcatggtccaacacggagagcgcccgcgcgcggtcggcgtccgacagcccggcggcgtggggcaggtaccgccgcagctcgttggcgtccagccgcacctgcgcctgctgggtgacgtggttacagatacggtccgccaggcggcgggcgatcgtcgccccctggttcgccgtcacacacagttcctcgaaacagaccgcgcaggggtgggacgggtcgctaagctccggggggacgataaggcccgaccccaccgcccccaccataaactcccgaacgcgctccagcgcggcggtggcgccgcgcgagggggtgatgaggtggcagtagtttagctgctttagaaagttctcgacgtcgtgcaggaaacacagctccatatggacggtcccgccatacgtatccagcctgacccgttggtgatacggacagggtcgggccaggcccatggtctccgtgaaaaacaccgcgacgtctcccgcggtcgcgaacgtctccaggctgcccaggagccgctcgccctcgcgccacgcgtactctagcagcaactccagggtgaccgacagcggggtgagaaaggccccggcctgggcctccaggcccggcctcagacgacgccgcagcgcccgcacctgaagcgcgttcagcttcagttgggggagcttcccccgtccgatgtgggggtcgcaccgccggagcagctctatctgaaacacataggtctgcacctgtccgagcagggctaacaacttttgacgggccacggtgggctcggacaccggggcggccatctcgcggcgccgatctgtaccgcggccggagtatgcggtggaccgaggcggtccgtacgctacccggcgtctggctgagccccggggtccccctattcggggcggcctcccgcgggcccgccgaccggcaagccgggagtcggcggcgcgtgcgtttctgttctattcccagacaccgcggagaggaatcacggcccgcccagagatatagacacggaacacaaacaagcacggatgtcgtagcaataatttattttacacacattccccgccccgccctaggttcccccaccccccaacccctcacagcatatccaacgtcaggtctccctttttgtcggggggcccctccccaaacgggtcatccccgtggaacgcccgtttgcggccggcaaatgccggtcccggggcccccgggccgccgaacggcgtcgcgttgtcgtcctcgcagccaaaatccccaaagttaaacacctccccggcgttgccgagttggctgactagggcctcggcctcgtgcgccacctccagggccgcgtccgtcgaccactcgccgttgccgcgctccagggcacgtgcggtcagctccatcatctcctcgcttaggtactcgtcctccaggagcgccagccagtcctcgatctgcagctgttgggtgcggggccccaggcttttcacggtcgccacgaacacgctactggcgacggccgccccgccctcggagataatgccccggagctgctcgcacagcgagctttcgtgcgctccgccgccgaggctcgaggccgcgcacacaaacccggcccggggacaggccaggacgaacttgcgggtgcggtcaaaaataaggagcgggcacgcgtttttgccgcccatcaggctggcccagttcccggcctgaaacacacggtcgttgccggccatgccgtagtatttgctgatgctcaaccccaacacgaccatggggcgtgccgccatgacgggccgcagcaggttgcagctggcgaacatggaggtccacgcgcccggatgcgcgtccacggcgtccatcagcgcgcgggccccggcctccaggcccgccccgccctgcgcggaccacgcggccgccgcctgcacgctggggggacggcgggaccccgcgatgatggccgtgagggtgttgatgaagtacgtcgagtgatcgcagtaccgcagaatctggtttgccatgtagtacatcgccagctcgctcacgttgttgggggccaggttaataaagttgatcgcgccgtagtccagggaaaactttttaatgaacgcgatggtctcgatgtcctcgcgcgacaggagccgggcgggaagctggttgcgttggagggccgtccagaaccactgcgggttcggctggttggaccccgggggcttgccgttggggaagatggccgcgtggaactgcttcagcagaaagcccagcggtccgaggaggatgtccacgcgcttgtcgggcttctggtaggcgctctggaggctggcgacccgcgccttggcggcctcggacgcgttggcgctcgcgcccgcgaacaacacgcggctcttgacgcgcagctccttgggaaaccccagggtcacgcgggcaacgtcgccctcgaagctgctctcggcgggggccgtctggccggccgtcaggctgggggcgcagatagccgcaccctccgagagcgcgaccgtcagcgttttggccgacagaaacccgttgttaaacatgtccatcacgcgccgccgcagcaccggttggaattgattgcgaaagttgcgcccctcgaccgactgcccggcgaacaccccgtggcactggctcagggccaggtcctggtacacggcgaggttggatcgccgcccgagaagctgaagcagggggcacggcccgcacgcgtacgggtccagcgtcagggacatggcgtggttggcctcgcccagaccgtcgcgaaacttgaagttcctcccctccaccaggttgcgcatcagctgctccacctcgcggtccacgacctgcctgacgttgttcaccaccgtatgcagggcctcgcggttggtgatgatggtctccagccgccccatggccgtggggaccgcctggtccacgtactgcagggtctcgagttcggccatgacgcgctcggtcgccgcgcggtacgtctcctgcatgatggtccgggcggtctcggatccgtccgcgcgcttcagggccgagaaggcggcgtagtttcccagcacgtcgcagtcgctgtacatgctgttcatggtcccgaagacgccgatggctccgcgggcggcgctggcgaacttgggatggcgcgcccggaggcgcatgagcgtcgtgtgtacgcaggcgtggcgcgtgtcgaaggtgcacaggttacagggcacgtcggtctggttggagtccgcgacgtatcgaaacacgtccatctcctggcgcccgacgatcacgccgccgtcgcagcgctccaggtaaaacagcatcttggccagcagcgccggggaaaacccacacagcatggccaggtgctcgccggcaaattcctgggttccgccgacgaggggcgcggtgggccgaccctcgaacccgggcaccacgtgtccctcgcggtccacctgtgggttggccgccacgtgggtcccgggcacgaggaagaagcggtaaaaggagggtttgctgtggtcctttgggtccgccgggccggcgtcgtccacctcggtgagatggagggccgagttggtgctaaataccatggcccccacgagtcccgcggcgcgcgccaggtacgccccgacggcgttggcgcgggccgcggccgtgtcctggccctcgaacagcggccacgcggagatgtcggtgggcggctcgtcaaagacggccatcgacacgatagactcgagggccagggcggcgtctccggccatgacggaggccaggcgctgttcgaacccgcccgcagggcccttgccgccgccgtcgcgcccgccccgcggggtcttaccctggctggcttcgaaggccgtgaacgtaatgtcggcggggagggcggcgccctcgtggttttcgtcaaacgccaggtgggcggccgcgcgggccacggcgtccacgtttcggcatcgcagtgccacggcggcgggtcccacgaccgcctcgaacaggaggcggtggagggggcggttaaaaaacggaagcgggtaggtaaaattctccccgatcgatcggtggttggcgttgaacggctctgcgatgacacggctaaaatccggcatgaacagctgcaacgggtacacgggtatgcggtgcacctccgccccgcctatggttaccttgtccgagcctcccaggtgcagaaaggtgttgttgatgcacacggcctccttgaagccctcggtaacgaccagatacaggagggcgcggtccgggtccaggccgaggcgctcacacagcgcctcccccgtcgtctcgtgtttgaggtcgccgggccggggggtgtagtccgaaaagccaaaatggcggcgtgcccgctcgcagagtcgcgtcaggttcggggcctgggtgctggggtccaggtgccggccgccgtgaaagacgtacacggacgagctgtagtgcgagggcgtcagtttcagggacaccgcggtacccccgagccccgtcgtgcgagaacccacgaccacggccacgttggcctcaaagccgctctccacggtcaggcccacgaccaggggcgccacggcgacgtcggcatcgccgctgcgcgccgacagtaacgccagaagctcgatgccttcggacggacacgcgcgagcgtacacgtatcccaggggcccgggggggaccttgatggtggttgccgtcttgggctttgtctccatgtccttctgtcaatcggtccgcgaacggaggtaatcccggcacgacgacggacgcccgacaaggtatgtctcccgagcgtcaaaatccgggggggggggcggcgacggtcaaggggagggttggagaccggggttggggaatgaatccctacccttcaccgacaaccccccgggtaatcacggggtgccgatgaaccccggcggccggcaacgcggggtccctgcgagaggcacagatgcttacggtcaggtgctccgggtcgggtgcgtctggtatgcggttggtatatgtacactttacctgggggcgtgcctggccgccccagcccctcccacgccctgcgcgtcatcagccggtgggcgtggccgctattataaaaaaagtgagaacgcgaagcgttcgcactttgtcctaataatatatatattattaggacaaagtgcgaacgcttcgcgttctcactttttttataatagcggccacgcccaccggctacgtcacgctcctgtcggccgccggcggtccataagcccggccggccgggccgacgcgaataaaccgggccgccggccggggcgccgcgcagcagctcgccgcccggatccgccagacaaacaaggcccttgcacatgccggcccgggcgagcctgggggtccggtaattttgccatcccacccaagcggctttttgggtttttctcttcccccctccccacatcccccctctttaggggttcgggtggtaacaaccgcgatgttttccggtggcggcggcccgctgtcccccggaggaaagtcggcggccagggcggcgtccgggttttttgcgcccgccggccctcgcggagccggccggggacccccgccttgcttgaggcaaaacttttacaacccctacctcgccccagtcgggacgcaacagaagccgaccgggccaacccagcgccatacgtactatagcgaatgcgatgaatttcgattcatcgccccgcgggtgctggacgaggatgcccccccggagaagcgcgccggggtgcacgacggtcacctcaagcgcgcccccaaggtgtactgcgggggggacgagcgcgacgtcctccgcgtcgggtcgggcggcttctggccgcggcgctcgcgcctgtggggcggcgtggaccacgccccggcggggttcaaccccaccgtcaccgtctttcacgtgtacgacatcctggagaacgtggagcacgcgtacggcatgcgcgcggcccagttccacgcgcggtttatggacgccatcacaccgacggggaccgtcatcacgctcctgggcctgactccggaaggccaccgggtggccgttcacgtttacggcacgcggcagtacttttacatgaacaaggaggaggtcgacaggcacctacaatgccgcgccccacgagatctctgcgagcgcatggccgcggccctgcgcgagtccccgggcgcgtcgttccgcggcatttccgcggaccacttcgaggcggaggtggtggagcgcaccgacgtgtactactacgagacgcgccccgctctgttttaccgcgtctacgtccgaagcgggcgcgtgctgtcgtacctgtgcgacaacttctgcccggccatcaagaagtacgagggtggggtcgacgccaccacccggttcatcctggacaaccccgggttcgtcaccttcggctggtaccgtctcaaaccgggccggaacaacacgctagcccagccgcgggccccgatggccttcgggacatccagcgacgtcgagtttaactgtacggcggacaacctggccatcgaggggggcatgagcgacctaccggcatacaagctcatgtgcttcgatatcgaatgcaaggcggggggggaggacgagctggcctttccggtggccgggcacccggaggacctggtcatccagatatcctgtctgctctacgacctgtccaccaccgccctggagcacgtcctcctgttttcgctcggttcctgcgacctccccgaatcccacctgaacgagctggcggccaggggcctgcccacgcccgtggttctggaattcgacagcgaattcgagatgctgttggccttcatgacccttgtgaaacagtacggccccgagttcgtgaccgggtacaacatcatcaacttcgactggcccttcttgctggccaagctgacggacatttacaaggtccccctggacgggtacggccgcatgaacggccggggcgtgtttcgcgtgtgggacataggccagagccacttccagaagcgcagcaagataaaggtgaacggcatggtgaacatcgacatgtacgggattataaccgacaagatcaagctctcgagctacaagctcaacgccgtggccgaagccgtcctgaaggacaagaagaaggacctgagctatcgcgacatccccgcctactacgccgccgggcccgcgcaacgcggggtgatcggcgagtactgcatacaggattccctgctggtgggccagctgttttttaagtttttgccccatctggagctctcggccgtcgcgcgcttggcgggtattaacatcacccgcaccatctacgacggccagcagatccgcgtctttacgtgcctgctgcgcctggccgaccagaagggctttattctgccggacacccaggggcgatttaggggcgccgggggggaggcgcccaagcgtccggccgcagcccgggaggacgaggagcggccagaggaggagggggaggacgaggacgaacgcgaggagggcgggggcgagcgggagccggagggcgcgcgggagaccgccggccggcacgtggggtaccagggggccagggtccttgaccccacttccgggtttcatgtgaaccccgtggtggtgttcgactttgccagcctgtaccccagcatcatccaggcccacaacctgtgcttcagcacgctctccctgagggccgacgcagtggcgcacctggaggcgggcaaggactacctggagatcgaggtgggggggcgacggctgttcttcgtcaaggctcacgtgcgagagagcctcctcagcatcctcctgcgggactggctcgccatgcgaaagcagatccgctcgcggattccccagagcagccccgaggaggccgtgctcctggacaagcagcaggccgccatcaaggtcgtgtgtaactcggtttacgggttcacgggagtgcagcacggactcctgccgtgcctgcacgttgccgcgacggtgacgaccatcggccgcgagatgctgctcgcgacccgcgagtacgtccacgcgcgctgggcggccttcgaacagctcctggccgatttcccggaggcggccgacatgcgcgcccccgggccctattccatgcgcatcatctacggggacacggactccatctttgtgctgtgccgcggcctcacggccgccgggctgacggccgtgggcgacaagatggcgagccacatctcgcgcgcgctgtttctgtcccccatcaaactcgagtgcgaaaagacgttcaccaagctgctgctgatcgccaagaaaaagtacatcggcgtcatctacgggggtaagatgctcatcaagggcgtggatctggtgcgcaaaaacaactgcgcgtttatcaaccgcacctccagggccctggtcgacctgctgttttacgacgataccgtatccggagcggccgccgcgttagccgagcgccccgcagaggagtggctggcgcgacccctgcccgagggactgcaggcgttcggggccgtcctcgtagacgcccatcggcgcatcaccgacccggagagggacatccaggactttgtcctcaccgccgaactgagcagacacccgcgcgcgtacaccaacaagcgcctggcccacctgacggtgtattacaagctcatggcccgccgcgcgcaggtcccgtccatcaaggaccggatcccgtacgtgatcgtggcccagacccgcgaggtagaggagacggtcgcgcggctggccgccctccgcgagctcgacgccgccgccccaggggacgagcccgccccccccgcggccctgccctccccggccaagcgcccccgggagacgccgttgcatgccgaccccccgggaggcgcgtccaagccccgcaagctgctggtgtccgagctggccgaggatcccgcatacgccattgcccacggcgtcgccctgaacacggactattacttctcccacctgttgggggcggcgtgcgtgacattcaaggccctgtttgggaataacgccaagatcaccgagagtctgttaaaaaggtttattcccgaagtgtggcaccccccggacgacgtggccgcgcggctccgggccgcagggttcggggcggtgggtgccggcgctacggcggaggaaactcgtcgaatgttgcatagagcctttgatactctagcatgagccccccgtcgaagctgatgtccctcattttacaataaatgtctgcggccgacacggtcggaatctccgcgtccgtgggtttctctgcgttgcgccggaccacgagcacaaacgtgctctgccacacgtgggcgacgaaccggtaccccgggcacgcggtgagcatccggtctatgagccggtagtgcaggtgggcggacgtgccgggaaagatgacgtacagcatgtggcccccgtaagtggggtccgggtaaaacaacagccgcgggtcgcacgccccgcctccgcgcaggatcgtgtggacgaaaaaaagctcgggttggccaagaatcccggccaagaggtcctggaggggggcgttgtggcggtcggccaacacgaccaaggaggccaggaaggcgcgatgctcgaatatcgtgttgatctgctgcacgaaggccaggattagggcctcgcggctggtggcggcgaaccgcccgtctcccgcgttgcacgcgggacagcaacccccgatgcctaggtagtagcccatcccggagagggtcaggcagttgtcggccacggtctggtccagacagaagggcagcgagacgggagtggtcttcaccaggggcaccgagagcgagcgcacgatggcgatctcctcggagggcgtctgggcgagggcggcgaaaaggccccgatagcgctggcgctcgtgtaaacacagctcctgtttgcgggcgtgaggcggcaggctcttccgggaggcccgacgcaccacgcccagagtcccgccggccgcagaggagcgcgaccgccggcgctccttgccgtgatagggcccgggccgggagccgcggcgatgggggtcggtgtcatacataggtacacagggtgtgctccagggacaggagcgagatcgagtggcgtctaagcagcgcgcccgcctcacggacaaatgtggcgagcgcggtgggctttggtacaaatacctgatacgtcttgaaggtgtagatgagggcacgcaacgctatgcagacacgcccctcgaactcgttcccgcaggccagcttggccttgtggagcagcagctcgtcgggatgggtggcggggggatggccgaacagaacccaggggtcaacctccatctccgtaatggcgcacatggggtcacagaacatgtgcttaaagatggcctcgggccccgcggcccgaagcaggctcacaaaccggcccccgtccccgggctgcgtctcggggtcagcctcgagctggtcgacgacgggtacgatacagtcgaagaggctcgtgttgttttccgagtagcggaccacggaggcccggagtctgcgcagggccagccagtaagcacgcaccagtaacaggttacacagcaggcattctccgccggtgcgcccgcgcccccggccgtgtttcagcacggtggccatcagagggcccaggtcgaggtcgggctgggcatcgggttcggtaaactgcgcaaagcgcggagccacgtcgcgcgtgcgtgccccgcgatgcgcttcccaggactggcggaccgtggcgcgacgggcctccgcggcagcgcgcagctggggccccgactcccagacggcgggggtgccggcgaggagcagcaggaccagatccgcgtacgcccacgtatccggcgactcctccggctcgcggtccccggcgaccgtctcgaattccccgttgcgagcggcggcgcgcgtacagcagctgtccccgcccccgcgccgaccctccgtgcagtccaggagacgggcgcaatccttccagttcatcagcgcggtggtgagcgacggctgcgtgccggatcccgccgaccccgccccctcctcgcccccggaggccaaggttccgatgagggcccgggtggcagactgcgccaggaacgagtagttggagtactgcaccttggcggctcccggggagggcgagggcttgggttgcttctgggcatgccgcccgggcaccccgccgtcggtacggaagcagcagtggagaaaaaagtgccggtggatgtcgtttatggtgagggcaaagcgtgcgaaggagccgaccagggtcgccttcttggtgcgcagaaagtggcggtccatgacgtacacaaactcgaacgcggccacgaagatgctagcggcgcagtggggcgcccccaggcatttggcacagagaaacgcgtaatcggccacccactgaggcgagaggcggtaggtttgcttgtacagctcgatggtgcggcagaccagacagggccggtccagcgcgaaggtgtcgatggccgccgcggaaaagggcccggtgtccaaaagcccctccccacagggatccgggggcgggttgcggggtcctccgcgcccgcccgaaccccctccgtcgcccgcccccccgcgggcccttgagggggcggtgaccacgtcggcggcgacgtcctcgtcgagcgtaccgacgggcggcacacctatcacgtgactggccgtcaggagctcggcgcagagagcctcgttaagagccaggaggctgggatcgaaggccacatacgcgcgctcgaacgcccccgccttccagctgctgccgggggactcttcgcacaccgcgacgctcgccaggaccccggggggcgaagttgccatggctgggcgggaggggcgcacgcgccagcgaactttacgggacacaatccccgactgcgcgctgcggtcccagaccctggagagtctagacgcgcgctacgtctcgcgagacggcgcgcatgacgcggccgtctggttcgaggatatgacccccgccgagctggaggttgtcttcccgactacggacgccaagctgaactacctgtcgcggacgcagcggctggcctccctcctgacgtacgccgggcctataaaagcgcccgacgacgccgccgccccgcagaccccggacaccgcgtgtgtgcacggcgagctgctcgcccgcaagcgggaaagattcgcggcggtcattaaccggttcctggacctgcaccagattctgcggggctgacgcgcgtgctgttgggcgggacggttcgcgaaccctttggtgggtttacgcgggcacgcacgctcccatcgcgggcgccatggcgggactgggcaagccctacaccggccacccaggtgacgccttcgagggtctcgttcagcgaattcggcttatcgtcccatctacgttgcggggcggggacggggaggcgggcccctactctccctccagcctcccctccaggtgcgcctttcagtttcatggccatgacgggtccgacgagtcgtttcccatcgagtatgtactgcggcttatgaacgactgggccgaggtcccgtgcaacccttacctgcgcatacagaacaccggcgtgtcggtgctgtttcaggggttttttcatcgcccacacaacgcccccgggggcgcgattacgccagagcggaccaatgtgatcctggggtccaccgagacgacggggttgtccctcggcgacctggacaccatcaaggggcggctcggcctggatgcccggccgatgatggccagcatgtggatcagctgctttgtgcgcatgccccgcgtgcagctcgcgtttcggttcatgggccccgaagatgccggacggacgagacggatcctgtgccgcgccgccgagcaggctattacccgtcgccgccgaacccggcggtcccgggaggcgtacggggccgaggccgggctgggggtggccggaacgggtttccgggccaggggggacggttttggcccgctccccttgttaacccaagggccctcccgcccgtggcaccaggccctgcggggtcttaagcacctacggattggcccccccgcgctcgttttggcggcgggactcgtcctgggggccgctatttggtgggtggttggtgctggcgcgcgcctataaaaaaggacgcaccgccgccctaatcgccagtgcgttccggacgccttcgccccacacagccctcccgaccgacacccccatatcgcttcccgacctccggtcccgatggccgtcccgcaatttcaccgccccagcaccgttaccaccgatagcgtccgggcgcttggcatgcgcgggctcgtcttggccaccaataactctcagtttatcatggataacaaccacccacacccccagggcacccaaggggccgtgcgggagtttctccgcggtcaggcggcggcactgacggaccttggtctggcccacgcaaacaacacgtttaccccgcagcctatgttcgcgggcgacgcaccggccgcctggttgcggcccgcgtttggcctgcggcgcacctattcaccttttgtcgttcgagaaccttcgacgcccgggaccccgtgaggcccagggagttccttctggggtgttttaatcaataaaagaccacaccaacgcacgagccttgcgtttaatgtcgtgtttattcaagggagtgggatagggttcgacggttcgaaacttaacacaccaaataatcgagcgcgtctagcccagtaacatgcgcacgtgatgtaggctggtcagcacggcgtcgctgtgatgaagcagcgcccggcgggtccgctgtaactgctgttgtaggcggtaacaggcgcggatcagcaccgccagggcgctacgaccggtgcgttgcacgtagcgtcgcgacagaactgcgtttgccgatacgggcggggggccgaattgtaagcgcgtcacctcttgggagtcatcggcggataacgcactgaatggttcgttggttatgggggagtgtggttccccagggagtgggtcgagcgcctcggcctcggaatccgagaggaacaacgaggtggcgtcggagtcttcgtcgtcagagacatacagggtctgaagcagcgacacgggcgggggggtagcgtcgatgtgtagcgcgagggaggatgcccacgaagacaccccagacaaggagctgcccgtgcgtggatttgtggaagacgcggaagccgggacggatgggcggttttgcggtgcccggaaccgaaccgccggatactccccgggtgctacatgcccgttttggggctggggttggggctggggttggggctggggttggggctggggttggggctggggttggggctggggttggggttggggttggggctggggttggggttggggctggggctggggctggggctggggctggggctggggctggggctggggctggggctggggctggggctggggctggggctggggctggggctggggttggggcgcggacaggcggctgacggtcaaatgcccccgggggcgcgcagatgtggtgggcgtggccaccggctgccgtgtagtggggcggcgggaaaccgggcctccgggcgtaacaccgccctccagcgtcaagtatgtggggggcgggcctgacgtcgggggcggggtgacgggttggaccgcgggaggcgggggagagggacctgcgggagaggatgaggtcggctcggccgggttgcggcctaaaacaggggccgtggggtcggcggggtcccagggtgaagggagggattcccgcgattcggacagcgacgcgacagcggggcgcgtaaggcgccgctgcggcccgcctacgggaaccctggggggggttggcgcgggacccgaggttagcggggggcggcggttttcgcccccgggcaaaaccgtgccggttgcgaccgggggcggaacgggatcgatagggagagcgggagaagcctggccggcggactggggaccgagcgggaggggcacaccagacaccaaagcgtggggcgctggctctgggggtttgggaggggccggggggcgcgcgaaatcggtaaccggggcgaccgtgtcggggagggcaggcggccgccaaccctgggtggtcgcggaagcctgggtggcgcgcgccagggagcgtgcccggcggtgtcggcgcgcgcgcgacccggacgaagaagcggtagaagcgcgggaggaggcgggggggcggggggcggtggcatcggggggcgccggggaactttggggggacggcaagcgccggaagtcgtcgcgggggcccacgggcgccggccgcgtgctttcggccgggacgcccggtcgtgcttcgcgagccgtgactgccggcccagggggccgcggtgcacactgggacgtggggacggactgatcggcggtgggcgaaagggggtccggggcaaggaggggcgcggggccgccggagtcgtcagacgcgagctcctccaggccgtgaatccatgcccacatgcgaggggggacgggctcgccgggggtggcgtcggtgaatagcgtgggggccaggcttccgggccccaacgagccctccgccccaacaaggtccgccgggccgggggtcgggttcgggaccgaggggctctggtcgtcgggggcgcgctggtacaccggatgccccgggaatagctcccccgacaggagggaggcgtcgaacggccgcccgaggatagctcgcgcgaggaaggggtcctcgtcggtggcgctcgcggcgaggacgtcctcgccgcccgccacaaacgggagctcctcggtggcctcgctgccaacaaaccgcatgtcgggggggccgggggggtccgggttttcccacaacaccgcgaccggggtcatggagatgtccacgagcaccaggcacggcgggccccgggcgaggggccgctcggcgatgagcgcggacaggcgcgggagctgtgccgccagacacgcgttttcgatcgggttaaggtcggcgtgcaggaggcggacggcccacgtctcgatgtcggacgacacggcatcgcgcaaggcggcgtccggcccgcgagcgcgtgagtcaaacagcgtgaggcacagctccagttccgactcgcgggaaaaggccgtggtgttgcggagcgccacgacgacgggcgcgcccaggagcactgccgccagcaccaggtccatggccgtaacgcgcgccgcgggggtgcggtgggtggcggcggccggcacggcgacgtgctggcccgtgggccggtagagggcgttggggggagcggggggtgacgcctcgcgcccccccgaggggctcagcgtctgcccagattccagacgcgcggtcagaagggcgtcgaaactgtcatactctgtgtagtcgtccggaaacatgcaggtccaaagagcggccagcgcggtgcttgggagacacatgcgcccgaggacgctcaccgccgccagcgcctgggcgggactcagctttcccagcgcggcgccgcgctcggttcccagctcggggaccgagcgccagggcgccagggggtcggtttcggacaacttgccgcggcgccagtctgccagccgcgtgccgaacatgaggccccgggtcggagggcctccggccgaaaacgctggcagcacgcggatgcgggcgtctggatgcggggtcaggcgctgcacgaatagcatggaatctgctgcgttctgaaacgcacgggggagggtgagatgcatgtactcgtgttggcggaccagatccaggcgccaaaaggtgtaaatgtgttccggggagctggccaccagcgccaccagcacgtcgttctcgttaaaggaaacgcggtgcctagtggagctctggggtccgagcggcggccccggggccgccgcgtcacccccccattccagctgggcccagcgacacccaaactcgcgcgtgagagtggtcgcgacgagggcgacgtagagctcggccgccgcatccatcgaggccccccatctcgcctggcggtggcgcacaaagcgtccgaagagctgaaagttggcggcctgggcgtcgctgagggccagctgaagccggttgatgacggtgaggacgtacatggccgtgacggtcgaggccgactccagggtgtccgtcggaagcggggggcgaatgcatgccgcctcgggacacatcagcagcgcgccgagcttgtcggtcacggccgggaagcagagcgcgtactgcagtggcgttccatccgggaccaaaaagctgggggcgaacggcctatccagcgtactggtggcctcgcgcagcaccaggggccccgggcctccgctcactcgcaggtacgcctcgccccggcggcgcagcatctgcgggtcggcctcttggccgggtggggcggacgcccgggcgcgggcgtctagggcgcgaagatccacgagcaggggcgcgggcgcggccgccgcgcccgcgcccgtctggcctgtggccttggcgtacgcgctatataagcccatgcggcgttggatgagctcccgcgcgccccggaactcctccaccgcccatggggccaggtccccggccaccgcgtccaattccgccaacaggccccccagggtgtcaaagttcatctcccaggccacccttggcaccacctcgtcccgcagccgggcgctcaggtcggcgtgttgggccacgcgccccccgagctcctccacggccccggcccgctcggcgctcttggcgcccaggacgccctggtacttggcgggaaggcgctcgtagtcccgctgggctcgcagccccgacacagtgttggtggtgtcctgcagggcgcgaagctgctcgcatgccgcgcgaaatccctcgggcgatttccaggcccccccgcgaacgcggccgaagcgaccccatacctcgtcccactccgcctcggcctcctcgaaagacctccgcagggcctcgacgcggcgacgggtgtcgaagagcgactgcaggcgcgcgccctgtcgcgtcaggaggcccgggccgtcgccgctggccgcgcttagcgggtgcgtctcaaaggtgcgctgggcatgttccaaccaggcgaccgcctgcacgtcgagctcgcgcgccttctccgtctggtccaacagaatctcgacctgatccgcgatctcctccgccgagcgcgcctggtccagcgtcttggccacggtcgccgggacggcaaccaccttcagcagggtcttcagattggccagaccctcggcctcgagctgggcccggcgctcgcgcgcggccagcacctcccgcaaccccgccgtgacccgctcggtggcttcggcgcgctgctgtttggcgcgcaccacggcgtccttggtatcggccaggtcctgtcgggtcacgaatgcgacgtagtcggcgtacgccgtgtccttcacggggctctggtccacgcgctccagcgccgccacacacgccaccagcgcgtcctcgctcgggcagggcagggtgacccctgcccggacaagctcggcggccgccgccgggtcgttgcgcaccgcggatatctcctccgcggcggcggccaggtccagcgccacgcttccgatcgcgcgccgcgcgtcggcccggagggcgtccaggcgatcgcggatatccacgtactcggcgtagcccttttgaaaaaacggcacgtactggcgcagggccggcacgccccccaagtcttccgacaggtgtaggacggcctcgtggtagtcgataaacccgtcgttcgcctgggcccgctccagcagcccccccgcgagccgcagaagccgcgccaggggctcggtgtccacccgaaacatgtcggcgtacgtgtcggccgcggccccgaaggccgcgctccagtcgatgcggtgaatggctgcgagcggggggagcatggggtggcgctggttctcgggggtgtatgggttaaacgcaagggccgtctccagggcaagggtcaccgccttggcgttggttcccagcgcctgctcggcccgctttcggaagtcccgggggttgtagccgtgcgtgcccgccagcgcctgcaggcgacggagctcgaccacgtcaaactcggcaccgctttccacgcggtccagcacggcctccacgtcggcggcccagcgctcgtggctactgcgggcgcgctgggccgccatcttctctctgaggtcggcggtggcggcctcaagttcgtcggcgcggcgtcgcgtggcgccgatgacctttcccagctcctgcagggcgcgcccgctgggggagtggtccccggccgtcccttcggcgtgcaacaggcccccgaacctgccctcgtggcccgcgaggctttcccgcgcgccggtggtcgcgcgcgtcgcggcctggatcagggaggcatgctctccctccggttggttggcggcccggcgcacctggacgacaaggtcggcggcagccgaccctaaggtcgtgagctgggcgatggccccccgcgcgtccagggccaaccgagtcgccttgacgtatcccgcggcgctgtcggccatggccgctaggaaggccaggggggaggccgggtcgctggcggccgcgcccagggccgtcactgcgtcgaccaggacgcggtgcgcccgcacggccgcatccaccgtcgacgcggggtctgccgtcgcgacggcggcgctgccggcgttgatggcgttcgagacggcgtgggctatgatcggggcgtgatcggcgaagaactgcaagagaaacggagtctcgggggcgttggcgaacaggttcttcagcaccaccacgaagctgggatgcaagccggacagagccgtcgccgtgtccggagtcgggtgctccagggcatctcggtactgccccagcagcccccacatgtccgcccgcagcgccgccgtaacctccgggggcgccccccgaacggcctcggggaggtccgaccagcccgccggcagggaggcccgcagggtcgtcaggacggccggacaggcctttagccccacaaagtcagggaggggccgcaggaccccctggagtttgtgcaagaacttctcccgggcgtcgcgggccaccttcgcccgctcccgcgctccctcgagcattgcctccagggagcgcgcgcgctcccgcaaacgggcacgcgcatcgggggcgagctctgccgtcagcttggcggcatccatggcccgcgcctgccgcagcgcttcctcggccatgcgcgtggcctctggcgacagcccgccgtcgtcggggtagggcgacgcgccgggcgcaggaacaaaggccgcgtcgctgtccagctgctggcccagggccgcatctagggcgtcgaagcgccgcagctcggccagacccgagctgcggcgcgcctgctggtcgttaatgtcgcggatgctgcgcgccagctcgtccagcggcttgcgttctatcagcccttggttggcggcgtccgtcaggacggagagccaggccgccaggtcctcgggggcgtccagcgtctggccccgctgtatcagatcccgcaacaggatggccgtggggctggtcgcgatcgggggcggggcgggaatggcggcgctctgcgcgatgtcccgcgtgtgctggtcgaagacaggcagggactctagcagctggaccacgggcacgacggcggccgaagccacgtgaaaccggcggtcgttgttgtcgctggcctgcagagccttggcgctgtatacggccccccggtaaaagtactccttaaccgcgccctcgatcgcccgacgggcctgggtccgcacctcctccagccgaacctgaacggcctcggggcccagggggggtgggcgcggagccccctgcggggccgccccggccggggcgggcattacgccgaggggcccggcgtgctgtgagaccgcgtcgaccccgcgagcgagggcgtcgagggcctcgcgcatctggcgatcctccgcctccaccctaatctcttcgccacgggcaaatttggccagagcctggactctatacagaagcggttctgggtgcgtcggggtggcgggggcaaaaagggtgtccgggtgggcctgcgagcgctccagaagccactcgccgaggcgtgtatacagattggccggcggggccgcgcgaagctgcagctccaggtccgcgagttccccgtaaaaggcgtccgtctcccgaatgacatccctagccacaaggatcagcttcgccagcgccaggcgaccgatcagagagttttcgtccagcacgtgctggacgaggggcagatgggcggccacgtcggccaggctcaggcgcgtggaggccagaaagtcccccacggccgttttccggggcagcatgctcagggtaaactccagcagggcggcggccgggccggccaccccggcctgggtgtgcgtccgggccccgttctcgatgagaaaggcgaggacgcgttcaaagaaaaaaataacacagagctccagcagccccggagaagccggatacggcgaccgtaaggcgctgatggtgagccgcgaacacgcggcgacctcgcgggccagggcggcggagcacgcggtgaacttaaccgccgtggcggccacgtttgggtgggcctcgaacagctgggcaaggtctgcgcccgggggctcgggtgagcggcgagtcttcagcgcctcgagggcctgcgaggacgccggaaccgtgggcccgtcgtcctcgcccgcctcggcgaccggcggcccggccgggtcggggggtgccgaggcgaggacaggctccggaacggaggcggggaccgcggccccgacgggggttttgcctttgggggtggatttcttcttggttttggcagggggggccgagcgtttcgttttctcccccgaagtcaggtcttcgacgctggaaggcggagtccaggtgggtcggcggcgcttgggaaggccggccgagtagcgtgcccggtgccgaccaaccgggacgacgcccatctccaggacccgcatgtcgtcgtcatcttcttcggccgcctctgcggcggggggcttgggggcggagggaggcggtggtgggatcgcggagggtgggtcggcggaggggggatccgtgggtggggtacccttcagggccaccgcccatacatcgtcgggcgcccgattcgggcgcttggcctctggttttgccgacggaccggccgtcccccgggatgtctcggaggccctgtcgtcgcgacgggcccgggtcggtggcggcgactgggcggctgtgggcgggtggggccccgtgccccctaccccctcccgggggcccacgccgacgcagggctcccccaggcccgcgatctcgccccgcagggggtgcgtgatggccacgcgccgttcgctgaacgcttcgtcctgcaggtaagtctcgctggccccgtaaagatgcagagccgcggccgtcaagtccgcaggagccgcgggttccgggcccgacggcacgaaaaacaccatggctcccgcccaccgtacgtccgggcgatcgcgggtgtaatacgtcaggtatggatacatgtcccccgcccgcactttggcgatgaacgcgggggtgccctccggaaggccgtgcgggtcaaaaaggtatgcggtgtcgccgtccctgaacagccccatccctagggggccaatggttaggagcgtgtacgacagggggcgcagggcccacgggccggcgaagaacgtgtgtgcggggcattgtgtctccagcaggcccgccgcgggctccccgaagaagcccacctcgccgtatacgcgcgagaagacacagcgcagtccgccgcgcgcccctgggtactcgaggaagttggggagctcgacgatcgaacacatgcgcggcggcccagggcccgcggtcgcgcgcgtccactcgcccccctcgaccaaacaaccctcgatggcctccgcggacagaacgtcgcgagggcccacatcaaatatgaggctgagaaaggacagcgacgagcgcatgcacgataccgacccccccggctccaggtcgggcgcgaactggttccgagcaccggtgaccacgatgtcgcgatcccccccgcgttccatcgtggagtgcggtggggtgcccgcgatcatatgtgccctactggccagagacccggcctgtttatggaccggacccccggggttagtgttgtttccgccacccatgcccccgtaccatggccccggttcccctgattaggctacgagtcgcggtgatcgcttcccaaaaaccgagctgcgtttgtctgtcttgatctttcccccccccgcccgcccgcccgcccgcacaccataacaccgagaacaacacacgggggtgggcgtaacataataaagctttattggtaactagttaacggcaagtccgtgggtggcgcgacggtgtcctccgggctcatctcgtcgtcctcgacgggggtgttggaatgaggcgccccctcgcggtccgcctggcgtgggccgtgcccataggcctccggcttctgtgcgtccatgggcataggcgcggggagactgtttccggcgtcgcggacctccaggtccctgggagactccggtccggctaacggacgaaacgcggaagcgcgaaacacgccgtcggtgacccgcaggagctcgttcatcagtaaccaatccatactcagcgtaacggccagcccctggcgagacagatccacggagtccggaaccgcggtcgtctggcccagggggccgaggctgtagtccccccaggcccctaggtcgcgacggctcgtaagcacgacgcggtcggccgcggggctttgcgggggggcgtcctcgggcgcatgcgccattacctctcggatggccgcggcgcgctggtcggccgagctgaccaagggcgccacgaccacggcgcgctccgtctgcaggcccttccacgtgtcgtggagttcctggacaaactcggccacgggctcgggtcccgcggccgcgcgcgcggcttgatagcaggccgagagacgccgccagcgcgctagaaactgacccatgaagcaaaacccggggacctggtctcccgacagcagcttcgacgcccgggcgtgaatgccggacacgacggacagaaacccgtgaatttcgcgccggaccacggccagcacgttgtcctcgtgcgacacctgggccgccagctcgtcgcacacccccaggtgcgccgtggtttcggtgatgacggaacgcaggctcgcgagggacgcgaccagcgcgcgcttggcgtcgtgatacatgctgcagtactgactcaccgcgtcccccatggcctcggggggccagggccccaggcggtcgggcgtgtccccgaccaccgcatacaggcggcgcccgtcgctctcgaaccgacactcgaaaaaggcggagagcgtgcgcatgtgcagccgcagcagcacgatggcgtcctccagttggcgaatcagggggtctgcgcgctcggcgaggtcctgcagcaccccccgggcggccagggcgtacatgctaatcaacaggaggctggtgcccacctcggggggcggggggggctgcagctggaccaggggccgcagctgctcgacggcacccctggagatcacgtacagctcccggagcagctgctctatgttgtcggccatctgcatagtggggccgaggccgccccgggcggccggttcgaggagggtaatcagcgcgcccagtttggtgcgatggccctcgaccgtggggagatagcccagcccaaagtcccgggcccaggccaacacacgcagggcgaactcgaccgggcgtggaaggtaggccgcgctacacgtggccctcaacgcgtccccgaccaccagggccagaacgtaggggacgaagcccgggtcggcgaggacgttggggtgaatgccctcgagggcggggaagcggatctgggtcgccgcggccaggtggacagagggggcgtggctgggctgcccgacggggagaagcgcggacagcggcgtggccggggtggtgggggtgatgtcccagtgggtctgaccatacacgtcgatccagatgagcgccgtctcgcggagaaggctgggttgaccggaactaaagcggcgctcggccgtctcaaactcccccacgagcgcccgccgcaggctcgccagatgttccgtcggcacggccggacccatgatacgcgccagcgtctggctcagaacgccccccgacaggccgaccgcctcgcagagccgcccgtgcgtgtgctcgctggcgccctggacccgcctgaaagtttttacgtagttggcatagtacccgtattcccgcgccagaccaaacacgttcgaccccgcgagggcaatgcacccaaagagctgctggacttcgccgagtccgtggccggcgggcgtccgcgcggggacgcccgccgccagaaacccctccagggccgaaaggtagtgcgtgcagtgcgagggcgtgaacccagcgtcgatcagggtgttgatcaccacggagggcgaattggtattctggatcaacgtccacgtctgctgcagcagagccagcagccgctgctgggcgccggcggagggctgctccccgagctgcagcaggctggagacggcaggctggaagactgccagtgccgacgaactcaggaacggcacgtcgggatcaaacacggccacgtccgtccgcacgcgcgccattagcgtccccgggggcgcacaggccgagcgcgggctgacgcggctgagggccgtcgacacgcgcacctcctcgcggctgcgaaccatcttgttggcctccagtggcggaatcattatggccgggtcgatctcccgcacggtgtgctgaaactgcgccaacaggggcggcgggaccacagccccccgctcgggggtcgtcaggtactcgtccaccagggccaacgtaaagagggcccgtgtgaggggagtgagggtcgcgtcgtctatgcgctggaggtgcgccgagaacagcgtcacccgattactcaccagggccaagaaccggaggccctcttgcacgaacggggcggggaagagcaggctgtacgccggggtggtaaggttcgcgctgggctgccccaacgggaccggcgccatcttgagcgacgtctccccaagggcctcgatggaggtccgcgggctcatggccaagcagctcttggtgacggtttgccagcggtctatccactccacggcgcactggcggacgcggaccggccccagggccgccgcggtgcgcaggccggcggaatccagcgcatgggacgtgtcggagccggtgaccgcgaggatggtgtccttgatgacctccatctcccggaaggcctggtcgggggcctcggggagagccaccaccaagcggtgtacgagcaacccggggaggttctcggccaagagcgccgtctccggaagcccgtgggcccggtggagcgcgcacaggtgttccagcagcggccgccagcatgcccgcgcgtctgccggggcgatggccgttcccgacaacagaaacgccgccatggcggcgcgcagcttggccgtggccagaaacgccgggtcgtccgccccgtttgccgtctcggccgtgggggttggcggttggcgaaggccggctaggctcgccaataggcgctgcataggtccgtccgagggcggaccggcgggtgaggtcgtgacgacgggggcctcggacgggagaccgcggtctgccatgacgcccggctcgcgtgggtgggggacagcgtagaccaacgacgagaccgggcgggaatgactgtcgtgcgctgtagggagcggcgaattatcgatcccctgcggccctccaggaaccccgcaggcgttgcgagtaccccgcgtcttcgcggggtgttatacggccacttaagtcccggcatcccgttcgcggacccaggcccgggggattgtccggatgtgcgggcagcccggacggcgtgggttgcggactttctgcggggcggcccaaatggccctttaaacgtgtgtatacggacgcgccgggccagtcggccaacacaacccaccggaggcggtagccgcgtttggctgtggggtgggtggttccgccttgcgtgagtgtcctttcgacccccccctcccccgggtcttgctaggtcgcgatctgtggtcgcaatgaagaccaatccgctacccgcaaccccttccgtgtggggcgggagtaccgtggaactcccccccaccacacgcgataccgcggggcagggcctgcttcggtgcgtcctgcgccccccgatctctcgccgcgacggcccagtgctccccagggggtcgggaccccggagggcggccagcacgctgtggttgcttggcctggacggcacagacgcgccccctggggcgctgacccccaacgacgataccgaacaggccctggacaagatcctgcggggcaccatgcgcgggggggcggccctgatcggctccccgcgccatcatctaacccgccaagtgatcctgacggatctgtgccaacccaacgcggatcgtgccgggacgctgcttctggcgctgcggcaccccgccgacctgcctcacctggcccaccagcgcgccccgccaggccggcagaccgagcggctgggcgaggcctggggccagctgatggaggcgaccgccctggggtcggggcgagccgagagcgggtgcacgcgcgcgggcctcgtgtcgtttaacttcctggtggcggcgtgtgccgcctcgtacgacgcgcgcgacgccgccgatgcggtacgggcccacgtcacggccaactaccgcgggacgcgggtgggggcgcgcctggatcgtttttccgagtgtctgcgcgccatggttcacacgcacgtcttcccccacgaggtcatgcggtttttcggggggctggtgtcgtgggtcacccaggacgagctagcgagcgtcaccgccgtgtgcgccgggccccaggaggcggcgcacaccggccacccgggccggccccgctcggccgtgatcctcccggcgtgtgcgttcgtggacctggacgccgagctggggctggggggcccgggcgcggcgtttctgtacctggtattcacttaccgccagcgccgggaccaggagctgtgttgtgtgtacgtgatcaagagccagctccccccgcgcgggttggagccggccctggagcggctgtttgggcgcctccggatcaccaacacgattcacggcaccgaggacatgacgcccccggccccaaaccgaaaccccgacttccccctcgcgggcctggccgccaatccccaaaccccgcgttgctcggctggccaggtcacgaacccccagttcgccgacaggctgtaccgctggcagccggaccttcgggggcgccccaccgcacgcacctgtacgtacgccgcctttgcagagctcggcatgatgcccgaggatagtccccgctgcctgcaccgcaccgagcgctttggggcggtcagcgtccccgttgttattctggaaggcgtggtgtggcgccccggcgagtggcgggcatgcgcgtgagcgtagcaaacgccccgcccacacaacgctccgcccccaaccccttccccgctgtcactcgtggttcgttgacccggacgtccgccaaataaagccactgaaacccgaaacgcgagtgttgtaacgtcctttgggcgggaggaagccacaaaatgcaaatgggatacatggaaggaacacacccccgtgactcaggacatcggcgtgtccttttgggtttcactgaaactggcccgcgccccacccctgcgcgatgtggataaaaagccagcgcgggtggtttagggtaccacaggtgggtgctttggaaacttgtcggtcgccgtgctcctgtgagcttgcgtccctccccggtttcctttgcgctcccgccttccggacctgctctcgcctatcttctttggctgtcggtgcgattcgtcaggcagcggccttgtcgaatctcgaccccaccactcgccggacccgccgacgtcccctctggagcccgccgaaacccgccgcgtctgttgaaatggccagccgcccagccgcatcctctcccgtcgaagcgcgggccccggttgggggacaggaggccggcggccccagcgcagccacccagggggaggccgccggggcccctctcgcccacggccaccacgtgtactgccagcgagtcaatggcgtgatggtgctttccgacaagacgcccgggtccgcgtcctaccgcatcagcgatagcaactttgtccaatgtggttccaactgcaccatgattatcgacggagacgtggtgcgcgggcgcccccaggacccgggggccgcggcatcccccgctcccttcgttgcggtgacaaacatcggagccggcagcgacggcgggaccgccgtcgttgcattcgggggaaccccacgtcgctcggcggggacgtctaccggtacccagacggccgacgtcccagccgaggcccttgggggcccccctcctcctccccgcttcaccctgggtggcggctgttgctcctgtcgcgacacacggcgccgctctgcggtattcgggggggagggggatcccgtcggccccgcggagttcgtctcggacgaccggtcgtccgattccgactcggatgactcggaggacaccgactcggagacgctgtcacacgcctcctcggacgtgtccggcggggccacgtacgacgacgcccttgactccgattcgtcatcggatgactccctgcagatagatggccccgtgtgtcgcccgtggagcaatgacaccgcgcccctggatgtttgccccgggacccccggcccgggcgccgacgccggtggtccctcagcggtagacccacacgcgccgacgacaggggccggcgctggtcttgcggccgatcccgccgtggcccgggacgacgcggaggggctttcggacccccggccacgtctgggaacgggcacggcctaccccgtccccctggaactcacgcccgagaacgcggaggccgtggcgcgctttctgggagatgccgtgaaccgcgaacccgcgctcatgctggagtacttttgccggtgcgcccgcgaggaaaccaagcgtgtcccccccaggacattctgcagcccccctcgcctcacggaggacgactttgggcttctcaactacgcgctcgtggagatgcagcgcctgtgtctggacgttcctccggtcccgccgaacgcatacatgccctattatctcagggagtatgtgacgcggctggtcaacgggttcaagccgctggtgagccggtccgctcgcctttaccgcatcctgggggttctggtgcacctgcggatccggacccgggaggcctcctttgaggagtggctgcgatccaaggaagtggccctggactttggcctgacggaaaggcttcgcgagcacgaagcccagctggtgatcctggcccaggctctggaccattacgactgtctgatccacagcacaccgcacacgctggtcgagcgggggctgcaatcggccctgaagtatgaggagttttacctaaagcgctttggcgggcactacatggagtccgtcttccagatgtacacccgcatcgccggctttttggcctgccgggccacgcgcggcatgcgccacatcgccctggggcgagaggggtcgtggtgggaaatgttcaagttctttttccaccgcctctacgaccaccagatcgtaccgtcgacccccgccatgctgaacctggggacccgcaactactacacctccagctgctacctggtaaacccccaggccaccacaaacaaggcgaccctgcgggccatcaccagcaacgtcagtgccatcctcgcccgcaacgggggcatcgggctatgcgtgcaggcgtttaacgactccggccccgggaccgccagcgtcatgcccgccctcaaggtcctcgactcgctggtggcggcgcacaacaaagagagcgcgcgtccgaccggcgcgtgcgtgtacctggagccgtggcacaccgacgtgcgggccgtgctccggatgaagggggtcctcgccggcgaagaggcccagcgctgcgacaatatcttcagcgccctctggatgccagacctgtttttcaagcgcctgattcgccacctggacggcgagaagaacgtcacatggaccctgttcgaccgggacaccagcatgtcgctcgccgactttcacggggaggagttcgagaagctctaccagcacctcgaggtcatggggttcggcgagcagatacccatccaggagctggcctatggcattgtgcgcagtgcggccacgaccgggagccccttcgtcatgttcaaagacgcggtgaaccgccactacatctacgacacccagggggcggccatcgccggctccaacctctgcaccgagatcgtccatccggcctccaagcgatccagtggggtctgcaatctgggaagcgtgaatctggcccgatgcgtctccaggcagacgtttgactttgggcggctccgcgacgccgtgcaggcgtgcgtgctgatggtgaacatcatgatcgacagcacgctacaacccacgccccagtgcacccgcggcaacgacaacctgcggtccatgggaatcggcatgcagggcctgcacacggcctgcctgaagctggggctggatctggagtctgccgaatttcaggacctgaacaaacacatcgccgaggtgatgctgctgtcggcgatgaagaccagcaacgcgctgtgcgttcgcggggcccgtcccttcaaccactttaagcgcagcatgtatcgcgccggccgctttcactgggagcgctttccggacgcccggccgcggtacgagggcgagtgggagatgctacgccagagcatgatgaaacacggcctgcgcaacagccagtttgtcgcgctgatgcccaccgccgcctcggcgcagatctcggacgtcagcgagggctttgcccccctgttcaccaacctgtttagcaaggtgacccgggacggcgagacgctgcgccccaacacgctcctgctaaaggaactggaacgcacgtttagcgggaagcgcctcctggaggtgatggacagtctcgacgccaagcagtggtccgtggcgcaggcgctcccgtgcctggagcccacccaccccctccggcgattcaagaccgcgtttgactacgaccagaagttgctgatcgacctgtgtgcggaccgcgccccctacgtcgaccatagccaatccatgaccctgtatgtcacggagaaggcggacgggaccctcccagcctccaccctggtccgccttctggtccacgcatataagcgcggactaaaaacagggatgtactactgcaaggttcgcaaggcgaccaacagcggggtctttggcggcgacgacaacattgtctgcacgagctgcgcgctgtgaccgacaaaccccctccgcgccaggcccgccgccactgtcgtcgccgtcccacgcgctcccccgctgccatggattccgcggccccagccctctcccccgctctgacggcccatacgggccagagcgcgccggcggacctggcgatccagattccaaagtgccccgaccccgagaggtacttctacacctcccagtgtcccgacattaaccacctgcgctccctcagcatccttaaccgctggctggaaaccgagcttgttttcgtgggggacgaggaggacgtctccaagctttccgagggcgagctcagcttttaccgcttcctcttcgctttcctgtcggccgccgacgacctggttacggaaaacctgggcggcctctccggcctgtttgagcagaaggacattctccactactacgtggagcaggaatgcatcgaagtcgtacactcgcgcgtgtacaacatcatccagctggtgctttttcacaacaacgaccaggcgcgccgcgagtacgtggccggcaccatcaaccacccggccatccgcgccaaggtggactggttggaagcgcgggtgcgggaatgcgcctccgttccggaaaagttcattctcatgatcctcatcgagggcatcttttttgccgcctcgtttgccgccatcgcctaccttcgcaccaacaaccttctgcgggtcacctgccagtcaaacgacctcatcagccgggacgaggccgtgcacacgacggcctcgtgttacatctacaacaactacctcggcgggcacgccaagcccccgcccgaccgcgtgtacgggctgttccgccaggcggtcgagatcgagatcggatttatccgatcccaggcgccgacggacagccatatcctgagcccggcggcgctggcggccatcgaaaactacgtgcgattcagcgcggatcgcctgttgggccttatccacatgaagccactgttttccgccccaccccccgacgccagctttccgctgagcctcatgtccaccgacaaacacaccaattttttcgagtgtcgcagcacctcctacgccggggcggtcgtcaacgatctgtgagggtcgcggcgcgcttctacccgtgtttgcccataataaacctctgaaccaaactttgggtctcattgtgattcttgtcagggacgcgggggtgggagaggataaaaggcggcgcaaaaagcagtaaccaggtccggccagattctgagggcataggataccataattttattggtgggtcgtttgttcggggacaagcgcgctcgtctgacgtttgggctactcgtcccagaatttggccaggacgtccttgtagaacgcgggtgggggggcctgggtccgcagctgctccagaaacctgtcggcgatatcaggggccgtgatatgccgggtcacgatagatcgcgccaggttttcgtcgcggatgtcctggtagataggcaggcgtttcagaagagtccacggcccccgctccttggggccgataagcgatatgacgtacttaatgtagcggtgttccaccagctcggtgatggtcatgggatcggggagccagtccagggactctggggcgtcgtggatgacgtggcgtcgccggctggccacataactgcggtgctcttccagcagctgcgcgttcgggacctggacgagctcgggcggggtgagtatctccgaggaggacgacctggggccggggtggcccccggtaacgtcccggggatccagggggaggtcctcgtcgtcttcgtatccgccggcgatctgttgggttagaatttcggtccacgagacgcgcgtctcggtgccgccggtggccggcggcagagggggcctggtttccgtggagcgcgagctggtgtgttcccggcggatggcccgccgggtctgagagcgactcgggggggtccagtgacattcgcgcagcacatcctccacggaggcgtaggtgttattgggatggaggtcggtgtggcagcggacaaagagggccaggaactgggggtagctcatcttaaagtacttcagtatatcgcgacagttgatcgtgggaatgtagcaggcgctaatatccaacacaatatcgcagcccatcaacaggaggtcagtgtccgtggtgtacacgtacgcgaccgtgttggtgtgatagaggttggcgcaggcatcgtccgcctccagctgacccgagttaatgtaggcgtaccccagggcccggagaacgcgaatacagaacagatgcgccagacgcagggccggcttcgagggcgcggcggacggcagcgcggctccggacccggccgtcccccgggtccccgaggccagagaggtgccgcgtcggcgcatgttggaaaaggcagagctgggtctggagtcggtgatgggggaaggcggtggagaggcgtccacgtcactggcctcctcgtccgtccggcactgggccgtcgtgcgggccaggatggccttggctccaaacacaaccggctccatacaattgaccccgcgatcggtaacgaagatggggaaaagggacttttgggtaaacacctttaataagcgacagaggcagtgtagcgtaatggcctcgcggtcgtaactggggtatcggcgctgatatttgaccaccaacgtgtacatgacgttccacaggtccacggcaatgggggtgaagtacccggccggggccccaaggccccggcgcttgaccagatggtgtgtgtgggcaaacttcatcatcccgaacaaacccatgtcaggtcgattgtaactgcggatcggcctaactaaggcgtggttggtgcgacggtccgggacacccgagcctgtctctctgtgtatggtgacccagacaacaacaccgacacaagaggacaataatccgttaggggacgctctttataatttcgatggcccaactccacgcggattggtgcagcaccctgcatgcgccggtgcgggccaaccttccccccgctcattgcctcttccaaaagggtgtggcctaacgagctgggggcgtatttaatcaggctagcgcggcgggcctgccgtagtttctggctcggtgagcgacggtccggttgcttgggtcccctggctgccatcaaaaccccaccctcgcagcggcatacgccccctccgcgtcccgcacccgagaccccggcccggctgccctcaccaccgaagcccacctcgtcactgtggggtgttcccagcccgcgttgggatgacggattcccctggcggtgtggcccccgcctcccacgtggaggacgcgtcggacgcgtccctcgggcagccggaggagggggcgccctgccaggtggtcctgcagggcgccgagcttaatggaatcctacaggcgtttgccccgctgcgcacgagccttctggactcgcttctggttatgggagaccggggcatccttatccataacacgatctttggggagcaggtgttcctgcccctggaacactcgcaattcagtcggtatcgctggcgcggacccacggcggcgttcctgtctctcgtggaccagaagcgctccctcctgagcgtgtttcgcgccaaccagtacccggacctacgtcgggtggagttggcgatcacgggccaggccccgtttcgcacgctggttcagcgcatatggacgacgacgtccgacggcgaggccgttgagctagccagcgagacgctgatgaagcgcgaactgacgagctttgtggtgctggttccccagggaacccccgacgttcagttgcgcctgacgaggccgcagctcaccaaggtccttaacgcgaccggggccgatagtgccacgcccaccacgttcgagctcggggttaacggcaaattttccgtgttcaccacgagtacctgcgtcacatttgctgcccgcgaggagggcgtgtcgtccagcaccagcacccaggtccagatcctgtccaacgcgctcaccaaggcgggccaggcggccgccaacgccaagacggtgtacggggaaaatacccatcgtaccttctctgtggtcgtcgacgattgcagcatgcgggcggtgctccggcgactgcaggtcgccgggggcaccctcaagttcttcctcacgacccccgtccccagtctgtgcgtcaccgccaccggtcccaacgcggtatcggcggtatttctcctgaaaccccagaagatttgcctggactggctgggtcatagccaggggtctccttccgccgggagctcggcctcccgggcctctgggagcgagccaacagacagccaggactccgcgtcggacgcggtcagccacggcgatccggaagacctcgatggcgctgcccgggcgggagaggcgggggcctcgtacgcctgtccgatgccgtcgtcgaccacgcgggtcactcccacgaccaagcgggggcgctcggggggcgaggatgcgcacgcggacacggccctaaagaaacctaagacggggtcgcccaccgcacccccgcccgcagatccagtccccctggacacggaggacgactccgatgcggcggacgggacggcggcccgtcccgccgctccagacgcccgaagcggaagccgttacgcgtgttactttcgcgacctcccgaccggagaagcaagccccggcgccttctccgccttccgggggggcccccaaaccccgtctggttttggattcccctgacggggcggggccttagcggccgcccaaccctcgcaacatcccggggttaatgtaaataaacttggtattgcccaacactctcccgcgtgtcgcgtgtggttcatgtgtgtgcctggcgcccccaccctcgggttcgtgtatttcctttccctgtccttataaaagccgtatgtggggcgctgacggaaccaccccgcgtgccatcacggccaaggcgcgggatgctccgcaacgacagccaccgggccgcgtccccggaggacggccagggacgggtcgacgacggacggccacacctcgcgtgcgtgggggccctggcgcgggggttcatgcatatctggcttcaggccgccacgctgggttttgcgggatcggtcgttatgtcgcgcgggccgtacgcgaatgccgcgtctggggcgttcgccgtcgggtgcgccgtgctgggctttatgcgcgcgccccctcccctcgcgcggcccaccgcgcggatatacgcctggctcaaactggcggccggtggagcggcccttgttctgtggagtctcggggagcccggcacgcagccgggggccctggccccgggcccggccacccagtgcctggcgctgggcgccgcctatgcggcgctcctggtgctcgccgatgacgtctatccgctctttctcctcgccccggggcccctgttcgtcggcaccctggggatggtcgtcggcgggctgacgatcggaggcagcgcgcgctactggtggatcggtgggcccgccgcggccgccctggccgcggcggtgttggcgggcccgggggcgaccaccgccagggactgcttctccagggcgtgccccgaccaccgccgcgtctgcgtcatcgtcgcaggcgagtctgtttcccgccgccccccggaggacccagagcgacccggggacccagggccaccgtcccccccgacaccccaacgatcccaggggccgccggccgatgaggtcgcaccggccggggtagcgcggcccgaaaacgtctgggtgcccgtggtcacctttctgggggctggcgcgctcgccgtcaagacggtgcgagaacatgcccggggaacgccgggcccgggcctgccgctgtggccccaggtgtttctcggaggccatgtggcggtggccctgacggagctgtgtcaggcgcttgcgccctgggaccttacggacccgctgctgtttgttcacgccggactgcaggtcatcaacctcgggttggtgtttcggttttccgaggttgtcgtgtatgcggcgctagggggtgccgtgtggatttcgttggcgcaggtgctggggctccggcgtcgcctgcacaggaaggaccccggggacggggcccggttggcggcgacgcttcggggcctcttcttctccgtgtacgcgctggggtttggggtgggggcgctgctgtgccctccggggtcaacgggcgggcggtcgggcgattgatatatttttcaataaaaggcattagtcccgaagaccgccggtgtgtgatgatttcgccataacacccaaaccccggatggggcccgggtataaattccggaaggggacacgggctaccttcactaccgagggcgcttggtcgggaggccgcatcgaacgcacacccccatccggtggtccgtgtggaggtcgtttttcagtgcccggtctcgctttgccgggaacgctagccgatccctcgcgagggggaggcgtcgggcatggccccggggcgggtgggccttgccgtggtcctgtggagcctggtgtggctcggggcgggggtgtccgggggctcggaaactgcctccaccgggcccacgatcaccgcgggagcggtgacgaacgcgagcgaggcccccacatcggggtcccccgggtcagccgccagcccggaggtcacccccacatcgaccccaaaccccaacaatgtcacacaaaaccaaaccacccccaccgagccggccagccccccaacaacccccaagcccacctccacacccaaaagcccccccacgtccacccccgaccccaaacccaagaacaacaccacccccgccaagtcggaccgccccactaaaccccccgggcccgtgtggtgcgaccgccgcgatttattggcccggtacggctcgcgggtgcagatccgatgccggtttcggaattccacccgcatggagttccgcctccagatatggcgttactccatgggtccgtcccccccaatcgctccggctcccgacctagaggaggtcctgacgaacatcaccgccccacccgggggactcctggtgtacgacagcgcccccaacctaacggacccccacgtgctctgggcggagggggccggcccgggcgccgaccctccgttgtattctgtcaccgggccgctgccgacccagcggctgattatcggcgaggtgacgcccgcgacccagggaatgtattacttggcctggggccggatggacagcccgcacgagtacgggacgtgggtgcgcgtccgcatgttccgccccccgtctctgaccctccagccccacgcggtgatggagggtcagccgttcaaggcgacgtgcacggccgccgcctactacccgcgtaaccccgtggagtttgtctggttcgaggacgaccaccaggtgtttaacccgggccagatcgacacgcagacgcacgagcaccccgacgggttcaccacagtctctaccgtgacctccgaggctgtcggcggccaggtccccccgcggaccttcacctgccagatgacgtggcaccgcgactccgtgacgttctcgcgacgcaatgccaccgggctggccctggtgctgccgcggccaaccatcaccatggaatttggggtccggcatgtggtctgcacggccggctgcgtccccgagggcgtgacgtttgcctggttcctgggggacgacccctcaccggcggctaagtcggccgttacggcccaggagtcgtgcgaccgccccgggctggctacggtccggtccaccctgcccatttcgtacgactacagcgagtacatctgtcggttgaccggatatccggccgggattcccgttctagagcaccacggcagtcaccagcccccacccagggaccccaccgagcggcaggtgatcgaggcgatcgagtgggtggggattggaatcggggttctcgcggcgggggtcctggtcgtaacggcaatcgtgtacgtcgtccgcacatcacagtcgcggcagcgtcatcggcggtaacgcgagacccccccgttacctttttaatatctatatagtttggtccccctctatcccgcccaccgctgggcgctataaagccgccaccctctcttccctcaggtcatccttggtcgatcccgaacgacacacggcgtggagcaaaacgcctccccctgagccgctttcctaccagcgcaacggcatgcctctgcgggcatcggaacacgcctaccggcccctgggccccgggacaccccccatgcgggctcggctccccgccgcggcctgggttggcgtcgggaccatcatcgggggagttgtgatcattgccgcgttggtcctcgtgccctcgcgggcctcgtgggcactttccccatgcgacagcggatggcacgagttcaacctcgggtgcatatcctgggatccgacccccatggagcacgagcaggcggtcggcggctgtagcgccccggcgaccctgatcccccgcgcggctgccaaacagctggccgccgtcgcacgcgtccagtcggcaagatcctcgggctactggtgggtgagcggagacggcattcgggcctgcctgcggctcgtcgacggcgtcggcggtattgaccagttttgcgaggagcccgcccttcgcatatgctactatccccgcagtcccgggggctttgttcagtttgtaacttcgacccgcaacgcgctggggctgccgtgaggcgcgtgtactgcggtctgtctcgtctcctcttctccccttccctccccctccgcatcccaggatcacaccggccaacgagggttggggggtccggcacggacccaaaataataaacacacaatcacgtgcgataaaaagaacacgcggtcccctgtggtgtttttggttatttttattaaatctcgtcgtcaaacagggggaaaggggcgtggtctagcgacggcagcacgggtggaggcgttcaccggctccggcgtccttcgcgtttaagcttggtcaggagggcgctcagggcggcgacgttggtcgggccgtcgttggtcagggcgttggctcgatggcgggcgaggacgggcgaggggctcaacggcgggggcgggggcccggtgcggcccgggggggaaaatagggcggatcccccccagtcgtacaggggattttccgcctcaatgtacggggaggccggcgctgcattcgccgtgttcgcgcagacgttttcgtagacccgcatccatggtatttcctcgtagacacgccccccgtcctcgctcacagtctcgtatattgactcgtcgtcctcgtagggggcgtgccgttcgcgggccgaggcggcgtgggtggctttgcggcgggcgtcgtcgtcgtcgtcggccgtcagatacgtggcttccatctggtcgggttctccctccggggcgggtccccacccccgtggccgatcgaggctccccagagacgcgcgccggacgaggagggggcacgtcgccgccggcggtcgcctgtcgggtcccgcgacgttacgggccgggaggcgcgggggcacctcccccatgtgcgtgtaatacgtggccggctgtgcggccgcagcggggggctcggcgaccgggtcgtccgcatccggaagcgggggcgccgcgccgtccgcgcggcgcctccggaaccgccgggtggccgcgggggtcgagtgtaggcgaggtcgggggaggggcgggggctcgttgtcgcgccgcgcccgctgaatcttttcccgacaggtcccaccccccgcgcgatgcccccccgggccgcgggccatgtcgtccgggggaggccccgcggaccacgtcgtccggcgagacgccacgagccgcaggatggactcgtagtggagcgacggcgccccgctgcggagcagatccgcggccagggcggccccgaaccaagccttgatgctcaactccatccgggcccagctgggggcggtcatcgtggggaacaggggggcggtggtccgacagaaacgctcctggctgtccaccgcggcccgcagatactcgttgttcaggctgtcggtggcccagacgccgtacccggtgagggtcgcgttgatgatatactgggcgtggtgatggacgatcgacagaacctccaccgtggatacgacggtatccacggtcccgtacgtaccgccgctccgcttgccggtctgccacaggttggctaggcgcgtcaggtggcccaggacgtcgctgaccgccgccctgagcgccatgcactgcatggagccggtcgtgccgctgggaccccggtccagatggcgcgcgaacgtttccgcgggcgcctccgggctgccgccgagcgggaggaaccggcgattggagggactcagccggtggcatacgtgcttgtctgtcgtccacagcatccaggacgcccaccggtacagcacggagacgtaggccaggagctcgttgagccgcagtgcggtgtcggtgctggggcggcttgggtccgccgggcgcataaagaacatgtactgctgaatccgatggagggcgtcgcgcaggccggccacggtggcggcgtacttggccgccgcggccccgctcttgaacggggtgcgcgccagcagctttggcgccagggtgggccgcagcagcacgtgaaggctggggtcgcagtcgcccacggggtcctcggggacgtccaggccgctgggcaccaccgtctgcaggtacttccagtactgcgtgaggatggcgcggctcaactggccgccggtgagctccacctcgcccagcgcctgggtggcggccgaagcgtagtgccggatgtactcgtagtgcgggtcgctggcgagcccgtccacgatcaaactctcgggaaccgtgttgtgttgccgcgcggccaaccggacgctgcgatcggtgcaggtcagaaacgccggctgcgcgtcgtcggagcgctgccgcaaggcgcccacggccgcgctaaggagcccctccggggtggggagcagacacccgccgaagatgcgccgctcgggaacgcccgcgttgtcgccgcggatcaggttggcaggcgtcaggcaccgcgccagccgcagggagctcgcgccgcgcgtccggcgctgcatggtgacgcccgttcggtcgggacccgccggtcggagttatgccgcgtccagggccatcggggcgctttttatcgggaggagcttatgggcgtggcgggcctcccagcccggtcgcgcgcctccccgacacgtgcgcccgcagggcggcggccccctcgtctcccatcagcagtttcctaaactgggacatgatgtccaccacgcggacccgcgggcccaacacggacccgccgcttacgggggcgggggggaagggctccaggtccttgagaagaaaggcggggtctgccgtcccggacacgggggcccggggcgctgaggaggcggggcgcagatccacgtgctccgcggccgcgcggacgtccgcccagaacttggcgggggtggtgcgcgcgtacaggggctgggtcgctcggaggacgcacgcgtagcgcaggggggtgtacgtgcccacctcgggggccgtgaatcccccgtcaaacgcggccagtgtcacgcacgccaccacggtgtcggcaaagcccagcagccgctgcaggacgagcccggcggccagaatggcgcgcgtggccgccgcgtcgtcccggcgccggtgcgcgtccccgcacgcccgggcgtactttaaggtcacggtcgccagggccgtgtgcagcgcgtacaccgcagcgcccagcacggcgttgagcccgctgttggcgagcagccggcgcgctgcggtgtcgcccagcgcctcgtgctcggcccccacgaccgcggggcttcccaggggcagggcgcgaaacagctcctcccgcgccacgtccgcaaaggcggggtggtgcacgtgcgggtgcaggcgcgcccccacgaccaccgagagccactggaccgtctgctccgccatcaccgccagcacatccagcacgcgccccaggaaggcggcctcccgcgtcaaaacgcaccggacggcgtcgggattgaagcgggcgagcagggccccggtggccaggtacgtcatgcggccggcatagcgggcggccacgcgacagtcgcggtccagcagcgcgcgcaccccgggccagtacagcagggaccccagcgagctgcgaaacaccgcggcgtcggggccggattggggggacactaacccccccgcgctcagtaacggcacggccgcggccccgacgggacgcaacgccgtgaggctcgcgaactgccgcctcagctcggcagccctgtcgtccaggtccgacccgcgcgcctctgcgtgaaggcgcgtcccgcacacccacccgttgatggccagccgcacgacggcatccgccaaaaagctcatcgcctgggcggggctggtttttgttcgacgatccgtcaggtcaagaatcccatcgcccgtgatataccaggccaacgcctcgccctgctgcagggtttggcggaaaaacaccgcggggttgtcgggggaggcgaagtgcatgacccccacgcgcgataacccgaacgcgctatccggacacgggtaaaacccggccggatgccccagggctagggcggagcgcacggactcgtcccacacggcaacctgaggggccagtcgatccaacgggaatgccgcccggagctccgggcccggcacgcgtccctccagaacctccaccttgggcggggaacgggccccgccgccgtcctccggcccgacgtcttccgggtagtcgtcctcctcgtactgcagctcctctaggaacagcggcgacggcgccacccgcgaaccgccgacccgccccaaaatagcccgcgcgtcgacgggacccaggtatcccccctgccgggcctgcggaggaccgcggggaacctcatcatcatcgtccaggcgaccgcgcaccgactggctacgggccgcatcgggcccggggcgctgccgggacgctcggcgatgggatgtgggcggggcttccgacgcgcgccgtcgtcgggctcgcgggccttcccgtcgacggcgcacgggcggctcgtcgcccgccatctcctccagagcctctagctcgctgtcgtcatccccgcggaacaccgcacgcaggtaccccatgaaccccaccccatcgcccgctggctcgtccgccacgggcgaggcgcgggggcgggtggatgcgcgcctcctacgccccgcgggttcgcgagccgacatggtggcgatagacgcgggttatcggatgtccgctaccccccaaaaaagaaaaagaccccacagcgcggatggaggccggggtaggtgccgccggaccccctcgcgatgggaatggacgggagcgacggggccggcgcaaaaaacgcagtatctcccgcgaaggctacccgccgccccagcccccggccaaatgcggaaacggtcccgcgctctcgcctttatacgcgggccgccctgcgacacaatcacccgtccgtggtttcgaatctacacgacaggcccgcagacgcggctaacacacacgccggcaacccagaccccagtgggttggttgcgcggtcccgtctcctggctagttcttttccccaccaccaaataatcagacgacaaccgcaggtttttgtaatgtatgtgctcgtgtttattgtggatacgaaccggggacgggaggggaaaacccagacgggggatgcgggtccggtcgcgccccctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcgtgttcccgcatgaccgcctcggagggcgaggtcgtgaagctggaatacgagtccaacttcgcccgaatcaacaccataaagtacccagaggcgcgggcctggttgccatgcagggtgggaggggtcgtcaacggcgcccctggctcctccgtagccgcgctgcgcaccagcgggaggttaaggtgctcgcgaatgtggtttagctcccgcagccggcgggcctcgattggcactccccggacggtgagcgctccgttgacgaacatgaagggctggaacagacccgccaactgacgccagctctccaggtcgcaacagaggcagtcaaacaggtcgggccgcatcatctgctcggcgtacgcggcccataggatctcgcgggtcaaaaatagatacaaatgcaaaaacagaacacgcgccagacgagcggtctctcggtagtacctgtccgtgatcgtggcgcgcagcatttctcccaggtcgcgatcgcgtccgcgcatgtgcgcctggcggtgcagctgccggacgctggcgcgcaggtaccggtacagggccgagcagaagttggccaacacggttcgatagctctcctcccgcgcccgtagctcggcgtggaagaaacgagagagcgcttcgtagtagagcccgaggccgtcgcgggtggccggaagcgtcgggaaggccacgtcgccgtgggcgcgaatgtcgatttgggcgcgttcggggacgtacgcgtccccccattccaccacatcgctgggcagcgttgataggaatttacactcccggtacaggtcggcgttggtcggtaacgccgaaaacaaatcctcgttccaggtatcgagcatggtacatagcgcggggcccgcgctaaagcccaagtcgtcgaggagacggttaaagagggcggcgggggggacgggcatgggcggggagggcatgagctgggcctggctcaggcgccccgttgcgtacagcggaggggccgccggggtgtttttgggacccccggccgggcgggggggtggtggcgaagcgccgtccgcgtccatgtcggcaaacagctcgtcgaccaagaggtccattgggtggggttgatacgggaaagacgatatcgggcttttgatgcgatcgtccccgcccgcccagagagtgtgggacgcccgacggcgcgggaagagaaaaacccccaaacgcgttagaggaccggacggaccttatggggggaagtgggcagcgggaaccccgtccgttcccgaggaatgacagcccgtggtcgccaccccgcatttaagcaacccgcacgggccgccccgtacctcgtgacttccccccacattggctcctgtcacgtgaaggcgaaccgagggcggctgtccaacccaccccccgccacccagtcacggtccccgtcggattgggaaacaaaggcacgcaacgccaacaccgaatgaacccctgttggtgctttattgtctgggtacggaagtttttcactcgacgggccgtctggggcgagaagcggagcgggctggggctcgaggtcgctcggtggggcgcgacgccgcagaacgccctcgagtcgccgtggccgcgtcgacgtcctgcaccacgtctggattcaccaactcgttggcgcgctgaagcaggtttttgccctcgcagaccgtcacgcggatggtggtgatgccaaggagttcgttgaggtcttcgtctgtgcgcggacgcgacatgtcccagagctggaccgccgccatccgggcatgcatggccgccaggcgcccgaccgcggcgcagaagacgcgcttgttaaagccggccacccggggggtccatggcgcgtcggggtttgggggggcggtgctaaagtgcagctttctggccagcccctgcgcgggtgtcttggatcgggttggcgccgtcgacgcgggggcgtctgggagtgcggcggattctggctgggccgatttcctgccgcgggtggtctccgccgccggggccgcgggggccttagtcgccacccgctgggttcggggggcccggggggcggtggtgggtgtgcgtccggcccctccggacccagcgggcggcggaggcgcccgcgcaggccccgggccggacaaaaccgccccggaaacgggacgccgcgtccgggggacctccgggtgttcgtcgtcttcggatgacgagcccccgtagagggcataatccgactcgtcgtactggacgaaacggacctcgcccctcgggcgcgcgcgtgtctgtagggcgccacggcgggaggtggcaggcggactatcgggactcgccatacatgaagacggggtgtagtacagatcctcgtactcatcgcgcggaacctcccgcggacccgacttcacggagcggcgagaggtcatggttccacgaacacgctagggtcggatgcgcggacaattaggcctgggttcggacggcggggggtggtgcaggtgtggagaggtcgagcgataggggcggcccgggagagaagagagggtccgcaaaacccactggggatgcgtgagtggccctctgtgggcggtgggggagagtcttataggaagtgcatataaccacaacccatgggtctaaccaatccccaggggccaagaaacagacacgccccaaacggtctcggtttccgcgaagaaggggaagtcctgggacaccctccacccccacccctcaccccacacagggcgggttcaggcgtgcccggcagccagtagcctctggcagatctgacagacgtgtgcgataatacacacgcccatcgaggccatgcctacataaaagggcaccagggcccccggggcagacatttggccagcgttttgggtctcgcaccgcgcgcccccgatcccatcgcgcccgccctcctcgccgggcggctccccgtgcgggcccgcgtctcccgccgctaaggcgacgagcaagacaaacaacaggcccgcccgacagacccttctgggggggcccatcgtccctaacaggaagatgagtcagtggggatccggggcgatccttgtccagccggacagcttgggtcgggggtacgatggcgactggcacacggccgtcgctactcgcgggggcggagtcgtgcaactgaacctggtcaacaggcgcgcggtggcttttatgccgaaggtcagcggggactccggatgggccgtcgggcgcgtctctctggacctgcgaatggctatgccggctgacttttgtgcgattattcacgcccccgcgctatccagcccagggcaccacgtaatactgggtcttatcgactcggggtaccgcggaaccgttatggccgtggtcgtagcgcctaaaaggacgcgggaatttgcccccgggaccctgcgggtcgacgtgacgttcctggacatcctggcgccccccccggccctcaccaagccgatttccctgcggcagttcccgcaactggcgcccccccctccaaccggggccgggatacgcgcagatccttggttggagggggcgctcggggacccaagcgtgactccggccctaccggcgcgacgccgagggcggtccctcgtctatgccggcgagctgacgccggttcagacggaacacggggacggcgtacgagaagccatcgccttccttccaaaacgcgaggaggatgccggtttcgacattgtcgtccgtcgcccggtcaccgtcccggcaaacggcaccacggtcgtgcagccatccctccgcatgctccacgcggacgccgggcccgcggcctgttatgtgttggggcggtcgtcgctcaacgcccgcggcctcctggtcgttcctacgcgctggctccccgggcacgtatgtgcgtttgttgtttacaaccttacgggggttcctgtgaccctcgaggccggcgccaaggtcgcccagctcctggttgcgggggcggacgctcttccttggatccccccggacaactttcacgggaccaaagcgcttcgaaactaccccaggggtgttccggactcaaccgccgaacccaggaacccgccgctcttggtgtttacgaacgagtttgacgcggaggcccccccgagcgagcgcgggaccgggggttttggctctaccggtatttagcccatagcttggggttcgttccgggcaataaaaaacgtttgtatctcatctttcctgtgtgtagttgtttctgttggaggcctgtgggtctatcacacccgcccctccatcccacaaacacagaacacacgggttggatgaaaacacgcatttattgacccaaaacacacggagctgctcgagatgggccagggcgaggtgcggttggggaggctgtaggtctgggaacggacacgcggggacacgattccggtttggggtccgggagggcgtcgccgtttcgggcggcaggcgccagcgtaacctccgggggcggcgtgtgggggtgccccaaggagggcgcctcggtcaccccaagcccccccaagcgggttcccccggcaaccccgaaggcggagaggccaagggcccgttcggcgatggccacatcctccatgaccacgtcgctctcggccatgctccgaatagcctgggagacgagcacatccgcggacttgtcagccgcccccacggacatgtacatctgcaggatggtggccatacacgtgtccgccaggcgccgcatcttgtcctgatgggccgccacggccccgtcgatcgtgggggcctcgagcccggggtggtggcgcgccagtcgttctaggttcaccatgcaggcgtggtacgtgcgggccaaggcgcgggccttcacgaggcgtcgggtgtcgtccagggaccccagggcgtcatcgagcgtgatgggggcgggaagtagcgcgttaacgaccaccagggcctcctgcagccgcggctccgcctccgagggcggaacggccgcgcggatcatctcatattgttcctcggggcgcgctccccagccacatatagccccgagaagagaagccatcgcgggcgggtactggcccttgggcgcgcggacgcaatggggcaggaagacgggaaccgcggggagaggcgggcggccgggactcccgtggaggtgaccgcgctttatgctaccgacgggtgcgttattacctcttcgatcgccctcctcacaaactctctactgggggccgagccggtttatatattcagctacgacgcatacacgcacgatggccgtgccgacgggcccacggagcaagacaggttcgaagagagtcgggcgctctaccaagcgtcgggcgggctaaatggcgactccttccgagtaaccttttgtttattggggacggaagtgggtgggacccaccaggcccgcgggcgaacccgacccatgttcgtctgtcgcttcgagcgagcggacgacgtcgccgcgctacaggacgccctggcgcacgggaccccgctacaaccggaccacatcgccgccaccctggacgcggaggccacgttcgcgctgcatgcgaacatgatcctggctctcaccgtggccatcaacaacgccagcccccgcaccggacgcgacgccgccgcggcgcagtatgatcagggcgcgtccctacgctcgctcgtggggcgcacgtccctgggacaacgcggccttaccacgctatacgtccaccacgaggcgcgcgtgctggccgcgtaccgcagggcgtattatggaagcgcgcagagtcccttctggtttcttagcaaattcgggccggacgaaaaaagcctggtgctcaccactcggtactacctgcttcaggcccagcgtctggggggcgcgggggccacgtacgacctgcaggccatcaaggacatctgcgccacctacgcgattccccacgccccccgccccgacaccgtcagcgccgcgtccctgacctcgtttgccgccatcacgcggttctgttgcacgagccagtacgcccgcggggccgcggcggccgggtttccgctttacgtggagcgccgtattgcggccgacgtccgcgagaccagtgcgctggagaagttcataacccacgatcgcagttgcctgcgcgtgtccgaccgtgaattcattacgtacatttacctggcccattttgagtgtttcagccccccgcgcctagccacgcatcttcgggccgtgacgacccacgaccccaaccccgcggccaacacggagcagccctcgcccctgggcagggaggccgtggaacaatttttttgccacgtgcgcgcccaactgaatatcggggagtacgtcaaacacaacgtgaccccccgggagaccgtcctggatggcgatacggccaaggcctacctgcgcgctcgcacgtacgcgcccggggccctgacgcccgcccccgcgtattgcggggccgtggactccgccaccaaaatgatggggcgtttggcggacgccgaaaagctcctggtcccccgcgggtggcccgcgtttgcgcccgccagtcccggggaggatacggcgggcggcacgccgcccccacagacctgcggaatcgtcaagcgcctcctgagactggccgccacggaacaacaggacaccacgcccccggcgatcgcggcgcttatccgtaatgcggcggtgcagactcccctgcccgtctaccggatatccatggtccccacgggacaggcatttgccgcgctggcctgggacgactgggcccgcataacgcgggacgctcgcctggccgaagcggtcgtgtccgccgaagcggcggcgcaccccgaccacggcgcgctgggcaggcggctcacggatcgcatccgcgcccagggccccgtgatgccccctggcggcctggatgccggggggcagatgtacgtgaatcgcaacgagatattcaacggcgcgctggcaatcacaaacatcatcctggatctcgacatcgccctgaaggagcccgtcccctttcgccggctccacgaggccctgggccactttaggcgcggggctctggctgcggttcagctcctgtttcccgcggcccgcgtggaccccgacgcatatccctgttattttttcaaaagcgcatgtcggcccggcccggcgtccgtgggttccggcagcggactcggcgacgacggggactggtttccctgctacgacgacgccggtgatgaggagtgggcggaggacccgggcgccatggacacatcccacgatcccccggacgacgaggttgcctactttgacctgtgccacgaagtcggccccacggcggaacctcgcgaaacggattcgcccgtgtgttcctgcaccgacaagatcggactgcgggtgtgcatgcccgtccccgccccgtacgtcgtccacggttctctaacgatgcggggggtggcacgggtcatccagcaggcggtgctgttggaccgagattttgtggaggccatcgggagctacgtaaaaaacttcctgttgatcgatacgggggtgtacgcccacggccacagcctgcgcttgccgtattttgccaaaatcgcccccgacgggcctgcgtgcggaaggctgctgccagtgtttgtgatcccccccgcctgcaaagacgttccggcgtttgtcgccgcgcacgccgacccgcggcgcttccattttcacgccccgcccacctatctcgcttccccccgggagatccgtgtcctgcacagcctgggtggggactatgtgagcttctttgaaaggaaggcgtcccgcaacgcgctggaacactttgggcgacgcgagaccctgacggaggtcctgggtcggtacaacgtacagccggatgcgggggggaccgtcgaggggttcgcatcggaactgctggggcggatagtcgcgtgcatcgaaacccactttcccgaacacgccggcgaatatcaggccgtatccgtccggcgggccgtcagtaaggacgactgggtcctcctacagctagtccccgttcgcggtaccctgcagcaaagcctgtcgtgtctgcgctttaagcacggccgggcgagtcgcgccacggcgcggacattcgtcgcgctgagcgtcggggccaacaaccgcctgtgcgtgtccttgtgtcagcagtgctttgccgccaaatgcgacagcaaccgcctgcacacgctgtttaccattgacgccggtacgccatgctcgccgtccgttccctgcagcacctctcaaccgtcgtcttgataacggcgtacggcctcgtgctcgtgtggtacaccgtcttcggtgccagtccgctgcaccgatgtatttacgcggtacgccccaccggcaccaacaacgacaccgccctcgtgtggatgaaaatgaaccagaccctattgtttctgggggccccgacgcacccccccaacgggggctggcgcaaccacgcccatatctgctacgccaatcttatcgcgggtagggtcgtgcccttccaggtcccacccgacgccatgaatcgtcggatcatgaacgtccacgaggcagttaactgtctggagaccctatggtacacacgggtgcgtctggtggtcgtagggtggttcctgtatctggcgttcgtcgccctccaccaacgccgatgtatgtttggtgtcgtgagtcccgcccacaagatggtggccccggccacctacctcttgaactacgcaggccgcatcgtatcgagcgtgttcctgcagtacccctacacgaaaattacccgcctgctctgcgagctgtcggtccagcggcaaaacctggttcagttgtttgagacggacccggtcaccttcttgtaccaccgccccgccatcggggtcatcgtaggctgcgagttgatgctacgctttgtggccgtgggtctcatcgtcggcaccgctttcatatcccggggggcatgtgcaatcacataccccctgtttctgaccatcaccacctggtgttttgtctccaccatcggcctgacagagctgtattgtattctgcggcggggcccggcccccaagaacgcagacaaggccgccgccccggggcgatccaaggggctgtcgggcgtctgcgggcgctgctgttccatcatcctctcgggcatcgcagtgcgattgtgttatatcgccgtggtggccggggtggtgctcgtggcgcttcactacgagcaggagatccagaggcgcctgtttgatgtatgacgtcacatccaggccggcggaaaccggaacggcatatgcaaattggaaactgtcctgtcttggggcccacccacccgacgcgtcatatgcaaatgaaaatcggtcccccgaggccacgtgtagcctggatcccaacgaccccgcccatgggtcccaattggccgtcccgttaccaagaccaacccagccagcatatccacccccgcccgggtccccgcggaagcggaacggtgtatgtgatatgctaattaaatacatgccacgtacttatggtgtctgattggtccttgtctgtgccggaggtggggcgggggccccgcccggggggcggaacgaggaggggtttgggagagccggccccggcaccacgggtataaggacatccaccacccggccggtggtggtgtgcagccgtgttccaaccacggtcacgcttcggtgcctctccccgattcgggcccggtcgctcgctaccggtgcgccaccaccagaggccatatccgacaccccagccccgacggcagccgacagcccggtcatggcgactgacattgatatgctaattgacctcggcctggacctctccgacagcgatctggacgaggacccacccgagccggcggagagccgccgcgacgacctggaatcggacagcagcggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcagcacgggggcaaggtggcccgcctccaacccccaccgaccaaagcccagcctgcccgcggcggacgccgcgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggggcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagctttggccgcagcgcacaggtcatgcacgacccctttggggggcagccgtttcccgccgcgaatagcccctgggccccggtgttggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggctgtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacacaccgccaggagtgttcgagtcgtgtctgcgagttgacggccagtcacatcgtcgcccccccgtacgtgcacggcaaatatttttattgcaactccctgttttaggtacaataaaaacaaaacatttcaaacaaatcgccccacgtgttgtccttctttgctcatggccggcggggcgtgggtcacggcagatggcgggggtgggcccggcgtacggcctgggtgggcggagggaactaacccaacgtataaatccgtccccgctccaaggccggtgtcatagtgcccttaggagcttcccgcccgggcgcatccccccttttgcactatgacagcgacccccctcaccaacctgttcttacgggccccggacataacccacgtggcccccccttactgcctcaacgccacctggcaggccgaaacggccatgcacaccagcaaaacggactccgcttgcgtggccgtgcggagttacctggtccgcgcctcctgtgagaccagcggcacaatccactgctttttctttgcggtatacaaggacacccaccacacccctccgctgattaccgagctccgcaactttgcggacctggttaaccacccgccggtcctacgcgaactggaggataagcgcggggtgcggctgcggtgtgcgcggccgtttagcgtcgggacgattaaggacgtctctgggtccggcgcgtcctcggcgggagagtacacgataaacgggatcgtgtaccactgccactgtcggtatccgttctcaaaaacatgctggatgggggcctccgcggccctacagcacctgcgctccatcagctccagcggcatggccgcccgcgcggcagagcatcgacgcgtcaagattaaaattaaggcgtgatctccaacccccccatgaatgtgtgtaaccccccaaaaaaataaacagccgtaacccaatcaaaccaggcgtggtgtgagtttgtggacccaaagccctcagagacaacgcgacaggccagtatggaccgtgatacttttatttattaactcacaggggcgcttaccgccacaggaataccagaataatgaccaccactatcgcgaccaccccaaatacagcatggcgccccaccacgccacaacagccctgtcgccggtatggggcatgatcagacgagccgcgagccgcgcgttgggccctgtacagctcgcgcgaattgaccctaggaggccgccacgcgcccgagttttgcgttcgtcgctggtcgtcgggcgccaaagccccggacggctgttcggtcgaacgaacggccacgacagtggcataggttggggggtggtccgacatagcctcggcgtacgtcgggaggcccgacaagaggtcccttgagatgtcgggtggggccacaagcctggtttccggaagaaacaggggggttgccaataacccgccagggccaaaactccggcgctgcgcacgtcgttcggcgcggcgccgggcgcgccgagcggctcgctgggcggcttggcgtgagcggccccgctccgacgcctcgccctctccggaggaggttggcggaattggcacggacgacaggggcccagcagagtacggtggaggtgggtccgtgggggtgtccagatcaataacgacaaacggcccctcgttcctaccagacaagctatcgtaggggggcgggggatcagcaaacgcgttccccgcgctccatagacccgcgtcgggttgcgccgcctccgaagccatggatgcgccccaaagccacgactcccgcgcgctaggtccttggggtaagggaaaaggccctactccccatccaagccagccaagttaacgggctacgccttcggggatgggactggcaccccggcggattttgttgggctggtacgcgttgcccaaccgagggccgcgtccacgggacgcgccttttataaccccgggggtcattcccaacgatcacatgcaatctaactggctcccctctccccccctctcccctctccccccctctcccctctccccccctctcccctctccccccctctcccctctccccccctctcccctctccccccctctcccctctccccccctctcccctctccccccctctcccctctccccccctctcccctctccccccctctcccctctccccccctgctctttccccgtgacacccgacgctggggggcgtggctgccgggaggggccgcggatgggcggggcctactcggtctcccgcccccgcccccgaaccgccccgccggccttgcccccctttgatcccctgctacccccaacccgtgctcgtggtgcgggttgggtggggggggggagtgtgggcgggggtgtgcgggaggtgtcggtggtggtggtggtggtaggaatggtggtgaggggggggggcgctggttggtcaaaaaagggagggacgggggccggcagaccgacggcgacaacgctccccggcggccgggtcgcggctcttacgagcggcccggcccgcgctcccaccccccgggccgtgtccttgctttccccccgtctccccccccctccttctcctcctcctcctcctcgtttttccaaaccccgcccacccggcccggcccggcccggccaccgccgcccacccacccacctcgggagacccagccccggtcccccgttccccgggggccgttatctccagcgccccgtccggcgcgccgccccccgccgctaaaccccatcccgcccccgggaccccacatataagcccccagccacacgcaagaacagacacgcagaacggctgtgtttatttaaataaaccgatgtcggaataaacaaacacaaacacccgcgacggggggacggagggaggggggtgacgggggacgggaacagccacaaaaaacacccacaaaaaaaaacagccacccccgacacccccccccacccccagtctcttcgccttttcccccccaccccacgcccccactgagcccggtcgatcgacgagcacccccgcccccgcccctgccccggcgacccccggcccgcacgatcccgacaacaataacaaccccaacggaaagcggcggggtgtgggggggggcgaggaacaaccgaggggaacgggggatggaaggacgggaagtggaagtcctgatacccatcctacacccccctgccttccaccctccggccccccgcgagtccacccgccggccggctaccgagaccgaacacggcggccgccgcagccgccgcagccgccgccgacaccgcagagccggcgcgcgcacacacaagcggcagaggcagaaaggcccagagtcattgtttatgtggccgcgggccagcagacggcccgcgacacccccccccccgcccgtgtgggtatccggccccccgccccgcgccggtccattaagggcgcgcgtgcccgcgagatatcaatccgttaagtgctctgcagacaggggcaccgcgcccggaaatccattaggccgcagacgaggaaaataaaattacatcacctacccacgtggtgctgtggcctgtttttgctgcgtcatctgagcctttataaaagcgggggcgcggccgtgccgatcgcgggtggtgcgaaagactttccgggcgcgtccgggtgccgcggctctccgggcccccctgcagccggggcggccaaggggcgtcggcgacatcctccccctaagcgccggccggccgctggtctgttttttgttttccccgtttcgggggtggggggggttgcggtttctgtttctttaacccgtctggggtgtttttcgttccgtcgccggaatgtttcgttcgtctgtcccctcacggggcgaaggccgcgtacggcccgggacgagggggcccccgaccgcggcggtccgggccccgtccgggcccgctcgccggcacgcgacgcgaaaaaggccccccggaggcttttccgggttcccggcccggggcctgagataaacaatcggggttaccgccaacggccggcccccgtggcggcccggcccggggccccggcggacccaaggggccccggcccggggccccacaacggcccggcgcatgcgctgtggtttttttttctcggtgttttgtcgggctccgtcgcctttcctgttctcgcttctccccccccccccttcttcacccccagtaccctcctccctcccttcctcccccgttatcccactcgtcaagggcgccccggtgtggttcaacaaagacgccgcgtttccaggtaggttagacacctgcttctccccaatagaggggggggacccaaacgacagggggcgccccagaggctaaggtcggccacgccactcgcgggtgggctcgtgttacagcacaccagcccgttcttttccccccctcccacccttagtcagactctgttacttacccgtccgaccaccaactgcccccttatctaagggccggctggaagaccgccagggggtcggccggtgtcgctgtaaccccccacgccaatgacccacgtactccaagaaggcatgtgtcccaccccgcctgtgtttttgtgcctggctctctatgcttgggtcttactgccgggggggggggagtgcgggggagggggggtgtggaaggaaatgcacggcgcgtgtgtaccccccctaaagttgttcctaaagcgaggatatggaggagtggcgggtgccgggggaccggggtgatctctggcacgcgggggtgggaagggtcgggggaggggggatggggtaccggcccacctggccgacgcgggtgcgcgtgcctctgcacaccaaccccacgtcccccggcggtctctaagaagcaccgccccccctccttcataccaccgagcatgcctgggtgtgggttggtaaccaacacgcccatcccctcgtctcctgtgattctctggctgcaccgcattcttgttttctaactatgttcctgtttctgtctccccccccacccctccgccccaccccccaacacccacgtctgtggtgtggccgacccccttttgggcgccccgtcccgccccgccacccctcccgtcctttgttgccctatagtgtagttaacccccccccgccctttgtggcggccagaggccaggtcagtccgggcgggcaggcgctcgcggaaacttaacacccacacccaacccactgtggttctggctccatgccagtggcaggatgctttcggggatcggtggtcaggcagcccgggccgcggctctgtggttaacaccagagcctgcccaacatggcacccccactcccacgcacccccactcccacgcacccccactcccacgcacccccactcccacgcacccccactcccacgcacccccactcccacgcacccccactcccacgcacccccactcccacgcacccccactcccacgcacccccgagatccatccaacacagacagggaaaagatacaaaagtaaacctttatttcccaatagacagcaaaaatcccctgagttttttattagggccaacactaaagacccgctggtgtgtggtgcccgtgtctttcacttttcccctccccgacacggattggctggtgtagtgggcgcggccagagaccacccagcgcccgacccccccctccccacaaacacggggggcgtcccttattgttttccctcgtcccgggtcgacgccccctgctccccggaccacgggtgccgagaccgcaggctgcggaagtccagggcgcccactagggtgccctggtcgaacagcatgttccccacgggggtcatccagaggctgttccactccgacgcgggggccgtcgggtactcggggggcatcacgtggttacccgcggtctcggggagcagggtgcggcggctccagccggggaccgcggcccgcagccgggtcgccatgtttcccgtctggtccaccaggaccacgtacgccccgatgttccccgtctccatgtccaggatgggcaggcagtcccccgtgatcgtcttgttcacgtaaggcgacagggcgaccacgctagagacccccgagatgggcaggtagcgcgtgaggccgcccgcgggggcggccccggaagtctccgcgtggcgcgtcttccgggcacacttcctcggcccccgcggcccagaagcagcgcgggggccgagggaggtttcctcttgtctccctcccagggcaccgacggccccgcccgaggaggcggaagcggaggaggacgcggccccggcggcggaagaggcggcccccgcgggagtcggggccgaggaggaagaggcagaggaggaagaggcggaggccgccgaggacgtcaggggggtcccgggcccaccctggccgcgcccccccggccctgagtcggagggggggtgcgtcgccgccctcttggcccctgccggcgcgagggggggacgcgtggactggggggaggggttttcctggcccgacccgcgcctcttcctcggacgcaccgccgcctcctgctcgacagaggcggcggaggggagcgggggggcgccggagggggcggcgccgcgggagggcccgtgtccaccctccacgcccggcccccccgagccgcgcgccaccgtcgcacgcgcccggcacagactctgttcttggttcgcggcctgagccagggacgagtgcgactggggcacacggcgcgcgtccgcgggggccggggcgcgggggccgggccccggaggcggcgctcgcacgcacggggccacggccgcgcgggggcgcgcgggtcccgacgcggccgaggacgcggtgggcccggggcggggggcggagcctggcatgggcgccgcggggggcctgtggggagaggccgggggggagtcgctgatcactatggggtctctgttgtttgcaaggggggcgggtctgttgacaagggggcccgtccggcccctcggccgccccgcctccgcttcaacaaccccaaccccaaccccaacccccccggaggggccagacgccccccgcggcgccgcggctcgcgactggcgggagccgccgccgccgccgctgctgttggtggtggtgttggtgttactgctgccgtgtggcccgatgggcgccgaggggggcgctgtccgagccgcggccggctggggggctgcgtgagacgccccgcccgtcacggggggcgcggcggcgcctctgcgtgggggggcgcggggcgtccggcggggggcgggcggtacgtagtctgctgcaagagacaacggggggcgcgatcaggttacgccccctcccaggccctccctttccgcgcccgccctttcctcgcccccccgcccgcctattcctccctcccccctcctcctcctcctcccccagggtcctcgccgcccccccgcctcaccgtcgtccaggtcgtcgtcatcctcgtccgtggtgggctcagggtgggtgggcgacagggccctcaccgtgtgcccccccagggtcaggtaccgcggggcgaaccgctgattgcccgtccagataaagtccacggccgtgcccgccctgacggcctcctcggcctccatgcgggtctgggggtcgttcacgatcgggatggtgctgaacgacccgctgggcgtcacgcccactatcaggtacaccagcttggcgttgcacagcgggcaggtgttgcgcaattgcatccaggttttcatgcacgggatgcagaagcggtgcatgcacgggaaggtgtcgcagcgcaggtggggcgcgatctcatccgtgcacacggcgcacacgtcgccctcgtcgctccccccgtcctctcgagggggggcgcccccgcaactgccggggtcttcctcgcggggggggctcccccccgagaccgcccccccatccacgccctgcggccccagcagccccgtctcgaacagttccgtgtccgtgctgtccgcctcggaggcggagtcgtcgtcatggtggtcggcgtccccccgcccccccacttcggtctccgcctcagagtcgctgctgtccggcaggtctcggtcgcagggaaacacccagacatccggggcgggctaaggggaaaaaaaggggggcgggtaagaatggggggatttcccgcgtcaatcagcgcccacgagttccccctctccccccccccgcctcacaaagtcctgcccccctgctggcctcggaagaggggggagaaaggggtctgcaaccaaaggtggtctgggtccgtcctttggatcccgacccctcttcttccctcttctcccgccctccagacgcaccggagtcgggggtcccacggcgtcccccaaatatggcgggcggctcctccccacccccctagatgcgtgtgagtaagggggccctgcgtatgagtcagtggggaccacgccccctaacacggcgaccccggtccctgtgtgtttgttgtgggggcgtgtctctgtgtatgagtcagggggtcccacggcgaccccgggccctgcgtctgagtcaaaggggccatgtgtatgtgttggggggtctgtatatataaagtcagggggtcacatggcgacccccaacagggcgaccccggtccctgtatatatagggtcagggggttccgcgccccctaacatggcgcccccggtccctgtatatatagtgtcacggggttccacgccccctaacatggcgcccggctcccgtgtatgagtgggggtcccccaacatggcggccggttccagtgtaagggtcgggggtcccccaacatggcgccccccaatatggcgccccccaatatggcgccccagacatggcgcccggcccctcacctcgcgctgggggcggccctcaggccggcgggtactcgctccggggcggggctccatgggggtcgtatgcggttggagggtcgcggacggagggtccctgggggtcgcaacgtaggcggggcttctgtggtgatgcggagagggggcggcccgagtctgcctggctgctgcgtctcgctccgagtgccgaggtgcaaatgcgaccagactgtcgggccagggctaacttataccccacgcctttcccctccccaaaggggcggcagtgacgattcccccaatggccgcgcgtcccaggggaggcaggcccaccgcggggcggccccgtccccggggaccaacccggcgcccccaaagaatatcattagcatgcacggcccggcccccgatttgggggaccaacccggtgtcccccaaagaaccccattagcatgcccctcccaccgacgcaacaggggcttggcctgcgtcggtgccccggggcttcccgccttcccgaagaaactcattaccatacccggaaccccaggggaccaatgcgggttcattgagcgacccgcgggccaatgcgcgaggggccgtgtgttccgccaaaaaagcaattagcataacccggaaccccaggggagtggttacgcgcggcgcgggaggcggggaataccggggttgcccattaagggccgcgggaattgccggaagcgggaagggcggccggggccgcccattaatgagtttctaattaccataccgggaagcggaacaaggcctcttgcaagtttttaattaccataccgggaagtgggcggcccggcccattgggcggtaactcccgcccaatgggccgggccccgaagactcggcggacgctggttggccgggccccgccgcgctggcggccgcgtttaaacctgcaggtcgactctagaggatctcgtactgaaggaaagaagtcagaaggcaaaaacgagagtaactccacagtagctccaaattctttataagggtcgatgtccatgccccaaagccacccaaggcacagcttggaggcttgaacagtaggacatgaacaagagatgattaggcagaggtgaaaaaagttgcatggtgctggtgcgcagaccaatttatgcctacagcctcctagtacaaagacctttaacctaatctcctcccccaactcctcccagtctttaaacaaacagtctttgaagtatgcctcaaggtcggtcgttgacattgctgagagtccaagagtccttcttatgtaagaccttgggcaatatttggtgggcgtttcacggtggtctcccatgcgacgtgcagaggtgaagcgaagtgcacacggtccggcagatgagaaggcacagacggggagtccgcgtaaagagaggtgcgccccgtggtcggtcggaacggcagacggagaaggggacgagagagtcccaagcgaccccgagaagggtcgtccgcaggattcagcgccgacgggacgtaaacaaaggacgtccccgcgcaggatccccggatcgtcgacagatctggtaccccgctcgagcggagctcttaagtatacgtcgacaggaacaggtggtggcggccctcggcgcgctcgtactgctccacgatggtgtagtcctcgttgtgggaggtgatgtccagcttggagtccacgtagtagtagccgggcagctgcacgggcttcttggccatgtagatggacttgaactccaccaggtagtggccgccgtccttcagcttcagggccttgtggatctcgcccttcagcacgccgtcgcgggggtacaggcgctcggtggaggcctcccagcccatagtcttcttctgcattacggggccgtcggaggggaagttcacgccgatgaacttcaccttgtagatgaaggagccgtcctgcagggaggagtcctgggtcacggtcaccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttggcggtctgggtgccctcgtaggggcggccctcgccctcgccctcgatctcgaactcgtggccgttcacggagccctccatgcgcaccttgaagcgcatgaactccttgatgacgtcctcggaggacgtcgacataggcccgggattctcttccacgtctccgcaggtgagcaggctccctctgccctccctcttagctcgcgaggaatcttcacttccgtgtgttctagaggacagatgctgatgaatcatcttttggagaagtgatttgaatctttctaggaattctttgggtggttttttctcataagaatcacatgaagggcatgttagtctgtgtttctgtcttctccctgcatttgtggaaggtggtttcctcttcagctttttaattgatacattgattatcctttcattgtttcctgtatttgctgactttagttgggccttctgaaagcaggaaaaagctgaccactcacagtttgtctctacatcttctggagctggcagaaattcagggaccaagtcattcacataatttttcagctgatcaacaatatctataagttgacgcattctaatcatgtggcgatcttgaccttgggagcttgatttgtggaccagtgtccccaagaagatgaccatcagacagatgacaatcctctccatgttgccaggactggatctcatgtcggatccgcgccgatccggtacggtaccactttgtgcggccagcttcctaggagatccagatctgtttaaacctagagtcgatcgacctgcagcccaagcttatcgataccgtcgacctcgacctcgactcgaccggaagctggcggcggcggcgaatcgcggcggcggctccccaagttctgcgcgctgggatctctcgcgactccccgaatcggcctctatttcaagggtctggaacagcacgggcccgcttccgcgtgacgtacatggccatatatgggaagcaggaagccctaatatggaaggaccggccgggaggacccggagngacgtgaagccctcccatccaagagtggtgggcacntccaccctgcgacccgctccggcatcgcgagcgctcaggntcctggaaagcctagtattggaacagcagcagtggcctcccgggccaggcgcctggagttcccagnttgcccccgccccaacccccgctcgcccntcctcccagagccggaagcggcagggccatattgggccactccatataaggcgctgcccaaataaggtctgttccggcgggggatccttcctgctccttatatggcgtttccgggtcggcgcatctagggtcgagggttacctgggactgtgcggttgggacggcgcccgtgggcccgggcggccgggggcggcgggggccgcgatggcggcggcggcgggccatggagacagagagcgtgccggggtggtagagtttgacaggcaagcatgtgcgtgcagaggcgagtagtgcttgcctgtctaactcgctcgtctcggccgcggggggcccgggctgcgccgccgcgctttaaagggccgcgcgcgacccccggggggtgtgtttcggggggggcccgttttccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctccccaacgcccgccgcgcgcgcgcacgccgcccggaccgccgcccgccttttttgcgcgccgccccgcccgcggggggcccgggctgccacaggtgtaacaacaccaacagaacaccaacagcacggcgcaccggcgactccggttcctcatccacacgtcacacgtcacgtcatccaccacacctgcccaccaacacaactcacagcgacaactcaccgcgcaacaactcctgttcctcatccacacgtcaccgcgcaccccccgctcctccagacgtcccccagcgcaacacgccgctcctgtcacacaccaccgccccagccctccccagccccagccctccccagccccagccctccccggccccagccctccccggccccagccctccccggccccagccctccccggccccagccctccccggccccagccctccccggccccagccctccccggccgcgtcccgcgctccctcgggggggttcgggcatctctacctcagtgccgccaatctcaggtcagagatccaaaccctccgggggcgcccgcgcaccaccaccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgaaataaacaacgctactgcaaaactaaatcaggtcgttgtcgtttattgcgtcttcgggtttcgcaagcgccccgccccgtcccggcccgttacagcaccccgtccccctcgaacgcgccgccgtcgtcgtcgtcccaggcgccttcccagtccacaacttcccgtcgcgggggcgtggccaagcccgcctccgcccccagcacctccacggcccccgccgccgccagcacggtgccgctgcggcccgtggccgaggcccagcgaatcccgggcaacgccggcggcagggcccccgggccgtcgtcgtcgtcgtcgccgccgcgcagcaccagcgggggggcgtcgtcgtcgggctccagcagggcgcgggcgcaaaagtccctccgcggcccgcgccaccgggccgggccggcgcgcaccgcctcgcgccccagcgccacgtacacgggccgcagcggcgcgcccaggccccagcgcgcgcaggcgcggtgcgagtgggcctcctcctcgcagaagtccggcgcgccgggcgccatggcgtcggtggtccccgaggccgccgcccggccgtccagcgccggcagcacggcccggcggtactcgcgcggggacatgggcaccggcgtgtccgggccgaagcgcgtgcgcacgcggtagcgcacgttgccgccgcggcacaggcgcagcggcggcgcgtcggggtacaggcgcgcgtgcgcggcctccacgcgcgcgaagacccccgggccgaacacgcggcccggggccagcaccgtgcggcgcaggtcccgcgccgccggccagcgcacggcgcactgcacggcgggcagcaggtcgcacgccaggtaggcgtgctgccgcgacaccgcgggcccgtcggcgggccagtcgcaggcgcgcacggtgttgaccacgatgagccgccggtcgccggcgctggcgagcagccccagaaactccacggccccggcgaaggccaggtcccgcgtggacagcagcagcacgccctgcgcgcccagcgccgacacgtcgggggcgccggtccagttgcccgcccaggcggccgtgtccggcccgcacagccggttggccagggccgccagcaggcaggacagcccgccgcgctcggcggaccactccggcggcccccccgaggccccgccgccggccaggtcctcgcccggcagcggcgagtacagcaccaccacgcgcacgtcctcggggtcggggatctggcgcatccaggccgccatgcggcgcagcgggcccgaggcgcgcagggggccaaagaggcggcccccggcggccccgtgggggtgggggttatcgtcgtcgtcgccgccgccgcacgcggcctgggcggcggcggcgggcccggcgcaccgcgcggcgatcgaggccagggcccgcgggtcaaacatgagggccggtcgccaggggacggggaacagcgggtggtccgtgagctcggccacggcgcgcggggagcagtaggcctccagggcggcggccgcgggcgccgccgtgtggctgggccccgggggctgccgccgccagccgcccagggggtcggggccctcggcgggccggcgcgacagcgccacggggcgcgggcgggcctgcgccgcggcggcccggggcgccgcgggctgggcgggggcgggctcgggccccgggggcgtggaggggggcgcggggaggggggcgcgggcgtccgagccgggggcgtccgcgccgctcttcttcgtcttcgggggtcgcgggccgccgcctccgggcggccgggccgggccgggactcttgcgcttgcgcccctcccgcggcgcggcggaggcggcggcggccgccagcgcgtcggcggcgtccggtgcgctggcggccgccgccagcagggggcgcaggctctggttctcaaacagcaggtccgcggcggcggcggccgcggagctcggcaggcgcgggtcccgcggcagcgcggggcccagggccccggcgaccaggctcacggcgcgcacggcggccacggcggcctcgctgccgccggccacgcgcaggtccccgcgcaggcgcatgagcaccagcgcgtcgcgcacgaaccgcagctcgcgcagccacgcgcgcaggcggggcgcgtcggcgtgcggcggcggcggggaagcggggcccgcgggtccctccggccgcggggggctggcgggccgggccccggccagccccgggacggccgccaggtcgccgtcgaagccctcggccagcgcctccaggatcccgcggcaggcggccaggcactccacggccacgcggccggcctgggcgcggcgcccggcgtcgtcgtcggcgtcggcgtggcgggcggcgtcggggtcgtcgccccccgcgggggaggcgggcgcggcggacagccgccccagggcggcgaggatccccgcggcgccgtacccggcgggcaccgcgcgctcgcccggtgcggcggcggcggcgacgacggcggcggcgaccccctcgtcatctgcgccggcgccggggctccccgcggcccccgtcagcgccgcgttctcgcgcgccaacaggggcgcgtaggcgcggcgcaggctggtcagcaggaagcccttctgcgcgcggtcgtatcggcggctcatggccacggcggccgccgcgtgcgccaggccccagccgaagcggccggccgccatggcgtagcccaggtggggcacggcccgcgccacgctgccggtgatgaaggagctgctgttgcgcgcggcgcccgagatccggaagcaggcctggtccagcgccacgtccccggggaccacgcgcgggttctggagccaccccatggcctccgcgtccggggtgtacagcagccgcgtgatcagggcgtactgctgcgcggcgtcgcccagctcgggcgcccacacggccgccggggcgcccgaggcctcgaaccggcgtcgcgcctcctccgcctcgggcgccccccagaggcccgggcggctgtcgcccaggccgccgtacagcacccgccccgggggcgggggcccggcgccgggccacggctccccgctgacgtacccgtcgcgatagcgcgcgtagaaggcgccggaggccgcgtcggcgtccagctcgacccgccggggctgcccggccgtgaagcggcccgtggcgtcgcggccggccaccgccgcgcgggcccggcggcgctcgatgcggcccgcggaggccgcgggggtcctcgccgccgcccggggcttgggcgcggcctcggagagggggggtggcccgggcgggggcggcgtccgcccgggggcttccggcgccgcgctcgacggaccccgcccgacggcccgcgcctcgcgtgcgcggtcggccgcgtcgttgccgtcgtcgtcctcgtcctcgtcggacgacgaggacgaagaggatgcggacgacgaggacgaggacccggagtccgacgaggtcgatgacgccgatggccgccgccggccgtgacgacgtctccgcggcggctgggccggcgggcgcggcgacaggcggtccgtggggtccggatacgcgccgcgtagcggggcctcccgtgcgcggccccgggccggggcccggtcgccggcggcgtcggctgcgtcgtcgtactcgtccccgtcatcgtcgtcggctcgaaaggcgggggtccggggcggcgaggccgcggggtcgggcgtcgggatcgtccggacggcctcctctaccatggaggccagcagggccagctgtcgcggcgagacggcgtccccggcgtcctcgccggcgtcggtgcccgccgcgggggccctcccgtcccgccgggcgtcgtcgaggtcgtgggggtggtcggggtcgtggtcggggtcgtccccgccctcctccgtctccgcgccccacccgagggccccccgctcgtcgcggtctgggctcggggtgggcggcggcccgtcggtggggcccggggagccggggcgctgcttgttctccgacgccatcgccgatgcggggcgatcctccggggatacggctgcgacggcggacgtagcacggtaggtcacctacggactctcgatggggagggggcgagacccacggaccccgacgacccccgccgtcgacgcggaactagcgcggaccggtcgatgcttgggtgggaaaaaggacagggacggccgatccccctcccgcgcttcgtccgcgtatcggcgtcccggcgcggcgagcgtctgacggtctgtctctggcggtcccgcgtcgggtcgtggatccgtgtcggcagccgcgctccgtgtggacgatcggggcgtcctcgggctcatatagtcccaggggccggcgggaaggaggagcagcggaggccgccggccccccgccccccaggcgggcccgccccgaacggaattccattatgcacgaccccgccccgacgccggcacgccgggggcccgtggccgcggcccgttggtcgaacccccggccccgcccatccgcgccatctgccatgggcggggcgcgagggcgggtgggcccgcgccccgccccgcatggcatctcattaccgcccgatccggtggtttccgcttccgttccgcatgctaacgaggaacgggccgggggcggggcccgggccccgacttcccggttcggcggtaatgagatacgagccccgcgcgcccgttggccgtccccgggcccccggtcccgcccgccggacgttgggaccaacgggacggcgggcggcccaagggccgcccgccttgccgcccccccattggccggcgggcgggaccgccccaagggggcggggccgccgggtaaaagaagtgagaacgcgaagcgttcgcacttcgtcccaatatatatatattattagggcgaagtgcgagcactggcgccgtgcccgactccgcgccggccccgggggcgggcccgggcggcggggggcgggtctctccggcgcacataaaggcccggcgcgaccgacgcccgcagacggcgccggccacgaacgacgggagcggctgcggagcacgcggaccgggagcgggactcgcagagggccgtcggagcggacggcgtcggcatcgcgacgccccggctcgggatcgggatcgcatcggaaagggacacgcggaaagacccacccaccccacccacgaaacacaggggacgcaccccgggggcctccgacgacagaaacccaccggtccgcctttgtgcacgggtaagcaccttgggtgggcggaggagggggggacgcgggggcggaggaggggggacgcgggggcggaggaggggggacgcgggggcggaggaggggggacgcgggggcggaggaggggggacgcgggggcggaggagggggctcacccgcgttcgtgccttcccgcaggaggaacgtcctcgtcgaggcgaccggcggcgaccgttgcgtggaccgcttcctgctcgtcgggcggggggaagccactgtggtcctccgggacgttttctggatggccgacatttccccaggcgcttttgcgccttgtgtaaaagcgcggcgtcccgctctccgatccccgcccctgggcacgcgcaagcgcaagcgcccttcccgccccctctcatcggagtctgaggtagaatccgatacagccttggagtctgaggtcgaatccgagacagcatcggattcgaccgagtctggggaccaggatgaagccccccgcatcggtggccgtagggccccccggaggcttggggggcggttttttctggacatgtcggcggaatccaccacggggacggaaacggatgcgtcggtgtcggacgaccccgacgacacgtccgactggtcttatgacgacattcccccacgacccaagcgggcccgggtaaacctgcggctcacgagctctcccgatcggcgggatggggttatttttcctaagatggggcgggtccggtctacccgggaaacgcagccccgggcccccaccccgtcggccccaagcccaaatgcaatgctacggcgctcggtgcgccaggcccagaggcggagcagcgcacgatggacccccgacctgggctacatgcgccagtgtatcaatcagctgtttcgggtcctgcgggtcgcccgggacccccacggcagtgccaaccgcctgcgccacctgatacgcgactgttacctgatgggatactgccgagcccgtctggccccgcgcacgtggtgccgtttgctgcaggtgtccggcggaacctggggcatgcacctgcgcaacaccatacgggaggtggaggctcgattcgacgccaccgcggaacccgtgtgcaagcttccttgtttggagaccagacggtacggcccggagtgtgatcttagtaatctcgagattcatctcagcgcgacaagcgatgatgaaatctccgatgccaccgatctggaggccgccggttcggaccacacgctcgcgtcccagtccgacacggaggatgccccctcccccgttacgctggaaaccccagaaccccgcgggtccctcgctgtgcgtctggaggatgagtttggggagtttgactggaccccccaggagggctcccagccctggctgtctgcggtcgtggccgataccagctccgtggaacgcccgggcccatccgattctggggcgggtcgcgccgcagaagaccgcaagtgtctggacggctgccggaaaatgcgcttctccaccgcctgcccctatccgtgcagcgacacgtttctccggccgtgagtccggtcgccccgacccccttgtatgtccccaaataaaagaccaaaatcaaagcgtttgtcccagcgtcttaatggcgggaagggcggagagaaacagaccacgcgtacatggggggtgtttgggggtttattgacatcggggctacagggtggtaaccggatagcagatgtgaggaagtctgggccgttcgccgcgaacggcgatcagagggtccgtttcttgcggaccacggcccggtgatgtgggttgctcgtctgggatctcgggcatgcccatacacgcacaacacggacgccgcaccggatgggacgtcgtaagggggcctggggtagctgggtggggtttgtgcagagcaatcagggaccgcagccagcgcatacaatcgcgctcccgtccgtttgtcccgggcagtaccacgccgtactggtattcgtaccggctgagcagggtctccagggggtggttgggggccgcggggaacggggtccacgccacggtccactcgggcaaaaaccgagtcggcacggcccacggttctcccacccacgcgtctggggtcttgatggcgataaatcttaccccgagccggattttttgggcgtattcgagaaacggcacacacagatccgccgcgcctaccacccacaagtggtagaggcgaggggggctgggttggtctcggtgcagcagtcggaagcacgccacggcgtccacgacctcggtgctctccaaggggctgtcctccgcaaacaggcccgtggtggtgtttggggggcagcgacaggacctagtgcgcacgatcgggcgggtgggtttgggtaagtccatcagcggctcggccaaccgtcgaaggttggccggacgaacgacgaccggggtacccaggggttctgatgccaaaatgcggcactgcctaagcaggaagctccacagggccgggcttgcgtcgacggaagtccggggcagggcgttgttctggtcaaggagggtcattacgttgacgacaacaacgcccatgttggtatattacaggcccgtgtccgatttggggcacttgcagatttgtaaggccacgcacggcggggagacaggccgacgcgggggctgctctaaaaatttaagggccctacggtccacagacccgccttcccgggggggcccttggagcgaccggcagcggaggcgtccgggggaggggagggtgatttacgggggggtaggtcagggggtgggtcgtcaaactgccgctccttaaaaccccggggcccgtcgttcggggtgctcgttggttggcactcacggtgcggcgaatggcctgtcgtaagttttgtcgcgtttacgggggacagggcaggaggaaggaggaggccgtcccgccggagacaaagccgtcccgggtgtttcctcatggccccttttataccccagccgaggacgcgtgcctggactccccgcccccggagacccccaaaccttcccacaccacaccacccggcgatgccgagcgcctgtgtcatctgcaggagatcctggcccagatgtacggaaaccaggactaccccatagaggacgaccccagcgcggatgccgcggacgatgtcgacgaggacgccccggacgacgtggcctatccggaggaatacgcagaggagctttttctgcccggggacgcgcccggtccccttatcggggccaacgaccacatccctcccccgtgtggcgcatctccccccggtatacgacgacgcagccgggatgagattggggccacgggatttaccgcggaagaactggacgccatggacagggaggcggctcgagccatcagccgcggcggcaagcccccctcgaccatggccaagctggtgactggcatgggctttacgatccacggagcgctcaccccaggatcggaggggtgtgtctttgacagcagccacccagattacccccaacgggtaatcgtgaaggcggggtggtacacgagcacgagccacgaggcgcgactgctgaggcgactggaccaccccgcgatcctgcccctcctggacctgcatgtcgtctccggggtcacgtgtctggtcctccccaagtaccaggccgacctgtatacctatctgagtaggcgcctgaacccgctgggacgcccgcagatcgcagcggtctcccggcagctcctaagcgccgttgactacattcaccgccagggcattatccaccgcgacattaagaccgaaaatatttttattaacacccccgaggacatttgcctgggggactttggtgccgcgtgcttcgtgcagggttcccgatcaagccccttcccctacggaatcgccggaaccatcgacaccaacgcccccgaggtcctggccggggatccgtataccaccaccgtcgacatttggagcgccggtctggtgatcttcgagactgccgtccacaacgcgtccttgttctcggccccccgcggccccaaaaggggcccgtgcgacagtcagatcacccgcatcatccgacaggcccaggtccacgttgacgagttttccccgcatccagaatcgcgcctcacctcgcgctaccgctcccgcgcggccgggaacaatcgcccgccgtacacccgaccggcctggacccgctactacaagatggacatagacgtcgaatatctggtttgcaaagccctcaccttcgacggcgcgcttcgccccagcgccgcagagctgctttgtttgccgctgtttcaacagaaatgaccgcccccagggggcggtgctgtttgcgggttggcacaaaaagaccccgacccgcgtctgtggtgtttttggcatcatgtcgccgggcgccatgcgtgccgttgttcccattatcccattccttttggttcttgtcggtgtatcgggggttcccaccaacgtctcctccaccacccaaccccaactccagaccaccggtcgtccctcgcatgaagcccccaacatgacccagaccggcaccaccgactctcccaccgccatcagccttaccacgcccgaccacacaccccccatgccaagtatcggactggaggaggaggaagaggaggagggggccggggacggcgaacatcttgaggggggagatgggacccgtgacaccctaccccagtccccgggcccagccttcccgttggctgaggacgtcgagaaggacaaacccaaccgtcccgtagtcccatcccccgatcccaacaactcccccgcgcgccccgagaccagtcgcccgaagacaccccccaccattatcgggccgctggcaactcgccccacgacccgactcacctcaaagggacgacccttggttccgacgcctcaacataccccgctgttctcgttcctcactgcctcccccgccctggacaccctcttcgtcgtcagcaccgtcatccacaccttatcgtttttgtgtattggtgcgatggcgacacacctgtgtggcggttggtccagacgcgggcgacgcacacaccctagcgtgcgttacgtgtgcctgccgtccgaacgcgggtagggtatggggcgggggatggggagagcccacacgcggaaagcaagaacaataaaggcggtggtatctagttgatatgcatctctgggtgtttttggggtgtggcggacgcggggcggtcattggacggggtgcagttaaatacatgcccgggacccatgaagcatgcgcgacttccgggcctcggaacccacccgaaacggccaacggacgtctgagccaggcctggctatccggagaaacagcacacgacttggcgttctgtgtgtcgcgatgtctctgcgcgcagtctggcatctggggcttttgggaagcctcgtgggggctgttcttgccgccacccatcggggacctgcggccaacacaacggaccccttaacgcacgccccagtgtcccctcaccccagccccctggggggctttgccgtccccctcgtagtcggtgggctgtgcgccgtagtcctgggggcggcgtgtctgcttgagctcctgcgtcgtacgtgccgcgggtgggggcgttaccatccctacatggacccagttgtcgtataatttcccccccccccccccttctccgcatgggtgatgtcgggtccaaactcccgacaccaccagctggtatggtataaatcaccggtgcgccccccaaaccatgtccggcagggggatgggggggcgaatgcggagggcacccaacaacaccgggctaaccaggaaatccgtggccccggcccccaataaagatcgcggtagcccggccgtgtgacactatcgtccataccgaccacaccgacgaatcccctaagggggaggggccattttacgaggaggaggggtataacaaagtctgtctttaaaaagcaggggttagggagttgttcggtcataagcttcagcgcgaacgaccaactaccccgatcatcagttatccttaaggtctcttttgtgtggtgcgttccggtatggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctgtccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacataccgtcgatccaggacgccgcgacgccttaccatcccccggccaccccgaacaacatgggcctgatcgccggcgcggtgggcggcagtctcctggcagccctggtcatttgcggaattgtgtactggatgcgccgccgcactcaaaaagccccaaagcgcatacgcctcccccacatccgggaagacgaccagccgtcctcgcaccagcccttgttttactagatacccccccttaatgggtgcgggggggtcaggtctgcggggttgggatgggaccttaactccatataaagcgagtctggaaggggggaaaggcggacagtcgataagtcggtagcgggggacgcgcacctgttccgcctgtcgcacccacagctttttttgcgaaccgtcccgttccgggatgccgtgccgcccgttgcagggcctggtgctcgtgggcctctgggtctgtgccaccagcctggttgtccgtggccccacggtcagtctggtatcaaactcatttgtggacgccggggccttggggcccgacggcgtagtggaggaagacctgcttattctcggggagcttcgctttgtgggggaccaggtcccccacaccacctactacgatggggtcgtagagctgtggcactaccccatgggacacaaatgcccacgggtcgtgcatgtcgtcacggtgaccgcgtgcccacgtcgccccgccgtggctttcgccctgtgtcgcgcaaccgacagcactcacagccccgcatatcccaccctggagctgaatctggcccaacagccgcttttgcgggtccggagggcgacgcgtgactatgccggggtgtacgtgttacgcgtatgggtcggggacgcaccaaacgccagcctgtttgtcctggggatggccatagccgccgaagggactctggcgtacaacggctcggcccatggctcctgcgacccgaaactgcttccgtattcggccccgcgtctggccccggcgagcgtataccaacccgcccctaacccggcctccaccccctcgaccaccacctccaccccctcgaccaccacctccaccccctcgaccaccatccccgctccccaagcatcgaccacacccttccccacgggagacccaaaaccccaacctcacggggtcaaccacgaacccccatcgaatgccacgcgagcgacccgcgactcgcgatacgcgctaacggtgacccagataatccagatagccatccccgcgtccattatagccctggtgtttctggggagctgtatttgctttatacacagatgtcaacgccgctaccgacgctcccgccgcccgatttacaacccccagatacccactggcatctcatgcgcggtgaacgaagcggccatggcccgcctcggagccgagctcaaatcgcatccgagcaccccccccaaatcccggcgccggtcgtcacgcacaccaatgccctccctgacggccatcgccgaagagtcggagcccgcgggggcggctgggcttccgacgccccccgtggaccccacgacatccaccccaacgcctcccctgttggtataggtccacggccactggccgggggcaccacataaccgaccgcagtcactgagttgggaataaaccggtattatttacctatatacgtgtatgtccatttcttcccccccccccccggaaaccaaagaaggaaacaaagaatggatgggaggagttcaggaagccggggagagggcccgcggcgcatttaaggcgttgttgtgttgactttggctcttctggcgggttggtgcggtgctgtttgttgggctcccattttacccgaagatcggctgctatccccgggacatggatcgcggggcggtggtggggtttcttctcggtgtttgtgttgtatcgtgcttggcgggaacgcccaaaacgtcctggagacgggtgagtgtcggcgaggacgtttcgttgcttccagctccggggcctacggggcgcggcccgacccagaaactactatgggccgtggaacccctggatgggtgcggccccttacacccgtcgtgggtctcgctgatgccccccaagcaggtgcccgagacggtcgtggatgcggcgtgcatgcgcgctccggtcccgctggcgatggcgtacgcccccccggccccatctgcgaccgggggtctacggacggacttcgtgtggcaggagcgcgcggccgtggttaaccggagtctggttatttacggggtccgagagacggacagcggcctgtataccctgtctgtgggcgacataaaggacccggctcgccaagtggcctcggtggtcctggtggtgcaaccggccccagttccgaccccacccccgaccccagccgattacgacgaggatgacaatgacgagggcgagggcgaggacgaaagtctagccggcactcccgccagcgggaccccccggctcccgcctccccccgcccccccgaggtcttggcccagcgcccccgaagtctcacacgtgcgtggggtgaccgtgcgtatggagactccggaagctatcctgttttcccccggggaggcgtttagcacgaacgtctccatccatgccatcgcccacgacgaccagacctacaccatggacgtcgtctggttgaggttcgacgtgccgacctcgtgtgccgagatgcgaatatacgaatcgtgtctgtatcacccgcagctcccagagtgtctgtccccggccgacgctccgtgcgccgcgagtacgtggacgtctcgcctggccgtccgcagctacgcggggtgttccagaacaaaccccccgccgcgctgttcggccgaggctcacatggagcccttcccggggctggcgtggcaggcggcctccgtcaatctggagttccgggacgcgtccccacaacactccggcctgtatctgtgcgtggtgtacgtcaacgaccatattcacgcatggggccacattaccatcagcaccgcggcgcagtaccggaacgcggtggtggaacagcccctcccacagcgcggcgcggatttggccgagcccacccacccgcacgtcggggcccctccccacgcgcccccaacccacggcgccctgcggttaggggcggtgatgggggccgccctgctgctgtctgcgctggggttgtcggtgtgggcgtgtatgacctgttggcgcaggcgtgcctggcgggcggttaaaagcagggcctcgggtaaggggcccacgtacattcgcgtggccgacagcgagctgtacgcggactggagctcggacagcgagggagaacgcgaccaggtcccgtggctggcccccccggagagacccgactctccctccaccaatggatccggctttgagatcttatcaccaacggctccgtctgtatacccccgtagcgatgggcatcaatctcgccgccagctcacaacctttggatccggaaggcccgatcgccgttactcccaggcctccgattcgtccgtcttctggtaaggcgccccatcccgaggccccacgtcggtcgccgaactgggcgaccgccggcgaggtggacgtcggagacgagctaatcgcgatttccgacgaacgcggacccccccgacatgaccgcccgcccctcgccacgtcgaccgcgccctcgccacacccgcgacccccgggctacacggccgttgtctccccgatggccctccaggctgtcgacgccccctccctgtttgtcgcctggctggccgctcggtggctccggggggcttccggcctgggggccgtcctgtgtgggattgcgtggtatgtgacgtcaattgcccgaggcgcataaagggccggtggtccgcctagccgcagcaaattaaaaatcgtgagtcactgcgaccgcaacttcccacccggagctttcttccggcctcgatgacgtcccggctctccgatcccaactcctcagcgcgatccgacatgtccgtgccgctttatcccacggcctcgccagtttcggtcgaagcctactactcggaaagcgaagacgaggcggccaacgacttcctcgtacgcatgggccgccaacagtcggtattaaggcgtcgacgcagacgcacccgctgcgtcggcatggtgatcgcctgtctcctcgtggccgttctgtcgggcggatttggggcgctcctgatgtggctgctccgctaaaagaccgcatcgacacgcgcgtccttcttgtcgtctctcttcccccccatcaccccgcaatttgcacccagcctttaactacattaaattgggttcgattggcaatgttgtctcccggttgatttttgggtgggtggggagtgggtgggtggggagtgggtgggtggggagtgggtgggtggggagtgggtgggtggggagtgggtgggtggggagtgggtgggtggggagtgggtgggtggggagtgggtgggtggggagtgggtgggtggggagtggcaaggaagaaacaagcccgaccaccagacagaaaatgtaaccatacccaaaccgactctgggggctgtttgtggggtcggaaccataggatgaacaaaccaccccgtacctcccgcacccttgggtgcgggtggctcatcggcatctgtccggtatgggttgttccccacccacttgcgttcggacgtcttagaatcatggcggttttctatgccgacatcggttttctcccccgcaataagacacgatgcgataaaatctgtttgtgaaatttattaagggtacaaattgccctagcacaggggtggggttagggccgggtccccacacccaaacgcaccaaacagatgcaggcagtgggtcgagtacagccccgcgtacgaacacgtcgatgcgtgtgtcagacagcaccagaaagcacaggccatcaacaggtcgtgcatatgtcggtgggtttggacgcggggggccatggtggtgataaagttaatggccgccgtccgccagggccacaggggcgacgtctcttggttggcccggagccactgggtgtggaccagccgcgcgtggcggcccaacatggcccctgtagccgggggcgggggatcgcgcacgtttgcagcgcacatgcgagacacctcgaccacggttcggaagaaggcccggtggtccgcgggcaacatcaccaggtgcgcaagcgcccgggcgtccagagggtagagccctgagtcatccgaggttggctcatcgcccgggtcatgccgcaagtgcgtgtgggttgggcttccggtgggcgggacgcgaaccgcggtgtggagccctacgcgggcccgagcgtacgctccatcttgtggggagaaggggtctgggctcgccaggggggcatacttgcccgggctatacagacccgcgagccgtacgtggttcgcggggggtgcgtggggtccggggctcccggggaggccggggctcccggggttgtcgtggatccctggggtcacgcggtaccctggggtctctgggagctcgcggtactctgggttccctaggttctcggggtggtcgcggaacccggggctcccggggaacacgcggtgtcctggggattgttggcggtcggacggcttcagatggcttcgagatcgtagtgtccgcaccgactcgtagtagacccgaatctccacattgccccgccgcttgatcattatcaccccgttgcgggggtccggagatcatgcgcgggtgtcctcgaggtgcgtgaacacctctggggtgcatgccggcggacggcacgccttttaagtaaacatctgggtcgcccggcccaactggggccgggggttgggtctggctcatctcgagagccacggggggaaccaccctccgcccagaaacttgggcgatggtcgtacccgggactcaacgggttaccggattacggggactgtcggtcacggtcccgccggttcttcgatgtgccacacccaaggatgcgttgggggcgattttgggcagcagcccgggagagcgcagcagaggacgctccgggtcgtgcacggcggttttggccgcctcccggtcctcacgcccccttttattgatctcatcgcgtacgtcggcgtacgtcctgggcccaacccgcatgttgtccaggaaggtgtccgccatttccagggcccacgacatgctcccccgcccgacgagcaggaagcggtccacgcaacggtcgccgccggtcgccccgacgagcaggaagcggtccacgcaacggtcgccgccggtcgcctcgacgaggacgttcctcctgcgggaaggcacgaacgcgggtgagccccctcctccgcccccgcgtcccccctcctccgcccccgcgtcccccctcctccgcccccgcgtcccccctcctccgcccccgcgtcccccctcctccgcccccgcgtcccccctcctccgcccccgcgtcccccctcctccacccccgcgtccccccctcctccgcccacccaaggtgcttacccgtgcacaaaggcggaccggtgggtttctgtcgtcggaggcccccggggtgcgtcccctgtgtttcgtgggtggggtgggtgggtctttccgcgtgtccctttccgatgcgatcccgatcccgagccggggcgtcgcgatgccgacgccgtccgctccgacggccctctgcgagtcccgctcccggtccgcgtgctccgcagccgctcccgtcgttcgtggccggcgccgtctgcgggcgtcggtcgcgccgggcctttatgtgcgccggagagacccgccccccgccgcccgggcccgcccccggggccggcgcggagtcgggcacggcgccagtgctcgcacttcgccctaataatatatatatattgggacgaagtgcgaacgcttcgcgttctcacttcttttacccggcggccccgcccccttggggcggtcccgcccgccggccaatgggggggcggcaaggcgggcggcccttgggccgcccgccgtcccgttggtcccaacgtccggcgggcgggaccgggggcccggggacggccaacgggcgcgcggggctcgtatctcattaccgccgaaccgggaagtcggggcccgggccccgcccccggcccgttcctcgttagcatgcggaacggaagcggaaaccaccggatcgggcggtaatgagatgccatgcggggcggggcgcgggcccacccgccctcgcgccccgcccatggcagatggcgcggatgggcggggccgggggttcgaccaacgggccgcggccacgggcccccggcgtgccggcgtcggggcggggtcgtgcataatggaattccgttcggggcgggcccgcctggggggcggggggccggcggcctccgctgctcctccttcccgccggcccctgggactatatgagcccgaggacgccccgatcgtccacacggagcgcggctgccgacacggatccacgacccgacgcgggaccgccagagacagaccgtcagacgctcgccgcgccgggacgccgatacgcggacgaagcgcgggagggggatcggccgtccctgtcctttttcccacccaagcatcgaccggtccgcgctagttccgcgtcgacggcgggggtcgtcggggtccgtgggtctcgccccctccccatcgagagtccgtaggtgacctaccgtgctacgtccgccgtcgcagccgtatccccggaggatcgccccgcatcggcgatggcgtcggagaacaagcagcgccccggctccccgggccccaccgacgggccgccgcccaccccgagcccagaccgcgacgagcggggggccctcgggtggggcgcggagacggaggagggcggggacgaccccgaccacgaccccgaccacccccacgacctcgacgacgcccggcgggacgggagggcccccgcggcgggcaccgacgccggcgaggacgccggggacgccgtctcgccgcgacagctggccctgctggcctccatggtagaggaggccgtccggacgatcccgacgcccgaccccgcggcctcgccgccccggacccccgcctttcgagccgacgacgatgacggggacgagtacgacgacgcagccgacgccgccggcgaccgggccccggcccggggccgcgcacgggaggccccgctacgcggcgcgtatccggaccccacggaccgcctgtcgccgcgcccgccggcccagccgccgcggagacgtcgtcacggccggcggcggccatcggcgtcatcgacctcgtcggactccgggtcctcgtcctcgtcgtccgcatcctcttcgtcctcgtcgtccgacgaggacgaggacgacgacggcaacgacgcggccgaccgcgcacgcgaggcgcgggccgtcgggcggggtccgtcgagcgcggcgccggaagcccccgggcggacgccgcccccgcccgggccaccccccctctccgaggccgcgcccaagccccgggcggcggcgaggacccccgcggcctccgcgggccgcatcgagcgccgccgggcccgcgcggcggtggccggccgcgacgccacgggccgcttcacggccgggcagccccggcgggtcgagctggacgccgacgcggcctccggcgccttctacgcgcgctatcgcgacgggtacgtcagcggggagccgtggcccggcgccgggcccccgcccccggggcgggtgctgtacggcggcctgggcgacagccgcccgggcctctggggggcgcccgaggcggaggaggcgcgacgccggttcgaggcctcgggcgccccggcggccgtgtgggcgcccgagctgggcgacgccgcgcagcagtacgccctgatcacgcggctgctgtacaccccggacgcggaggccatggggtggctccagaacccgcgcgtggtccccggggacgtggcgctggaccaggcctgcttccggatctcgggcgccgcgcgcaacagcagctccttcatcaccggcagcgtggcgcgggccgtgccccacctgggctacgccatggcggccggccgcttcggctggggcctggcgcacgcggcggccgccgtggccatgagccgccgatacgaccgcgcgcagaagggcttcctgctgaccagcctgcgccgcgcctacgcgcccctgttggcgcgcgagaacgcggcgctgacgggggccgcggggagccccggcgccggcgcagatgacgagggggtcgccgccgccgtcgtcgccgccgccgccgcaccgggcgagcgcgcggtgcccgccgggtacggcgccgcggggatcctcgccgccctggggcggctgtccgccgcgcccgcctcccccgcggggggcgacgaccccgacgccgcccgccacgccgacgccgacgacgacgccgggcgccgcgcccaggccggccgcgtggccgtggagtgcctggccgcctgccgcgggatcctggaggcgctggccgagggcttcgacggcgacctggcggccgtcccggggctggccggggcccggcccgccagccccccgcggccggagggacccgcgggccccgcttccccgccgccgccgcacgccgacgcgccccgcctgcgcgcgtggctgcgcgagctgcggttcgtgcgcgacgcgctggtgctcatgcgcctgcgcggggacctgcgcgtggccggcggcagcgaggccgccgtggccgccgtgcgcgccgtgagcctggtcgccggggccctgggccccgcgctgccgcgggacccgcgcctgccgagctccgcggccgccgccgccgcggacctgctgtttgagaaccagagcctgcgccccctgctggcggcggccgccagcgcaccggacgccgccgacgcgctggcggccgccgccgcctccgccgcgccgcgggaggggcgcaagcgcaagagtcccggcccggcccggccgcccggaggcggcggcccgcgacccccgaagacgaagaagagcggcgcggacgcccccggctcggacgcccgcgcccccctccccgcgcccccctccacgcccccggggcccgagcccgcccccgcccagcccgcggcgccccgggccgccgcggcgcaggcccgcccgcgccccgtggcgctgtcgcgccggcccgccgagggccccgaccccctgggcggctggcggcggcagcccccggggcccagccacacggcggcgcccgcggccgccgccctggaggcctactgctccccgcgcgccgtggccgagctcacggaccacccgctgttccccgtcccctggcgaccggccctcatgtttgacccgcgggccctggcctcgatcgccgcgcggtgcgccgggcccgccgccgccgcccaggccgcgtgcggcggcggcgacgacgacgataacccccacccccacggggccgccgggggccgcctctttggccccctgcgcgcctcgggcccgctgcgccgcatggcggcctggatgcgccagatccccgaccccgaggacgtgcgcgtggtggtgctgtactcgccgctgccgggcgaggacctggccggcggcggggcctcgggggggccgccggagtggtccgccgagcgcggcgggctgtcctgcctgctggcggccctggccaaccggctgtgcgggccggacacggccgcctgggcgggcaactggaccggcgcccccgacgtgtcggcgctgggcgcgcagggcgtgctgctgctgtccacgcgggacctggccttcgccggggccgtggagtttctggggctgctcgccagcgccggcgaccggcggctcatcgtggtcaacaccgtgcgcgcctgcgactggcccgccgacgggcccgcggtgtcgcggcagcacgcctacctggcgtgcgacctgctgcccgccgtgcagtgcgccgtgcgctggccggcggcgcgggacctgcgccgcacggtgctggccccgggccgcgtgttcggcccgggggtcttcgcgcgcgtggaggccgcgcacgcgcgcctgtaccccgacgcgccgccgctgcgcctgtgccgcggcggcaacgtgcgctaccgcgtgcgcacgcgcttcggcccggacacgccggtgcccatgtccccgcgcgagtaccgccgggccgtgctgccggcgctggacggccgggcggcggcctcggggaccaccgacgccatggcgcccggcgcgccggacttctgcgaggaggaggcccactcgcaccgcgcctgcgcgcgctggggcctgggcgcgccgctgcggcccgtgtacgtggcgctggggcgcgaggcggtgcgcgccggcccggcccggtggcgcgggccgcggagggacttttgcgcccgcgccctgctggagcccgacgacgacgcccccccgctggtgctgcgcggcggcgacgacgacgacgacggcccgggggccctgccgccggcgttgcccgggattcgctgggcctcggccacgggccgcagcggcaccgtgctggcggcggcgggggccgtggaggtgctgggggcggaggcgggcttggccacgcccccgcgacgggaagttgtggactgggaaggcgcctgggacgacgacgacggcggcgcgttcgagggggacggggtgctgtaacgggccgggacggggcggggcgcttgcgaaacccgaagacgcaataaacgacaacgacctgatttagttttgcagtagcgttgtttatttcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcggtggtggtgcgcgggcgcccccggagggtttggatctctgacctgagattggcggcactgaggtagagatgcccgaacccccccgagggagcgcgggacgcggccggggagggctggggccggggagggctggggccggggagggctggggccggggagggctggggccggggagggctggggccggggagggctggggccggggagggctggggctggggagggctggggctggggagggctggggcggtggtgtgtgacaggagcggcgtgttgcgctgggggacgtctggaggagcggggggtgcgcggtgacgtgtggatgaggaacaggagttgttgcgcggtgagttgtcgctgtgagttgtgttggtgggcaggtgtggtggatgacgtgacgtgtgacgtgtggatgaggaaccggagtcgccggtgcgccgtgctgttggtgttctgttggtgttgttacacctgtggcagcccgggccccccgcgggcggggcggcgcgcaaaaaaggcgggcggcggtccgggcggcgtgcgcgcgcgcggcgggcgttgggggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggaaaacgggccccccccgaaacacaccccccgggggtcgcgcgcggccctttaaagcg

(19) United States
(12) Patent Application Publication
(10) Pub. No.: US 2025/0295718 A1
(43) Pub. Date: Sep. 25, 2025

(54) CHIMERIC HSV EXPRESSING HIL21 TO BOOST ANTI-TUMOR IMMUNE ACTIVITY

(71) Applicant: Research Institute at Nationwide Children's Hospital, Columbus, OH (US)

(72) Inventor: Kevin A. Cassady, Columbus, OH (US)

(21) Appl. No.: 18/712,159

(22) PCT Filed: Nov. 23, 2022
(86) PCT No.: PCT/US2022/050899

§ 371 Date: May 21, 2024

Related U.S. Application Data
(60) Provisional application No. 63/283,131, filed on Nov. 24, 2021.
(60) Provisional application No. 63/311,854, filed on Feb. 18, 2022.

(51) Int. Cl. A61K 35/763; A61K 38/20; A61K 45/06; A61K 48/00; A61P 35/00; C07K 14/54; C12N 15/86

(52) U.S. Cl. CPC A61K 35/763; A61K 38/20; A61K 45/06; A61K 48/005; A61P 35/00; C07K 14/54; C12N 15/86

(57) ABSTRACT

Provided herein are non-natural herpes simplex virus (“HSV”) vectors and one or more polynucleotides encoding IL-21 or a biologically active fragment of IL-21 for use in the treatment of cancer.

### CROSS-REFERENCE TO RELATED APPLICATIONS

This application claims the benefit under 35 U.S.C. § 119(e) and the Paris Convention of U.S. Provisional Application Ser. No. 63/283,131, filed Nov. 24, 2021 and 63/311,854, filed Feb. 18, 2022, the contents of each of which are hereby incorporated by reference in their entireties.

### STATEMENT OF GOVERNMENT SUPPORT

This invention was made with government support under Grant Nos. CA232561 and CA222903 awarded by the National Institutes of Health. The government has certain rights in the invention.

### BACKGROUND

The antitumor efficacy of oncolytic herpes simplex viruses (oHSVs) such as Imlygic™, recently FDA approved to treat melanoma, is very promising. These vectors have two major mechanisms of action: (1) a lytic phase, determined by direct infection and lysis of cells, and (2) an immunologic phase, driven by the stimulation of antitumor immunity. However, not all cancers respond similarly as virus spread is intrinsically slow in some cancers. In culture, cells vary in their levels of permissivity to viruses. In animals, variations in the tumor's stromal and immune cell composition lead to variations in the capacity for virus spread and immune reactions. Therefore, strategies to improve the potency of the lytic phase to reach optimal therapeutic benefit are still needed. This disclosure satisfies these needs and provides related advantages as well.

### SUMMARY OF THE DISCLOSURE

This disclosure provides a non-natural herpes simplex virus (“HSV”) vector and a polynucleotide(s) encoding one or more IL-21 or a biologically active fragment thereof. If more than one IL-21 polypeptide is encoded, the polynucleotides can be the same or different from each other. In one aspect, the HSV comprises a C134 HSV viral vector. In a further aspect, the IL-21 polypeptide comprises human IL-21 polypeptide or a biologically active fragment thereof. In a further aspect, the polynucleotide(s) encoding the one or more IL-21 polypeptide or a biologically active fragment thereof is inserted at an ICP34.5 locus of the HSV. Alternatively, the one or more are inserted in RL1/g134.5 gene loci within the virus located in both repeat long genetic region of the virus. See, e.g., FIGS.: 2A and 2B. In a yet further aspect, the one or more IL-21 polynucleotide(s) is under the control of a modified retroviral promoter region (“MND”). The polynucleotide can be a DNA or an RNA molecule. In a yet further aspect, non-natural HSV vector of this disclosure has the IL-21 polynucleotide is under the control of a promoter selected from modified retroviral promoter region (“MND”), a cytomegalovirus (CMV) IE promoter, optionally located in the UL3/UL4 intergenic region, or an EGR1 promoter, and further optionally the HSV vector as shown in FIG. 12.

In one aspect, provided herein is a host cell comprising the non-natural HSV vector as described herein. The host cell can be a eukaryotic cell or a prokaryotic cell.

Yet further provided are compositions comprising or alternatively consisting of, or yet further consisting of the non-natural HSV vector as described herein as the active agent and a carrier. In one aspect, the carrier comprises a pharmaceutically acceptable carrier. The compositions can be formulated or lyophilized for storage or administration. In one aspect, the composition is formulated for intratumoral injection.

The vectors and compositions can be used to deliver IL-21 to cell or tissue and/or to inhibit the growth of cancer cells or treating a cancer in a subject in need thereof. In one aspect, provide is a method of inhibiting the growth of a cancer cell comprising contacting the cell with an effective amount of the non-natural HSV or the composition as described herein. The contacting is in vitro or in vivo, and optionally wherein the cancer cell is as described herein, e.g. a glioblastoma cell.

Also provided is a method to treat cancer or inhibit the growth or metastasis of a cancer in a subject in need thereof by administering to the subject an effective amount of the non-natural HSV vector as described herein or the composition containing same as disclosed herein. Administration can be systemic or local, optionally infusion, injection or intratumoral injection. In a further aspect, the method further comprises subsequent administration to the subject an effective amount of an immunotherapy. Non-limiting examples of such include adoptive cell therapy, immune checkpoint inhibition, immune system modulation, or engineered cellular therapy (e.g., CAR T or CAR NK therapy). The method is useful to treat a solid tumor or a blood cancer, such as for example a sarcoma, carcinoma, or a glioblastoma (GBM). In one aspect, the glioblastoma is mesenchymal GBM or classical/proneural subtype. It can be administered as a first line, second line, third line, fourth line or fifth line therapy.

Applicant has discovered that the HSV vector constructs are not only transcriptionally active (making abundant mRNA) but these transcripts are translated into actual cytokine in the tumor. This leads to higher levels of cytokine production in the tumor environment. The expression of IL-21 leads to improved immune mediated anti-tumor activity in Applicant's immune competent mouse tumor models and increases Natural Killer and T cell activity in the tumor both in flank and in orthotopic brain tumor models. The vector also increased the IFNgamma response of NK and CAR-NK cells and is anticipated to have similar activity on CAR-T cells. Thus, the vector is useful as a monotherapy or in tandem with adoptive cellular or engineered cellular therapies.

Further provided is a kit comprising the non-natural HSV vector as described herein or the composition as described herein and optionally, instructions for use.

### BRIEF DESCRIPTION OF THE DRAWING

FIG. 1 depicts a polynucleotide of the non-natural HSV vector and a polynucleotide encoding human IL-21.

FIGS.: 2A-2B are images of the IL-21 (e.g., human IL-21) cassette in the HSV genome. Both copies are inserted in both RL1/g134.5 loci within the virus located in both repeat long genetic region of the virus. (FIG. 2A) is an image of the first copy in the “left” side of the genome and (FIG. 2B) is an image when it is represented linearly (the second copy runs in the anti-sense direction).

FIGS. 3A-3F is a composite summary of oncolytic virus Phase Ib studies and rationale for constructing C021 (oHSV-hIL21). As described in (Miller et al., Clinical Cancer Research 2022, Feb. 1; 28(3):498-506, gene expression analysis from the G207 oncolytic Phase Ib glioma clinical trial suggested that hIL21 expression in the oHSV treated tumors correlated with improved survival. As used herein, the term “G207” intends a conditionally replication-competent Δγ₁34.5 oncolytic herpes virus that contains UL39 gene mutation. (FIG. 3A) shows a summary of the trial design. Participants with suspected glioma progression after initial treatment, underwent biopsy. After biopsy confirmation of glioma progression, the participants then received a fractional dose (1.5×108 Plaque forming units [PFU]) of the oHSV. Subjects then underwent surgical resection of the oHSV treated tumors (d2 or d5 post treatment), followed by administration of the remaining maximum tolerated dose of the oHSV (1×10⁹ PFU) in the resected tumor margins. Participants were followed for clinical response, survival, and the oHSV-treated resected tumor analyzed in correlative biologic studies as shown (histology, RNA/DNA extraction for gene expression and sequencing, and immune cell characterization). (FIG. 3B) is a survival summary. (FIG. 3C) the results showed that for 502 identified genes there was a direct positive (342 genes) or negative (160 genes) relationship between gene expression and survival in the participants' oHSV treated glioma samples. Over 12 of the significant genes identified were related to immune response to the virus. (FIG. 3D) A biological clustering analysis was used to identify the predictive biological processes and upstream regulatory pathways from these differentially expressed genes as shown. The top panel heat map shows the observed fold change (log 2) from the best responder (PT107) relative to the 2 participants with the shortest survival post-oHSV treatment. The lower panel represents heat map (pathways Z score >or <2 from ingenuity pathway analysis) results show predicted upstream activating gene pathways associated with the 502 statistically significant identified genes in the dataset. A box surrounding IL1b, shows an example of a gene with discordant results from the observed and predicted upstream activity. (FIG. 3E) The pathway analysis results suggested that early intrinsic pathway response (Left side of the figure in green) to the virus and adaptive immune activity to the oHSV (shown on the right) that increased IFNgamma activity in the treated tumor was associated with improved therapeutic response. The gene expression analysis also suggested that 6 cytokines were positively associated with improved oHSV therapeutic response and included IL21. (FIG. 3F) Summary of patient clinical trial identifiers, GBM tumor subtype, IDH1/2 hotspot mutational status, survival post-G207 treatment (in days), and available samples for Applicant's study. Timepoint of when the sample was taken is shown as 2 or 5 days (‘d’) PFU=plaque-forming units.

FIGS. 4A-4B Principal component analysis (PCA) and hierarchical clustering of RNA-seq gene expression values is driven by G207 treatment in pre- and post-treated biopsies. (FIG. 4A) The top 500 most variable protein-coding genes were used to perform unsupervised PCA. Samples are colored by clinical trial identifiers while shapes designate timepoint relative to G207 treatment. (FIG. 4B) Unsupervised hierarchical clustering of RNA-seq expression values, using the topmost 500 variable genes between all samples. Each column represents a gene, while rows represent individual samples. The legend represents the log 2 calculated relative expression scaled per column (gene). Sample annotations are displayed on left and include cluster ID (based on hierarchical clustering), treatment timepoint (pre or post G207), and GBM tumor subtype.

FIGS. 5A-5C Immune cell type scores using PanCancer gene expression panel are higher in post-G207 vs. pre-G207 samples. (FIG. 5A) Heatmap displaying immune cell types (rows) and samples (columns). Annotations for treatment timepoint and response are displayed as colors at top. Relative abundance of cell types is estimated based on marker genes identified in PanCancer immune panel for each sample, and values are scaled per row. ‘Responders’ are defined as patients who survived beyond the median survival of 150 days. (FIG. 5B) Boxplots for specific immune cell type measurements against timepoint are shown. Cell type measurements are derived from expression of marker genes in the PanCancer immune panel (see methods). ‘Total TILs’ score was calculated as the average of all cell type scores whose correlations with CD45 exceeded 0.6. P-values were calculated using unpaired, two-sided t-test. (FIG. 5C) Cell types scores in post-G207 samples relative to pre-G207 samples. Displayed is the difference in mean-centered cell type scores for each cell type, shown as the difference in post-G207 relative to pre-G207. Red indicates increase after G207 treatment while blue indicates decrease after G207 treatment. CD45=CD45-positive cells; NK=natural killer cells; DC=dendritic cells; Th1=T helper cells; TILs=tumor-infiltrating lymphocytes.

FIGS. 6A-6C Spearman correlative analysis reveals significant set of genes associated with patient survival. (FIG. 6A) Schematic correlative approach (TPM per gene vs. days of survival). (FIG. 6B) Summary of the significant genes whose spearman p-values were ≤0.05. (FIG. 6C) Example of three of the 502 genes analyzed and their relationship between gene expression and survival; black dots represent mesenchymal tumors while red dots represent proneural or other subtype.

FIGS. 7A-7B Principal component analysis and hierarchical clustering of NanoString PanCancer gene expression values for G207-treated samples. Applicant studied RNA extracted from pre- and post-G207-treatment samples by multiplex gene expression analysis focusing on 770 immune response genes representing 24 different immune cell types (NanoString™ PanCancer Immune Profiling Panel). (FIG. 7A) PCA analysis using PanCancer immune gene expression data. (FIG. 7B) Unsupervised clustering using NanoString expression values.

FIGS. 8A-8B CIBERSORT analysis of post-G207 samples. (FIG. 8A) RNA-seq expression values from post-G207 tumor samples were used as input for CIBERSORT, which provides in silico prediction of 22 different immune cell types. The height of each bar represents the total immune score calculated by CIBERSORT, while each shade of gray represents individual cell type scores contributing to the total. (FIG. 8B) RNA-seq deconvolution results from different algorithms using TIMER 2.0 (http://timer.cistrome.org/). Non-log transformed transcripts per million (TPM) values derived from RNA-seq were used.

FIGS. 9A-9B Ingenuity Pathway-based upstream regulatory analysis of the 502 significant genes correlating with G207-survival. (FIG. 9A) Upstream regulators predicted to be activated or repressed based on Applicant's IPA-uploaded spearman gene list. Log 2 fold-change reflects relative change in TPM value of best responder (PT107) compared to non-responders (PT101 and PT108); activation Z score is calculated in IPA software and infers the activation state (“increased” or “decreased”) of implicated upstream regulators. Genes with significant p-values are shown. (FIG. 9B) Schematic representation indicates the components involved in the most significant upstream responses. Applicant determined that ˜50% of the predicted upstream regulator genes map to immune response pathways including both intrinsic, antiviral response pathways (indicated by red box and arrows) and adaptive immune response pathways (indicated by blue color). PRR=pathogen recognition receptors; IFN=interferon; APC=antigen-presenting cells; NK=natural killer cells.

FIG. 10 Network and regulator analysis of the significant genes identified in the G207 and syngeneic models. Leukocyte recruitment, antigen presentation, and T-cell response are associated with improved survival following oHSV therapy.

FIG. 11 Network/Regulator Analysis for the shared 165 genes present in all treated patients (N=6) and those with Mesenchymal tumors (N=4). Common genes are involved in immune-related functions (namely Leukocyte recruitment, antigen presentation, and T-cell response).

FIGS. 12A-12B oHSV IL21 construct schematic and example of IL21 expression levels. Based upon the gene expression analysis results from the Phase Ib clinical trial, Applicant constructed (ICP34.5[-]) oncolytic HSV (oHSV) recombinants that express the IL21 cytokine to test whether IL21 improves outcome during oHSV treatment of gliomas using pre-clinical models. (FIG. 12A) As shown in the schematic representation of the HSV-1 genome, the IL21 gene was inserted into the g134.5 (also called RL1) gene coding domain as shown and in the schematic and C021 (Δγ₁34.5, hIL21), C023 (Δγ₁134.5, mIL21), and C025 (Δγ₁134.5, mCXCL10 & mIL21) and these plasmids used to make the corresponding recombinant viruses by homologous recombination after co-transfection and serial plaque selection. (FIG. 12B) The bar graph shows hIL21 protein detection in vero cell supernatant samples from 9 different plaque isolates of C021 (2-14.5 ng/ml) with appropriate (+controls hIL21 transfected 293T cells) and different negative controls (media alone or PBS alone). Flank tumors were established by subcutaneous flank injection of CT2A tumor cells similar to Applicant's past studies (Ghonime et al., Journal of Immunotherapy of Cancer 2021, 9:e002939. doi: 10.1136/jitc-2021-002939, hereinafter Ghonime et al. JITC, 2021). After tumors established and were 50-200 mm³ in size (based upon digital caliper measurements), the CT2A tumors were treated with 3×10⁷ PFU. Tumors were harvested 2d post-treatment and IL21 measured by ELISA from the homogenized tumor samples. C021 generated hIL21 levels (600 ng-2000 ng/ml) 2d post treatment.

FIGS. 13A-13C 67C-4 Sarcoma Studies: C021 reduces tumor growth in a C134 oHSV resistant murine MPNST sarcoma model (see Ghonime et al. Cancer Immunotherapy, Dec. 6(12):1499-1510, doi: 10.1158/232.6-6066.CIR-18-0014. Epub 2018 Oct. 23. PMID: 30352799.) (FIG. 13A) summary of overall tumor growth (FIG. 13A) relative tumor growth after treatment, and (FIG. 13C) individual tumors growth kinetics after treatment.

FIGS. 14A-14C CT2A flank tumor studies: C021 reduces tumor growth in a C134 oHSV resistant murine malignant glioma tumor model (Ghonime et al. Journal of JITC, 2021) (FIG. 14A) summary of overall tumor growth Saline vs C134 (FIG. 14B). Overall tumor growth of C134 based IL21 virus (C021), (FIG. 14C) individual tumors growth kinetics after treatment.

FIG. 15 Tumor infiltrating Leukocyte (TIL) analysis by flow cytometry of disaggregated and homogenized Mock (PBS), oHSV (C134) or C021 (C134+IL21) treated tumors shows differences in the immune infiltrate 7d post treatment. C021 Increased CD3 infiltrates, MHCII, and CD4>CD8 population balance. C021 treated tumors demonstrated less CTL activity compared to parent virus C134 treated tumors suggesting it exerts a unique immune mediated mechanism in these flank studies. Panels show flow cytometry (D6 post oHSV or saline treatment) of homogenized CT2A tumors show that C021 Increased CD3 infiltrates, MHCII, and CD4>CD8 population balance. C021 treated tumors also demonstrated less CTL activity compared to parent virus C134 treated tumors suggesting it exerts a unique immune mediated mechanism.

FIG. 16 shows that both C021 (hIL21) and C025 (mil21 based recombinant) improves survival in a C134 CT2A orthotopic glioma model in B6 mice (Log Rank **p=0.0046: C021 vs Saline, **p=0.0023 C025 vs C134 and C025 vs Saline).

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques of tissue culture, immunology, molecular biology, microbiology, cell biology, and recombinant DNA, which are within the skill of the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used herein, the term “comparable” refers to having a level same with that of the reference or within a variation of +/−50%, or alternatively 45%, or alternatively 40%, or alternatively 35%, or alternatively 30%, or alternatively 25%, or alternatively 20%, or alternatively 15%, or alternatively 10%, or alternatively 5%, or alternatively 2% compared to the reference level

Throughout this disclosure, various publications, patents and published patent specifications may be referenced by an identifying citation or by an Arabic numeral. The full citation for the publications identified by an Arabic numeral are found immediately preceding the claims. The disclosures of these publications, patents and published patent specifications are hereby incorporated by reference into the present disclosure in their entirety to more fully describe the state of the art to which this invention pertains.

### Definitions

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the description of the invention and the appended claims, the singular forms “a,” “an” and “the” are intended to include the plural forms as well, unless the context clearly indicates otherwise.

As used herein, the term “comprising” is intended to mean that the compositions and methods include the recited elements, but do not exclude others. As used herein, the transitional phrase consisting essentially of (and grammatical variants) is to be interpreted as encompassing the recited materials or steps and those that do not materially affect the basic and novel characteristic(s) of the recited embodiment. These features are recited in the method embodiments. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

As used herein, “and/or” refers to and encompasses any and all possible combinations of one or more of the associated listed items, as well as the lack of combinations when interpreted in the alternative (“or”).

The term “cell” as used herein may refer to either a prokaryotic or eukaryotic cell, optionally obtained from a subject or a commercially available source.

“Eukaryotic cells” all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human, e.g., HEK293 cells and 293T cells.

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

The terms “equivalent” or “biological equivalent” are used interchangeably when referring to a particular molecule, biological, or cellular material and intend those having minimal homology while still maintaining desired structure or functionality. Non-limiting examples of equivalent polypeptides, include a polypeptide having at least 60%, or alternatively at least 65%, or alternatively at least 70%, or alternatively at least 75%, or alternatively 80%, or alternatively at least 85%, or alternatively at least 90%, or alternatively at least 95% identity thereto or for polypeptide sequences, or a polypeptide which is encoded by a polynucleotide or its complement that hybridizes under conditions of high stringency to a polynucleotide encoding such polypeptide sequences. Conditions of high stringency are described herein and incorporated herein by reference. Alternatively, an equivalent thereof is a polypeptide encoded by a polynucleotide or a complement thereto, having at least 70%, or alternatively at least 75%, or alternatively 80%, or alternatively at least 85%, or alternatively at least 90%, or alternatively at least 95% identity, or at least 97% sequence identity to the reference polynucleotide, e.g., the wild-type polynucleotide. In one aspect, the equivalent polypeptide or polynucleotide has the same or substantially similar biological function as the reference polypeptide or polynucleotide, respectively, e.g., cytolytic function, anti-tumor, anti-metastatic, or anti-cancer function, as determined by the appropriate cell assay or animal model as described herein.

Non-limiting examples of equivalent polypeptides, include a polynucleotide having at least 60%, or alternatively at least 65%, or alternatively at least 70%, or alternatively at least 75%, or alternatively 80%, or alternatively at least 85%, or alternatively at least 90%, or alternatively at least 95%, or alternatively at least 97%, identity to a reference polynucleotide. An equivalent also intends a polynucleotide or its complement that hybridizes under conditions of high stringency to a reference polynucleotide.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters. In particular, exemplary programs include BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity can be determined by incorporating them into clustalW (available at the web address: genome.jp/tools/clustalw/, last accessed on Jan. 13, 2017).

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

“Homology” or “identity” or “similarity” can also refer to two nucleic acid molecules that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise, or alternatively consist essentially of, or yet further consist of comprise, or alternatively consist essentially of, or yet further consist of two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

A “gene” refers to a polynucleotide containing at least one open reading frame (ORF) that is capable of encoding a particular polypeptide or protein after being transcribed and translated. A “gene product” or alternatively a “gene expression product” refers to the amino acid (e.g., peptide or polypeptide) generated when a gene is transcribed and translated.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell. In one aspect, this invention provides promoters operatively linked to the downstream sequences, e.g., HSV virulence genes or their mutants.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “isolated” as used herein refers to molecules or biologicals or cellular materials being substantially free from other materials.

As used herein, the term “functional” may be used to modify any molecule, biological, or cellular material to intend that it accomplishes a particular, specified effect.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising, or alternatively consisting essentially of, or yet further consisting of purine and pyrimidine bases or other natural, chemically or biochemically modified, non-natural, or derivatized nucleotide bases.

The term “wild-type” refers to a gene or gene product having characteristics of that gene or gene product when isolated from a naturally occurring source. In some embodiments, the wild type genes or gene products, even for one viral strain, contain slight different sequences.

The term “mutant” refers to a gene or gene product which displays modifications in sequence and or functional properties (i.e., altered characteristics) when compared to the wild-type gene or gene product or the gene or gene product from other mutant strain(s). In one embodiment, the other mutant strain comprise a 17TermA or an rR450 strain.

The term “mutation” refers to a DNA sequence variation from a wild type or other mutant strain (s). A mutation produces or does not produce a function property in an organism. There are multiple types of mutations, including but not limited to an insertion, a deletion, a truncation, a frameshift, a substitution, or a point mutation.

The term “point mutation” refers to a mutation with a single nucleotide base change, insertion, or deletion of the genetic material, DNA or RNA.

“Deletion” refers to a mutation in which a part of chromosome or a sequence of DNA is missing.

“Frameshift” refers to a mutation caused by indels (insertions or deletions) of a number of nucleotides in a DNA sequence that is not divisible by three.

“Substitution” refers to a mutation with a substitution of one or a few nucleotides of a gene.

“Truncation” refers to a mutation with elimination of the N- or C-terminal portion of a protein by proteolysis or manipulation of the structural gene, or premature termination of protein elongation due to the presence of a termination codon in its structural gene as a result of a nonsense mutation.

In some embodiment, the mutation is a nonsynonymous mutation. The term “nonsynonymous mutation” refers to a mutation that alters the amino acid sequence of a protein, which is contrasted with a synonymous mutation that do not alter amino acid sequences.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting exemplary promoters include Rous sarcoma virus (RSV) LTR promoter (optionally with the RSV enhancer), a cytomegalovirus (CMV) promoter, an SV40 promoter, a dihydrofolate reductase promoter, a β-actin promoter, a phosphoglycerol kinase (PGK) promoter, a U6 promoter, an EF1 promoter, a chicken β-actin (“CBA”) promoter, a HCMV IRS1 gene under control of the CMV IE promoter in the UL3/UL4 intergenic region, or an EGR1 promoter.

Additional non-limiting exemplary promoters with certain target specificity are provided herein below including but not limited to modified retroviral promoter region (“MND”), CMV, EF1a, SV40, PGK1 (human or mouse), P5, Ubc, human beta actin, CAG, TRE, UAS, Ac5, Polyhedrin, CaMKIIa, Gal1, TEF1, GDS, ADH1, CaMV35S, Ubi, H1, U6, and Alpha-1-antitrypsin. Synthetically-derived promoters may be used for ubiquitous or tissue specific expression. Further, virus-derived promoters, some of which are noted above, may be useful in the methods disclosed herein, e.g., CMV, HIV, adenovirus, and AAV promoters. In some embodiments, the promoter is coupled to an enhancer to increase the transcription efficiency.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

Non-limiting examples of tumor-specific promoters to be used in the present invention include the telomerase reverse transcriptase promoter, the glial fibrillary acidic protein promoter, an E2F promoter; a survivin promoter, a COX-2 promoter, an EGD-2 promoter; an ELF-1 promoter; a hypoxia-specific promoter; a carcinoembryonic antigen promoter, and the stromelysin 3 promoter. Additional promoters are recited herein.

The term “cryopreservative” refers to a compound or material that is capable of, protecting the one or more tissues, virus, or other biological agents from being damaged or compromised. Examples of cryopreservatives include, but are not limited to, chondroitin sulfate, glycosaminoglycan dimethylsulfoxide, cell penetrating organic solutes, polysaccharides, glycerol, Dulbecco's minimum essential medium (DMEM), glutamine, D-glucose, sodium pyruvate, fetal calf serum, papaverine, DMSO, glycerol, trehalose, KH2PO4, K2HPO4, KCl, mannitol, NaHCO₃, sodium ascorbate, 1,2-propanediol, formamide, 2,3-butanediol, probuchol, curcumin and mixtures thereof.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise, or alternatively consist essentially of, or yet further consist of comprise, or alternatively consist essentially of, or yet further consist of a protein's or peptide's sequence. As used herein the term “amino acid” refers to either natural and/or unnatural or synthetic amino acids, including glycine and both the D and L optical isomers, amino acid analogs and peptidomimetics.

As used herein, the term “vector” refers to a non-chromosomal nucleic acid comprising, or alternatively consisting essentially of, or yet further consisting of an intact replicon such that the vector may be replicated when placed within a cell, for example by a process of transformation. Vectors may be viral or non-viral. Viral vectors include retroviruses, adenoviruses, herpes simplex virus (“HSV”), baculoviruses, modified baculoviruses, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising, or alternatively consisting essentially of, or yet further consisting of DNA condensed with cationic polymers such as heterogeneous polylysine, defined-length oligopeptides, and polyethylene imine, in some cases contained in liposomes; and the use of ternary complexes comprising, or alternatively consisting essentially of, or yet further consisting of a virus and polylysine-DNA.

A “viral vector” is defined as a recombinantly produced virus or viral particle that comprises, alternatively consists essentially of, or yet further consists of a polynucleotide to be delivered into a host cell, either in vivo, ex vivo or in vitro. Examples of viral vectors include retroviral vectors, HSV vectors, AAV vectors, lentiviral vectors, adenovirus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

As used herein, the term “recombinant expression system” or “recombinant vector” refers to a genetic construct or constructs for the expression of certain genetic material formed by recombination.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene.

The term “herpes simplex virus” or “HSV” as used herein means a herpes simplex virus that produces the effect of the present invention, which includes a wild type or mutant herpes simplex virus. The HSV-1 is an enveloped, double-stranded DNA virus. In one embodiment, the HSV-1 can infect a human cell. In another embodiment, a sequence, a gene or multiple genes can be incorporated to the HSV-1. The size of incorporated sequence can be approximate 1 base, 5 bases, 10 bases, 100 bases, 1 kb, 10 kb, 100 kb, or 150 kb. HSV-1 can induce cell lysis at a relatively low multiplicity of infection (MOI), and its proliferation can be inhibited by anti-viral drugs. In one embodiment, the HSV viral DNA stays outside the chromosomes without being incorporated into the genome of host cells. The HSV-1 can encompass a variety of strains (e.g., KOS and McKrae). See Wang et al., (2013) Virus Res. 173(2):436-440. In one embodiment, the HSV-1 is an HSV-1 KOS strain. In another embodiment, the HSV-1 is an HSV-1 McKrae strain.

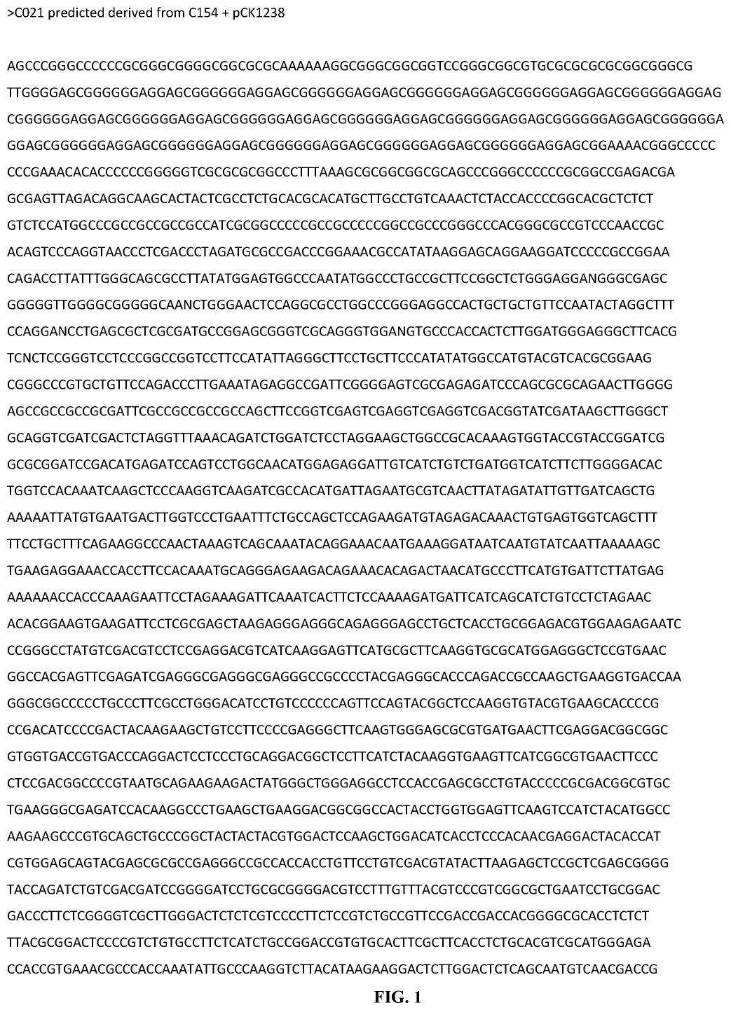

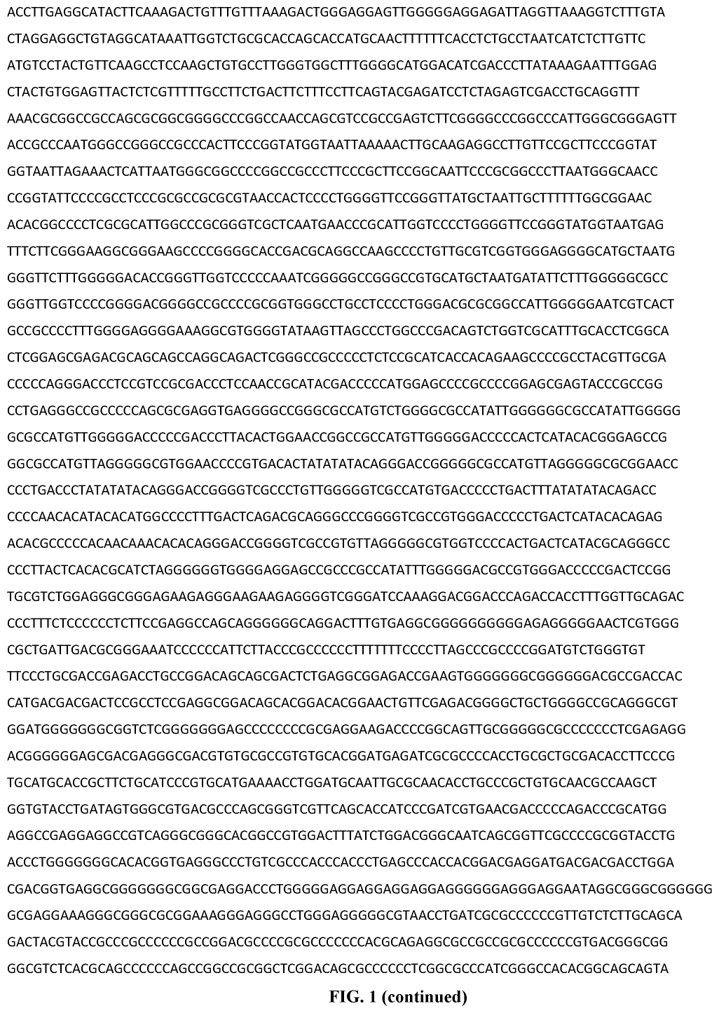

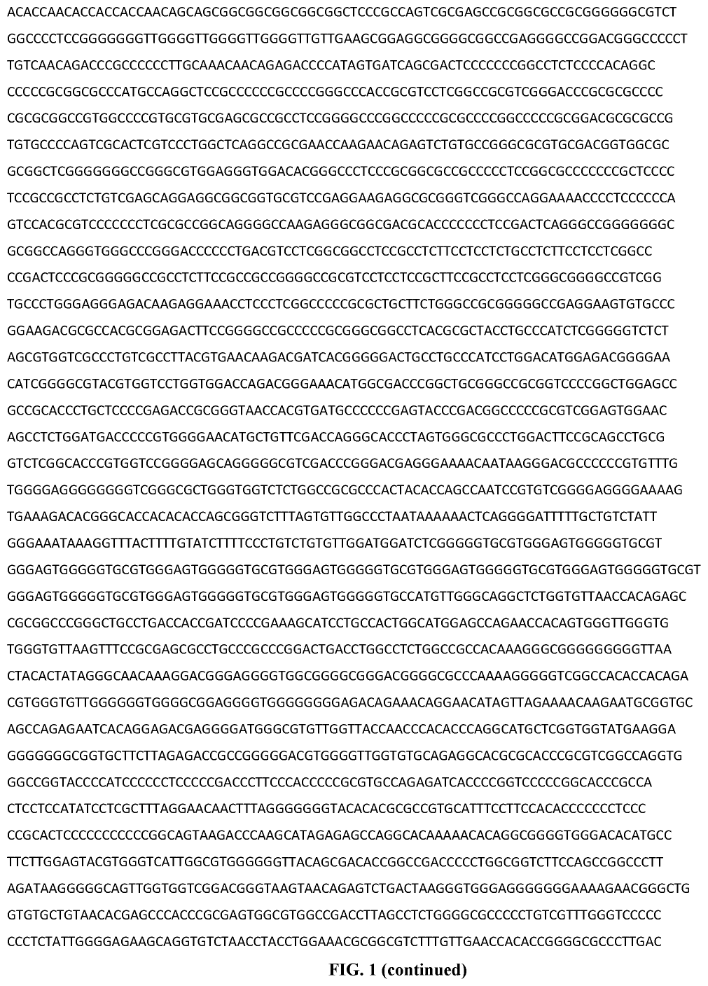

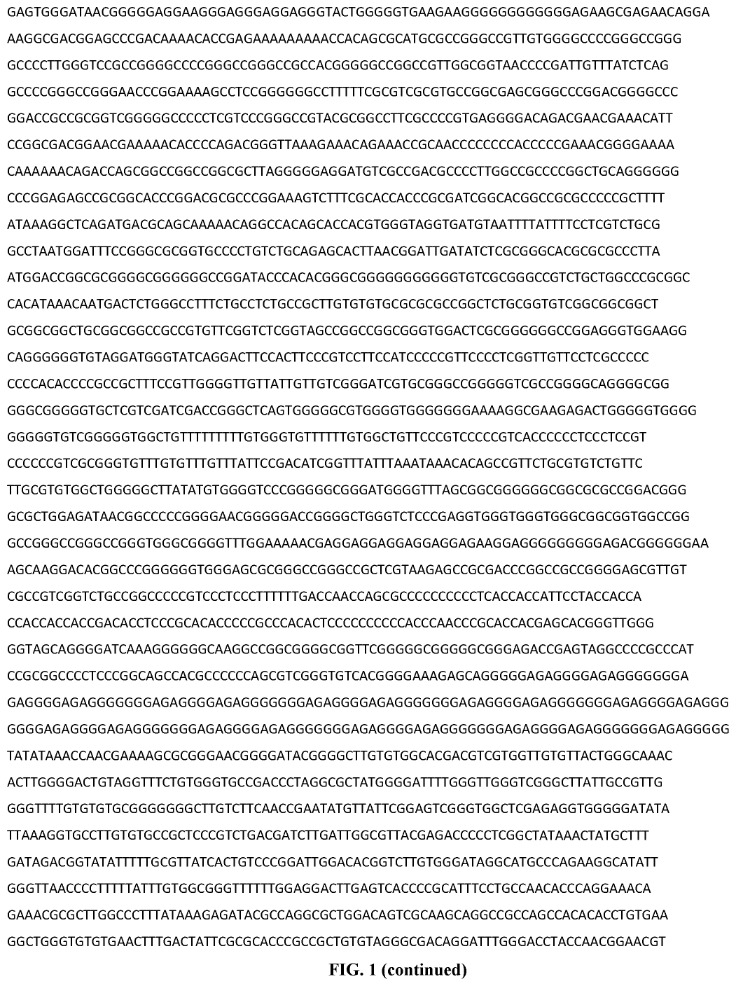

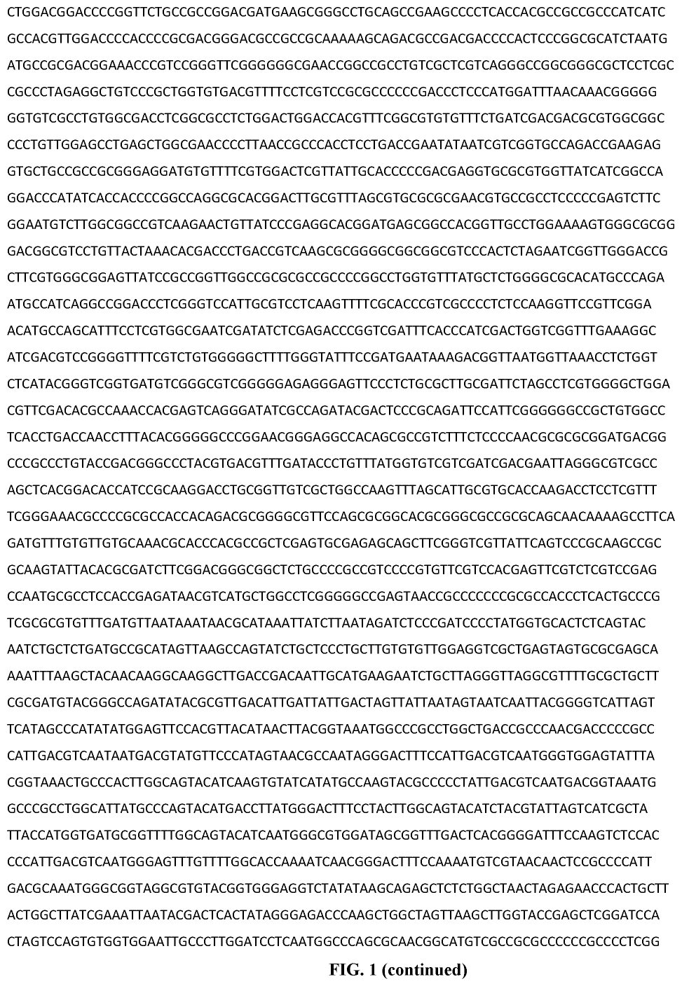

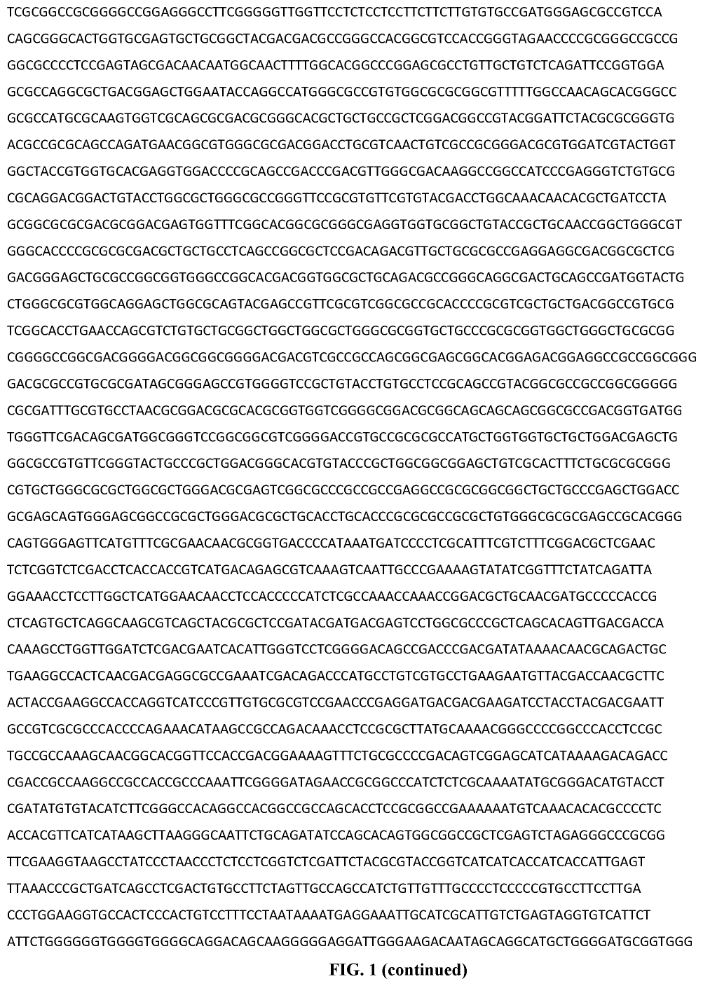

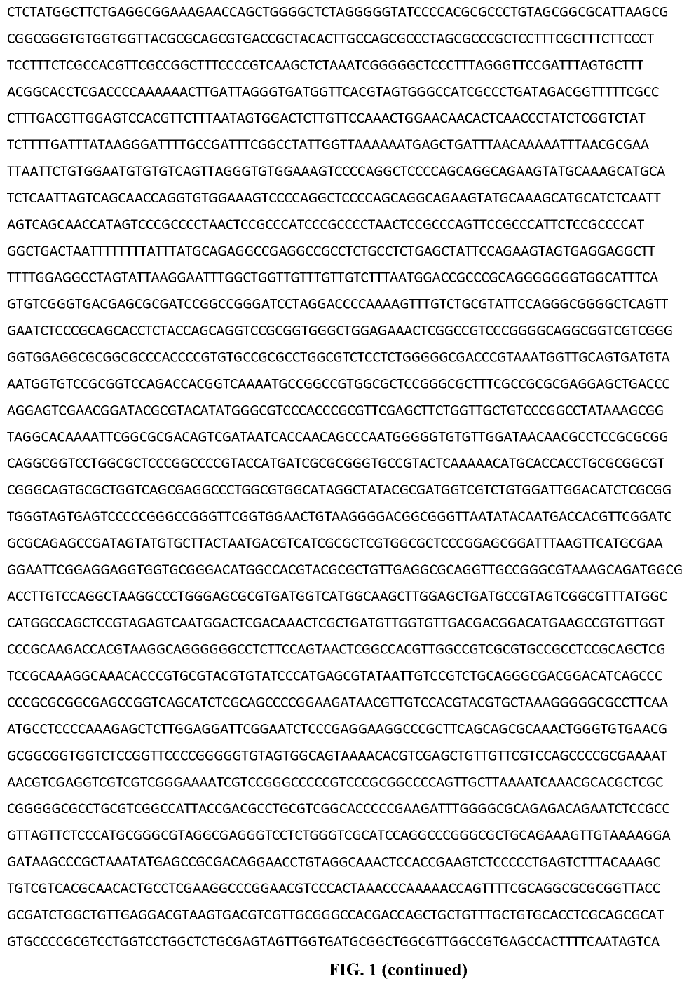

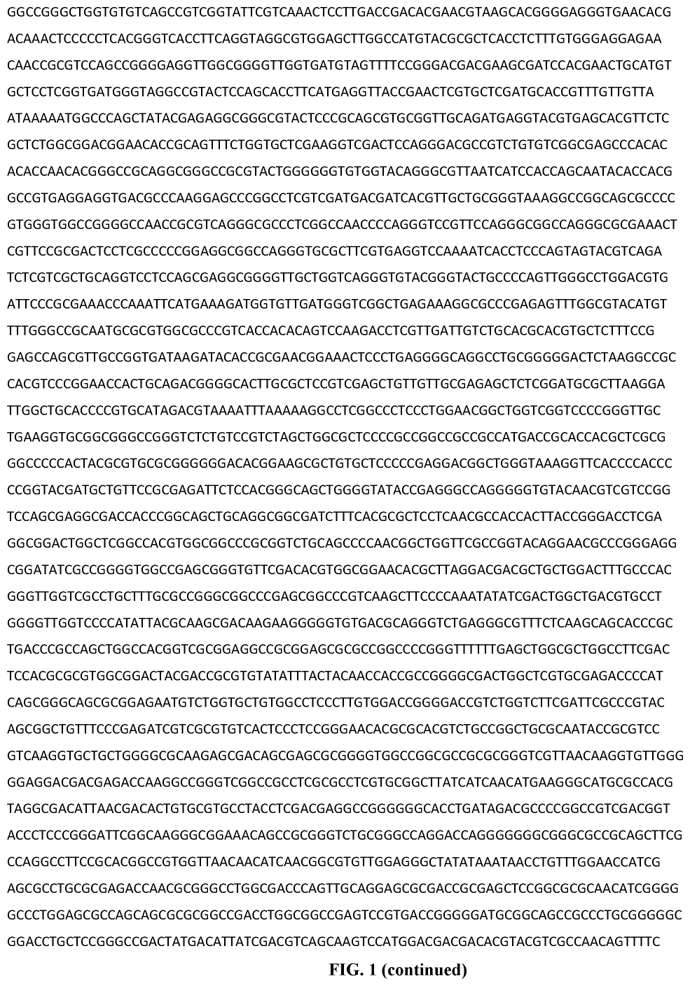

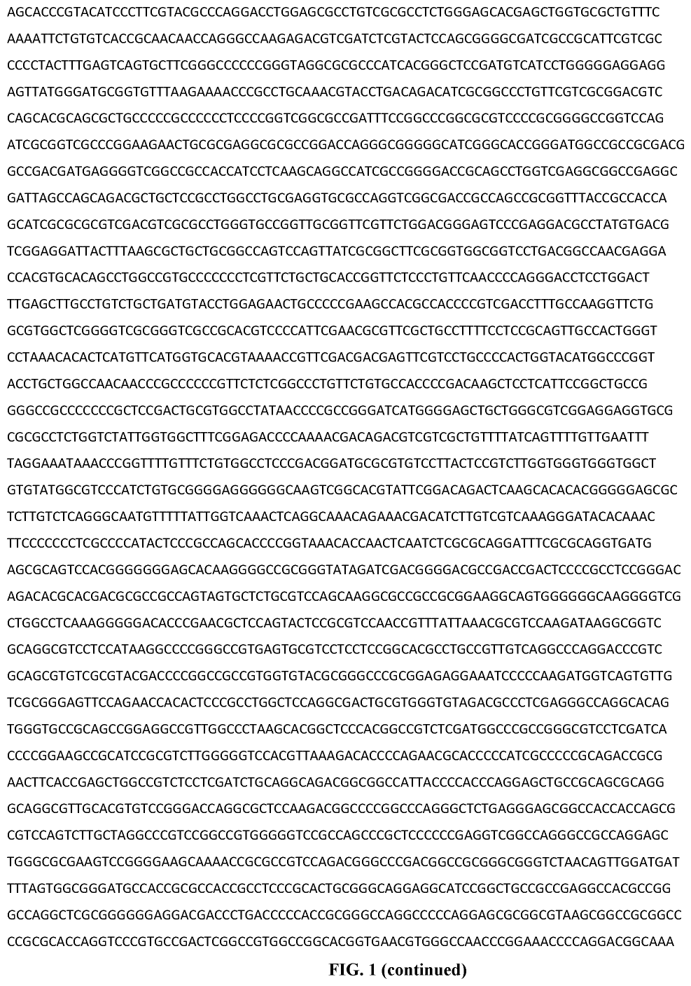

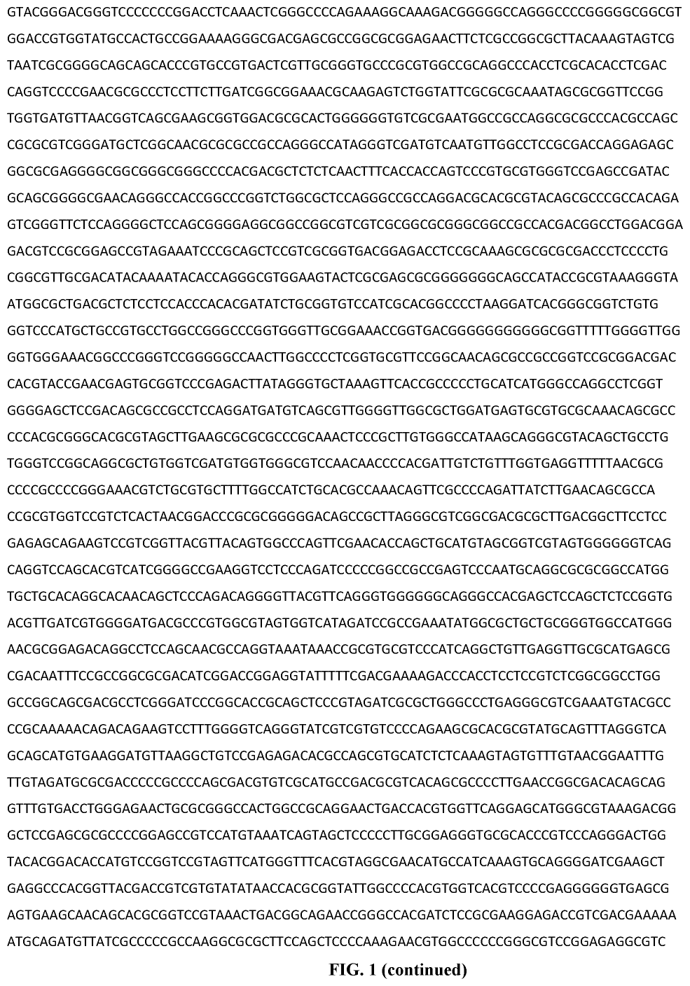

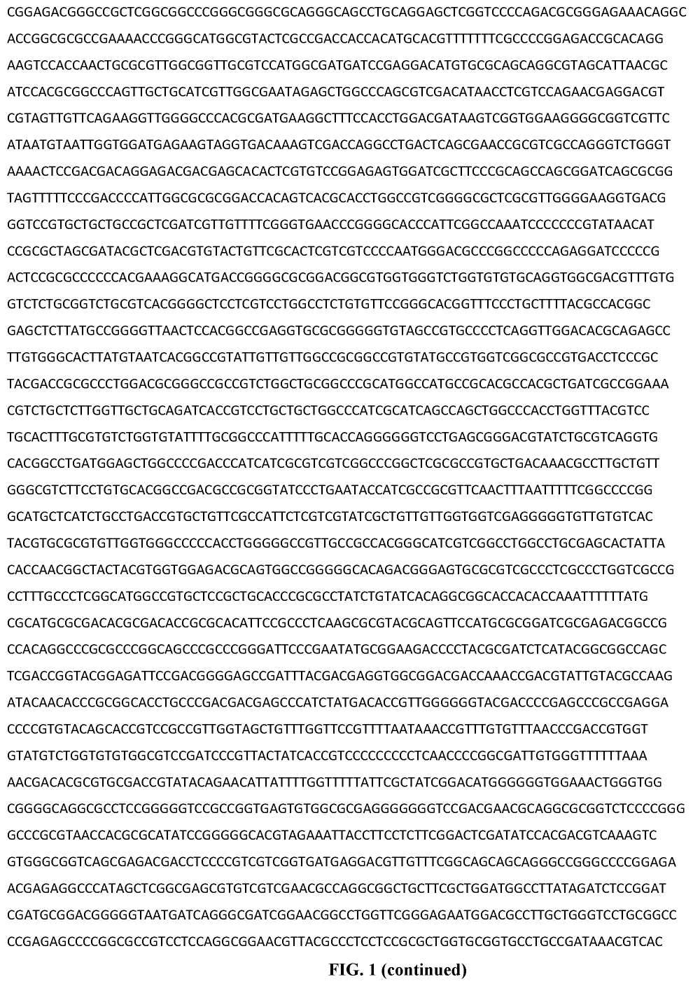

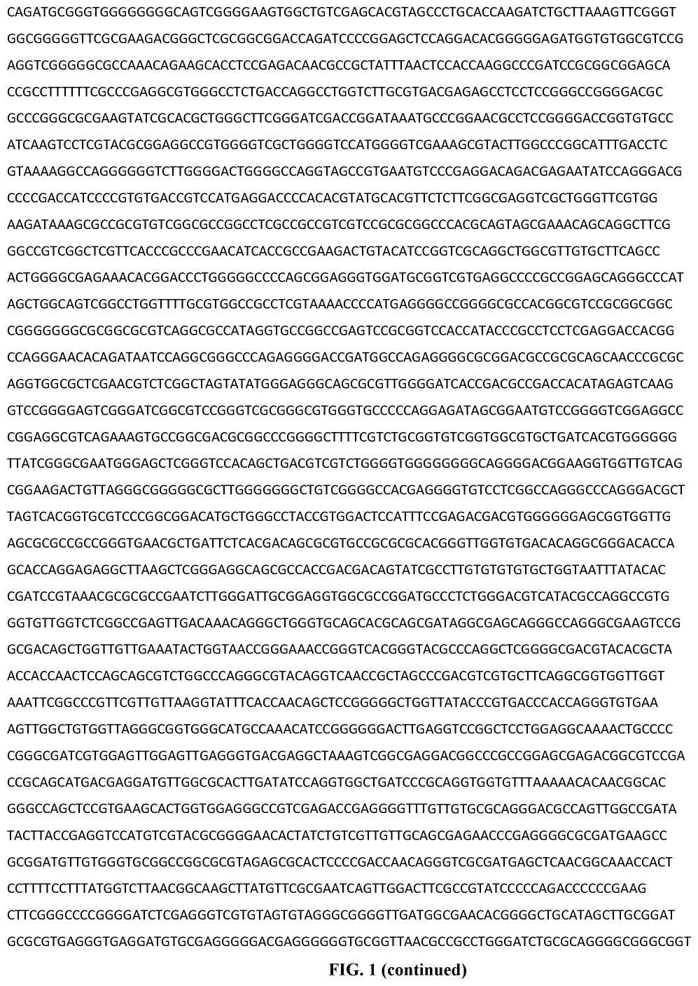

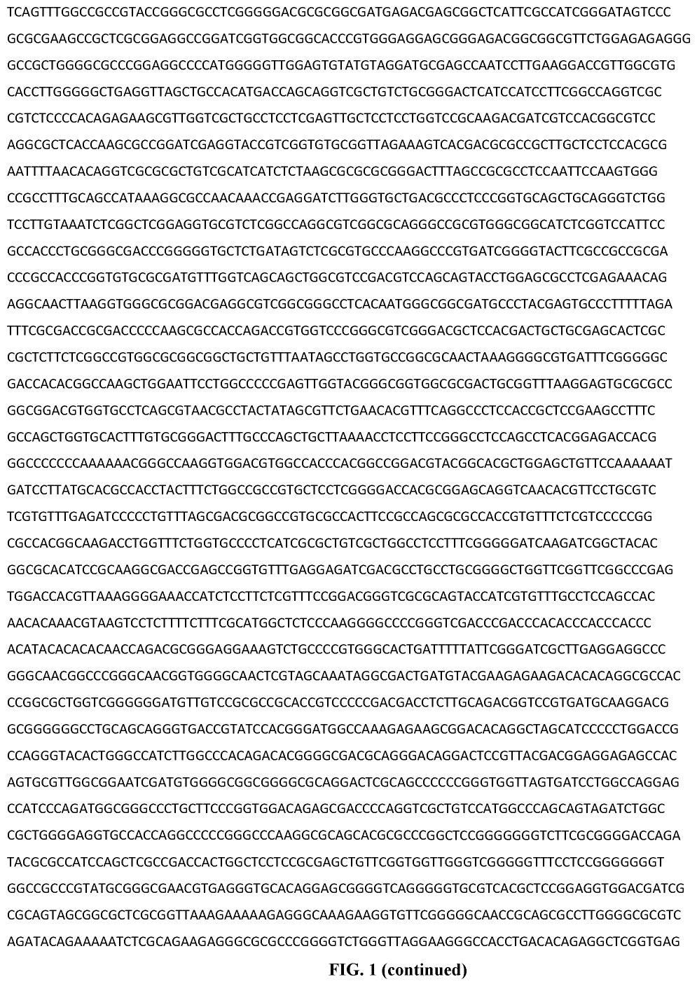

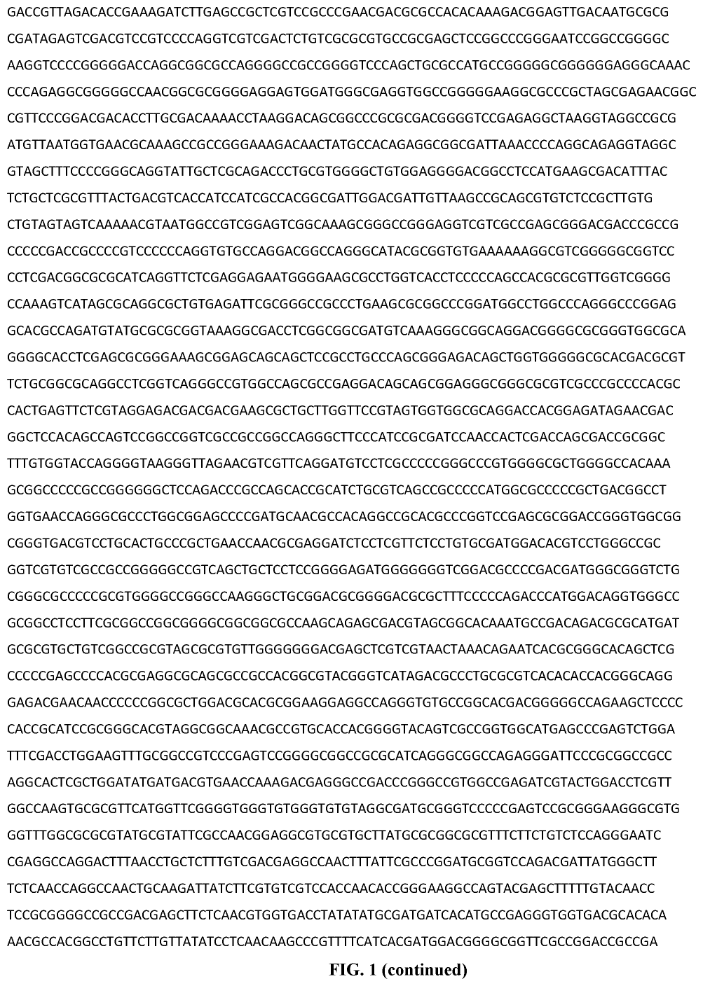

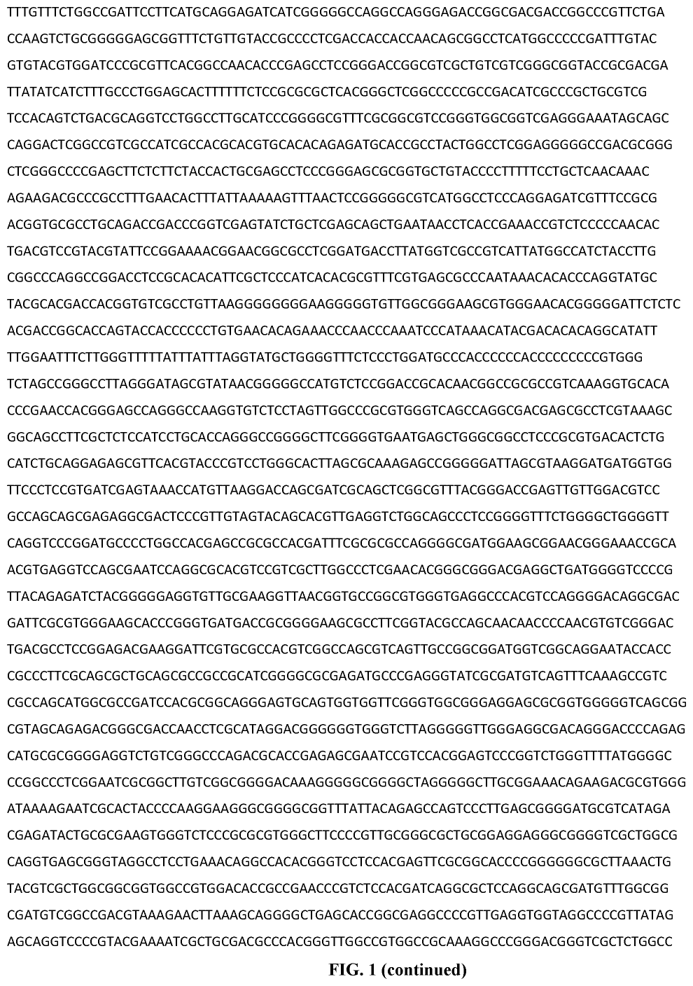

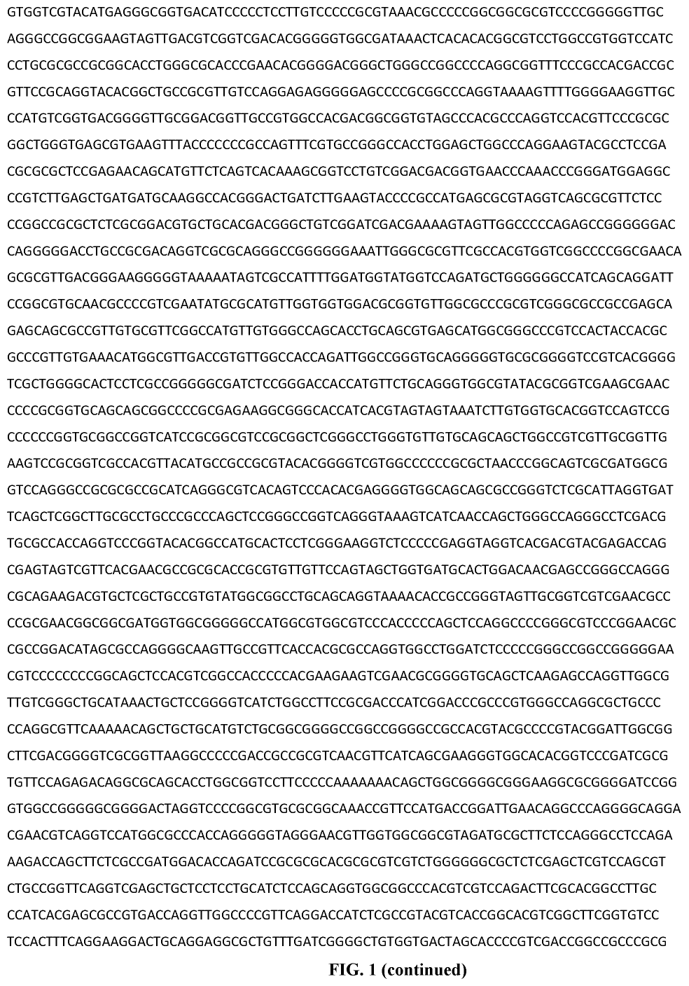

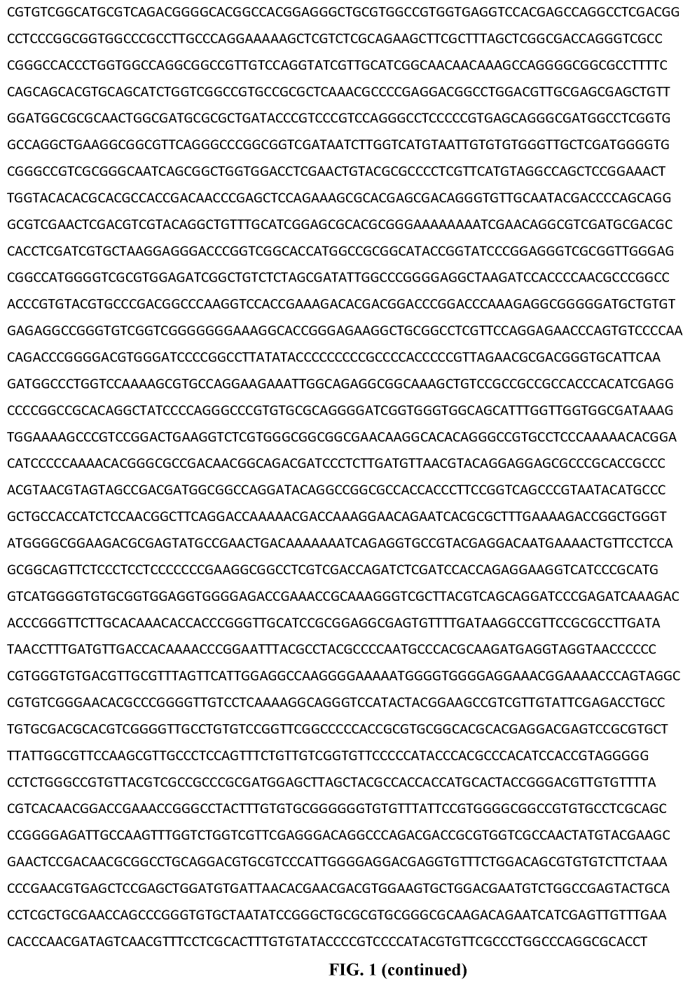

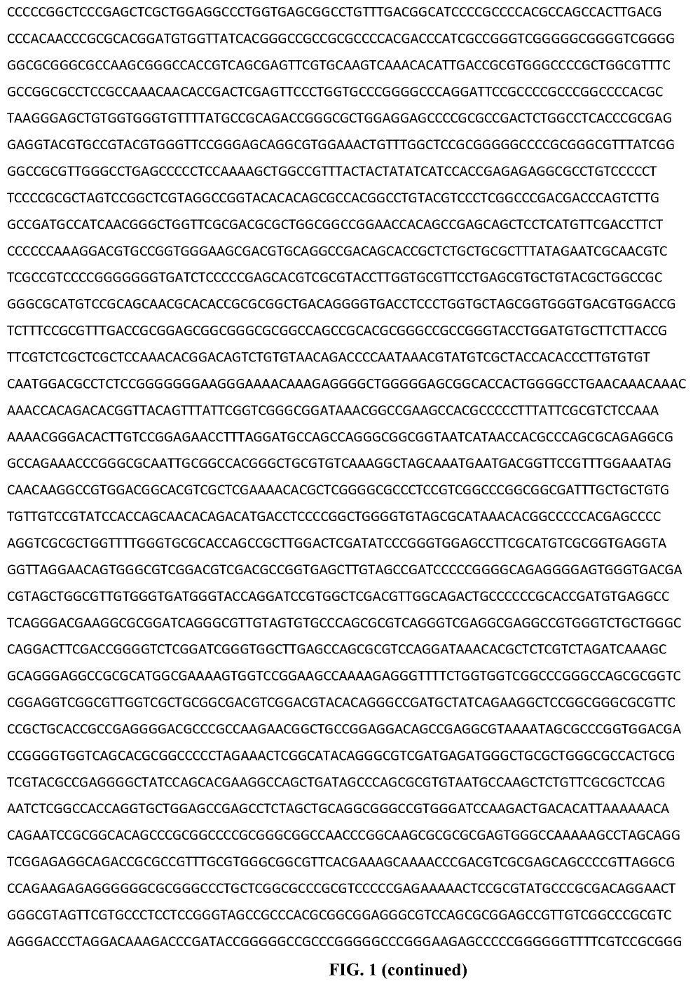

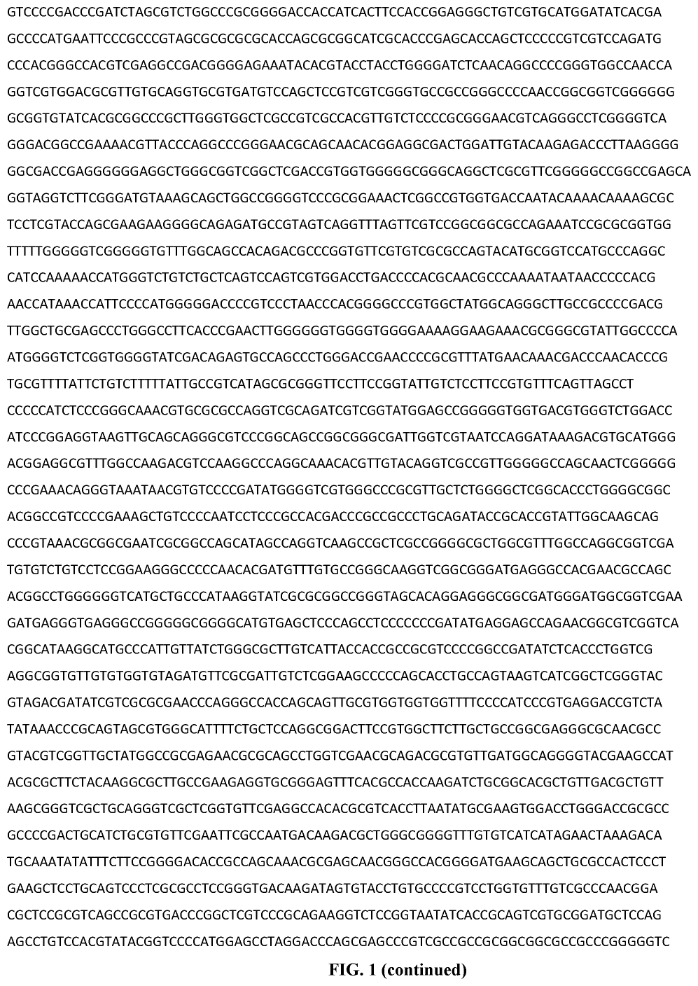

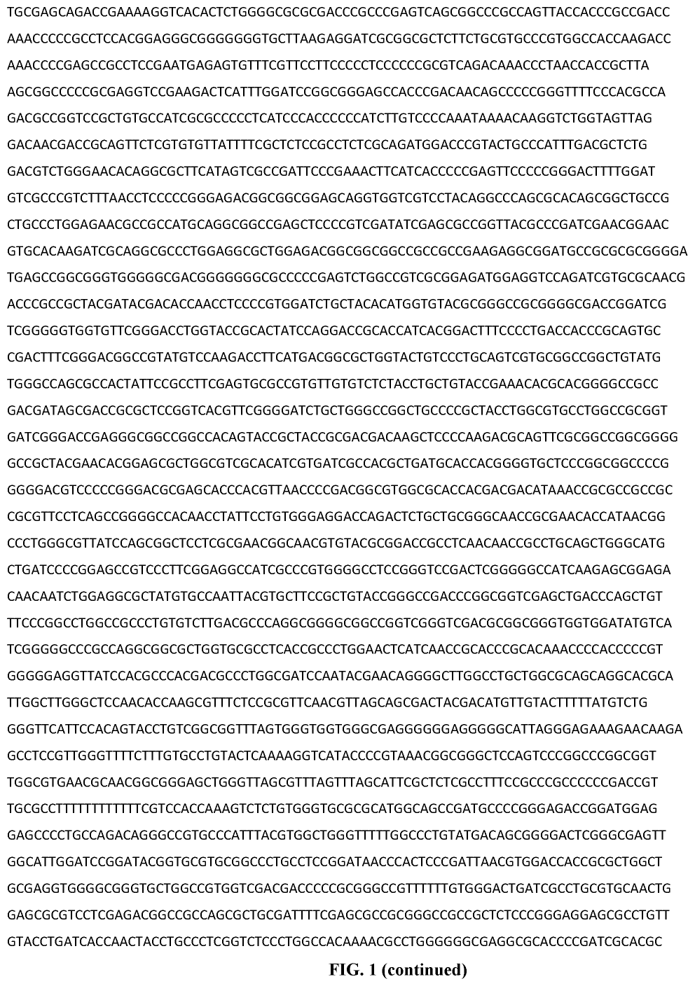

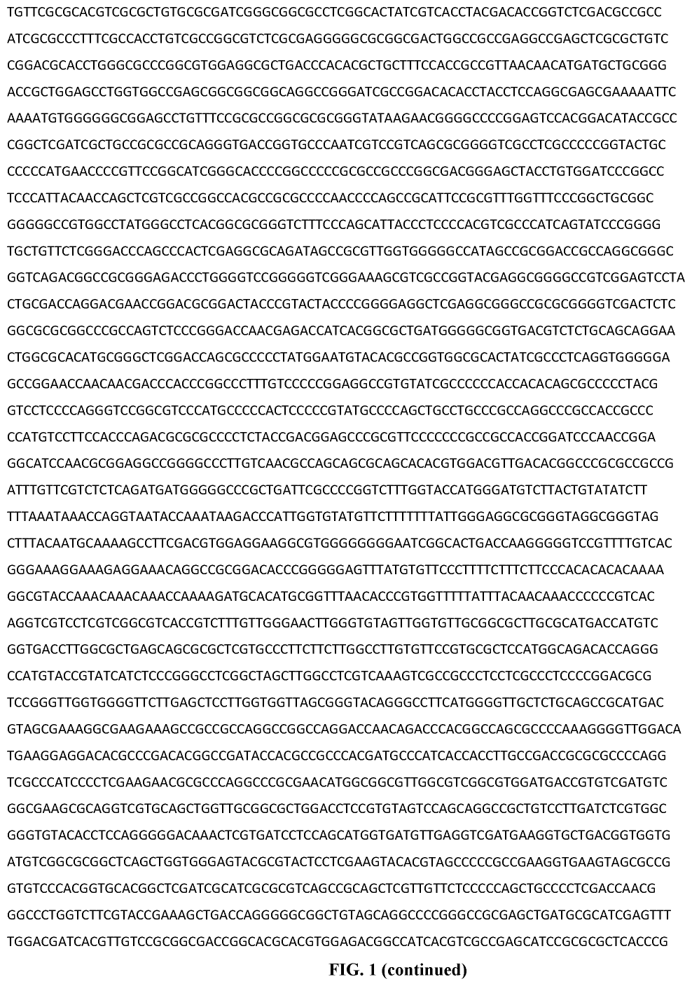

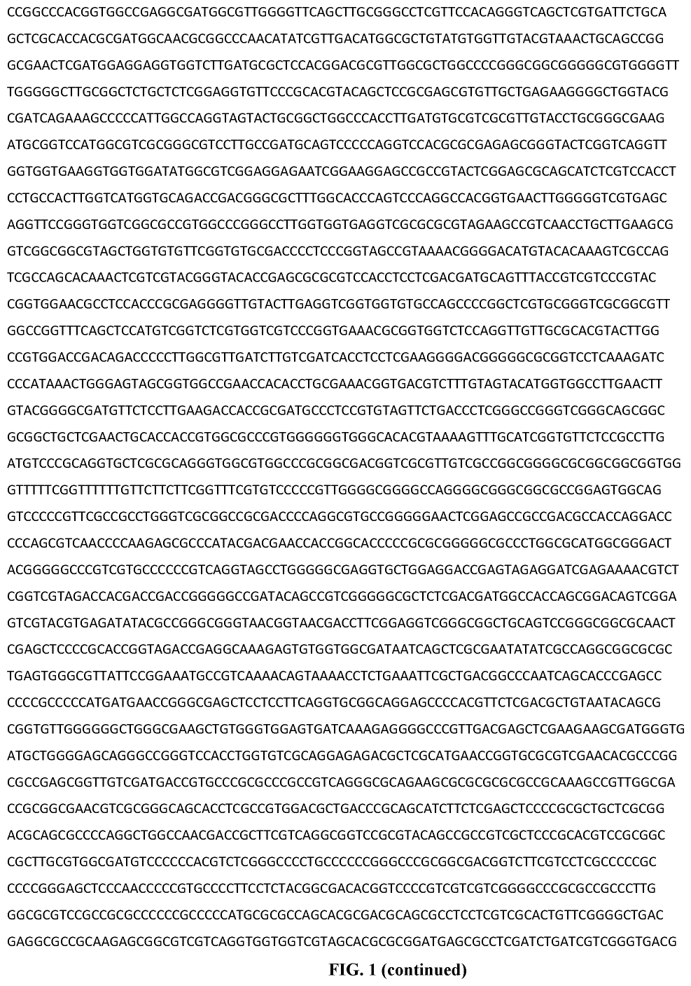

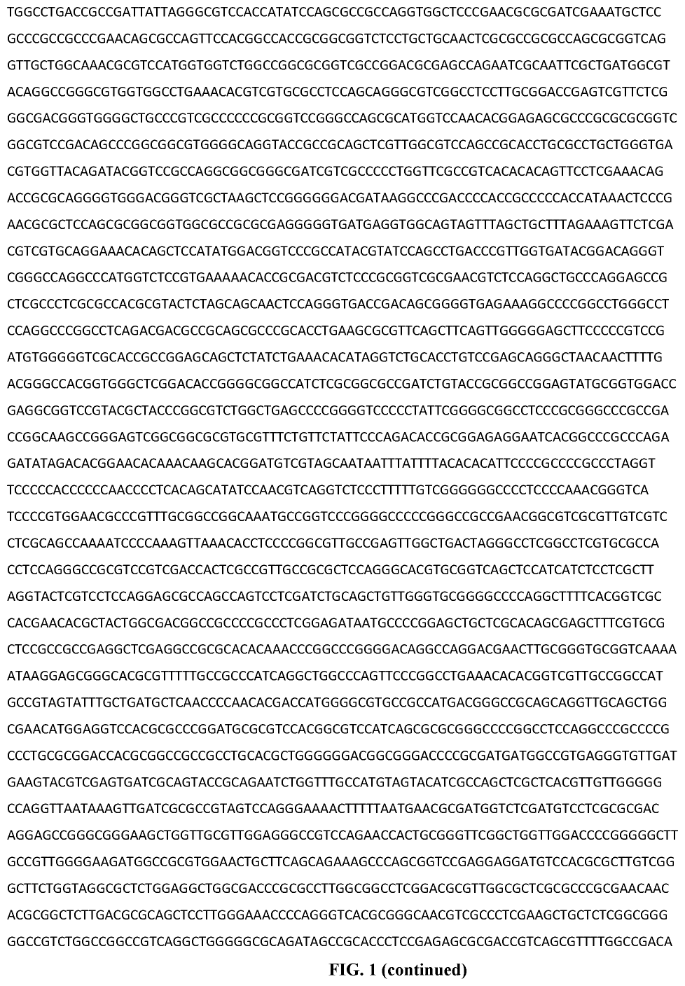

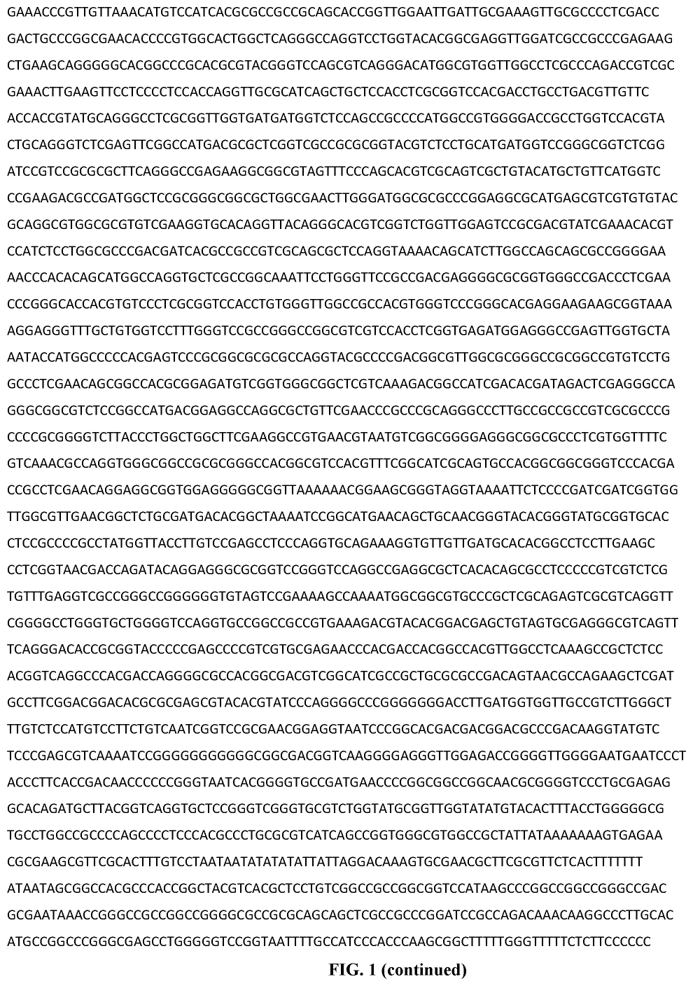

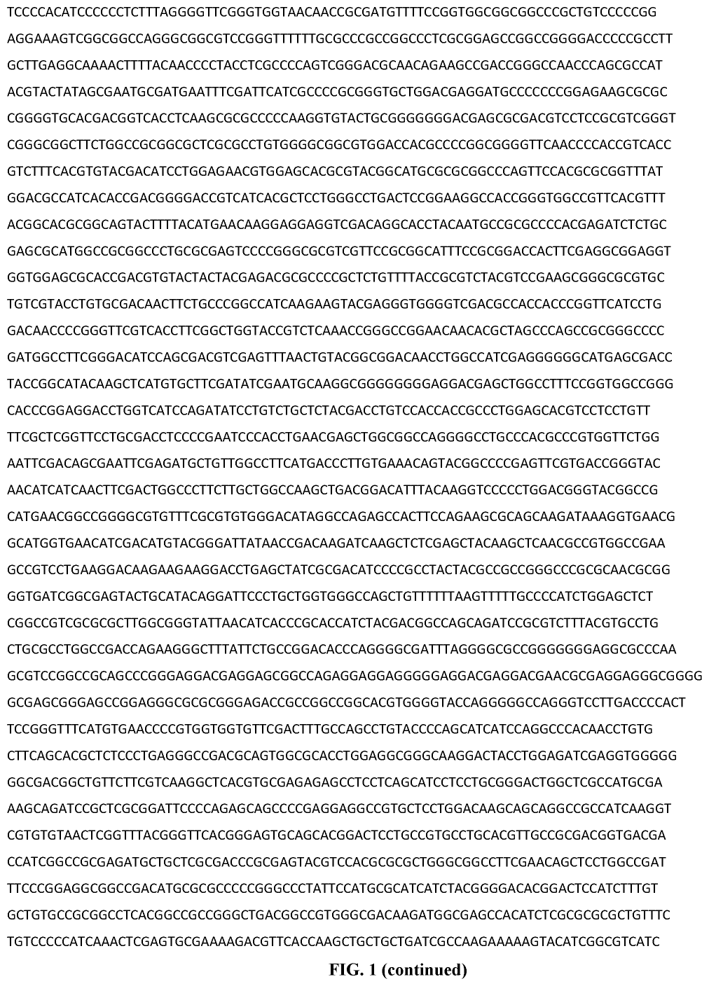

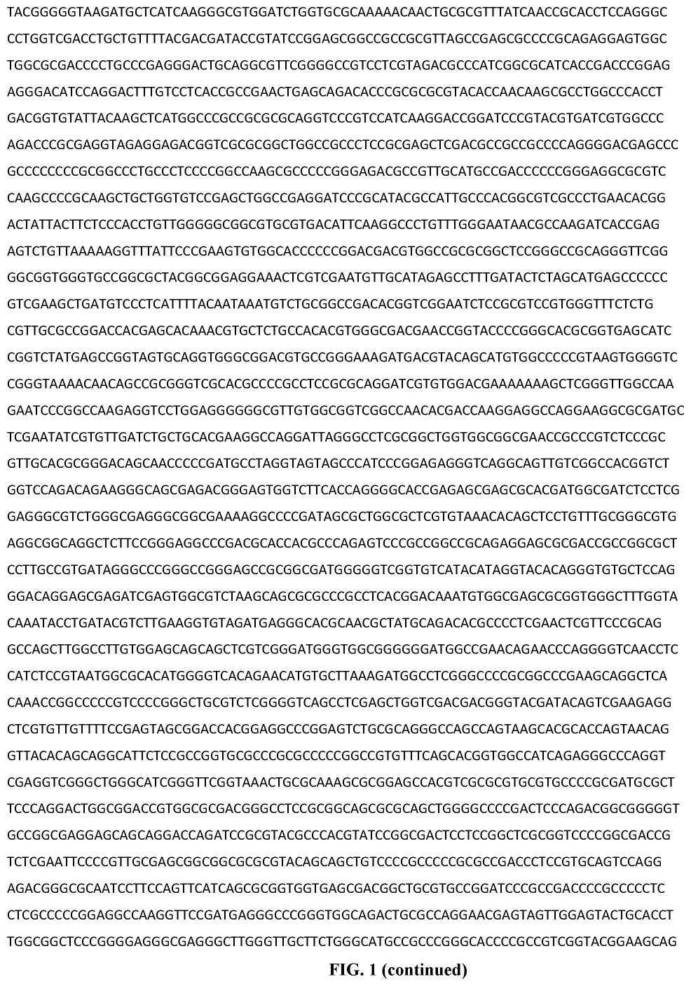

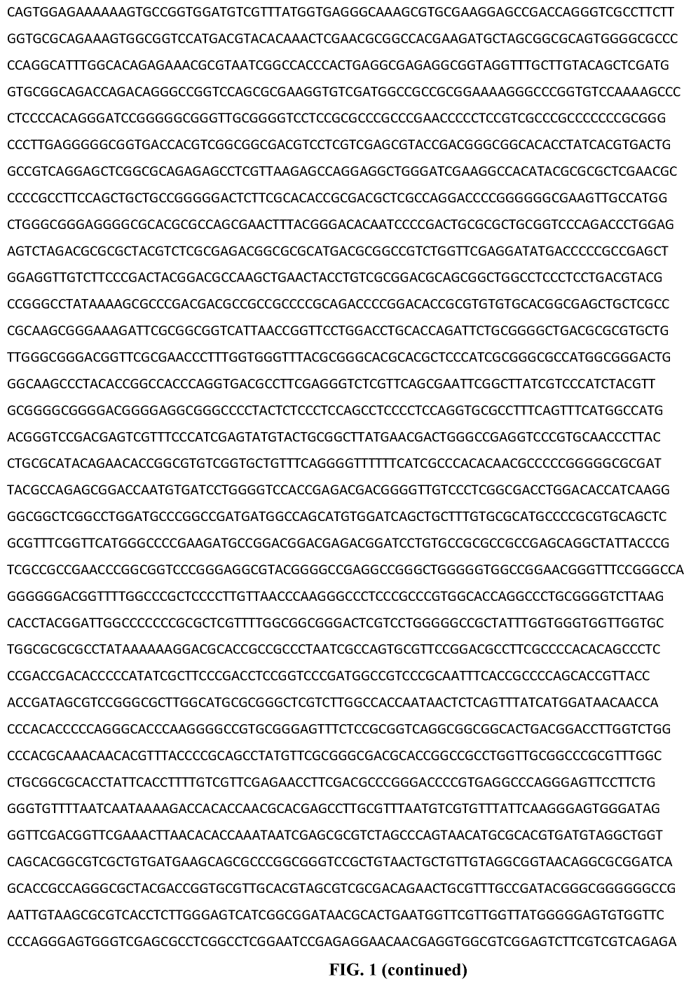

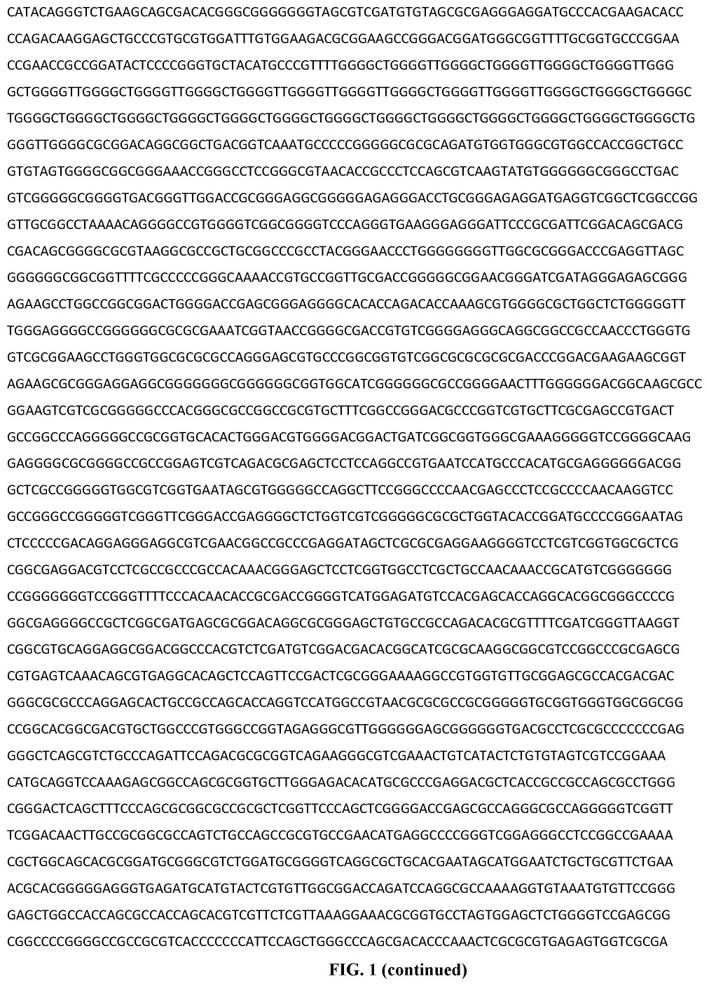

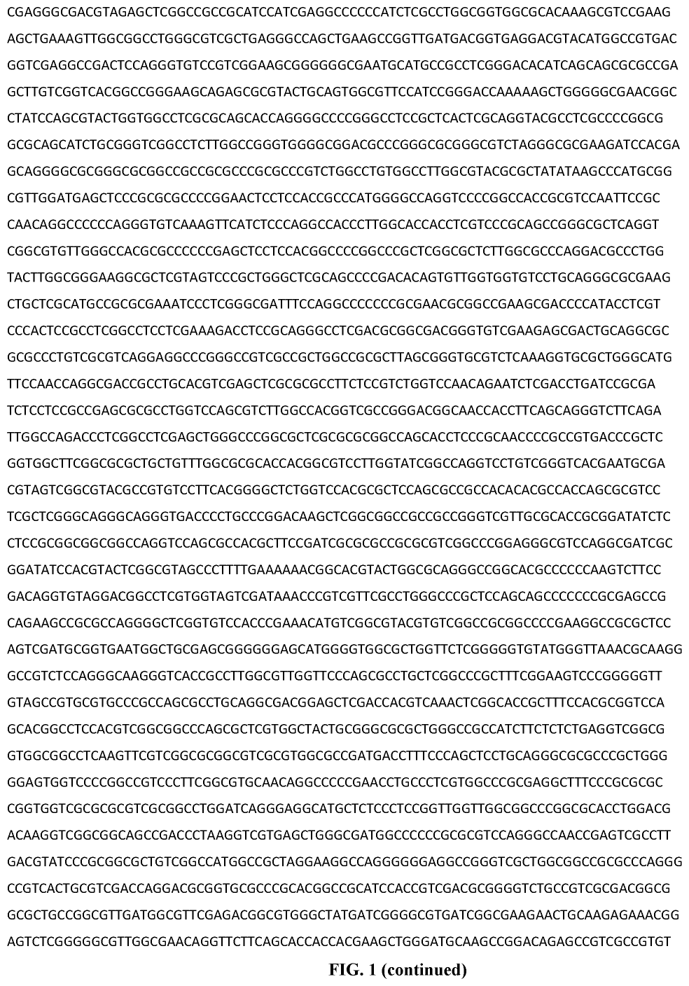

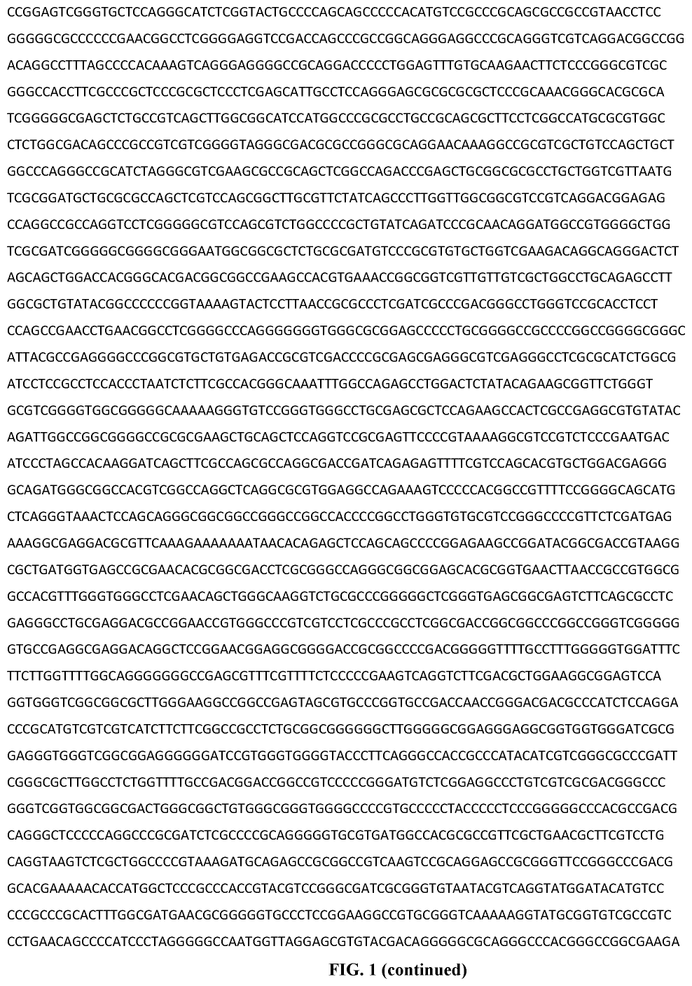

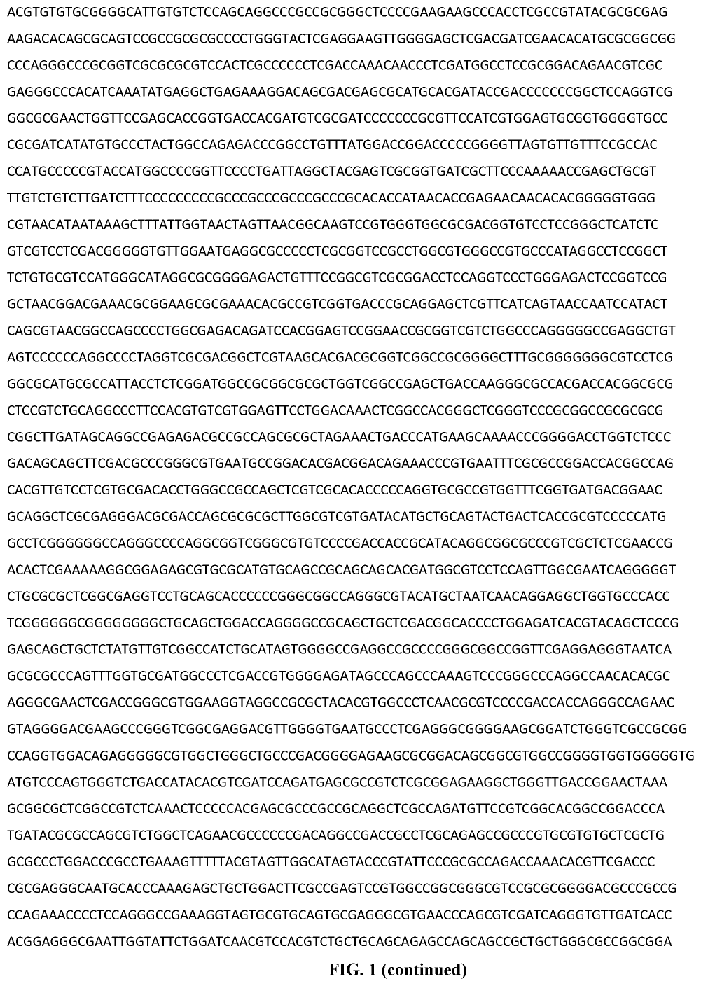

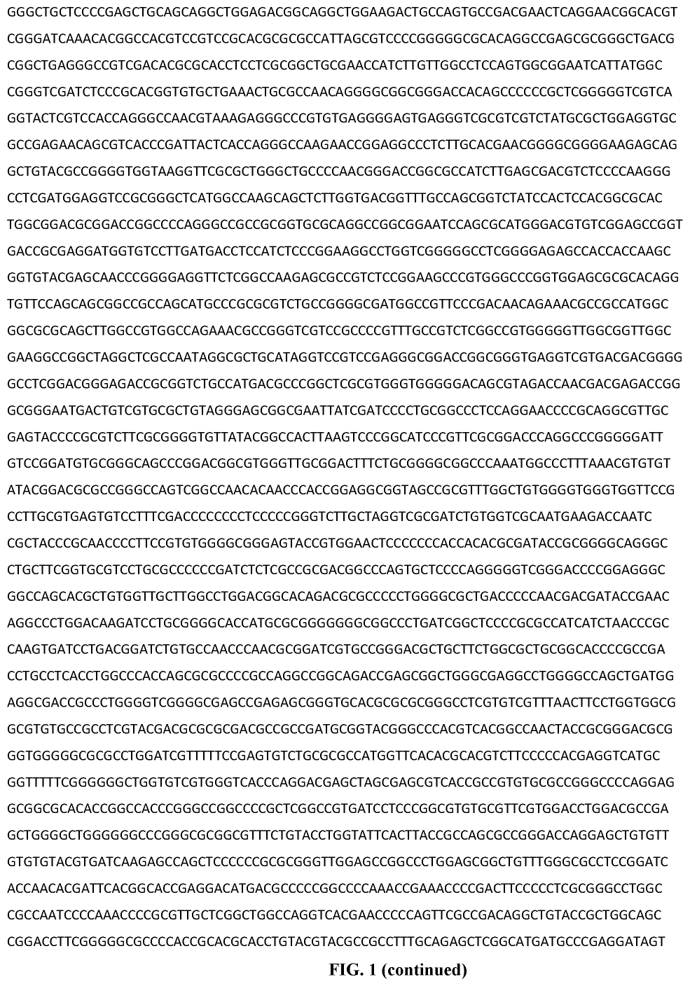

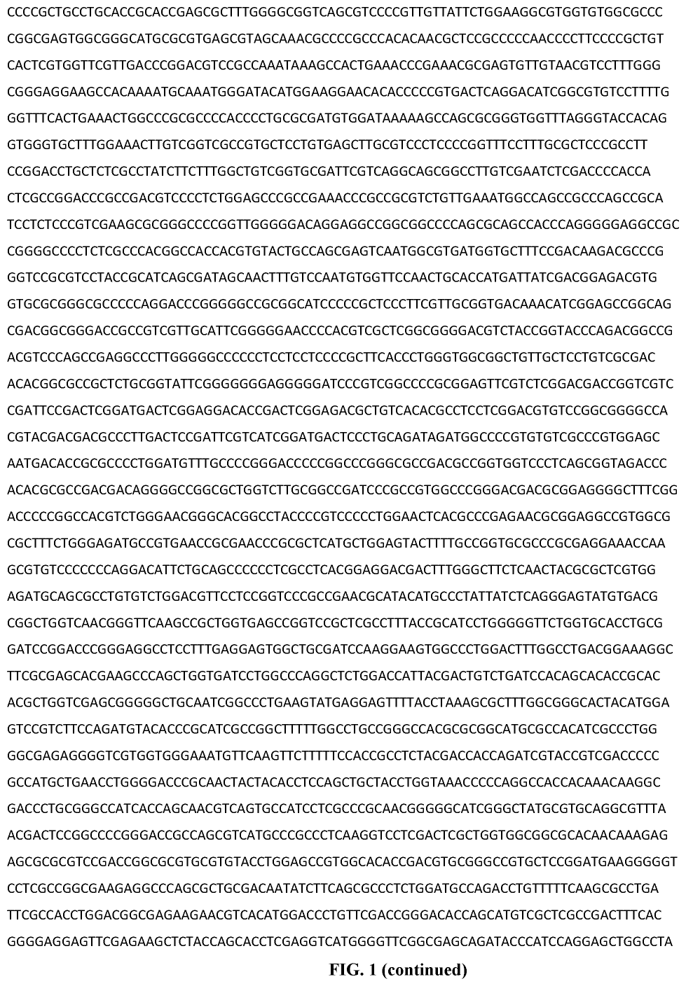

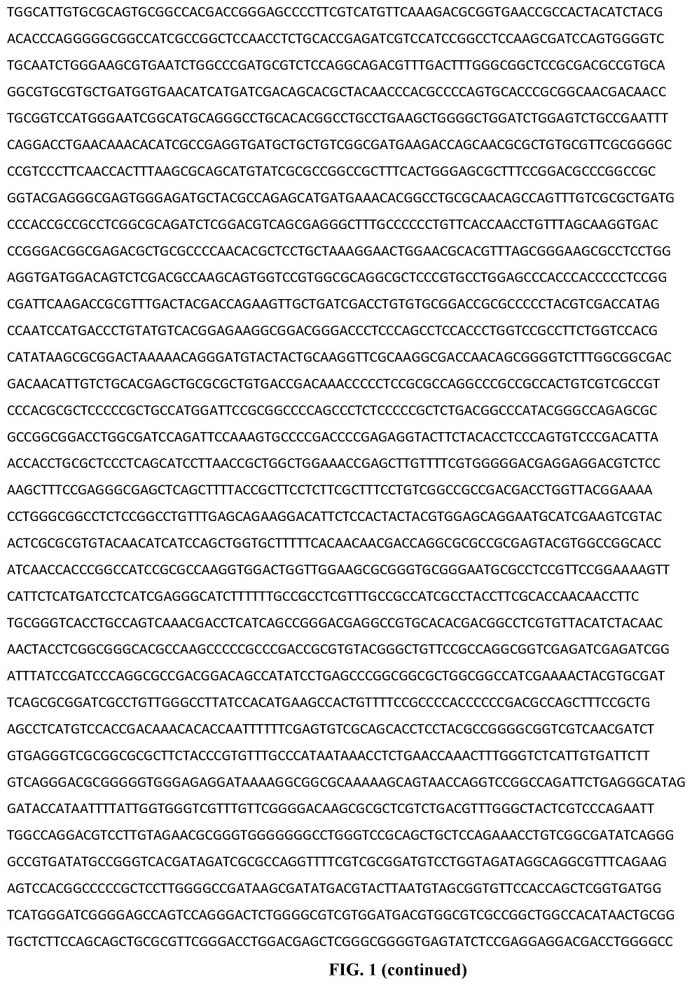

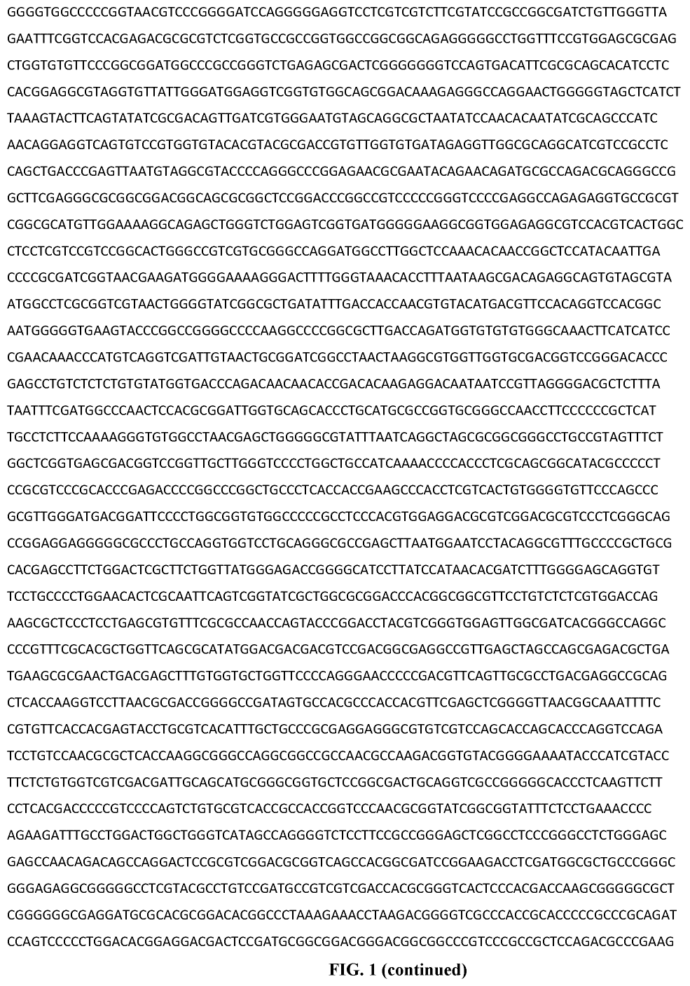

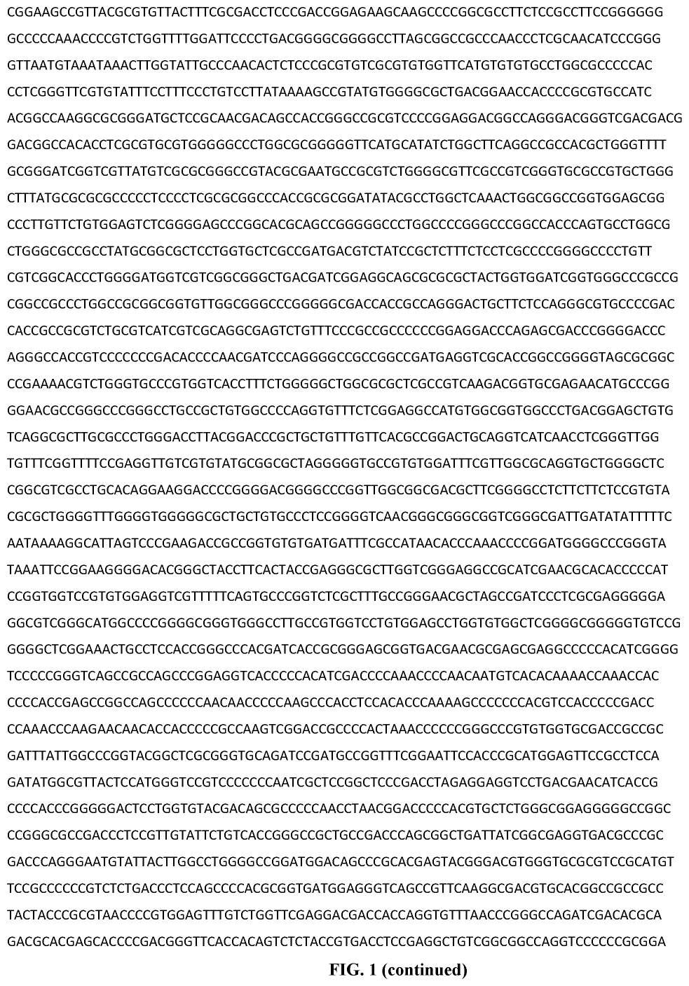

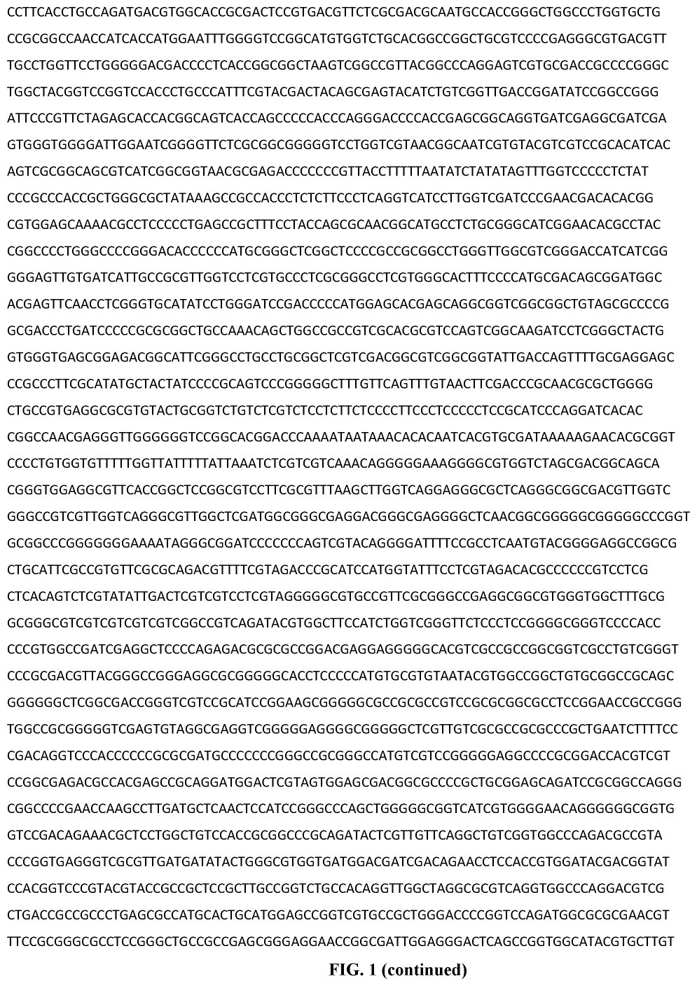

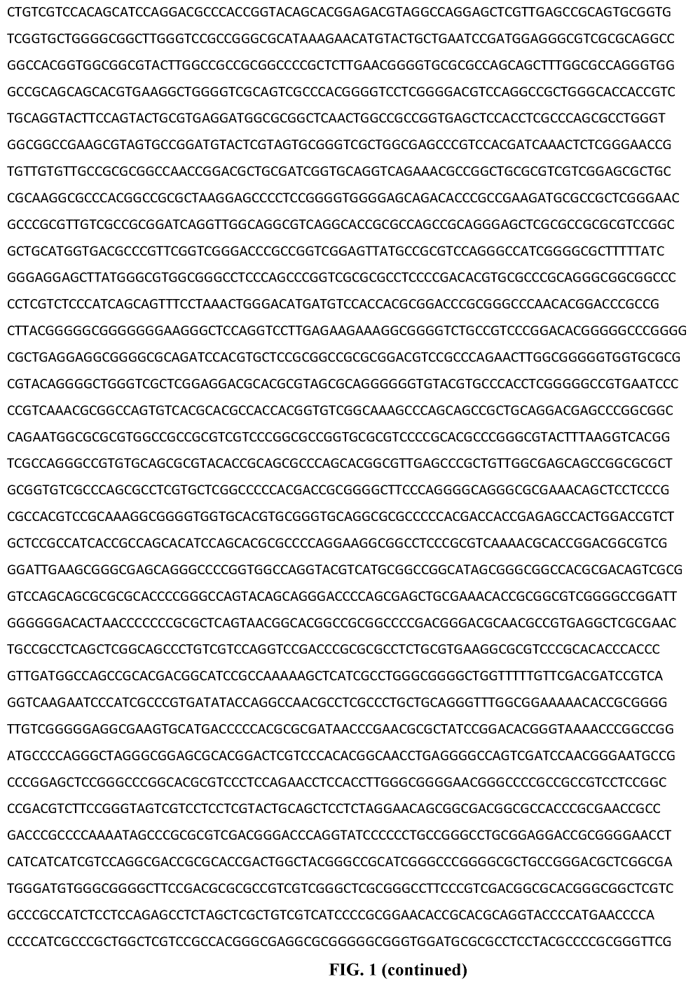

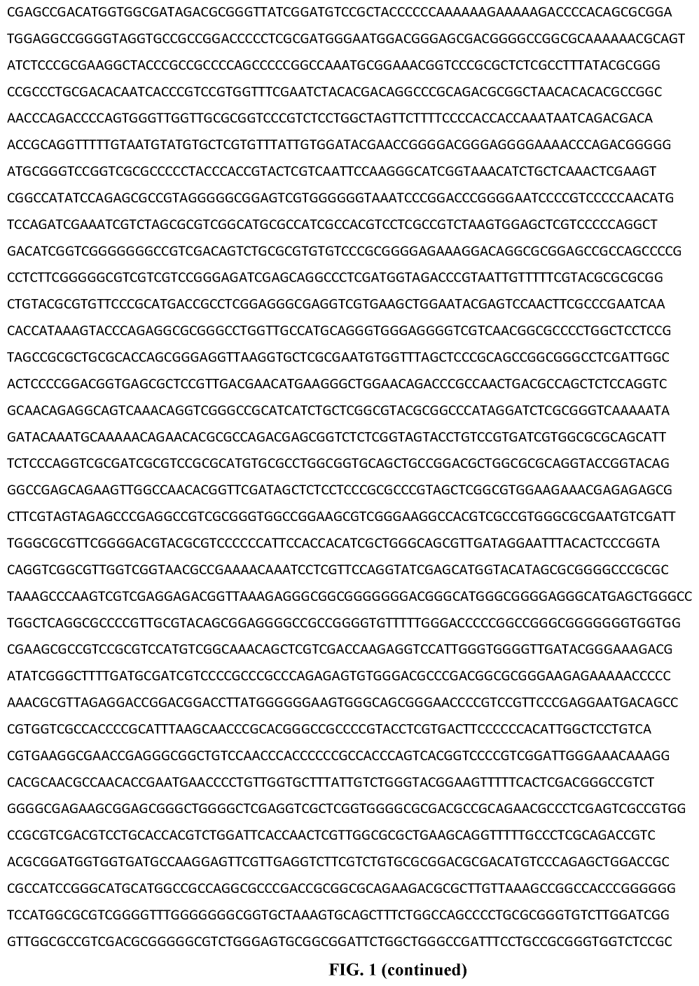

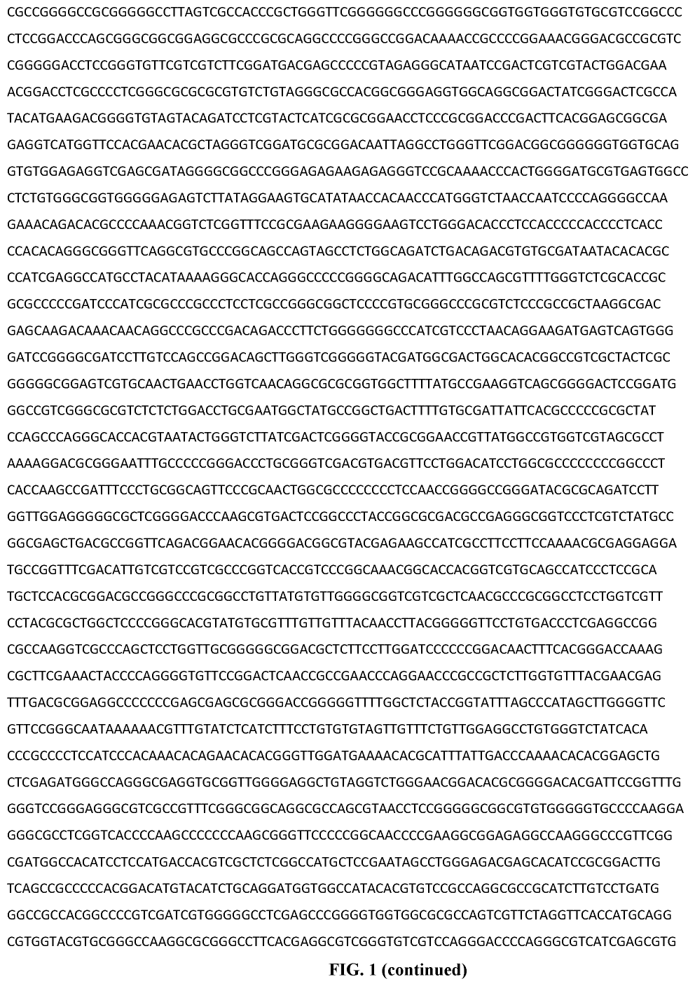

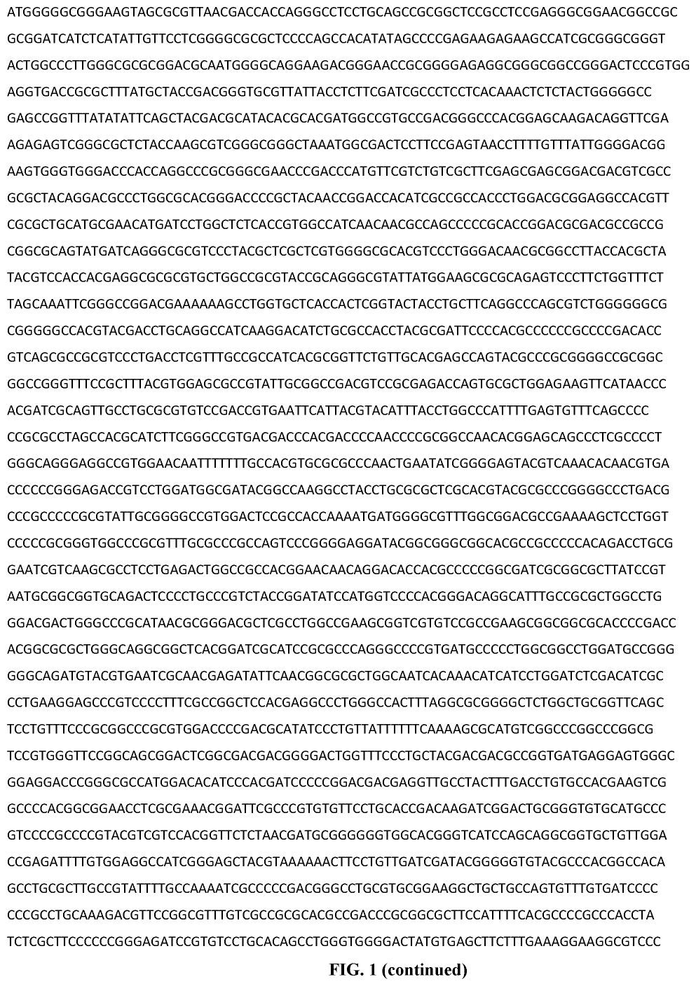

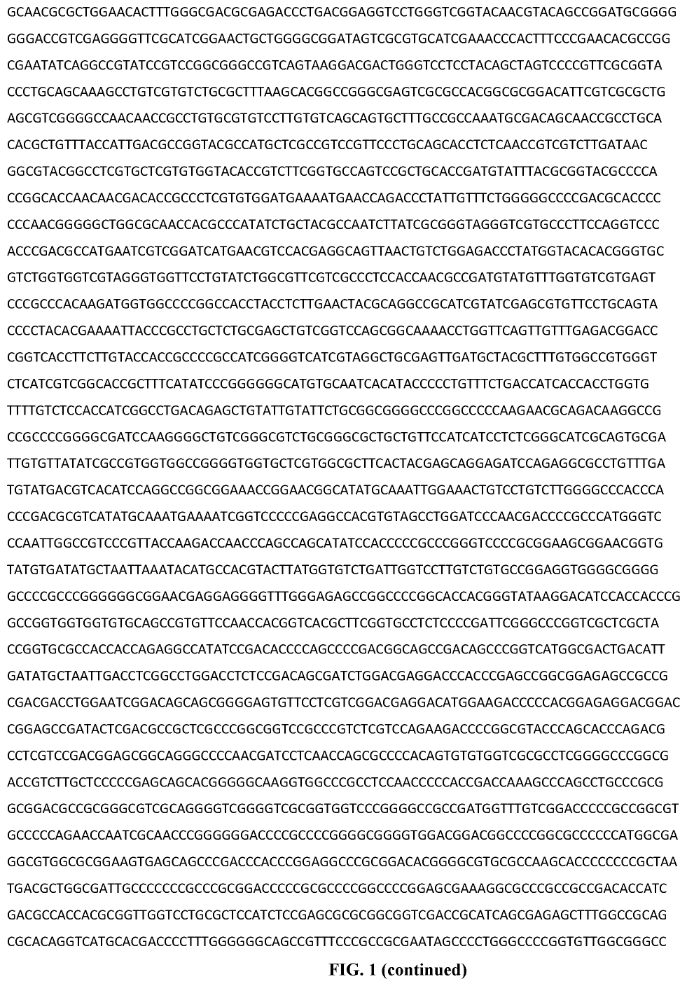

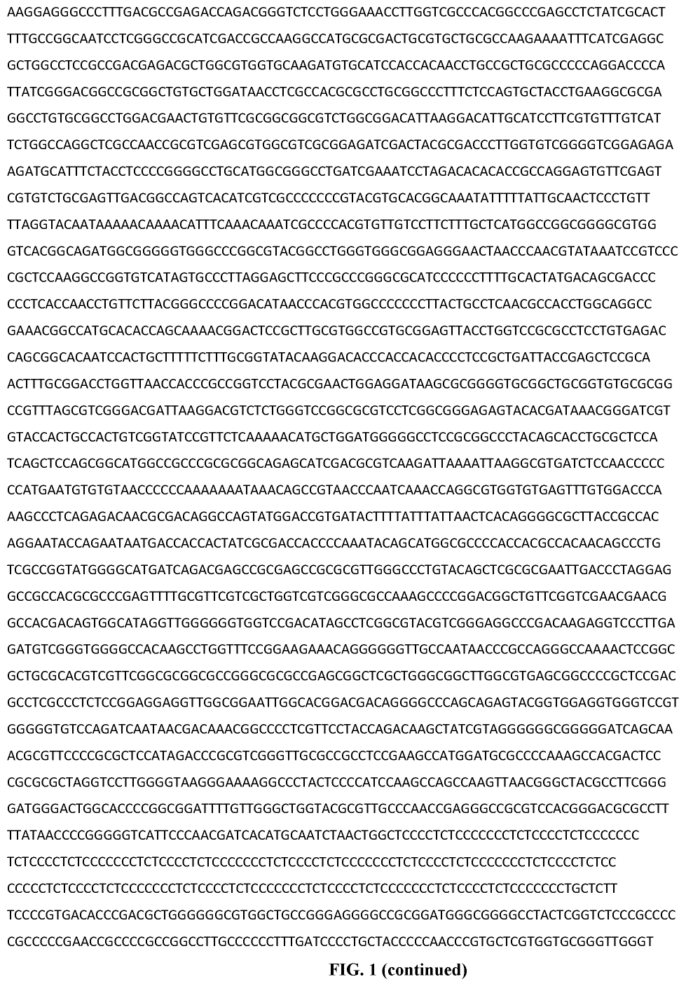

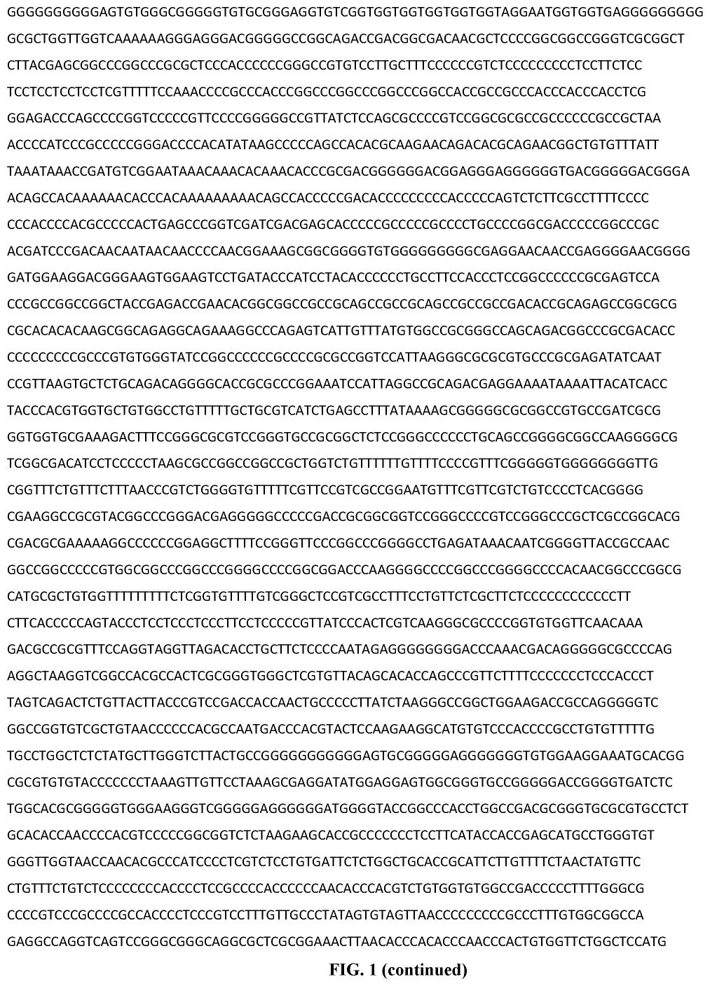

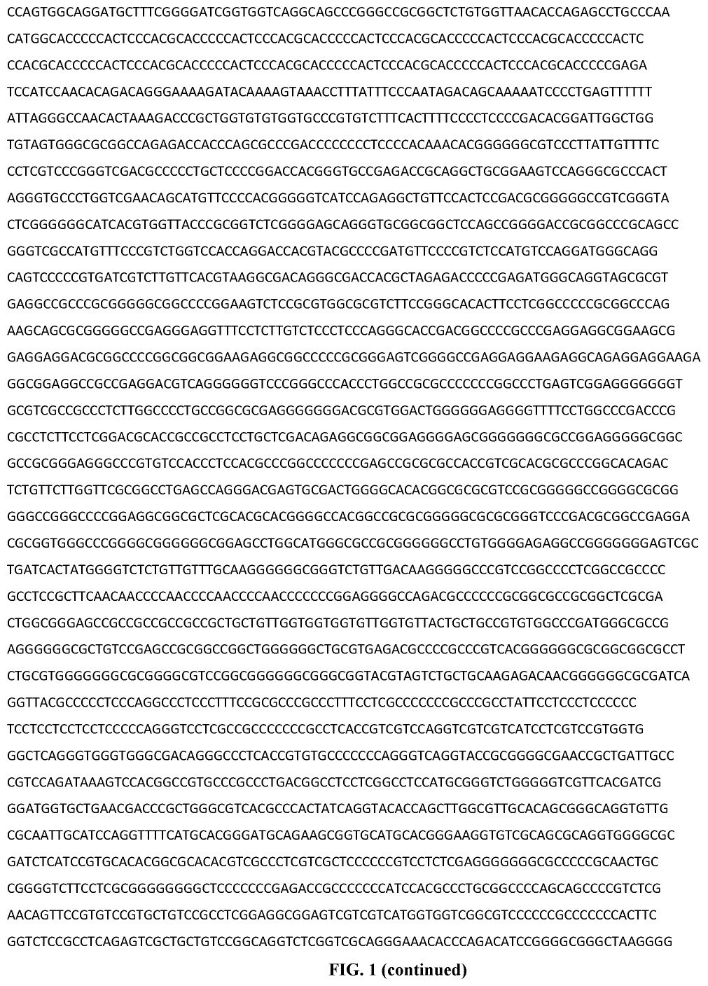

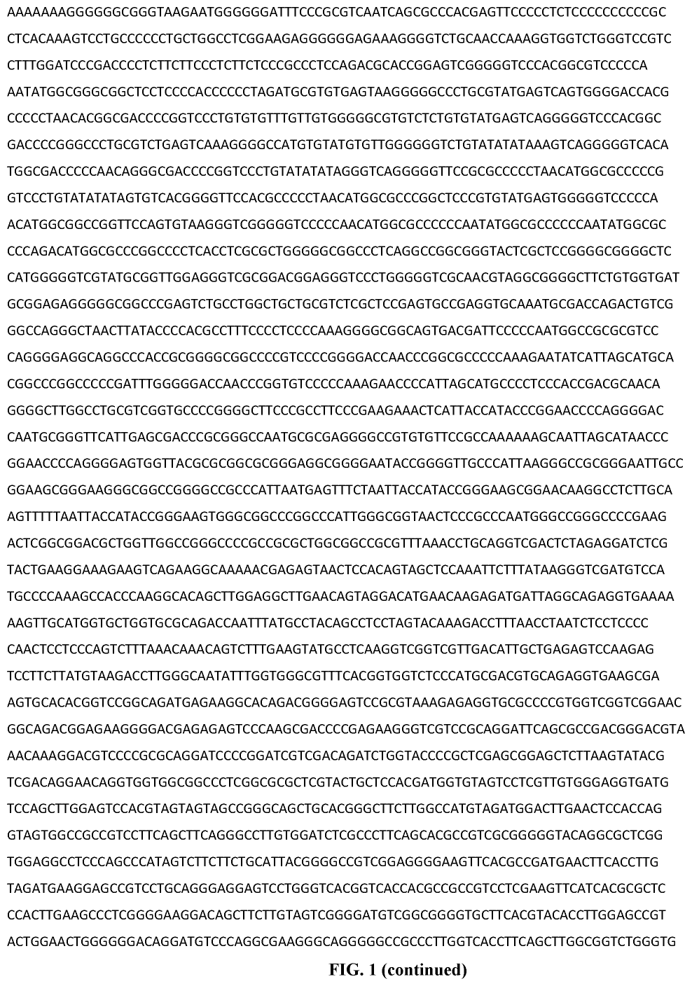

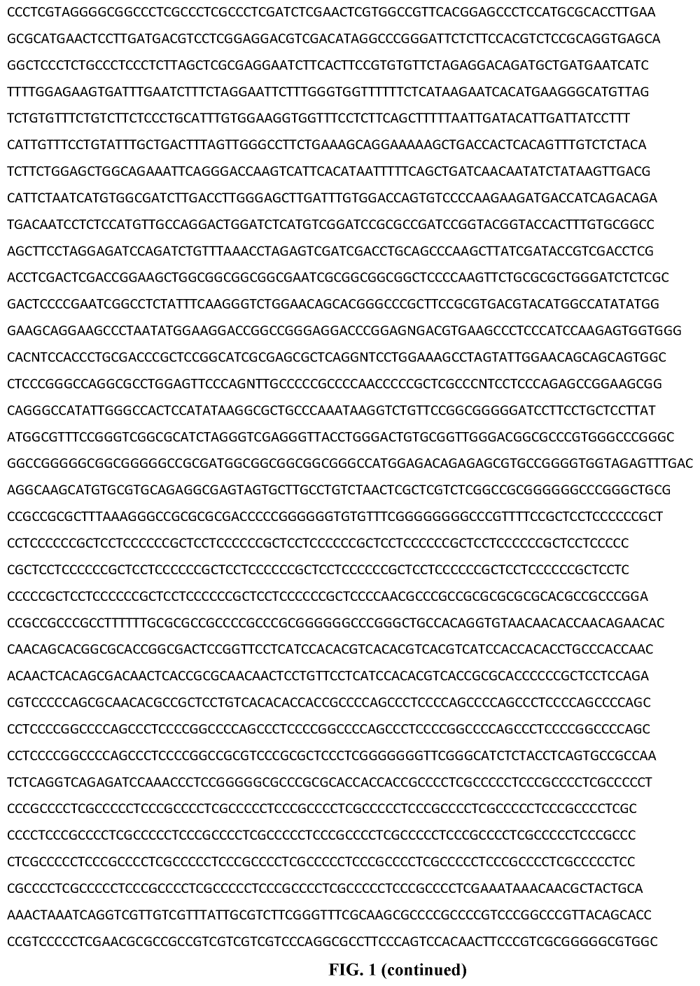

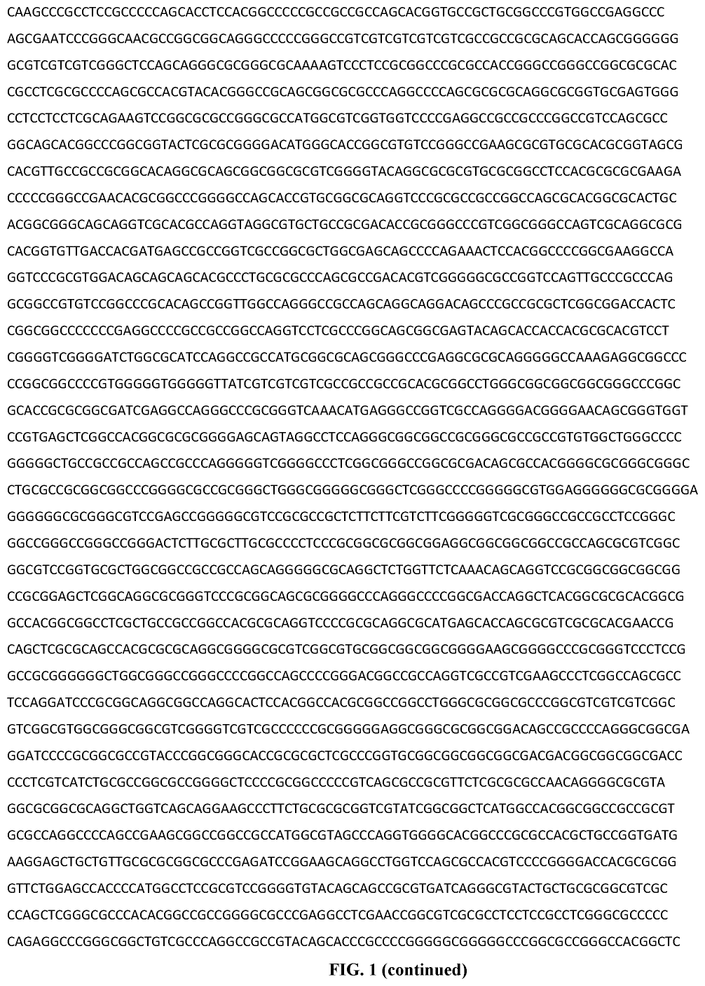

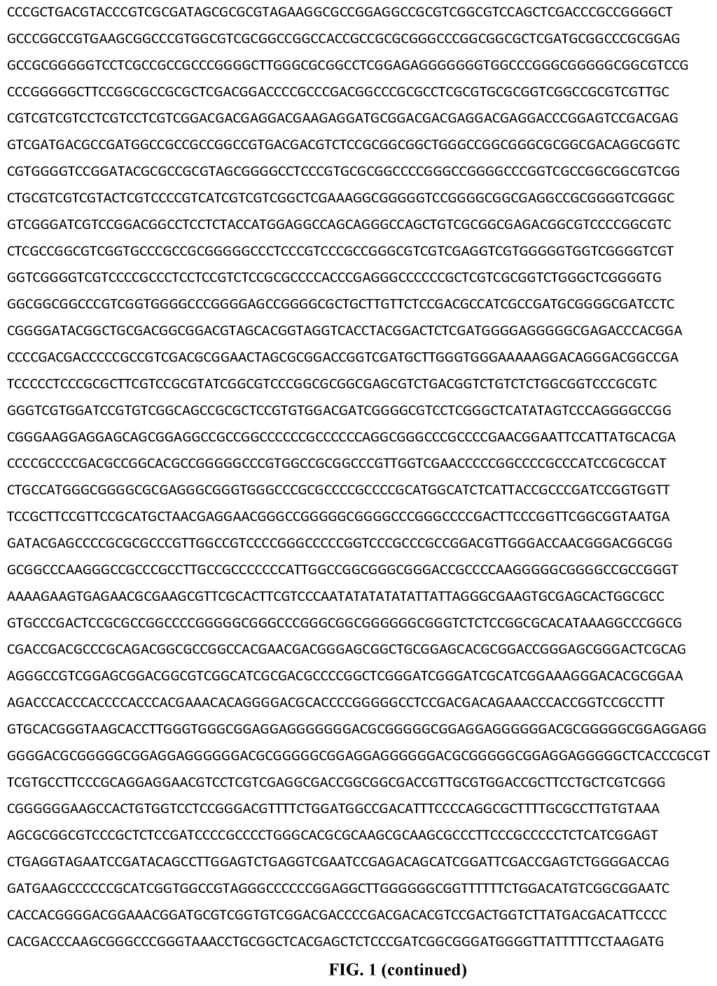

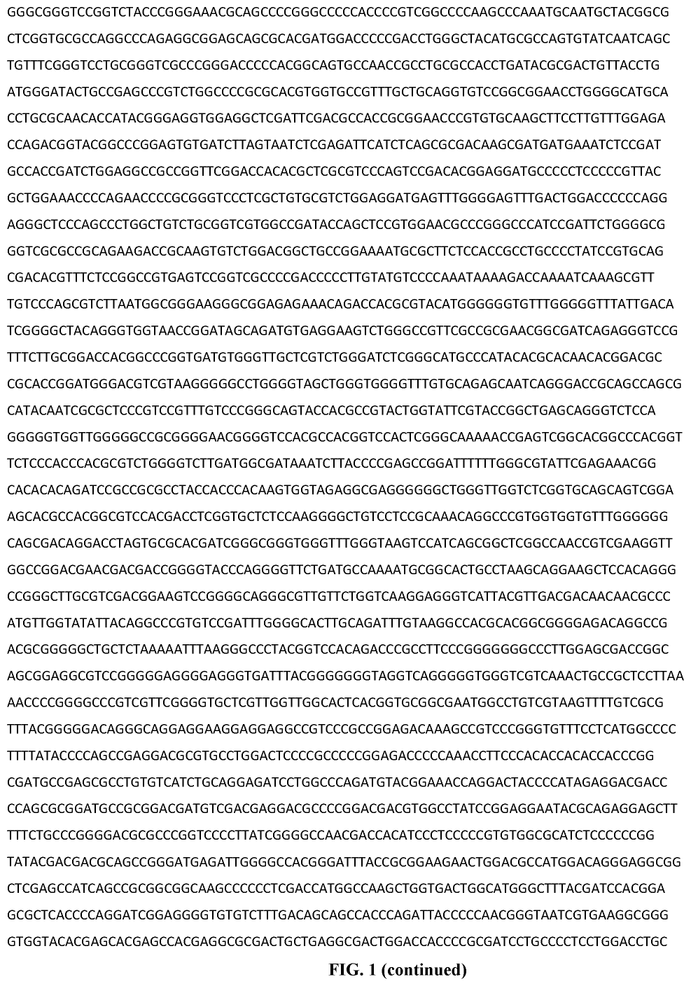

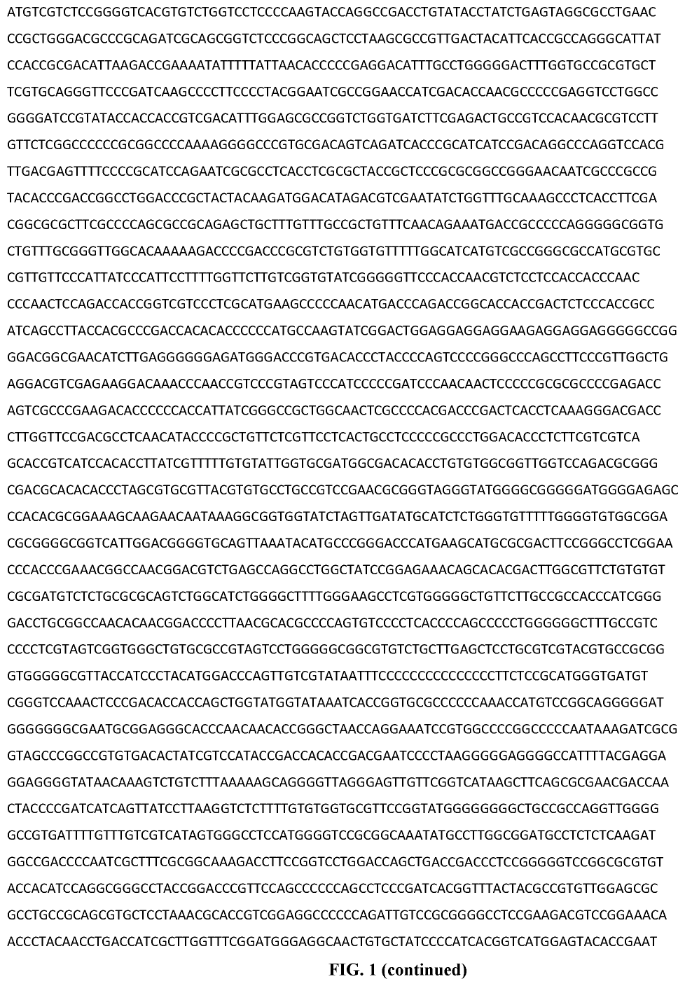

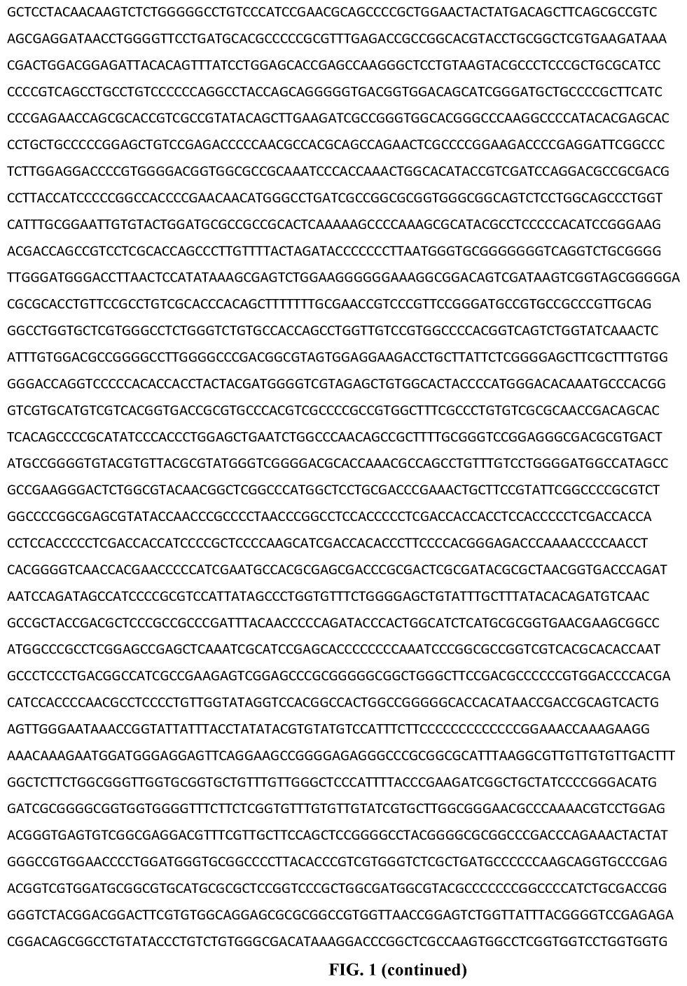

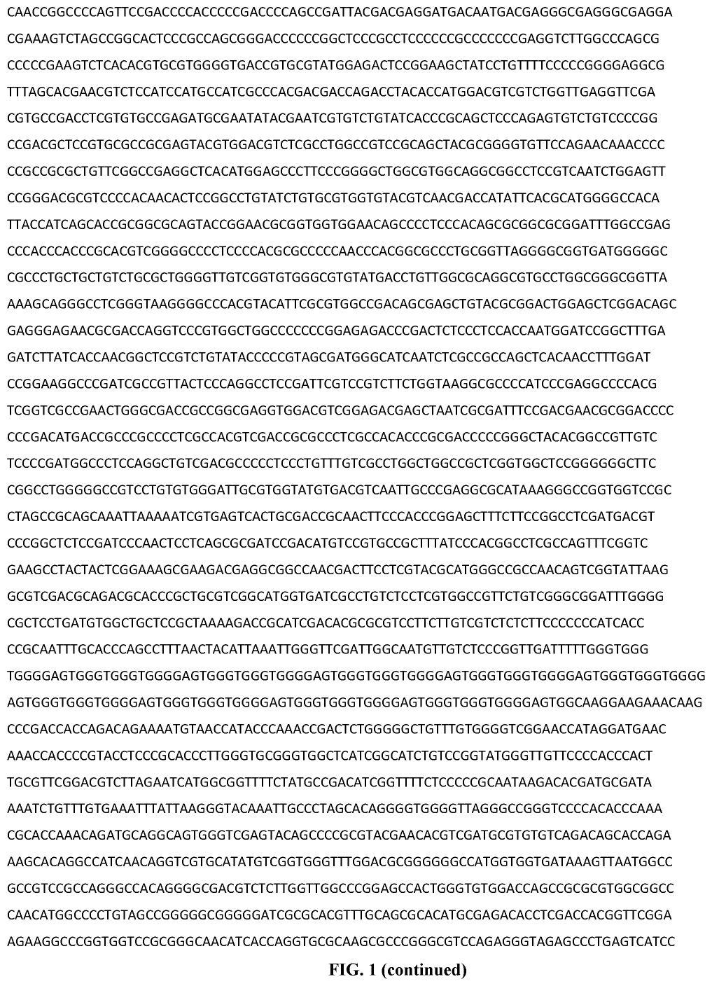

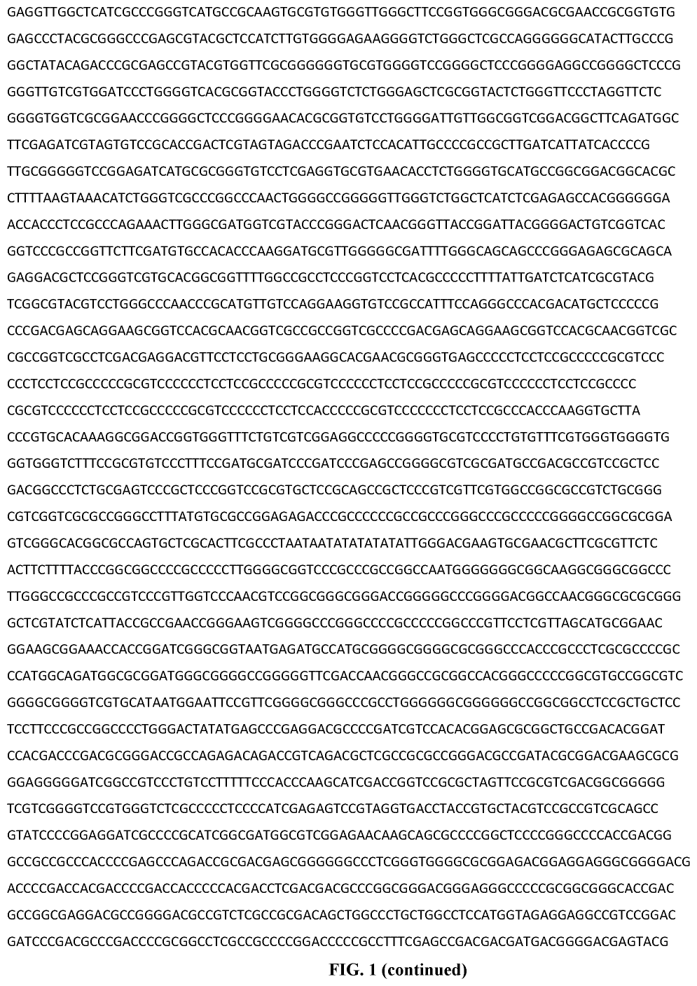

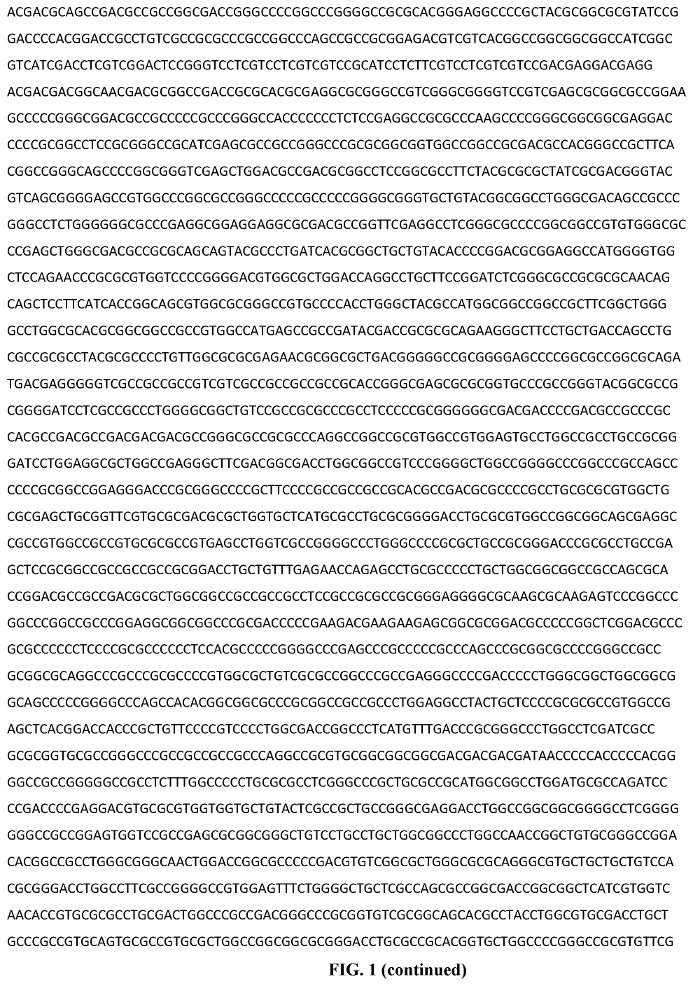

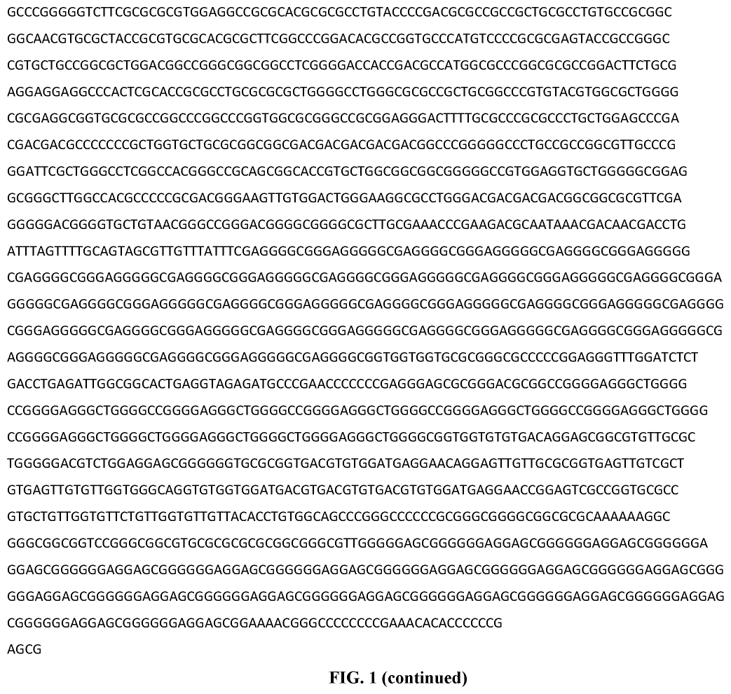

In one embodiment, the non-natural HSV is obtained by mutating or modifying any of the genes of wild-type HSV or by inserting any of exogenous genes. The serum type of HSV comprises, alternatively consists essentially of, or yet further consists of a type 1 HSV (or HSV-1), a type 2 HSV (or HSV-2) or C134 HSV, known in the literature, see Cassidy et al., Pre-clinical Assessmnent of C134, a Chimeric Oncolytic Herpes Simplex Virus, in Mice and Non-human Primates. Mol Ther Oncolytics. 2017; 5:1-10. Published 2017 Mar L doi: 10.1016/j.omto.2017.02.001. PCT Publication WO 202/047398 (incorporated herein by reference) describes Applicant's C134 HSV and provides the sequence and methods of making and using the C134 HSV. The sequence of C134 with the IL-21 polynucleotide is provided in FIG. 1.

The HSV vector of this disclosure also encompasses HSV mutants, for example, 17TermA HSV and rRp450 HSV. The term “17TermA HSV” refers to mutant HSV-1 virus that comprises the entire ICP34.5 gene, but with a termination codon inserted before 100 bp of coding region, resulting in early termination of protein expression and expression of a 30 amino acid truncated protein. The 17TermA HSV mutant displays a growth defect because of the truncated ICP34.5 protein. See Orvedahl et al., (2007) Cell Host & Microbe, 1:1, 23-25. The term “rRp450” refers to an attenuated herpes simplex 1 vector deficient in the viral-encoded ribonucleotide reductase or ICP6. See Aghi M et al., (1999) Cancer Res., 59(16):3861-5.

The HSV genome encodes multiple virulence proteins, which include but are not limited to glycoprotein E (“gE”), Infected Cell Protein 0 (“ICP0”), Infected Cell Protein 6 (“ICP6”), DNA packaging terminase subunit 1, Infected Cell Protein 8 (“ICP8”), and Infected Cell Protein 34.5 (“ICP34.5”). An exemplary HSV1 genome can be found at NCBI Reference Sequence: NC_001806.2, last accessed on Mar. 13, 2020.

The term “gE-encoding gene” refers to a gene or its DNA fragment encoding a gE protein. An exemplary gE-encoding gene can be identified at positions 33-2555 of the HSV-1 genome sequence at NCBI Reference Sequence: NC_001806.2. The term “ICP6 protein” refers to an infected cell protein 6 encoded by the HSV genome. ICP6 is a subunit of ribonucleotide reductase (“RR”) and a key enzyme for nucleotide metabolism and viral DNA synthesis in non-dividing cells.

As used herein, the term “G207” intends a conditionally replication-competent Δγ₁34.5 oncolytic herpes virus that contains UL39 gene mutation.

The term “IL-21” refers to the pleiotropic cytokine whose functions in protection and immunopathology during parasitic diseases. It also is produced by T cells and natural killer (NK cells). The protein and encoding polynucleotide are known in the art, e.g., //www.uniprot.org/uniprot/Q9HBE4. Additional exemplary sequences include: IL-21 sequence:

(SEQ ID NO: )
MRSSPGNMERIVICLMVIFLGTLVHKSSSQGQDRHMIRMRQLIDIVDQL
KNYVNDLVPEFLPAPEDVETNCEWSAFSCFQKAQLKSANTGNNERIINV
SIKKLKRKPPSTNAGRRQKHRLTCPSCDSYEKKPPKEFLERFKSLLQKM
IHQHLSSRTHGSEDS;
(SEQ ID NO: )
MRSSPGNMERIVICLMVIFLGTLVHKSSSQGQDRHMIRMRQLIDIVDQL
KNYVNDLVPEFLPAPEDVETNCEWSAFSCFQKAQLKSANTGNNERIINV
SIKKLKRKPPSTNAGRRQKHRLTCPSCDSYEKKPPKEFLERFKSLLQKM
IHQHLSSRTHGSEDS;
and
((SEQ ID NO: )
MRSSPGNMERIVICLMVIFLGTLVHKSSSQGQDRHMIRMRQLIDIVDQL
KNYVNDLVPEFLPAPEDVETNCEWSAFSCFQKAQLKSANTGNNERIINV
SIKKLKRKPPSTNAGRRQKHRLTCPSCDSYEKKPPKEFLERFKSLLQKV
STLSFI.

The term “deletion or inactivation of a gene” means deletion of the whole or portion of the gene or suppression of expression of the gene through substitution of some bases, modification, insertion of an unnecessary sequence or the like. The deletion or inactivation of the HSV gene (e.g., gE, ICP0, and ICP8) can be conducted by those skilled in the art in a known method or a method based thereon. For example, a method using homologous recombination can be employed. For example, it is possible to divide and inactivate the HSV gene by cloning a DNA fragment containing a portion of the HSV gene and a sequence unrelated to the HSV gene in a suitable plasmid vector and then introducing it into HSV to cause homologous recombination in some region of the HSV gene. Alternatively, the mutation or deletion of an HSV gene can be caused by spontaneous mutation in the viral passage.

In aspects where gene transfer is mediated by a DNA viral vector, such as an herpes simplex virus, a vector construct refers to the polynucleotide comprising, or alternatively consisting essentially of, or yet further consisting of the viral genome or part thereof, and a transgene. Thus, in one aspect, the non-natural HSV further comprises a transgene coding for a therapeutic polynucleotide or protein.

Vectors that contain both a promoter and a cloning site into which a polynucleotide can be operatively linked are well known in the art. Such vectors are capable of transcribing RNA in vitro or in vivo, and are commercially available from sources such as Agilent Technologies (Santa Clara, Calif) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise, or alternatively consist essentially of, or yet further consist of comprise, or alternatively consist essentially of, or yet further consist of a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

As used herein, the term “signal peptide” or “signal polypeptide” intends an amino acid sequence usually present at the N-terminal end of newly synthesized secretory or membrane polypeptides or proteins. It acts to direct the polypeptide to a specific cellular location, e.g. across a cell membrane, into a cell membrane, or into the nucleus. In some embodiments, the signal peptide is removed following localization. Examples of signal peptides are well known in the art. Non-limiting examples are those described in U.S. Pat. Nos. 8,853,381, 5,958,736, and 8,795,965.

In one aspect, the HSV are detectably labeled. As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected, or it may be quantified. A response that is simply detected generally comprises, alternatively consists essentially of, or yet further consists of a response whose existence merely is confirmed, whereas a response that is quantified generally comprises, alternatively consists essentially of, or yet further consists of a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include but are not limited to bioluminescence and chemiluminescence. Detectable luminescence response generally comprises, alternatively consists essentially of, or yet further consists of a change in, or an occurrence of, a luminescence signal. Suitable methods and luminophores for luminescent labeling assay components are known in the art and described for example in Haugland, Richard P. (1996) Handbook of Fluorescent Probes and Research Chemicals (6th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/antibodies, e.g., rhodamine/anti-rhodamine, biotin/avidin and biotin/strepavidin.

The phrase “solid support” refers to non-aqueous surfaces such as “culture plates” “gene chips” or “microarrays.” Such gene chips or microarrays can be used for diagnostic and therapeutic purposes by a number of techniques known to one of skill in the art. In one technique, oligonucleotides are attached and arrayed on a gene chip for determining the DNA sequence by the hybridization approach, such as that outlined in U.S. Pat. Nos. 6,025,136 and 6,018,041. The polynucleotides of this invention can be modified to probes, which in turn can be used for detection of a genetic sequence. Such techniques have been described, for example, in U.S. Pat. Nos. 5,968,740 and 5,858,659. A probe also can be attached or affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

A “composition” is intended to mean a combination of active polypeptide, polynucleotide or antibody and another compound or composition, inert (e.g., a detectable label) or active (e.g., a gene delivery vehicle).

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

The term “tissue” is used herein to refer to tissue of a living or deceased organism or any tissue derived from or designed to mimic a living or deceased organism. The tissue may be healthy, diseased, and/or have genetic mutations. The biological tissue may include any single tissue (e.g., a collection of cells that may be interconnected) or a group of tissues making up an organ or part or region of the body of an organism. The tissue may comprise, or alternatively consist essentially of, or yet further consist of comprise, or alternatively consist essentially of, or yet further consist of a homogeneous cellular material or it may be a composite structure such as that found in regions of the body including the thorax which for instance can include lung tissue, skeletal tissue, and/or muscle tissue. Exemplary tissues include, but are not limited to those derived from liver, lung, thyroid, skin, pancreas, blood vessels, bladder, kidneys, brain, biliary tree, duodenum, abdominal aorta, iliac vein, heart and intestines, including any combination thereof.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, prophylaxis is excluded from treatment.

As used herein the term “effective amount” intends to mean a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of gene therapy, in some embodiments the effective amount is the amount sufficient to result in regaining part or full function of a gene that is deficient in a subject. In one aspect, an effective amount is an amount to provide a multiplicity of infection (MOI) of from 0.001 to 1 infectious viral particles per cell in ranges in between. Non-limiting examples include a multiplicity of infection (MOI) of at least 0.001, or at least 0.01, or at least 0.1 or at least 1, or from 0.01 to 1, or from 0.1 to 1, or from about 0.01 to 0.1, or less than 1, or less than 0.1, or less than 0.01 infectious viral particles per cell. In other embodiments, the effective amount of an HSV viral particle is the amount sufficient to result in cell lysis in a subject. In some embodiments, the effective amount is the amount required to increase galactose metabolism in a subject in need thereof. The skilled artisan will be able to determine appropriate amounts depending on these and other factors.

In some embodiments the effective amount will depend on the size and nature of the application in question. It will also depend on the nature and sensitivity of the target subject and the methods in use. The skilled artisan will be able to determine the effective amount based on these and other considerations. The effective amount may comprise, or alternatively consist essentially of, or yet further consist of comprise, or alternatively consist essentially of, or yet further consist of one or more administrations of a composition depending on the embodiment.

As used herein, the term “administer” or “administration” intends to mean delivery of a substance to a subject such as an animal or human. Administration can be affected in one dose, continuously or intermittently throughout the course of treatment, e.g. intratumorally or intravenously. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and animals, treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and the target cell or tissue. Non-limiting examples of route of administration include direct and systemic, e.g., intravenous, intra-arterial, intramuscular, intracardiac, intrathecal, subventricular, epidural, intracerebral, intratumorally, intracranially, intracerebroventricular, sub-retinal, intravitreal, intraarticular, intraocular, intraperitoneal, intrauterine, intradermal, subcutaneous, transdermal, transmuccosal, and inhalation.

The term administration shall include without limitation, administration by oral, parenteral (e.g., intramuscular, intraperitoneal, intravenous, intracerebroventricular (ICV), intrathecal, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The disclosure is not limited by the route of administration, the formulation or dosing schedule.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A composition as disclosed herein can be a pharmaceutical composition. A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

As used herein, the term “excipient” refers to a natural or synthetic substance formulated alongside the active ingredient of a medication, included for the purpose of long-term stabilization, bulking up solid formulations, or to confer a therapeutic enhancement on the active ingredient in the final dosage form, such as facilitating drug absorption, reducing viscosity, or enhancing solubility.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

A combination as used herein intends that the individual active ingredients of the compositions are separately formulated for use in combination, and can be separately packaged with or without specific dosages. The active ingredients of the combination can be administered concurrently or sequentially.

“Therapeutically effective amount” of an agent refers to an amount of the agent that is an amount sufficient to obtain a pharmacological response; or alternatively, is an amount of the agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

The terms “treating,” “treatment,” and the like, as used herein, mean ameliorating a disease, so as to reduce, ameliorate, or eliminate its cause, its progression, its severity, or one or more of its symptoms, or otherwise beneficially alter the disease in a subject. Reference to “treating,” or “treatment” of a patient is in one aspect, intended to include prophylaxis. Treatment may also be preemptive in nature, i.e., it may include prevention of disease in a subject exposed to or at risk for the disease or disease progression. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease. In one aspect, the term “treatment” excludes prevention or prophylaxis.

The terms “subject” and “patient” are used interchangeably herein to mean all mammals including humans. Examples of subjects include, but are not limited to, humans, monkeys, dogs, cats, horses, cows, goats, sheep, pigs, and rabbits. In one embodiment, the subject or patient is a human.

As used herein, the term “cancer” is meant to include all types of cancerous growths or oncogenic processes, metastatic tissues or malignantly transformed cells, tissues, or organs, irrespective of histopathologic type or stage of invasiveness. Examples of cancerous disorders include, but are not limited to, solid tumors, hematological cancers, soft tissue tumors, and metastatic lesions. Examples of solid tumors include malignancies, e.g., sarcomas, and carcinomas (including adenocarcinomas and squamous cell carcinomas), of the various organ systems, such as those affecting brain, liver, lung, breast, lymphoid, gastrointestinal (e.g., colon), genitourinary tract (e.g., renal, urothelial cells) prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. Squamous cell carcinomas include malignancies, e.g., in the lung, esophagus, skin, head and neck region, oral cavity, anus, and cervix. In one embodiment, the cancer is a melanoma, e.g., an advanced stage melanoma. Metastatic lesions of the aforementioned cancers can also be treated using the methods and compositions of the instant disclosure. Exemplary cancers whose growth can be treated, e.g., reduced, using the antibodies molecules disclosed herein include cancers typically responsive to immunotherapy.

As used herein, an “effective dosage” or “effective amount” of drug, compound, or pharmaceutical composition is an amount sufficient to affect any one or more beneficial or desired results. In specific aspects, an effective amount prevents, alleviates or ameliorates symptoms of disease, e.g., a cancer, and/or prolongs the survival of the subject being treated. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing one or more symptoms of a αvβ8 integrin-mediated disease, disorder or condition, decreasing the dose of other medications required to treat the disease, enhancing the effect of another medication, and/or delaying the progression of the disease of patients. An effective dosage can be administered in one or more administrations. For purposes of this disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

A “synergistic combination” or a combination that acts “synergistically,” is a combination that exhibits increased effects that are not predicted when compared with a merely additive effect of the individual therapies combined.

An “anti-cancer therapy,” as used herein, includes but is not limited to surgical resection, chemotherapy, cryotherapy, radiation therapy, immunotherapy and targeted therapy. Agents that act to reduce cellular proliferation are known in the art and widely used. Chemotherapy drugs that kill cancer cells only when they are dividing are termed cell-cycle specific. These drugs include agents that act in S-phase, including topoisomerase inhibitors and anti-metabolites.

### Modes For Carrying Out the Disclosure

### Vector, Cells and Compositions

This disclosure provides a non-natural herpes simplex virus (“HSV”) vector and one or more polynucleotides encoding a IL-21 polypeptide or a biologically active fragment of the IL-21 polypeptide. In one aspect, the HSV comprises a C134 HSV viral vector. In a further aspect, the IL-21 polypeptide comprises human IL-21 polypeptide or a biologically active fragment thereof. In a further aspect, the polynucleotide encoding IL-21 polypeptide or a biologically active fragment thereof is inserted at an ICP34.5 locus of the HSV or one or both RL1/g134.5 loci within the virus located in both repeat long genetic region of the virus.

In a yet further aspect, the IL-21 polynucleotide is under the control of a modified retroviral promoter region (“MND”). In a yet further aspect, the one or more IL-21 polynucleotide(s) is under the control of a modified retroviral promoter region (“MND”). The polynucleotide can be a DNA or an RNA molecule. In a yet further aspect, non-natural HSV vector of this disclosure has the IL-21 polynucleotide under the control of a promoter selected from modified retroviral promoter region (“MND”), a cytomegalovirus (CMV) IE promoter, optionally located in the UL3/UL4 intergenic region, or an EGR1 promoter.

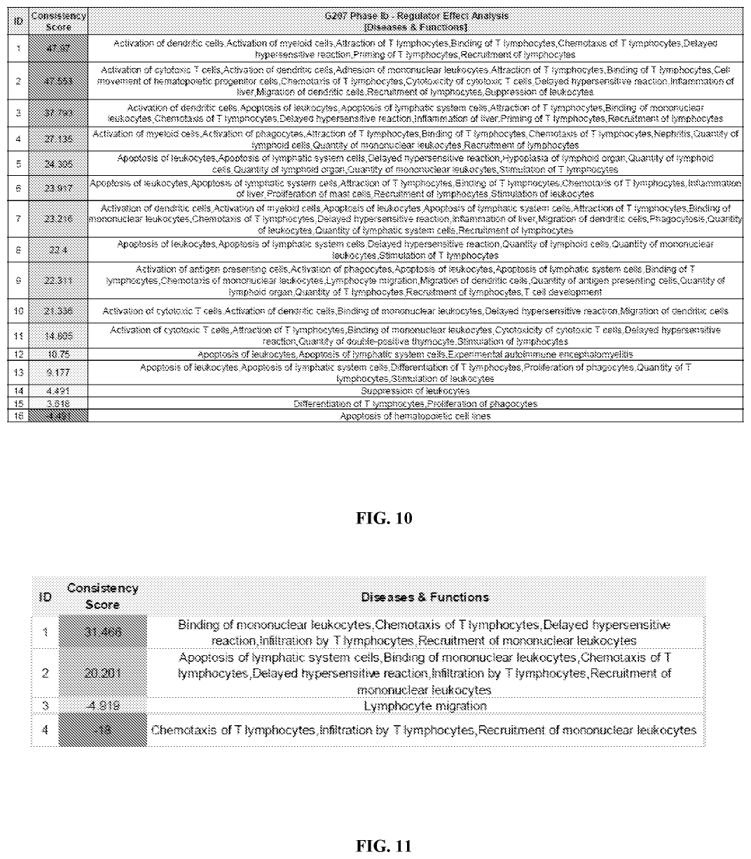

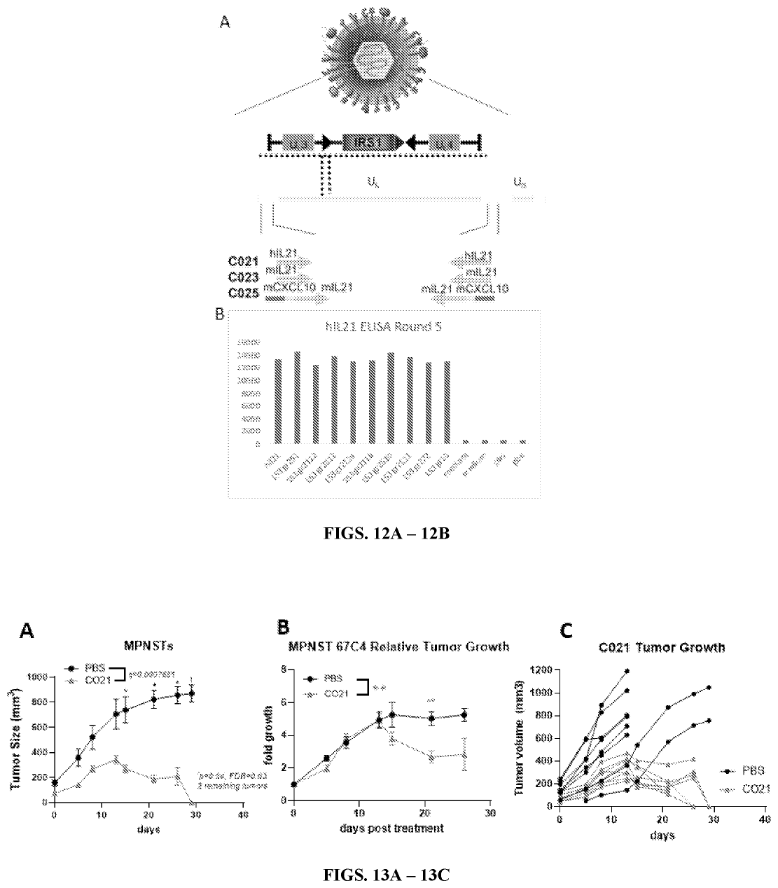

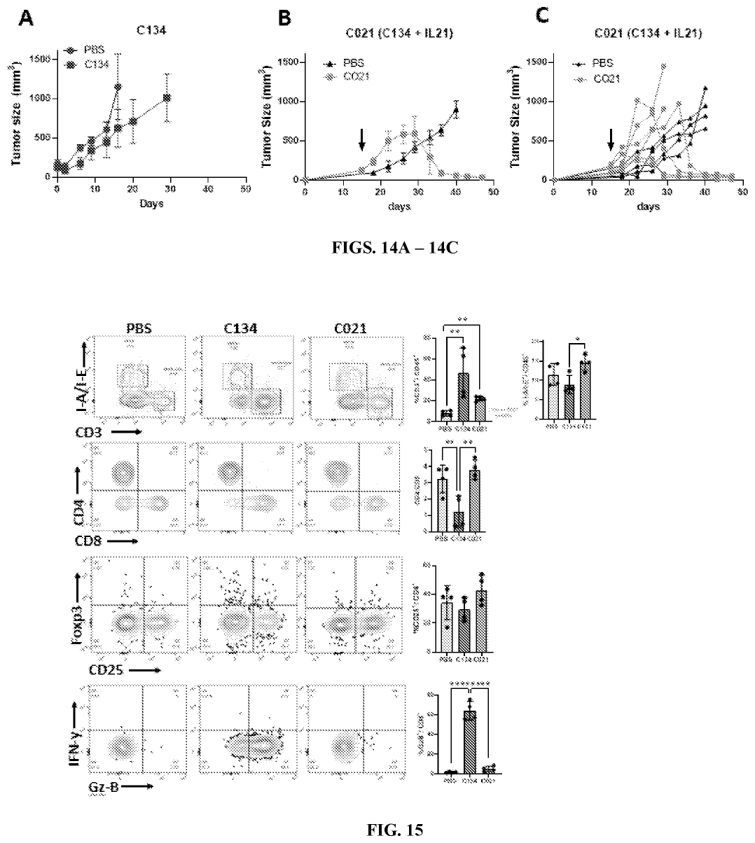

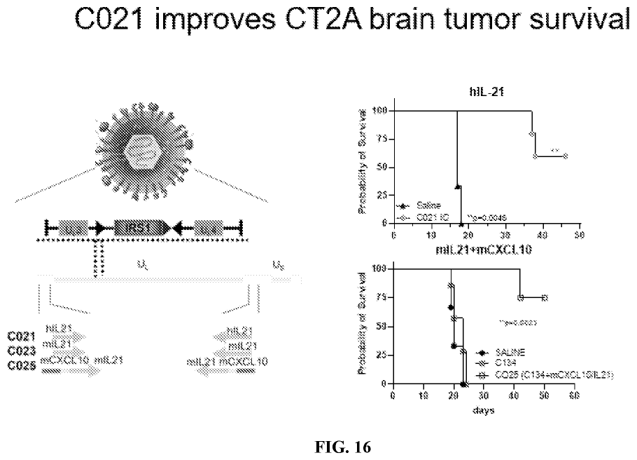

In a further aspect, the HSV vector of this disclosure is constructed as shown in FIG. 12.

The polynucleotide can be a DNA or an RNA molecule.

In one aspect, provided herein is a host cell comprising the non-natural HSV vector as described herein. The host cell can be a eukaryotic cell or a prokaryotic cell.

Yet further provided are compositions comprising or alternatively consisting of, or yet further consisting of the non-natural HSV vector as described herein as the active agent and a carrier. In one aspect, the carrier comprises a pharmaceutically acceptable carrier. The compositions can be formulated or lyophilized for storage or administration. In one aspect, the composition is formulated for intratumoral injection.

The vectors and compositions can be used to deliver IL-21 and to inhibit the growth of cancer cells or treating a cancer in a subject in need thereof by administering to the subject an effective amount of the non-natural HSV vector as described herein or the composition containing same as disclosed herein. Administration can be systemic or local, optionally infusion, injection or intratumoral injection. In a further aspect, the method further comprises subsequent administration to the subject an effective amount of an immunotherapy. Non-limiting examples of such include adoptive cell therapy, immune checkpoint inhibition, immune system modulation, or engineered cellular therapy (e.g., CAR T or CAR NK therapy). The method is useful to treat a solid tumor or a blood cancer, such as for example a sarcoma, carcinoma, or a glioblastoma. It can be administered as a first line, second line, third line, fourth line or fifth line therapy.

Applicant has discovered that the vector constructs are not only transcriptionally active (making abundant mRNA) but these transcripts are translated into actual cytokine in the tumor. This leads to higher levels of cytokine production in the tumor environment. The expression of IL-21 leads to improved immune mediated anti-tumor activity in Applicant's immune competent mouse tumor models and increases Natural Killer and T cell activity in the tumor both in flank and in orthotopic brain tumor models. The vector also increased the IFNgamma response of NK and CAR-NK cells and is anticipated to have similar activity on CAR-T cells. Thus, the vector is useful as a monotherapy or in tandem with adoptive cellular or engineered cellular therapies.

### Therapeutic Methods

Provided is a method for inhibiting the growth or metastasis of a cancer cell or a metastatic cancer cell, the method comprising, or consisting essentially of, or yet further consisting of, contacting the cell with an effective amount of the non-natural HSV vector or a composition or a pharmaceutical composition containing the non-natural HSV vector as described herein. The contacting is in vitro or in vivo. In one aspect, the contacting is in vivo by administration of the non-natural HSV or a composition or a pharmaceutical composition to a subject. In vitro, the method is practiced by placing the non-natural HSV in contact with the cell. The in vitro method can be used to test for new therapies or as a personalized assay to determine if the therapy is suitable for the cancer to be treated. Additional cancer therapies can be combined with the therapy which can be concurrent or sequential to the disclosed methods.

In one aspect, the non-natural herpes simplex virus (“HSV”) vector comprises one or more polynucleotides encoding an IL-21 polypeptide or a biologically active fragment of the IL-21 polypeptide. In one aspect of the method, the HSV comprises a C134 HSV viral vector. In a further aspect, the IL-21 polypeptide of the vector comprises human IL-21 polypeptide or a biologically active fragment thereof. In a further aspect, the polynucleotide encoding IL-21 polypeptide or a biologically active fragment thereof is inserted at an ICP34.5 locus of the HSV or one or both RL1/g134.5 loci within the virus located in both repeat long genetic region of the virus.

In a yet further aspect, the IL-21 polynucleotide used in the method is under the control of a modified retroviral promoter region (“MND”). The polynucleotide can be a DNA or an RNA molecule. In a yet further aspect, non-natural HSV vector of this disclosure has the IL-21 polynucleotide is under the control of a promoter selected from modified retroviral promoter region (“MND”), a cytomegalovirus (CMV) IE promoter, optionally located in the UL3/UL4 intergenic region, or an EGR1 promoter, and further optionally the HSV vector as shown in FIG. 12.

The cancer cell to be treated can be a solid tumor or blood cancer, e.g., carcinoma or sarcoma and non-limiting examples of such include pancreatic cancer, renal cancer, small cell lung cancer, brain cancer, neuroblastoma, glioblastoma, neural cancer, bone cancer, lymphoma, myeloma, colon cancer, uterine cancer, breast cancer, leukemia, liver cancer, prostate cancer, skin cancer, or melanoma. In one aspect, the glioblastoma is mesenchymal GBM or classical/proneural subtype. The cell is of any species, e.g., mammalian and human and when performed in vitro, it can be from a cultured cell line or a primary cell, e.g., from a tissue biopsy. The cell can be an adult or juvenile cell or a cancer stem cell (i.e., cancer cells possessing characteristics associated with normal stem cells, specially the ability to give rise to all cell types found in a particular cancer sample) or a cancer cell without such characteristics associated with normal stem cells.

In another aspect, also provided in this disclosure is a method for treating cancer or a metastatic cancer, or inhibiting the growth or metastasis of a cancer cell in a subject in need thereof, comprising, or alternatively consisting essentially of, or yet further consisting of, administering to the subject an effective amount of the non-natural HSV, the composition or the pharmaceutical composition of this disclosure. In one aspect, the non-natural herpes simplex virus (“HSV”) vector comprises one or more polynucleotides encoding an IL-21 polypeptide or a biologically active fragment of the IL-21 polypeptide. In one aspect of the method, the HSV comprises a C134 HSV viral vector. In a further aspect, the IL-21 polypeptide of the vector comprises human IL-21 polypeptide or a biologically active fragment thereof. In a further aspect, the polynucleotide encoding IL-21 polypeptide or a biologically active fragment thereof is inserted at an ICP34.5 locus of the HSV or one or both RL1/g134.5 loci within the virus located in both repeat long genetic region of the virus.

In a yet further aspect, the IL-21 polynucleotide used in the method is under the control of a modified retroviral promoter region (“MND”). The polynucleotide can be a DNA or an RNA molecule.

The subject to be treated can be of any species, e.g., mammalian and human, e.g., canine, equine, bovine, feline, simian, rat or murine. The administration can be as a first line therapy, a second line therapy, a third line therapy, a fourth line therapy, or a fifth line therapy. Additional cancer therapies can be combined with the therapy which can be concurrent or sequential to the disclosed methods. The cancer to be treated can be a solid tumor or blood cancer, e.g., carcinoma or sarcoma and non-limiting examples of such include pancreatic cancer, renal cancer, small cell lung cancer, brain cancer, neuroblastoma, neural cancer, bone cancer, glioblastoma, lymphoma, myeloma, colon cancer, uterine cancer, breast cancer, leukemia, liver cancer, prostate cancer, skin cancer, or melanoma.

Administration can be systemic or local, optionally infusion, injection or intratumoral injection. In a further aspect, the method further comprises subsequent administration to the subject an effective amount of an immunotherapy. Non-limiting examples of such include adoptive cell therapy, immune checkpoint inhibition, immune system modulation, or engineered cellular therapy (e.g., CAR T or CAR NK therapy). The method is useful to treat a solid tumor or a blood cancer, such as for example a sarcoma, carcinoma, or a glioblastoma. It can be administered as a first line, second line, third line, fourth line or fifth line therapy.

The method of this disclosure can be combined with appropriate diagnostics to monitor disease remission or progression. Several methods for such monitoring are known in the art.

In one aspect, the disclosure provides a method of inducing cell lysis, comprising, or alternatively consisting essentially of, or yet further consisting of, contacting the cell with an effective amount of the non-natural HSV, the composition, and/or the pharmaceutical composition of this disclosure. In one aspect, the non-natural herpes simplex virus (“HSV”) vector comprises one or more polynucleotides encoding an IL-21 polypeptide or a biologically active fragment of the IL-21 polypeptide. In one aspect of the method, the HSV comprises a C134 HSV viral vector. In a further aspect, the IL-21 polypeptide of the vector comprises human IL-21 polypeptide or a biologically active fragment thereof. In a further aspect, the polynucleotide encoding IL-21 polypeptide or a biologically active fragment thereof is inserted at an ICP34.5 locus of the HSV or one or both RL1/g134.5 loci within the virus located in both repeat long genetic region of the virus.

In a yet further aspect, the IL-21 polynucleotide used in the method is under the control of a modified retroviral promoter region (“MND”). In a yet further aspect, the non-natural HSV vector of this disclosure has the IL-21 polynucleotide under the control of a promoter selected from modified retroviral promoter region (“MND”), a cytomegalovirus (CMV) IE promoter, optionally located in the UL3/UL4 intergenic region, or an EGR1 promoter. In a further aspect, the HSV vector is constructed as shown in FIG. 12.

The polynucleotide can be a DNA or an RNA molecule.

The contacting is in vitro or in vivo. In one aspect, the contacting is in vivo by administration of the non-natural HSV or a composition or a pharmaceutical composition to a subject. In vitro, the method is practiced by placing the non-natural HSV in contact with the cell. The in vitro method can be used to test for new therapies or as a personalized assay to determine if the therapy is suitable for the subject to be treated. Additional cell lytic therapies can be combined with the therapy which can be concurrent or sequential to the disclosed methods.

Administration can be systemic or local, optionally infusion, injection or intratumoral injection. In a further aspect, the method further comprises subsequent administration to the subject an effective amount of an immunotherapy. Non-limiting examples of such include adoptive cell therapy, immune checkpoint inhibition, immune system modulation, or engineered cellular therapy (e.g., CAR T or CAR NK therapy). The method is useful to treat a solid tumor or a blood cancer, such as for example a sarcoma, carcinoma, or a glioblastoma. It can be administered as a first line, second line, third line, fourth line or fifth line therapy.

The cell to be treated can be a solid tumor or blood cancer, e.g., carcinoma or sarcoma and non-limiting examples of such include pancreatic cancer, renal cancer, small cell lung cancer, brain cancer, neuroblastoma, glioblastoma, neural cancer, bone cancer, lymphoma, myeloma, colon cancer, uterine cancer, breast cancer, leukemia, liver cancer, prostate cancer, skin cancer, or melanoma. The cell is of any species, e.g., mammalian and human and when performed in vitro, it can be from a cultured cell line or a primary cell, e.g., from a tissue biopsy. The cell can be an adult or juvenile cell or a cancer stem cell or a cancer cell without characteristics associated with normal stem cells. The therapy can be combined with an appropriate assay to test for the effectiveness of the therapy, e.g., cancer remission or progression.

In another aspect, the disclosure also provides a kit comprising, or alternatively consisting essentially of, or yet further consisting of the non-natural HSV, the composition, and/or the pharmaceutical composition of this disclosure.

The following experimental details are provided to illustrate but not limit the inventions as claimed herein.

### Experimental

Glioblastoma multiforme (GBM) are central nervous system (CNS) tumors that are uniformly fatal with median survival of 12-15 months from initial diagnosis and 4-6 months after recurrence, despite all modalities of therapeutic intervention (1, 2). Standard of care, including tumor resection, chemotherapy, and radiation currently offers limited survival benefit. The immunosuppressive microenvironment of GBM tumors contributes to this dismal prognosis (3-5). High grade gliomas, including GBMs, express increased levels of immunosuppressive cytokines and demonstrate immune infiltrates that promote immune evasion and tumor progression (6, 7). Upon recurrence, GBMs commonly transition to a mesenchymal subtype characterized by even more enhanced immune suppression and a subsequent worse prognosis (8, 9). Designed to stimulate the immune system to attack tumor cells, immunotherapies are an evolving strategy for cancer treatment and have demonstrated remarkable responses in many malignancies. Oncolytic virotherapy, in particular, involves genetically engineered viruses designed to selectively replicate in tumor cells, relieving immunosuppression in the tumor microenvironment and enhancing anti-tumor immune responses.

Three phase 1 clinical trials investigated the safety of the oncolytic herpes simplex virus (oHSV), G207, in a cohort of 36 patients with recurrent GBM (10-12). As this was one of the first oHSVs tested in humans, the G207 trials were designed conservatively for safety. G207 lacks both copies of the γ₁34.5 neurovirulence gene and contains a lacZ gene insertion that inactivates the viral ribonucleotide reductase (UL39 gene) and therefore has restricted replication in vitro and in vivo (10, 13). In a traditional 3+3 dose-escalation Phase 1 trial, treatment was reported safe and produced no dose-limiting toxicities (10). Several G207 treated patients in this trial had impressive clinical responses: two subjects were long term survivors (5.5 and 7.5 years in the pre-temozolomide era), while a third subject died from an unrelated cause at 10 months post-G207 injection but had no viable tumor cells detected upon autopsy (10).

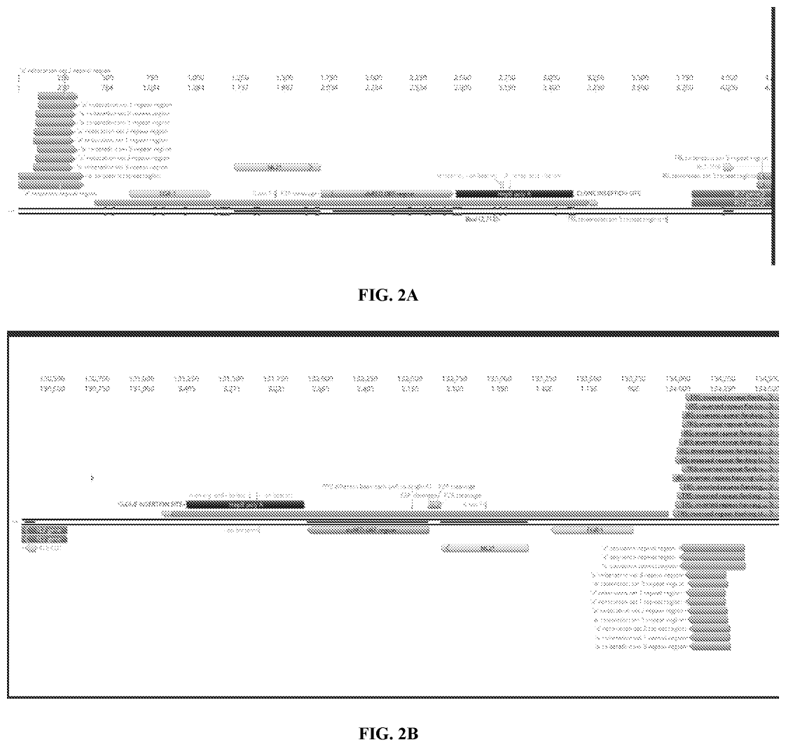

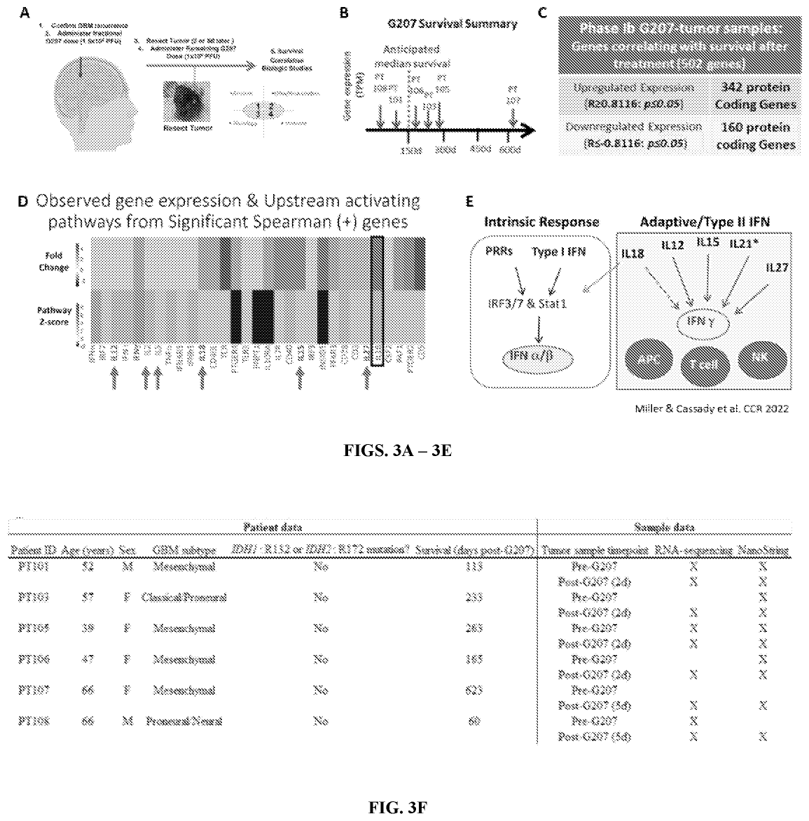

Following the initial safety study, a Phase 1b trial enrolled six adult patients diagnosed with either GBM recurrence or progression. Following surgical resection but prior to oHSV injection, all patients received radiation therapy and all but one patient received chemotherapy. Patients were then treated twice with G207—an initial intratumoral stereotactic injection of the virus, followed two or five days later by an en bloc resection and infusion of the virus again into the resection cavity (FIG. 3A) (11). The Phase 1b trial demonstrated safety in administering multiple oHSV doses into the brain including into the resection cavity. Analyses of resected tumor tissue including immunohistochemical staining and PCR of viral genes revealed marked cytotoxic lymphocyte infiltration and the presence of HSV DNA, respectively, indicating an active anti-tumor response. Overall, the highly attenuated G207 modestly extended median survival (6.6 months). Survival varied between patients with one patient deriving no discernable benefit from virotherapy (60 days survival post-G207) and another living for 21 months after G207 therapy.

### Experiment No. 1

### Materials and Methods

### Clinical Trial Specimens

Fixed tumor specimens were obtained from the Phase 1b G207 clinical trial (NCT00028158), in accordance with IRB approval. GBM subtypes for each tumor were defined based on analysis of microarray data to assign subtypes at the time of initial diagnosis, more than 10 years ago.

### RNA Extraction, Library Preparation, and Sequencing

RNA was extracted using Covaris truXTRAC FFPE total nucleic acid kit. For pre-G207 samples, RNA was subjected to DNase treatment and ribodepletion using Illumina TruSeq Stranded total RNA kit; paired-end 151 bp reads were generated on Illumina HiSeq 4000. For post-G207 samples, RNA libraries were prepared using Illumina TruSeq stranded mRNA kit; paired-end 50 bp reads were generated on Illumina GAIIx.

### RNA-Seq Data Processing, Visualization, and Differential Expression

Normalized expression values were generated (transcript per million; (TPM) values as follows: FASTQ files were used as input for Salmon in mapping-based mode using human reference transcriptome RefSeq GRCh38 (14). Applicant used standard Salmon parameters with bootstrapping set to 100. Gene-level TPMs were generated using tximport (15). When indicated, Applicant filtered genes for gene type “protein_coding” and used the resulting genes for downstream analyses (16). For principal component analysis (PCA), the top 500 most variable genes were identified using rowVars from matrixStats R package and then evaluated by unsupervised two-dimensional PCA using the R stats function prcomp (17). Applicant used pheatmap R package for unsupervised hierarchical clustering. CIBERSORT was used to predict immune cell abundance from RNA-seq data; TPM expression values and the provided LM22 gene expression signature matrix were used as input for absolute mode with parameters set to 1,000 permutations and quantile normalization disabled before processing (18). Differential gene expression analysis was performed using DESeq2 R package with longest term survivor compared to a reference group composed of other responders (PT103 and PT105) who survived longer than the average 150 days expected for a diagnosis of recurrent glioblastoma (19).

### Functional Annotation and Pathway Analysis

Significant genes identified in Applicant's spearman correlation analysis were used for functional annotation analysis using The Database for Annotation, Visualization, and Integrated Discovery (DAVID) version 6.8 and for pathway analysis using Qiagen Ingenuity Pathway Analysis (IPA) software (20). For IPA analysis input, Applicant included p-values from Applicant's spearman analysis as well as fold-changes for each gene; fold-changes were calculated using TPM from the longest survivor (PT107) divided by average TPM from the two shortest term survivors (PT101 and PT108).

### Nanostring Gene Expression Profiling and Cell Type Prediction

Multiplexed gene expression profiling was performed using the PanCancer Immune Profiling Panel (version 1.1) from NanoString Technologies. Tumor-extracted RNA (150 ng) was prepped according to manufacturer's protocol. Data were normalized using housekeeping genes noted within the panel and analyzed using the advanced analysis module of nSolver software. The advanced analysis module uses a previously published method to measure the abundance of various cell populations, using marker genes which are expressed stably and specifically in given cell types (21). These values are referred to as cell type ‘scores’ herein.

### IDH1R132 and IDH2 R172 High-Depth Sequencing

Tumor DNA (1-10 ng) was used as a template for PCR. IDH1 primers (GRCh38): forward (5′-ACCTTGCTTAATGGGTGTAGAT-3′) and reverse (5′-CTGCAAAAATATCCCCCGGC-3′) and IDH R172 primers (GRCh38): forward (5′-CAGAGACAAGAGGATGGCTAGG-3′) and reverse (5′-TTCCGGGAGCCCATCATCTG-3′). PCR was performed using 2×Q5 MM (New England BioLabs) and 200 nM primers with the following conditions: 30″ at 98° C., 30 cycles of 10″ at 98° C., 20″ at 58° C., 20″ at 72° C., and a final extension of 5′ at 72° C. Amplified products were purified using 1.8×SPRIselect and then amplified for an additional 30 cycles followed by end-repair and dA-tailing using NEBNext Ultra II DNA Library Prep kit reagents. The reaction was followed by adapter ligation with unique molecular identifier (UMI)-IDT-indexed adaptors (Integrated DNA Technologies). Adaptor-ligated samples were purified using 1.0×SPRIselect followed by 0.9×SPRIselect and used for library amplification with Q5 MM and Illumina P5/P7 primer mix. A post-PCR 1.2×SPRIselect cleanup was performed, and libraries were pooled and sequenced on iSeq100 to achieve high-depth sequencing coverage >4,500×.

### Statistical Analysis

Applicant calculated Spearman rank-order correlation coefficients as follows: For each gene, Applicant calculated rank of TPM values for each sample and calculated rank for patient survival (measured in days post-treatment, i.e. 1=longest survival and 6=shortest survival). Applicant then calculated Spearman's rank-order correlation for each gene using TPM rank vs. survival duration in all six patients. A two-tailed t-test was performed using calculated t-value for each correlation value [t=√((r²*df)/(1−r²)), where df is degrees of freedom=4] and the TDIST function in Microsoft Excel. Applicant then filtered by p≤0.05 to get a final set of genes (n=502) for analyses. Significance testing between cell types (displayed in FIG. 5B) was performed using unpaired, two-side t-test. Adjusted p-values in Tables 1&2 are Benjamini false discovery rate (FDR) and were output from DAVID. Adjusted p-values in Table 3 are Benjamini FDR and were output from DESeq2.

Table 1 (below). Top gene ontology terms associated with G207-induced gene expression changes. The top 500 most variable genes identified through RNA-seq analysis were uploaded to the Database for Annotation, Visualization, and Integrated Discovery (DAVID). The most significant biological processes associated with the 500 identified genes are displayed in the table. ^{a}FDR=Benjamini false discovery rate; ^{b}Count=number of genes in Applicant's uploaded dataset of 500 that match with number of genes for that biological process; ^{c}describes percentage of genes in Applicant's uploaded dataset that match with number of genes for that biological process.

### Tables

| Term | FDR^{a} | Count^{b} | %^{c} |
|---|---|---|---|
| immune response | 4.80E−09 | 28 | 12.0 |
| neutrophil chemotaxis | 3.80E−07 | 12 | 5.1 |
| chemokine-mediated signaling pathway | 5.80E−07 | 12 | 5.1 |
| monocyte chemotaxis | 1.50E−05 | 9 | 3.8 |
| inflammatory response | 9.00E−05 | 20 | 8.5 |
| antigen processing and presentation | 9.20E−05 | 9 | 3.8 |
| lymphocyte chemotaxis | 2.20E−04 | 7 | 3.0 |
| cell chemotaxis | 2.60E−04 | 9 | 3.8 |
| chemical synaptic transmission | 3.30E−04 | 15 | 6.4 |
| positive regulation of inflammatory response | 5.00E−04 | 9 | 3.8 |
| cellular response to interferon-gamma | 8.70E−04 | 8 | 3.4 |

TABLE 2
Biological processes which correlate with survival in post-G207 samples
are associated with immune response. Genes with significant p-values in
Applicant's Spearman rank-order analysis were uploaded into the DAVID
database. The top 20 results sorted by decreasing FDR p-value are displayed.

| ID^{a} | Term | FDR^{b} | Count^{c} | %^{d} |
|---|---|---|---|---|
| GO:00006955 | immune response | 2.08E−14 | 35 | 14.7 |
| GO:0042110 | T-cell activation | 6.84E−07 | 11 | 4.6 |
| GO:0032496 | response to lipopolysaccharide | 8.04E−07 | 17 | 7.1 |
| GO:0002250 | adaptive immune response | 1.26E−04 | 14 | 5.9 |
| GO:0006954 | inflammatory respouse | 2.28E−04 | 21 | 8.8 |
| GO:0050776 | regulation of immune response | 6.79E−03 | 13 | 5.5 |
| GO:0031295 | T-cell cestimulation | 1.36E−02 | 9 | 3.8 |
| GO:0045060 | negative thymic T-cell selection | 1.46E−02 | 5 | 2.1 |
| GO:0050852 | T-cell receptor signaling pathway | 4.25E−02 | 11 | 4.6 |
| GO:0032715 | negative regulation of interleukin-6 | 4.71E−02 | 6 | 2.3 |
| | production | | | |
| GO:0032088 | negative regulation of NF-kappaB | 6.53E−02 | 8 | 3.4 |
| | transcription factor activity | | | |
| GO:0030816 | positive regulation of cAMP metabolic | 7.15E−02 | 4 | 1.7 |
| | process | | | |
| GO:0007166 | cell surface receptor signaling pathway | 1.14E−01 | 14 | 5.9 |
| GO:0042130 | negative regulation of T-cell proliferation | 1.89E−01 | 6 | 2.5 |
| GO:0010818 | T-cell chemotaxis | 1.96E−01 | 4 | 1.7 |
| GO:0032720 | negative regulation of tumor necrosis factor | 2.16E−01 | 6 | 2.5 |
| | production | | | |
| GO:0019835 | cytolysis | 2.39E−01 | 5 | 2.1 |
| GO:0006935 | chemotaxis | 3.39E−01 | 9 | 3.8 |
| GO:0007169 | transmembrane receptor protein tyrosine | 4.43E−01 | 8 | 3.4 |
| | kinsse signaling pathway | | | |
| GO:0070098 | chemokine-mediated signaling pathway | 5.51E−01 | 7 | 2.9 |

^{a}ID = Gene ontology (GO) database identifier;
^{b}FDR = Benjamini false discovery rate;
^{c}Count = number of genes in Applicant's uploaded dataset of 500 that match with number of genes for that biological process;
^{d}describes percentage of genes in Applicant's uploaded dataset that match with number of genes for that biological process.

TABLE 3
Differentially expressed genes in patient with longest survival
vs. all other responders are associated with antigen presentation
and cytotoxic response. Differential expression analysis was
performed using DESeq2 to compare the patient with longest
survival post-G207 (PT107) vs. all other responders. Genes
with FDR ≤ 0.25 are displayed. Immune function(s) for
each gene, if any, are displayed and were curated manually
from both Entrez gene and UniProtKB/Swiss-Prot summaries.

| | | Log2 | |
|---|---|---|---|
| | | Fold- | |
| Gene | Immune function | change | FDR^{a} |
| CCL13 | Chemotactic factor that attracts | 6.97 | 0.000 |
| | monocytes, lymphocytes, basophils, | | |
| | and eosinophils | | |
| IL2RA | Constitutively expressed in resting | 4.52 | 0.000 |
| | memory T-cells | | |
| FOSB | | 3.36 | 0.000 |
| C1QTNF1 | | 3.46 | 0.000 |
| GFAP | | −3.07 | 0.000 |
| MMP12 | | 5.87 | 0.003 |
| IGFBP6 | | 3.55 | 0.008 |
| CLEC4G | Plays a role in the T-cell immune | 4.17 | 0.030 |
| | response and viral entry into cell | | |
| CHAT | | 6.69 | 0.038 |
| CXCL9 | Chemotactic for activated T-celle; | 3.76 | 0.053 |
| | binds to CXCR3 | | |
| POSTN | | 3.83 | 0.064 |
| CALB1 | | 3.50 | 0.064 |
| COL13A1 | | 3.20 | 0.064 |
| TRH | | 4.59 | 0.098 |
| ADAMTS9 | | 2.16 | 0.098 |
| COL19A1 | | 3.13 | 0.128 |
| SIK1 | | 3.59 | 0.130 |
| HLA- | Antigen presentation/processing | 2.69 | 0.130 |
| DQA1 | | | |
| TIMD4 | Involved in regulating T-cell | 3.40 | 0.204 |
| | proliferation and lymphotoxin signaling | | |
| GZMB | Secreted by NK cells and cytotoxic T- | 2.79 | 0.204 |
| | lymphocytes and proteolytically processed | | |
| | to generate the active protease, which | | |
| | induces target cell apoptosis | | |
| CD8A | Found on most cytotoxic T-lymphocytes; | 2.61 | 0.207 |
| | T-cell coreceptor | | |
| FAM110C | | 2.74 | 0.223 |
| HMGA2 | | 3.37 | 0.227 |
| LDLR | | 1.82 | 0.231 |
| STUM | | −1.88 | 0.231 |
| ITK | Encodes an intracellular tyrosine kinase | 2.71 | 0.236 |
| | expressed in T-cells; thought to play a role | | |
| | in T-cell proliferation and differentiation | | |
| FAM46C | May be involved in induction of cell death | 2.35 | 0.236 |
| HMOX1 | | 2.19 | 0.236 |
| THBS1 | | 1.57 | 0.239 |
| COL5A1 | | 1.62 | 0.242 |

^{a}FDR = Benjamini false discovery rate.

### Results

As shown in FIG. 3, six patients underwent tumor resection two (PT101, PT103, PT105, PT106) or five days (PT107 and PT108) after receiving the first of two inoculations of G207. Most patients (PT101, PT105, PT106, PT107) had a mesenchymal GBM while two patients (PT103, PT108) had a classical/proneural subtype. Tumors were genotyped for IDH1:R132 and IDH2:R172 mutations, but no driver mutations were detected in any samples. To identify whether certain gene expression changes correlated with virotherapeutic response and improved survival, Applicant analyzed tumor-extracted RNA via RNA-seq. Applicant also evaluated the immune-specific expression profiles as a proxy for estimating types and abundance of infiltrating immune cells using a pre-designed commercially available gene expression panel (PanCancer Immune Profiling Panel, NanoString Technologies, Inc., Seattle, WA).

### Gene Expression Differences in Pre- Versus Post-G207-Treated Tumor Specimens

Applicant first compared pre- and post-G207 treatment RNA-seq using unsupervised principal component analysis (PCA). The PCA results clearly segregated pre- and post-treatment groups with a PC1 separation of 58% (FIG. 4A). G207 treatment had a greater impact on gene expression-based sample clustering than did GBM subtype (PC2; 18%, Y axis FIG. 4A). Similarly, unsupervised, hierarchical clustering separated the samples into two distinct clusters, indicating that G207 virotherapy was the primary driver of transcriptome-based clustering. (FIG. 4B).

Next, Applicant sought to determine any biological functions associated with the post-G207 treatment gene expression cluster. Applicant performed Gene Ontology (GO) analysis using the PC1 component genes. As shown in Table 4, Applicant's analysis revealed that genes enriched in G207-treated samples characterized biological processes involving immune response, including immune cell recruitment, antigen processing and presentation, and positive regulation of the inflammatory response. GO classification by ‘molecular function’ revealed significant association with chemokine activity while pathway mapping using GO analysis and the Kyto Encyclopedia of Genes and Genomes (KEGG) database revealed enrichment for cytokine and chemokine signaling pathways (data not shown).

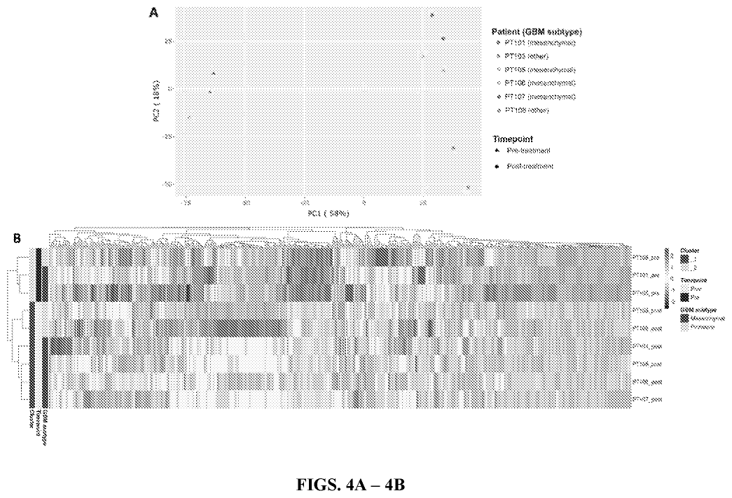

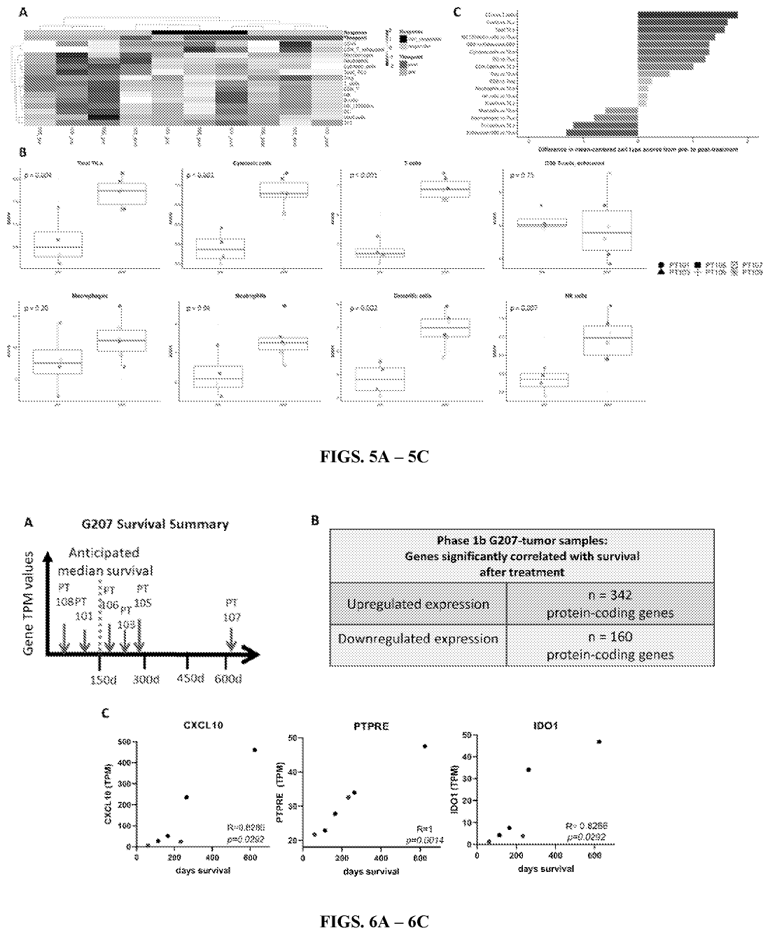

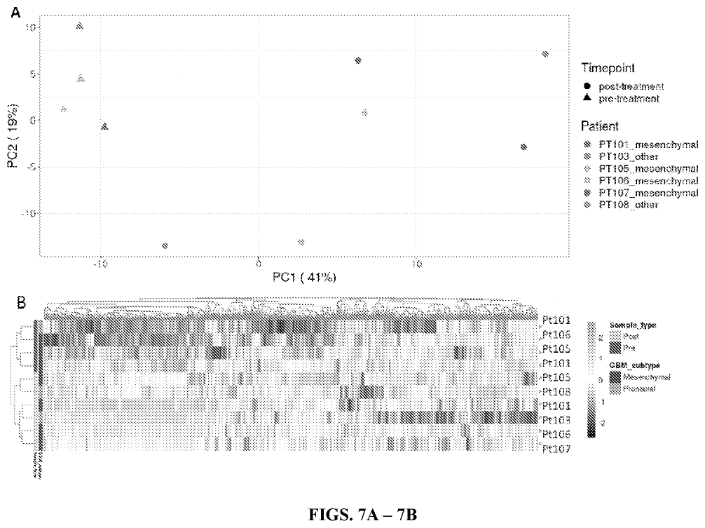

Applicant further characterized RNA extracted from pre- and post-G207-treated samples by multiplex gene expression analysis focusing on a panel of 770 immune response genes representing 24 different immune cell types (PanCancer Immune Panel). Again, PCA analysis with this data revealed that G207 treatment, more than any other component including GBM subtype, had the greatest effect on immune gene expression changes (PC1=41%) (FIG. 7A). Similar to the RNA-seq analysis described above, unsupervised hierarchical clustering using the PanCancer immune gene expression data revealed two distinct clusters of pre- and post-treatment samples (FIG. 7B).

Applicant then examined which immune cell types in particular were enriched in the post-G207 samples. Here, 109 marker genes specific to 24 major immune cell populations were used to assign “cell type scores” (see methods). Nearly all post-G207 samples have higher abundance of immune cells relative to pre-G207 samples (FIG. 5A). Specifically, G207 treatment significantly increased the cytotoxic, T-cell, natural killer (NK and NK-CD56dim), macrophage, neutrophil, and dendritic cell (DC) scores (FIG. 5B). In addition to comparing the individual cell type absolute scores that changed significantly post-G207, Applicant also compared the relative immune cell scores, measured as a proportion of total immune score (FIG. 5C). These results indicated G207 reduced the proportion of infiltrating exhausted T-cells, in that the largest change was the ratio of exhausted T-cells to total tumor-infiltrating lymphocytes (FIG. 5C). The greatest relative changes following G207 treatment involved an increased proportion of T-cells (CD4 and CD8), CD8+ T cell to exhausted CD8+ T-cell ratio, and the NK CD56 dim to total tumor-infiltrating lymphocyte (TIL) ratio. These results also demonstrated that T-cell:TIL and dendritic cell:TIL ratios increased following G207 treatment, and that the exhausted cytotoxic T-cell population relative to TIL score declined. Applicant's analysis also showed relatively minor changes in the CD8 to T-regulatory cells and T-regulatory cells to TIL ratios when comparing pre- and post-G207 treatment data.

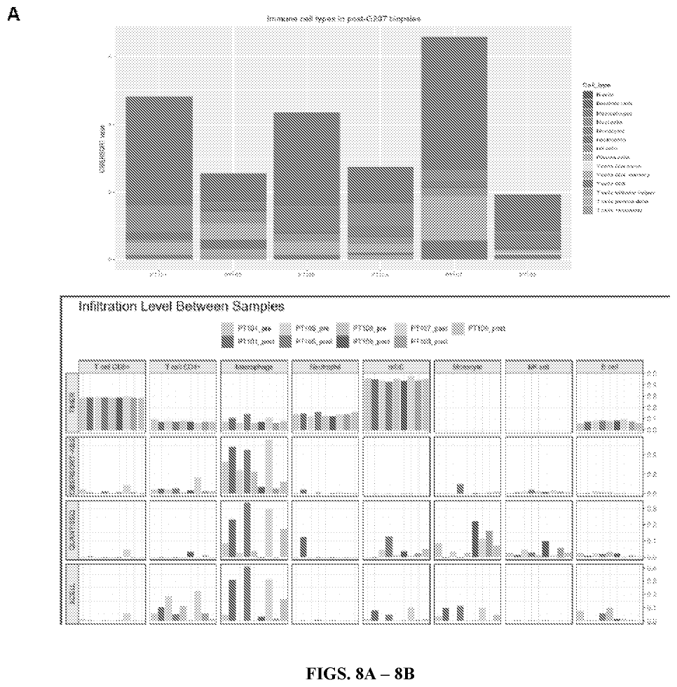

Next, RNA-seq results from the pre- and post-G207 treatment samples were analyzed using CIBERSORT-based deconvolution. CIBERSORT scores indicated that PT107 (best responder, as defined by longest survival following G207 treatment) had the highest proportion of TILs post-G207 treatment. In particular, PT107 had a higher proportion of memory CD4 T-cells, CD8 T-cells, and macrophages compared to all other patients. PT108 (worst responder, as defined by shortest survival following G207 treatment) had the lowest infiltration of TILs present in the post-treatment biopsy (FIG. 8A). Applicant sought to validate deconvolution results orthogonally using two additional algorithms: quanTIseq and xCell via TIMER2.0 web server (22-24). The additional algorithms yielded similar predictions in that PT107 post-G207 had higher CD8 T-cells than any other sample (FIG. 8B).

### Correlation Analysis of Gene Expression and Survival Duration

Applicant sought to test Applicant's hypothesis that immune expression changes enriched in post-G207 treatment RNA were associated with survival following treatment. Median survival of GBM recurrence is four months (150 days), and while four of six G207-treated patients from the Phase 1b study survived longer than this, their survival duration was quite variable (FIG. 3B).

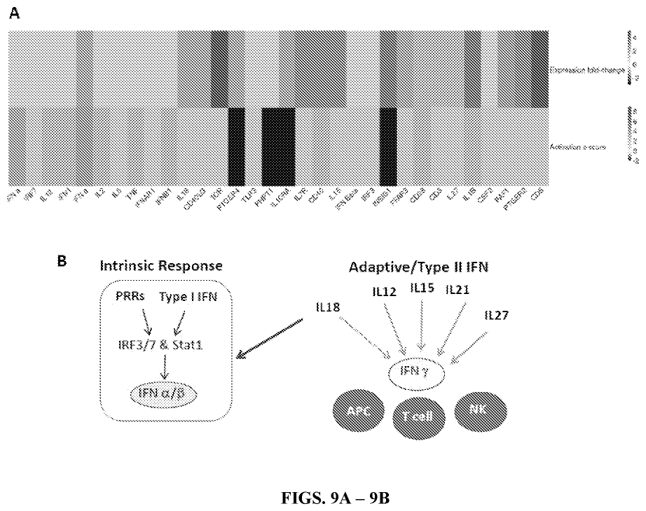

Applicant tested whether there was a direct correlation between the post-G207 treatment gene expression levels (TPM) and survival (days) using a Spearman rank-order correlation analysis. The goal was to identify genes associated with oHSV anti-tumor response in terms of overall survival (FIG. 6A) and to assess whether these genes were enriched for treatment-related biological or immune functions. Applicant's initial spearman correlation analysis identified 502 protein-coding genes that directly correlated with overall survival (FIG. 6B and data not shown). IPA analysis revealed over half of these “survival-related” genes participate in immune response pathways (Table 2 and FIG. 6C). When Applicant identified upstream regulatory pathways in IPA using the same set of 502 genes, both the intrinsic antiviral response (pattern recognition receptor activation of a type I IFN response) and adaptive immune response (T-cell & NK cell stimulatory cytokine production—IL21, IL27, IL12, IFN-γ) correlated with overall survival (FIG. 9). IPA regulatory effect analysis of the spearman significant genes revealed the top regulatory networks regulate immune cell chemotaxis (mononuclear and lymphocytic) and antigen presentation to T-cells (FIG. 10).

As expected, most enrolled patients (four of six) in the Phase 1b clinical trial harbored the more common mesenchymal subtype of GBM based on microarray analysis. The patient with the longest survival duration post-G207 (PT107) had a mesenchymal GBM whereas the patient with the shortest survival post-G207 (PT108) had a proneural GBM. Applicant sought to identify whether differences between GBM subtypes biased Applicant's initial gene expression findings. Therefore, Applicant re-analyzed only the four patients with mesenchymal GBMs using the same spearman correlation approach described above and identified 655 genes (p≤0.05) that directly correlated with survival (data not shown). Of these survival-related genes in the mesenchymal subset, 165 overlapped with Applicant's spearman gene list from Applicant's entire cohort (data not shown). IPA analysis of the overlapping genes (n=165) revealed an association with immune functions including T-cell chemotaxis, recruitment of lymphocytes, and immune cell migration in these tumors (FIG. 11). The remaining survival-related genes (n=490) did not overlap with the larger cohort survival genes and were unique to the mesenchymal subgroup. These genes, upon IPA analysis, were associated with metabolic pathways (e.g., Calcium signaling, Sphingosine metabolism, GDP glucose biosynthesis, Glucose and Glucose-1-phosphate degradation, and Lipoate, Alanine, and Taurine biosynthesis) but not with any immune-related pathways (data not shown). These results suggest overall survival correlated with immune response to oHSV treatment and was not related to an immune response unique to the mesenchymal subgroup that potentially biased Applicant's survival analysis.

Identification of Immune-Specific Genes Enriched in the Patient with Longest Survival Following G207 Treatment

This analysis compared gene expression and pathway analysis differences between PT107 and other responders, defined by a survival time of longer than four months following G207 administration. Applicant sought to identify differentially expressed genes in PT107 that might explain their extraordinary response (623 days survival post-G207). Using DESeq2, a total of 15 significant (|log₂fold-change|>2 and FDR≤0.1) differentially expressed genes were identified between PT107 compared to all other responders (Table 3). Several of the overexpressed genes in PT107 relative to other responders, including CCL13, IL12RA, CXCL9, GZMB, and HLA-DQA1 are associated with recruitment of T-cells and other lymphocytes, antigen presentation, and apoptosis.

TABLE 4

| Term | FDR^{a} | Count^{b} | %^{c} |
|---|---|---|---|
| Immune response | 4.80E−09 | 28 | 12.0 |
| Neutrophil chemotaxis | 3.80E−07 | 12 | 6.1 |
| Chemokine-mediated signalling pathway | 5.80E−07 | 12 | 6.1 |
| Monocyte chemotaxis | 1.50E−05 | 9 | 3.8 |
| Inflammatory response | 9.00E−05 | 20 | 8.5 |
| Antigen processing and presentation | 9.20E−05 | 9 | 3.8 |
| Lymphocyte chemotaxis | 2.20E−04 | 7 | 3.0 |
| Cell chemotaxis | 2.60E−04 | 9 | 3.8 |
| Chemical synaptic transmission | 3.30E−04 | 15 | 8.4 |
| Positive regulation of inflammatory response | 5.00E−04 | 9 | 3.8 |
| Cellular response to IFNγ | 8.70E−04 | 8 | 3.4 |

Note:
The top 500 most variable genes identified through RNA-seq analysis were uploaded to the DAVID. The most significant biological processes associated with the 500 identified genes are displayed in the table
^{a}FDR Benjamini FDR
^{b}Count, number of genes in our uploaded dataset of 500 that match with number of genes for that biological process.
cPercentage of genes in our uploaded dataset that match with number of genes for that biological process.

### Experimental Discussion

The first replication competent oHSV Applicant tested in humans, G207, was conservatively designed for safety and therefore replicated poorly (25). Yet, it had demonstrable anti-glioma effects, including improved survival in some patients. The results of these trials showed that an oHSV: 1) could be safely injected into the brains of patients with GBM, 2) could be safely injected repeatedly, and 3) did not produce dose-limiting toxicities.

Using next-generation RNA-sequencing techniques and other gene expression assays, Applicant's studies identified biological pathways that illustrate differences in immune-mediated responses to virus treatment as a function of the duration of survival across patients. Applicant detected approximately 500 genes that significantly correlated with patient survival and demonstrated that ˜50% of these genes were related to immune response pathways and functions. Network IPA analysis and RNA-seq deconvolution of immune cell populations after treatment with oHSV revealed associations with patient survival and identified important mechanistic events related to cellular infiltrate changes including an increase in the myeloid, cytotoxic, and T-cell populations, suggesting a relationship between immune gene response and survival duration. Previously published data from this clinical trial cohort examined CD4 and CD8 infiltrate primarily but did not examine potential immune function response differences on a more global scale between samples (11). The original studies examining PT107 (best responder) IHC staining of CD3 or CD8 described limited changes to the infiltrate. However, Applicant speculate that for the tumor sections, heterogeneity plus proximity to the injection site could pose bias when performing IHC on a single tumor section. Applicant's current RNA-based analyses sample an entire paraffin embedded section of tumor tissue and therefore offer an integrated analysis from the entire biopsy sample. Applicant's gene expression analyses provide a more nuanced interpretation of immune cell and tumor associated gene expression changes over the entirety of the tumor section and a snapshot of the bulk RNA profile of the tumor resected after oHSV injection.

Applicant aligned the RNA-seq data from post-G207 samples to the oHSV G207 transcriptome and identified G207 transcripts present only in PT107 but no other samples (data not shown). Other patients in this study, however, had survival durations beyond the median survival expected for GBM recurrence and had immune activation identified in gene expression pattern changes, indicating Applicant's analyses show immune response changes independent of viral activity.

Applicant demonstrates herein that immune gene expression was higher in post-G207 samples relative to the pre-G207 samples available (see Figures). To further demonstrate that GBMs prior to any immunotherapy have low immune infiltration at baseline, Applicant analyzed RNA-seq data from TCGA and other datasets available through the Gene Expression Omnibus (35). The fraction of GBM tumors predicted to have significant immune infiltration was lower than any other brain cancer and as expected, was lower than other extracranial solid tumors; furthermore, the fraction of those immune cells predicted to be CD8 T-cells was ˜2% (data not shown). These analyses the reported results that the overexpression of CD8 T-cell transcripts (and therefore inference of infiltration of CD8 T-cells) are relatively low in GBMs at baseline and the increase is likely due to the G207 treatment in Applicant's patients. Other studies have previously demonstrated via RNA-sequencing, immunofluorescent staining, and flow cytometry that recurrent GBMs had low representation of immune infiltration—particularly low in interferon- and T-cell-related pathways, like what Applicant observed in Applicant's pre G207-treated tumors (36, 37). Lastly, in a recent report from NCT02457845, a phase 1 clinical trial to determine the safety of injecting G207 into pediatric recurrent high-grade gliomas, Applicant demonstrated few immune-related cells in the initial core biopsies prior to G207 treatment via immunohistochemical staining for CD8 (38).

Taken together, Applicant's transcriptional analyses demonstrate that treatment with the oHSV G207 in recurrent GBMs significantly shifted tumor-associated gene expression and profoundly altered the intratumoral cellular immune populations and their activities. One patient had an extraordinary clinical response, almost 2-year survival after treatment of recurrent GBM, that corresponded with these post-treatment immune-related sequelae which included higher expression of many T-cell and interferon-related genes. Using RNA-seq and gene expression data, Applicant's analysis suggests that responsive patients exhibited increase in genes involved in antigen presentation as well as improved cytotoxic and T-cell adaptive immune activity following virotherapy, all of which corresponded with improved survival.

### Experiment No. 2

Applicant expanded upon the materials and methods described above, the details of which are provided in the figures and the following experiments.

### Materials and Methods

Clinical trial specimen RNA extraction, RNA-seq data processing, visualization, and differential expression, Functional annotation and pathway analysis, NanoString Gene Expression Profiling and Cell Type Prediction, were performed as described in Experiment No. 1.

### Data Availability

The RNA-sequencing and NanoString gene expression data presented herein has been deposited in NCBI's Gene Expression Omnibus and are accessible through GEO Series accession number GSE164105 (https://www.ncbi.nlm.nih.gov/geo/query/acc.cgi?acc=GSE164105).

### Statistical Analysis

Applicant calculated Spearman rank-order correlation coefficients as follows: For each gene, we calculated rank of TPM values for each sample and calculated rank for patient survival (measured in days post-treatment, i.e. 1=longest survival and 6=shortest survival). Applicant, then calculated Spearman's rank-order correlation for each gene using TPM rank vs. survival duration in all six patients. A two-tailed t-test was performed using calculated t-value for each correlation value [t=√((r2*df)/(1−r2)), where df is degrees of freedom=4] and the TDIST function in Microsoft Excel. Applicant, then filtered by p≤0.05 to get a final set of genes (n=502) for analyses. Significance testing between cell types (displayed in FIG. 3F) was performed using unpaired, two-side t-test. Adjusted p-values in Tables 1 and 4 are Benjamini false discovery rate (FDR) and were output from DAVID. Adjusted p-values in Table 3 are Benjamini FDR and were output from DESeq2.

### Experiment No. 3

### Materials and Methods

### Viruses

C134 has been described previously (Cassady 2005, J Virol., July; 79(14):8707-15., doi: 10.1128/JVI.79.14.8707-8715.2005. PMID: 15994764; PMCID: PMC1168740). Briefly, C134 is a Δγ₁34.5 virus that contains the HCMV IRS1 gene under control of the CMV IE promoter in the UL3/UL4 intergenic region. C154 is an EGFP-expressing version of C134 with EGFP encoded in the 7134.5 locus. C021 was created from C154 and encodes the EGR1 promoter driven hIL21 in both RL1 copies (γ₁34.5 loci) of the virus. C021, therefore, is a Δγ₁34.5, IRS1, and hIL21 recombinant (schematic shown in FIG. 12). Viruses were confirmed genetically by DNA hybridization studies, and hIL21 secretion from media supernatant samples by ELISA (example shown FIG. 12B).

C021 reduces tumor growth in different flank models (CT2A glioma and 67C4 MPNST).

### Cell Lines

CT2A cells were kindly provided by Dr. Thomas Seyfried (Boston College, Chestnut Hill, MA) and were propagated in Dulbecco's modified eagle medium (DMEM) supplemented with 10% fetal bovine serum (FBS). 67C-4 was kindly provided by Dr. Nancy Ratner (University of Cincinnati, Cincinnati, OH) and maintained in DMEM supplemented with 10% FBS. Rabbit Skin Cells (kindly provided by Bernard Roizman University of Chicago, Chicago IL) were grown in DMEM supplemented with 5% FBS and were used for the initial C134+targeting plasmid DNA co-transfection/homologous recombination studies to generate C021. Vero cells (ATCC, Manassas, VA) were used for C021 selection, stock preparations, and limiting plaque dilution titration studies. Tumor lines were tested negative for mycoplasma contamination by PCR and in vivo detect (Inviviogen, San Diego, CA). Tumor cells with relative low passage numbers (<12 passages) were used in the study before returning for a “low” passage form of the cell line to minimize genetic drift in our studies.

### Animal Tumor Studies

Animal studies were approved by the Nationwide Children's Hospital Institutional Animal Care and Use Committee (IACUC; protocol number AR16-00088 and AR16-00069) and performed in accordance with guidelines established by NIH Guide for the Care and Use of Laboratory Animals. Syngeneic C57BL/6 tumor models were used in these studies with tumors implanted stereotactically in an intracerebral orthotopic CT2A glioma tumor model and using a flank based CT2A tumor model and a flank 67C-4 malignant peripheral nerve sheath tumor (MPNST) model.

### Orthotropic Tumors

For the intracerebral studies, 6- to 8-week-old C57BL/6 mice were obtained from (Envigo, Frederick, MD) and were implanted with 1×10⁵ CT2A in 5% methylcellulose using a stereotactic frame, similar to Applicant's earlier studies. (37), 39) Five days later, mice were randomized and treated with vehicle or virus (1×10⁷ PFU/10 μl) using the same stereotactic coordinates. Mice were sacrificed on D6 post-treatment and immunophenotypic analysis performed as described. Other mice underwent CT2A flank tumor implantation also in tumor growth and immunophenotypic analysis similar to our previous studies. (39)

### Flank Tumors

To assess tumor growth characteristics and response to virotherapy, CT2A tumors were independently implanted in 6- to 8-week-old C57BL/6 mice (Envigo, Frederick, MD) by injecting 2×10⁶ CT2A cells in 50 ul of PBS/flank. Once tumors were 25-200 mm3 in size, mice were randomized and treated with vehicle (10% glycerol in PBS) or 3×10⁷ PFU of C134 or C021 in 50 ul of vehicle. For MPNST tumor studies, a similar flank tumor approach was used. Murine 67C-4 MPNST tumor cells (2×106) were injected subcutaneously into the flanks of 6- to 8-week-old C57BL/6 mice (Envigo, Frederick, MD) similar to our earlier studies. (Ghonime, et al., Cancer Immunol. Res. 2018, Dec. 6(12):1499-1510). When tumors reached 25-200 mm³ in size, animals were pooled and randomized into the specified groups, discussed below, with comparable average tumor size. Mice were treated with vehicle, C134 or C021 (3.5×10⁷ PFU in 50 μL 10% glycerol in PBS) intratumorally (ITu) Studies were repeated to ensure biological validity. Tumor measurements (length, width, and depth) were performed at least twice weekly using digital calipers and tumor size calculated. Data are shown from repeated studies and include a composite image (Left) and individual animal tumor responses relative to vehicle treated animals (spaghetti plots, FIG. 3, sarcoma) and FIG. 4 (CT2A). For flank-based studies, animals were monitored for tumor volumes at least twice weekly after treatment until an individual tumor was >1500 mm3 or in some cases total tumor volume/mouse exceeded 2000 mm³. Once tumor size exceeded these criteria, mice were sacrificed based upon IACUC requirements.

### Tissue Preparation and Flow Cytometry

For the CT2A flank and brain tumor-based studies, tumor-bearing mice were sacrificed D6 post-treatment and their flank tumors or brains isolated as described previously. (14) The isolated tumors or brains were placed in RPMI and were homogenized by mechanical dissociation and filtered over 40 μm membrane. The mononuclear cell infiltrate was then isolated from the homogenate by centrifugation over 40% gradient and the isolated mononuclear cell immunophenotypic analysis after fluorescent antibody incubation and multiparameter flow cytometry.

Single-cell suspensions from tumors were lysed with RBC lysis buffer (Sigma, St. Louis, MO) and blocked with 5% mouse Fc blocking reagent (2.4G2, BD Biosciences, San Jose, CA) in FACS buffer (10% FBS and 1 mM EDTA in PBS). Cells were labeled with Fluorescent conjugated antibodies for assessment of NK and T cell response differences between the C021 and vehicle treated cohort: CD19 PE-Cy7, CD4-BV785 (GK1.5), CD25-PE-Dazzle, CD127a PerCP5.5, CD8a-BV510 (53-6.7), CD3F-FITC, CD45-BV605 (30-F11), CD11c-APC, NK1.1-BV421 from Bio-Legend (San Diego, CA). Dead cells were excluded by staining with Live/Dead Near/IR staining (APC-Cy7) (Thermo Fisher Scientific, Charlotte, NC). Single samples were stained with the above staining panels for 30 minutes on ice and washed one time with FACS buffer. After labeling, cells were fixed in 1% paraformaldehyde and analyzed on an Attune NxT Acoustic Focusing Cytometer (Thermo Fisher). Analysis was carried out using the FlowJo software, version 10.5.2 (Tree Star Inc., Ashland, OR) or using Attune NxT Software Version 4.2.1627.1 (Thermo Fisher—Life Technologies, Carlsbad, CA) and data are normalized by showing % of live CD45 population.

### Experiment No. 4

### Materials and Methods

Syngeneic C57BL/6 tumor models were used in these studies with tumors implanted stereotactically in an intracerebral orthotopic CT2A glioma tumor model.

### Orthotropic Tumors

For the intracerebral studies, 6- to 8-week-old C57BL/6 mice were obtained from (Envigo, Frederick, MD) and were implanted with 1×10⁵ CT2A cells in 5% methylcellulose (5 ul) using a stereotactic frame, similar to Applicant's earlier studies. (Shah et al. Gene Therapy, 2007, 14:1045-54 and Ghonime, et al. JITC, 2021) Five days later, mice were randomized and treated with vehicle or virus (1×10⁷ PFU/10 μl) using the same stereotactic coordinates. Mice were followed for focal neurological changes weight loss (<20% IBW) or other signs of neurologic dysfunction (signs of hydrocephalus or obtunded behavior) as objective signs of reaching endpoint (leading to sacrifice) or death.

### Statistical Analysis

Data are summarized by mean+/−standard error. The immune cell population data were analyzed by using one-way analysis of variance (ANOVA) or Kruskal-Wallis test in Prism 8 (GraphPad Software, San Diego, CA) or in SAS. Tumor volumes were first cubic root transformed to ensure normality distribution and then analyzed using mixed effect model by using SAS software (SAS, Inc; Cary, NC). Survival curves were determined by Kaplan-Meier method and log-rank test was conducted to compare survival between groups using SAS. Studies were repeated at least twice to ensure biologic validity. Multiplicity was adjusted by Holm's procedure to control the type I error rate at 0.05. 43 For all analyses, the cutoff for statistical significance was set at P<0.05 and the following notation used: (ns=P>0.05, *P<0.05, **P<0.01, ***P<0.001, ****P<0.0001).

### EQUIVALENTS

The preceding merely illustrates the principles of the disclosure. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles and concepts of the disclosure, further the art, and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the appended claims.

All references cited herein are incorporated into the present disclosure to more fully describe the state of the art.

### REFERENCES

1. Stupp R, Mason W P, van den Bent M J, Weller M, Fisher B, Taphoorn M J, et al. Radiotherapy plus concomitant and adjuvant temozolomide for glioblastoma. N Engl J Med. 2005; 352(10):987-96.
2. Zhao Y H, Wang Z F, Pan Z Y, Peus D, Delgado-Fernandez J, Pallud J, et al. A Meta-Analysis of Survival Outcomes Following Reoperation in Recurrent Glioblastoma: Time to Consider the Timing of Reoperation. Front Neurol. 2019; 10:286.
3. James C D, He J, Carlbom E, Nordenskjold M, Cavenee W K, Collins V P. Chromosome 9 deletion mapping reveals interferon alpha and interferon beta-1 gene deletions in human glial tumors. Cancer Res. 1991; 51(6):1684-8.
4. Kohanbash G, McKaveney K, Sakaki M, Ueda R, Mintz A H, Amankulor N, et al. GM-CSF promotes the immunosuppressive activity of glioma-infiltrating myeloid cells through interleukin-4 receptor-alpha. Cancer Res. 2013; 73(21):6413-23.
5. Miyakoshi J, Dobler K D, Allalunis-Turner J, McKean J D, Petruk K, Allen P B, et al. Absence of IFNA and IFNB genes from human malignant glioma cell lines and lack of correlation with cellular sensitivity to interferons. Cancer Res. 1990; 50(2):278-83.
6. Kennedy B C, Maier L M, D'Amico R, Mandigo C E, Fontana E J, Waziri A, et al. Dynamics of central and peripheral immunomodulation in a murine glioma model. BMC Immunol. 2009; 10:11.
7. Cassady K A, Haworth K B, Jackson J, Markert J M, Cripe T P. To Infection and Beyond: The Multi-Pronged Anti-Cancer Mechanisms of Oncolytic Viruses. Viruses. 2016; 8(2).
8. Behnan J, Finocchiaro G, Hanna G. The landscape of the mesenchymal signature in brain tumours. Brain. 2019; 142(4):847-66.
9. Wang Q, Hu B, Hu X, Kim H, Squatrito M, Scarpace L, et al. Tumor Evolution of Glioma-Intrinsic Gene Expression Subtypes Associates with Immunological Changes in the Microenvironment. Cancer Cell. 2017; 32(1):42-56 e6.
10. Markert J M, Gillespie G Y, Weichselbaum R R, Roizman B, Whitley R J. Genetically engineered HSV in the treatment of glioma: a review. Rev Med Virol. 2000; 10(1):17-30.
11. Markert J M, Liechty P G, Wang W, Gaston S, Braz E, Karrasch M, et al. Phase 1b trial of mutant herpes simplex virus G207 inoculated pre- and post-tumor resection for recurrent GBM. Mol Ther. 2009; 17(1):199-207.
12. Markert J M, Razdan S N, Kuo H C, Cantor A, Knoll A, Karrasch M, et al. A phase 1 trial of oncolytic HSV-1, G207, given in combination with radiation for recurrent GBM demonstrates safety and radiographic responses. Mol Ther. 2014; 22(5):1048-55.
13. Martuza R L, Malick A, Markert J M, Ruffner K L, Coen D M. Experimental therapy of human glioma by means of a genetically engineered virus mutant. Science. 1991; 252(5007):854-6.
14. Patro R, Duggal G, Love M I, Irizarry R A, Kingsford C. Salmon provides fast and bias-aware quantification of transcript expression. Nat Methods. 2017; 14(4):417-9.
15. Soneson C, Love M I, Robinson M D. Differential analyses for RNA-seq: transcript-level estimates improve gene-level inferences. F1000Res. 2015; 4:1521.
16. Zerbino D R, Achuthan P, Akanni W, Amode M R, Barrell D, Bhai J, et al. Ensembl 2018. Nucleic Acids Res. 2018; 46(D1):D754-D61.
17. Wickham H. ggplot2: Elegant Graphics for Data Analysis. Cham: Springer International Publishing: Imprint: Springer; 2016.
18. Newman A M, Liu C L, Green M R, Gentles A J, Feng W, Xu Y, et al. Robust enumeration of cell subsets from tissue expression profiles. Nat Methods. 2015; 12(5):453-7.
19. Love M I, Huber W, Anders S. Moderated estimation of fold change and dispersion for RNA-seq data with DESeq2. Genome Biol. 2014; 15(12):550.
20. Dennis G, Jr., Sherman B T, Hosack D A, Yang J, Gao W, Lane H C, et al. DAVID: Database for Annotation, Visualization, and Integrated Discovery. Genome Biol. 2003; 4(5):P3.
21. Danaher P, Warren S, Dennis L, D'Amico L, White A, Disis M L, et al. Gene expression markers of Tumor Infiltrating Leukocytes. J Immunother Cancer. 2017; 5:18.
22. Li T, Fu J, Zeng Z, Cohen D, Li J, Chen Q, et al. TIMER2.0 for analysis of tumor-infiltrating immune cells. Nucleic Acids Res. 2020; 48(W1):W509-W14.
23. Aran D, Hu Z, Butte A J. xCell: digitally portraying the tissue cellular heterogeneity landscape. Genome Biol. 2017; 18(1):220.
24. Finotello F, Mayer C, Plattner C, Laschober G, Rieder D, Hackl H, et al. Molecular and pharmacological modulators of the tumor immune contexture revealed by deconvolution of RNA-seq data. Genome Med. 2019; 11(1):34.
25. Markert J M, Medlock M D, Rabkin S D, Gillespie G Y, Todo T, Hunter W D, et al. Conditionally replicating herpes simplex virus mutant, G207 for the treatment of malignant glioma: results of a phase I trial. Gene Ther. 2000; 7(10):867-74.
26. Leddon J L, Chen C Y, Currier M A, Wang P Y, Jung F A, Denton N L, et al. Oncolytic HSV virotherapy in murine sarcomas differentially triggers an antitumor T-cell response in the absence of virus permissivity. Molecular therapy oncolytics. 2015; 1:14010.
27. Ghonime M G, Jackson J, Shah A, Roth J, Li M, Saunders U, et al. Chimeric HCMV/HSV-1 and Deltagammal34.5 oncolytic herpes simplex virus elicit immune mediated antigliomal effect and antitumor memory. Transl Oncol. 2018; 11(1):86-93.
28. Saha D, Martuza R L, Rabkin S D. Macrophage Polarization Contributes to Glioblastoma Eradication by Combination Immunovirotherapy and Immune Checkpoint Blockade. Cancer Cell. 2017; 32(2):253-67 e5.
29. Kurozumi K, Hardcastle J, Thakur R, Yang M, Christoforidis G, Fulci G, et al. Effect of tumor microenvironment modulation on the efficacy of oncolytic virus therapy. J Natl Cancer Inst. 2007; 99(23):1768-81.
30. Chen C Y, Wang P Y, Hutzen B, Sprague L, Swain H M, Love J K, et al. Cooperation of Oncolytic Herpes Virotherapy and PD-1 Blockade in Murine Rhabdomyosarcoma Models. Scientific reports. 2017; 7(1):2396.
31. Currier J R, Galley L M, Wenschuh H, Morafo V, Ratto-Kim S, Gray C M, et al. Peptide impurities in commercial synthetic peptides and their implications for vaccine trial assessment. Clin Vaccine Immunol. 2008; 15(2):267-76.
32. Fulci G, Breymann L, Gianni D, Kurozomi K, Rhee S S, Yu J, et al. Cyclophosphamide enhances glioma virotherapy by inhibiting innate immune responses. Proc Natl Acad Sci USA. 2006; 103(34):12873-8.
33. Friedman A, Tian J P, Fulci G, Chiocca E A, Wang J. Glioma virotherapy: effects of innate immune suppression and increased viral replication capacity. Cancer Res. 2006; 66(4):2314-9.
34. Wakimoto H, Johnson P R, Knipe D M, Chiocca E A. Effects of innate immunity on herpes simplex virus and its ability to kill tumor cells. Gene Ther. 2003; 10(11):983-90.
35. Gentles A J, Newman A M, Liu C L, Bratman S V, Feng W, Kim D, et al. The prognostic landscape of genes and infiltrating immune cells across human cancers. Nature medicine. 2015; 21(8):938-45.
36. Schalper K A, Rodriguez-Ruiz M E, Diez-Valle R, Lopez-Janeiro A, Porciuncula A, Idoate M A, et al. Neoadjuvant nivolumab modifies the tumor immune microenvironment in resectable glioblastoma. Nature medicine. 2019; 25(3):470-6.
37. Cloughesy T F, Mochizuki A Y, Orpilla J R, Hugo W, Lee A H, Davidson T B, et al. Neoadjuvant anti-PD-1 immunotherapy promotes a survival benefit with intratumoral and systemic immune responses in recurrent glioblastoma. Nature medicine. 2019; 25(3):477-86.
38. Friedman G K, Johnston J M, Bag A K, Bernstock J D, Li R, Aban I, et al. Oncolytic HSV-1 G207 Immunovirotherapy for Pediatric High-Grade Gliomas. N Engl J Med. 2021; 384(17):1613-22.

1. A non-natural herpes simplex virus (“HSV”) vector and one or more polynucleotides encoding an IL-21 polypeptide or a biologically active fragment of IL-21 polypeptide.

2. The non-natural HSV vector of claim 1, wherein the HSV comprises a C134 HSV virus.

3. The non-natural HSV vector of claim 1, wherein the IL-21 polypeptide comprises human IL-21 polypeptide or a biologically active fragment thereof.

4. The non-natural HSV vector of claim 1, wherein the polynucleotide encoding IL-21 polypeptide or a biologically active fragment thereof is inserted at an ICP34.5 locus of the HSV or in one or both RL1/g134.5 loci within the virus located in both repeat long genetic region of the virus.

5. The non-natural HSV vector of claim 4, wherein the IL-21 polynucleotide is under the control of a promoter selected from modified retroviral promoter region (“MND”), a cytomegalovirus (CMV) IE promoter, optionally located in the UL3/UL4 intergenic region, or an EGR1 promoter, and further optionally the HSV vector as shown in FIG. 12.

6. A host cell comprising the non-natural HSV vector of claim 1.

7. A host cell of claim 6, wherein the host cell is a eukaryotic cell or a prokaryotic cell.

8. A composition comprising the non-natural HSV vector of claim 1, and a carrier.

9. The composition of claim 8, wherein the carrier comprises a pharmaceutically acceptable carrier.

10. The composition of claim 8, wherein the composition is formulated for intratumoral injection.

11. A method of inhibiting the growth of a cancer cell comprising contacting the cell with an effective amount of the non-natural HSV vector of claim 1.

12. The method of claim 11, wherein the contacting is in vitro or in vivo.

13. A method of inhibiting the growth of cancer cells or treating cancer in a subject in need thereof by administering to the subject an effective amount of the non-natural HSV vector of claim 1.

14. The method of claim 13, wherein the administration is selected from systemic or local, optionally wherein the administration is an infusion, an injection or an intratumoral injection.

15. The method of claim 13, further comprising subsequent administration of an effective amount of an immunotherapy to the subject.

16. The method of claim 15, wherein the immunotherapy is selected from adoptive cell therapy, immune checkpoint inhibition, immune system modulation, or engineered cellular therapy.

17. The method of claim 13, wherein the cancer is selected from a solid tumor or a blood cancer.

18. The method of claim 17, wherein the solid tumor is a sarcoma, carcinoma, or glioblastoma.

19. A kit comprising the non-natural HSV vector of claim 1, and instructions for use.

20. The method of claim 11, wherein the cancer cell is a glioblastoma cell.
